(19) United States
(12) Patent Application Publication
(10) Pub. No.: US 2026/0159525 A1
(43) Pub. Date: Jun. 11, 2026

(54) TRICYCLIC HETEROCYCLES

(71) Applicants: Merck Patent GmbH, Darmstadt (DE); Cancer Research Technology Ltd., London (GB)

(72) Inventors: Timo Heinrich, Darmstadt (DE); Sarah Schlesiger, Darmstadt (DE); Jakub Gunera, Darmstadt (DE); Carl Petersson, Darmstadt (DE); Lisa Albers, Darmstadt (DE); Emma Carswell, London (GB); Andrea Unzue Lopez, Darmstadt (DE)

(73) Assignees: Merck Patent GmbH, Darmstadt (DE); Cancer Research Technology Ltd., London (GB)

(21) Appl. No.: 18/705,194

(22) PCT Filed: Oct. 26, 2022
(86) PCT No.: PCT/EP2022/079848

§ 371 Date: Apr. 26, 2024

(30) Foreign Application Priority Data
Oct. 29, 2021 (EP) 21205456.3

(51) Int. Cl. C07D 495/14; A61K 31/4162; A61K 31/4188; A61K 31/4192; A61K 31/4245; A61K 31/429; A61K 31/4439; A61K 31/444; A61K 31/5377; A61P 35/00; C07D 513/14

(52) U.S. Cl. CPC C07D 495/14; A61K 31/4162; A61K 31/4188; A61K 31/4192; A61K 31/4245; A61K 31/429; A61K 31/4439; A61K 31/444; A61K 31/5377; A61P 35/00; C07D 513/14

(57) ABSTRACT

Tricyclic heterocycles are disclosed herein. The heterocyclic compounds include two five-membered heteroaromatic rings. These heterocyclic compounds are useful as TEAD binders and/or inhibitors of YAP-TEAD and TAZ-TEAD protein-protein interaction or binding and for the prevention and/or treatment of several medical conditions including hyperproliferative disorders and diseases, in particular cancer. A method disclosed herein manufactures the compound.

### FIELD OF THE INVENTION

The present invention relates to tricyclic heterocycles. These heterocyclic compounds are useful as TEAD binders and/or inhibitors of YAP-TEAD protein-protein interaction or binding and for the prevention and/or treatment of several medical conditions including hyperproliferative disorders and diseases, in particular cancer.

### BACKGROUND OF THE INVENTION

In recent years the Hippo pathway has become a target of interest for the treatment of hyperproliferative disorders and diseases, in particular cancer (S. A. Smith et al., J. Med. Chem. 2019, 62, 1291-1305; K. C. Lin et al., Annu. Rev. Cancer Biol. 2018, 2: 59-79; C.-L. Kim et al., Cells (2019), 8, 468; K. F. Harvey et al., Nature Reviews Cancer, Vol. 13, 246-257 (2013)). The Hippo pathway regulates cell growth, proliferation, and migration. It is assumed that in mammals the Hippo pathway acts as a tumor suppressor, and dysfunction of Hippo signaling is frequently observed in human cancers.

Furthermore, as the Hippo pathway plays a role in several biological processes—like in self-renewal and differentiation of stem cells and progenitor cells, wound healing and tissue regeneration, interaction with other signaling pathways such as Wnt—its dysfunction may also play a role in human diseases other than cancer (C.-L. Kim et al., Cells (2019), 8, 468; Y. Xiao et al., Genes & Development (2019) 33: 1491-1505; K. F. Harvey et al., Nature Reviews Cancer, Vol. 13, 246-257 (2013)).

While several aspects of the pathway activity and regulation are still subject to further research, it is already established that in its “switched-on”-state the Hippo pathway involves a cascade of kinases (including Mst 1/2 and Lats 1/2) in the cytoplasm which results in the phosphorylation of two transcriptional co-activators, YAP (Yes-associated protein) and TAZ (Transcription co-activator with PDZ binding motif). Phosphorylation of YAP/TAZ leads to their sequestration in the cytoplasm and eventually to their degradation. In contrast, when the Hippo pathway is “switched-off” or dysfunctions, the non-phosphorylated, activated YAP/TAZ co-activators are translocated into the cell nucleus. Their major target transcription factors are the four proteins of the Transcriptional enhanced associate domain (TEAD) transcription factor family (TEAD1-4). Binding of YAP or TAZ to and activation of TEAD (or other transcription factors) have shown to induce the expression of several genes many of which mediate cell survival and proliferation. Thus, activated, non-phosphorylated YAP and TAZ may act as oncogenes, while the activated, switched-on Hippo pathway may act as a tumor suppressor by deactivating, i.e. phosphorylating YAP and TAZ.

Furthermore, the Hippo pathway may also play a role in resistance mechanisms of cancer cells to oncology and immune-oncology therapy (R. Reggiani et al., BBA—Reviews on Cancer 1873 (2020) 188341, 1-11).

Consequently, the dysfunction or aberrant regulation of the Hippo pathway as a tumor suppressor is believed to be an important event in the development of a wide variety of cancer types and diseases.

Therefore, inhibition of YAP, TAZ, TEAD, and YAP-TEAD or TAZ-TEAD protein-protein interaction by pharmacological intervention appears to be a reasonable and valuable strategy to prevent and/or treat cancer and other hyperproliferative disorders and diseases associated with the dysfunction of the Hippo pathway.

### DESCRIPTION OF THE INVENTION

The present invention provides compounds that are useful in the prevention and/or treatment of medical conditions, disorders and/or diseases, in particular of hyperproliferative disorders or diseases, which compounds are TEAD binders and/or inhibitors of YAP-TEAD or TAZ-TEAD protein-protein interaction. Some of the compounds of the present invention may be useful for making other compounds of the present invention.

The present invention refers in one embodiment to a heteroaromatic compound of formula I

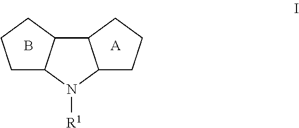

wherein
Ring A represents a five-membered heteroaromatic ring selected from the group consisting of the following ring moieties:

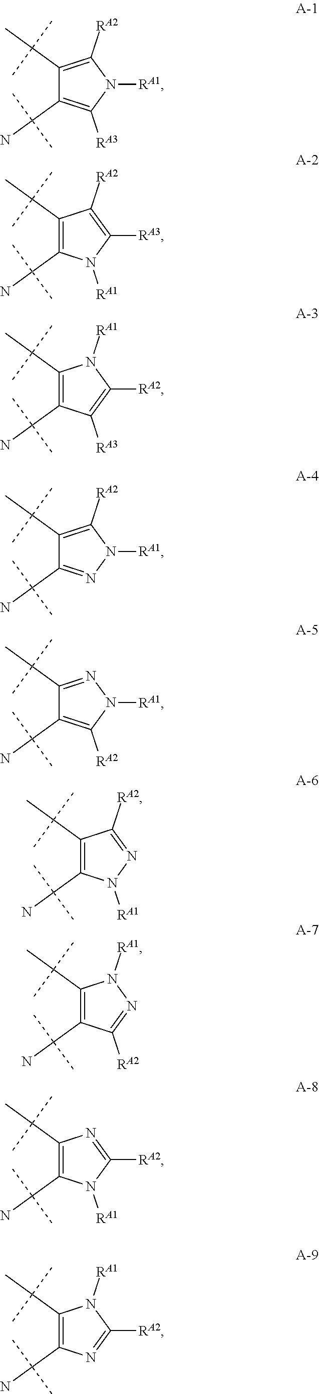

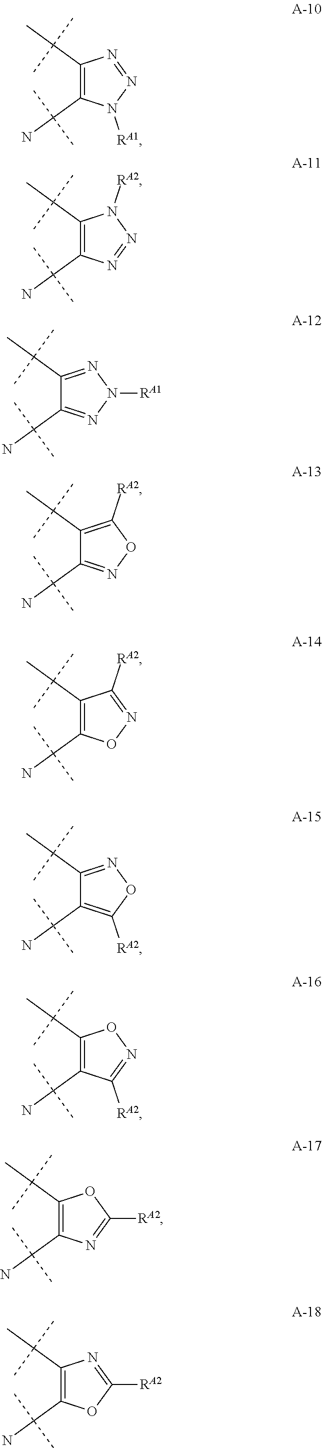

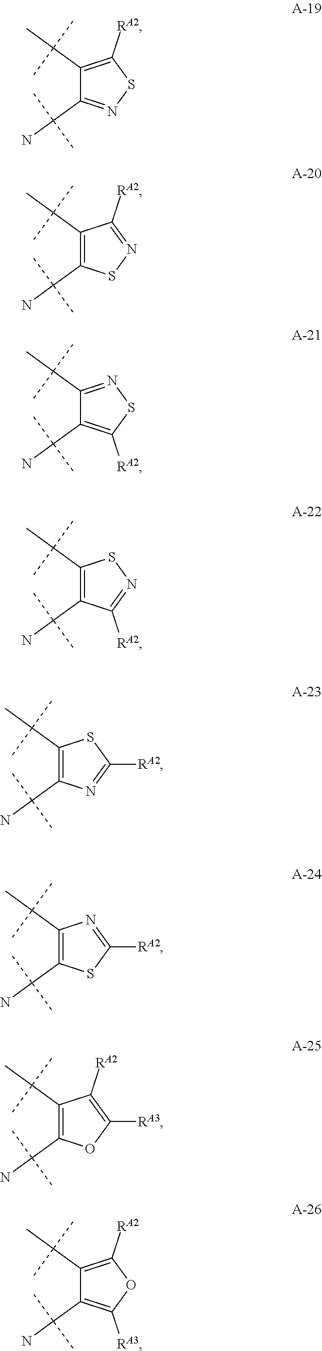

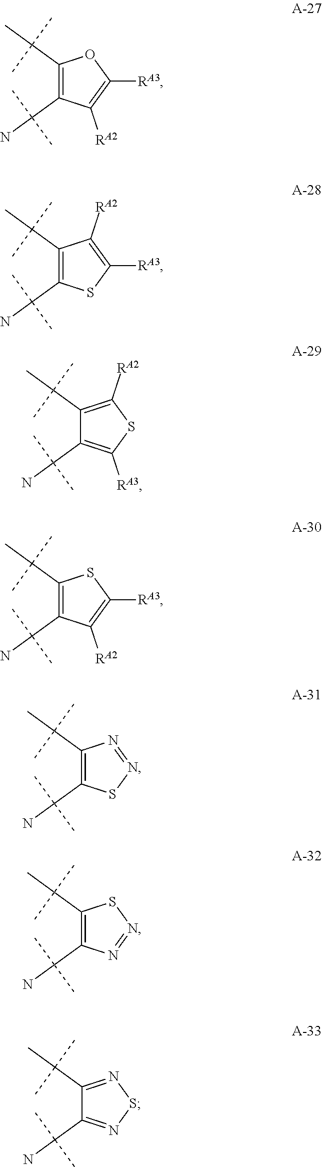

Ring B represents a five-membered heteroaromatic ring selected from the group consisting of the following ring moieties:

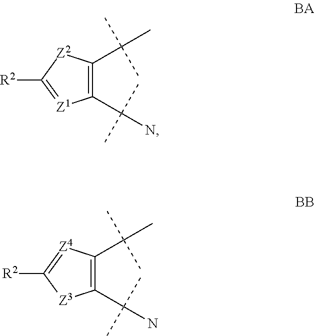

wherein
Z¹ is CR^{Z1} or N;
Z² is O, S, or NR^{Z2};
Z³ is O, S, or NR^{Z3};
Z⁴ is CR^{Z4} or N;
R¹ represents H, Ar¹, Hetar¹, Cyc¹, Hetcyc¹, L¹-Ar¹, L¹-Hetar¹, L²-Cyc¹, L²-Hetcyc¹, or unsubstituted or substituted C₁₋₈-aliphatic;
R² represents —C(═O)—OR^{2a}, —C(═O)—NR^{2b}R^{2c}, —(CH₂)_{w}—C(═O)—NR^{2b}R^{2c}, —(CH₂)ₓ—NR^{2d}—C(═O)—R^{2e}, —S—R^{2f}, —S(═O)—R^{2f}, —S(═O)₂—R^{2g}, —S(═O)₂—NR^{2h}R²ⁱ, —S(═O)₂—OH, —S(═O)(═NR^{2j})—OH, —S(═O)(═NR^{2j})—R^{2g}, —S(═O)(═NR^{2k})—NR^{2l}R^{2m}, F, Cl, Br, I, —CN, —(CH₂)ᵥ—CN, —P(═O)(OR^{2o})(OR^{2p}), —(CH₂)_{y}—NR^{2q}R^{2r}, —(CH₂)_{z}—NR^{2d}—S(═O)₂—R^{2g}, —N═S(═O)—R^{2s}R^{2t}, —C(═O)—N═S(═O)—R^{2s}R^{2t}, —C(═O)—N═S(═N—R^{2u})—R^{2s}R^{2t}, —B(OH)₂ or Hetcyc^{X};
R^{A1} represents Ar³, Hetar³, Cyc³, Hetcyc³, L³-Ar³, L³-Hetar³, L⁴-Cyc³, L⁴-Hetcyc³, unsubstituted or substituted C₁₋₈-aliphatic;
R^{A2}, R^{A3} represent independently from each other H, halogen, Ar³, Hetar³, Cyc³, Hetcyc³, L³-Ar³, L³-Hetar³, L⁴-Cyc³, L⁴-Hetcyc³, unsubstituted or substituted C₁₋₈-aliphatic;
R^{Z1} represents H, C₁₋₆-aliphatic or halogen; or forms together with R² a divalent radical —S(═O)₂—N(H)—C(═O)—
R^{Z2} represents H or C₁₋₆-aliphatic;
R^{Z3} represents H or C₁₋₆-aliphatic;
R^{Z4} represents H, C₁₋₆-aliphatic or halogen;
Ar¹, Ar³ are independently from each other a mono-, bi- or tricyclic aryl with 5, 6, 7, 8, 9, 10, 11, 12, 13, 14 ring carbon atoms, wherein that aryl may be unsubstituted or substituted with substituents R^{B1}, R^{B2}, R^{B3}, R^{B4}, R^{B5}, R^{B6} and/or R^{B7} which may be the same or different;
Ar^{2a}, Ar^{2b}, Ar⁴ are independently from each other a mono- or bicyclic aryl with 5, 6, 7, 8, 9, 10 ring carbon atoms, wherein that aryl may be unsubstituted or substituted with substituents R^{D1}, R^{D2}, R^{D3}, R^{D4} and/or R^{D5} which may be the same or different;
Ar^{X}, Ar^{Z} are independently from each other an un-substituted or substituted benzo ring;
Ar^{Y} is an un-substituted or mono- or di-substituted phenyl;
Hetar¹, Hetar³ are independently from each other a mono-, bi- or tricyclic heteroaryl with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that heteroaryl may be unsubstituted or substituted with substituents R^{B1}, R^{B2}, R^{B3}, R^{B4}, R^{B5}, R^{B6} and/or R^{B7} which may be the same or different;
Hetar^{2a}, Hetar^{2b}, Hetar⁴, Hetar^{Y1} are independently from each other a mono- or bicyclic heteroaryl with 5, 6, 7, 8, 9, 10 ring atoms wherein 1, 2, 3, 4, 5 of said ring atoms is/are a hetero atom(s) selected from N, O and/or S and the remaining are carbon atoms, wherein that heteroaryl may be unsubstituted or substituted with substituents R^{D1}, R^{D2}, R^{D3}, R^{D4} and/or R^{D5} which may be the same or different;
Hetar^{Z} is pyrrole, N-methyl-pyrrole, pyrazole, imidazole, triazole;
Cyc¹, Cyc³ are independently from each other a saturated or partially unsaturated, mono-, bi- or tricyclic carbocycle with 3, 4, 5, 6, 7, 8, 9, 10, 11, 12, 13, 14, 15 ring carbon atoms, wherein that carbocycle may be unsubstituted or substituted with R^{B8}, R^{B9}, R^{B10}, R^{B11}, R^{B12} and/or R^{B13} which may be the same or different; and wherein that carbocycle may optionally be fused to Ar^{X} via 2 adjacent ring atoms of said Ar^{X} and wherein that fused carbocycle may be unsubstituted or substituted with R^{C1}, R^{C2}, R^{C3}, R^{C4}, R^{C5}, R^{C6} which may be the same or different;
Cyc^{2a}, Cyc⁴ are independently from each other a saturated or partially unsaturated monocyclic carbocycle with 3, 4, 5, 6 or 7 ring carbon atoms, wherein that carbocycle may be unsubstituted or substituted with R^{D6}, R^{D7}, R^{D8}, R^{D9} and/or R^{D10} which may be the same or different;
Cyc^{2b} is a saturated or partially unsaturated monocyclic carbocycle with 3, 4, 5, 6 or 7 ring carbon atoms, wherein that carbocycle may be unsubstituted or substituted independently from each other with R^{D6}, R^{D7}, R^{D8}, R^{D9} and/or R^{D10} wherein that carbocycle may optionally be fused to Ar^{Z} or Hetar^{Z} via 2 adjacent ring atoms and wherein that fused carbocycle may optionally further be substituted with independently from each other R^{C1}, R^{C2} and/or R^{C3};
Cyc^{Y1} is a saturated or partially unsaturated monocyclic carbocycle with 3, 4, 5, 6 or 7 ring carbon atoms, wherein that carbocycle may be unsubstituted or substituted with halogen, hydroxy, unsubstituted or substituted C₁₋₆-aliphatic;
Hetcyc¹, Hetcyc³ are independently from each other a saturated or partially unsaturated, mono-, bi- or tricyclic heterocycle with 3, 4, 5, 6, 7, 8, 9, 10, 11, 12, 13, 14, 15 ring atoms wherein 1, 2, 3, 4, 5 of said ring atoms is/are a hetero atom(s) selected from N, O and/or S and the remaining are carbon atoms, wherein that heterocycle may be unsubstituted or substituted with R^{B8}, R^{B9}, R^{B10}, R^{B11}, R^{B12} and/or R^{B13} which may be the same or different;
Hetcyc^{2a}, Hetcyc⁴ are independently from each other a saturated or partially unsaturated, monocyclic heterocycle with 3, 4, 5, 6, 7 ring atoms wherein 1 or 2 of said ring atoms is/are a hetero atom(s) selected from N, O and/or S and the remaining are carbon atoms, wherein that heterocycle may be unsubstituted or substituted with R^{D6}, R^{D7}, R^{D8}, R^{D9} and/or R^{D10} which may be the same or different;
Hetcyc^{2b} is a saturated monocyclic heterocycle with 5 or 6 ring atoms wherein 1 or 2 of said ring atoms is/are a hetero atom(s) selected from N, O and/or S and the remaining are carbon atoms, wherein that heterocycle may be unsubstituted or substituted independently from each other with R^{D6}, R^{D7}, R^{D8}, R^{D9} and/or R^{D10} wherein that heterocycle may optionally be fused to Ar^{Z} or Hetar^{Z} and wherein that fused heterocycle may optionally further be substituted with independently from each other R^{C1}, R^{C2} and/or R^{C3};
Hetcyc^{X} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O and/or S and the remaining are carbon atoms, wherein said heterocycle may be unsubstituted or substituted with R^{X1}, R^{X2}, R^{X3}, R^{X4}, R^{X5}, R^{X6}, R^{X7} and/or R^{X8} which may be the same or different, and wherein that unsubstituted or substituted heterocycle is optionally a carboxylic acid bioisostere;
Hetcyc^{Y} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O and/or S and the remaining are carbon atoms;
Hetcyc^{Y1} is a saturated or partially unsaturated monocyclic heterocycle with 5 or 6 ring atoms wherein 1 or 2 of said ring atoms are heteroatoms selected from N, O, and/or S and the remaining are carbon atoms;
L¹, L³ are independently from each other a divalent radical selected from the group consisting of —S(═O)₂—, —C(═O)—, un-substituted or substituted, straight-chain or branched C₁₋₆-alkylene or C₂₋₆-alkenylene, in both of which one of the carbon units of the alkylene or alkenylene chain may be replaced by —O—;
L², L⁴ are independently from each other a divalent radical selected from the group consisting of —C(═O)—, un-substituted or substituted, straight-chain or branched C₁₋₆-alkylene or C₂₋₆-alkenylene, in both of which one of the carbon units of the alkylene or alkenylene chain may be replaced by —O—;
R^{2a} represents H, un-substituted or substituted C₁₋₈-aliphatic, Ar^{2a}, Hetar^{2a}, Cyc^{2a}, Hetcyc^{2a}, or Cat;
Cat represents a monovalent cation;
R^{2b}, R^{2c} both represent H;
   or one of R^{2b} and R^{2c} represents H or unsubstituted or substituted C₁₋₈-aliphatic, while the other of R^{2b} and R^{2c} represents unsubstituted or substituted C₁₋₁₀-aliphatic, —OH, —O—C₁₋₆-alkyl, —CN, —S(═O)₂—R^{2g}, Ar^{2b}, Hetar^{2b}, Cyc^{2b} or Hetcyc^{2b};
   or R^{2b} and R^{2c} form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms;
R^{2d}, R^{2j}, R^{2k}, R^{2o}, R^{2p} represent independently from each other H, un-substituted or substituted C₁₋₈-aliphatic;
R^{2e} represents H, halogen, un-substituted or substituted C₁₋₈-aliphatic, aryl, heteroaryl; saturated or partially unsaturated heterocyclyl;
R^{2f}, R^{2g} represent independently from each other un-substituted or substituted C₁₋₈-aliphatic;
R^{2h}, R²ⁱ represent independently from each other H, un-substituted or substituted C₁₋₈-aliphatic, Ar^{2b}, Hetar^{2b}, Cyc^{2b} or Hetcyc^{2b}; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms;
R^{2l}, R^{2m}, R^{2a}, R^{2r} represent independently from each other H, un-substituted or substituted C₁₋₈-aliphatic; or R^{2l} together with R^{2m} and/or R^{2q} together with R^{2r} form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms;
R^{2s}, R^{2t} represent independently from each other unsubstituted or substituted C₁₋₈-aliphatic; or form together an unsubstituted or substituted divalent C₃₋₆-alkylene radical;
R^{2u} represents hydrogen or unsubstituted or substituted C₁₋₆-aliphatic;
R^{B1}, R^{B2}, R^{B3}, R^{B4}, R^{B5}, R^{B6}, R^{B7} represent independently from each other un-substituted or substituted, straight-chain or branched C₁₋₆-aliphatic, C₁₋₆-aliphatoxy, —S—C₁₋₆-aliphatic; halogen, —CN, —SF₅, —S(═O)—R^{b1}, S(═O)₂—R^{b1}, —NR^{b2}R^{b3}, Ar⁴, —CH₂—Ar⁴, Hetar⁴, Cyc⁴, Hetcyc⁴; or two adjacent R^{B1}, R^{B2}, R^{B3}, R^{B4}, R^{B5}, R^{B6} and/or R^{B7} form together a divalent —C₂₋₄-alkylene radical in which one of the alkylene carbon units may be replaced by a carbonyl unit (—C(═O)—), or a divalent —O—C₁₋₃-alkylene radical or a divalent —O—C₁₋₃-alkylene-O— radical;
R^{B8}, R^{B9}, R^{B10}, R^{B11}, R^{B12}, R^{B13} represent independently from each other halogen, un-substituted or substituted C₁₋₆-aliphatic, C₁₋₆-aliphatoxy, Ar^{Y}; or
   two of R^{B8}, R^{B9}, R^{B10}, R^{B11}, R^{B12}, R^{B13} which are attached to the same carbon atom of said carbocycle or said heterocycle form a divalent oxo (═O) group; or
   two of R^{B8}, R^{B9}, R^{B10}, R^{B11}, R^{B12}, R^{B13} or four of R^{B8}, R^{B9}, R^{B10}, R^{B11}, R^{B12}, R^{B13} which are attached to the same sulfur atom of said heterocycle form a divalent oxo (═O) group thereby forming either an —S(═O)— or an —S(═O)₂— moiety;
R^{C1}, R^{C2}, R^{C3}, R^{C4}, R^{C5}, R^{C6} represent independently from each other un-substituted or substituted C₁₋₆-aliphatic;
R^{D1}, R^{D2}, R^{D3}, R^{D4}, R^{D5} represent independently from each other halogen, un-substituted or substituted C₁₋₆-aliphatic;
R^{D6}, R^{D7}, R^{D8}, R^{D9}, R^{D10} represent independently from each other halogen, hydroxy, un-substituted or substituted C₁₋₆-aliphatic, unsubstituted or substituted —O—C₁₋₆-aliphatic, Hetar^{Y1}, CH₂-Hetar^{Y1}, Cyc^{Y1}, Hetcyc^{Y1}, —CH₂-Hetcyc^{Y1};
   and/or two of R^{D6}, R^{D7}, R^{D8}, R^{D9}, R^{D10} which are attached to the same ring atom of that carbocycle or heterocycle form a divalent C₂₋₆-alkylene radical wherein optionally one or two non-adjacent carbon units of that alkylene radical may be replaced by independently from each other O, NH, N—C₁₋₄-alkyl, and wherein that alkylene radical may optionally be substituted with OH, C₁₋₆-aliphatic or —O—C₁₋₆-aliphatic;
   and/or two of R^{D6}, R^{D7}, R^{D8}, R^{D9}, R^{D10} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl;
R^{X1}, R^{X2}, R^{X3}, R^{X4}, R^{X5}, R^{X6}, R^{X7}, R^{X8} represent independently from each other un-substituted or substituted C₁₋₆-aliphatic, C₁₋₆-aliphatoxy, halogen, —OH, —NR^{2d}—S(═O)₂—R^{2g}, Hetcyc^{Y}, —O-Hetcyc^{Y}; and/or
   two of R^{X1}, R^{X2}, R^{X3}, R^{X4}, R^{X5}, R^{X6}, R^{X7}, R^{X8} which are attached to the same carbon atom of said heterocycle form a divalent oxo (═O) group; and/or two of R^{X1}, R^{X2}, R^{X3}, R^{X4}, R^{X5}, R^{X6}, R^{X7}, R^{X8} or four of R^{X1}, R^{X2}, R^{X3}, R^{X4}, R^{X5}, R^{X6}, R^{X7}, R^{X8} which are attached to the same sulfur atom of said heterocycle form a divalent oxo (═O) group thereby forming either an —S(═O)— or an —S(═O)₂— moiety;
R^{b1} represents un-substituted or substituted C₁₋₈-aliphatic;
R^{b2}, R^{b3} represent independently from each other H, un-substituted or substituted C₁₋₈-aliphatic; or
   form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms;
halogen is F, Cl, Br, I;
v is 1 or 2;
w is 1 or 2;
x is 0, 1 or 2;
y is 0, 1 or 2;
z is 0, 1 or 2;
or any N-oxide, solvate, tautomer or stereoisomer thereof and/or any pharmaceutically acceptable salt of each of the foregoing, including mixtures thereof in all ratios.

In general, all residues, radicals, substituents, groups, moieties, etc. which occur more than once may be identical or different, i.e. are independent of one another. Above and below, the residues and parameters have the meanings indicated for formula I, unless expressly indicated otherwise. Accordingly, the invention relates, in particular, to the compounds of formula I in which at least one of the said residues, radicals, substituents has one of the preferred meanings indicated below.

Any of those particular or even preferred embodiments of the present invention as specified below and in the claims do not only refer to the specified compounds of formula I but to N-oxides, solvates, tautomers or stereoisomers thereof as well as the pharmaceutically acceptable salts of each of the foregoing, including mixtures thereof in all ratios, too, unless indicated otherwise.

In a particular embodiment, PE1, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein in Ring B
Z¹ is CH or N; and
Z² is S;
or
Z³ is S; and
Z⁴ CH or N;
and the remaining radicals and residues are as defined for formula I above or for any of the further particular embodiments described herein above or below.

In other words, in PE1 Ring B is either derived from a thiophene or from a thiazole ring.

In another particular embodiment, PE1a, of PE1, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein Ring B is derived from a thiophene ring, i.e.

Ring B is

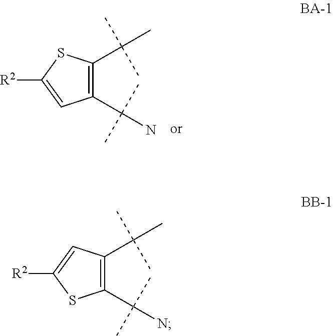

and the remaining radicals and residues are as defined for formula I above or for any of the further particular embodiments described herein above or below.

In a particular embodiment PE1aa of PE1a, Ring B is ring BA-1. In an alternative particular embodiment, PE1ab, of PE1a Ring B is ring BB-1.

In a further particular embodiment of the present invention, PE2, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
Ring A represents a five-membered heteroaromatic ring selected from the group consisting of the following ring moieties:

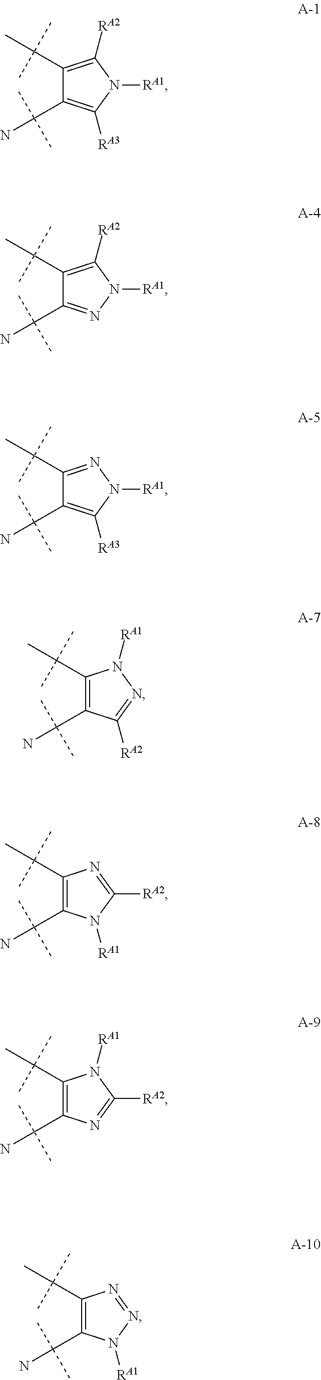

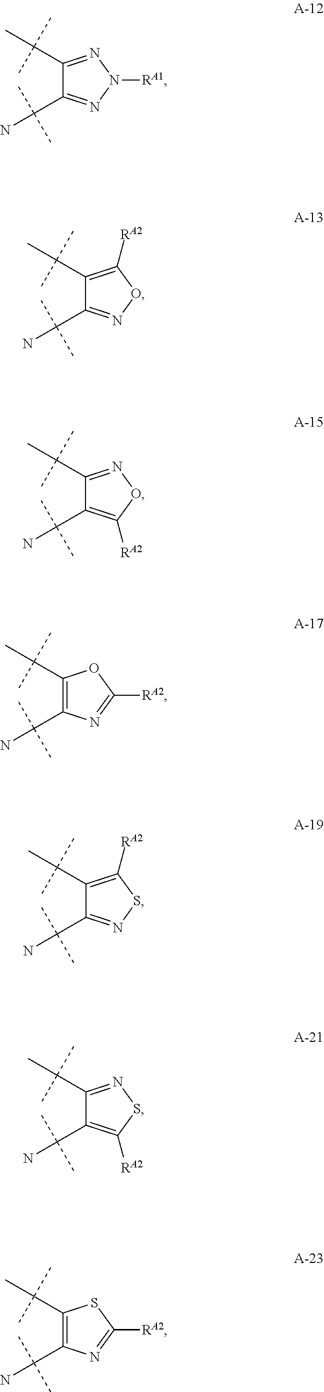

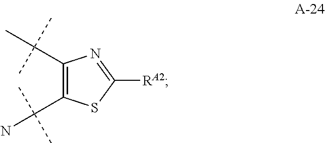

and the remaining radicals and residues are as defined for formula I above or for any of the further particular embodiments described herein above or below.

In a particular embodiment, PE2a, of PE2
R^{A1} represents Ar³, L³-Ar³, straight-chain or branched C₁₋₄-alkyl which is optionally substituted with independently from each other 1, 2 or 3 halogen, straight-chain or branched C₂₋₄-alkenyl, or C₂₋₄-alkinyl;
R^{A2} represents H;
R^{A3} represents H;
Ar³ represents phenyl which is optionally substituted with independently from each other R^{B1}, R^{B2} and/or R^{B3}
L³ represents —CH₂—;
R^{B1}, R^{B2}, R^{B3} are independently from each other halogen, in particular F, or —CN;
and the remaining radicals and residues are as defined for formula I above or for any of the further particular embodiments described herein above or below.

In still another particular embodiment, PE2b, of PE2, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
Ring A represents a five-membered heteroaromatic ring selected from the group consisting of the following ring moieties:

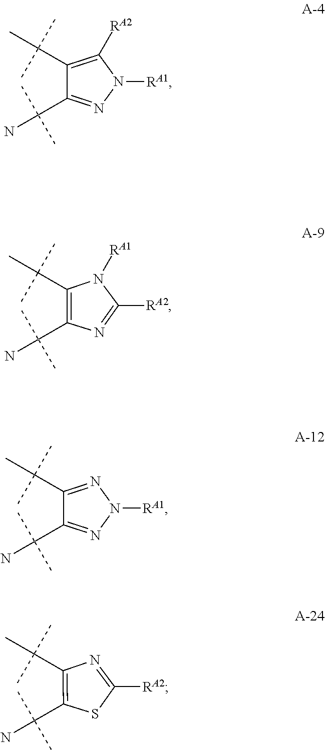

R^{A1} represents Ar³, L³-Ar³, straight-chain or branched C₁₋₄-alkyl which is optionally substituted with independently from each other 1, 2 or 3 halogen, straight-chain or branched C₂₋₄-alkenyl, or C₂₋₄-alkinyl;
R^{A2} represents H;
Ar³ represents phenyl which is optionally substituted with independently from each other R^{B1}, R^{B2} and/or R^{B3}
L³ represents —CH₂—;
R^{B1}, R^{B2}, R^{B3} are independently from each other halogen, in particular F, or —CN;
and the remaining radicals and residues are as defined for formula I above or for any of the further particular embodiments described herein above or below.

In a particular embodiment, PE2aa, of PE2a or in another particular embodiment, PE2ba, of PE2b the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R^{A1} represents —CH₂-phenyl (benzyl); 2-fluorobenzyl, 3-fluorobenzyl, 4-fluorobenzyl; methyl, ethyl, n-propyl, prop-2-yn-1-yl;
and the remaining radicals and residues are as defined for formula I above or for any of the further particular embodiments described herein above or below.

In a further particular embodiment, PE2baa, of PE2ba,
R^{A1} represents methyl;
and the remaining radicals and residues are as defined for formula I above or for any of the further particular embodiments described herein above or below.

In another particular embodiment of the present invention, PE3, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ represents Ar¹, Hetar¹, Cyc¹, Hetcyc¹, L¹-Ar¹, L¹-Hetar¹, L²-Cyc¹, L²-Hetcyc¹, C₁₋₆-alkyl, C₂₋₆-alkenyl or C₂₋₆-alkynyl, wherein said C₁₋₆-alkyl, C₂₋₆-alkenyl or C₂₋₆-alkynyl is straight-chain or branched and unsubstituted or substituted with 1, 2 or 3 halogen;
Ar¹ is a mono- or bicyclic aryl with 6 or 10 ring carbon atoms, wherein that aryl may be unsubstituted or substituted with substituents R^{B1}, R^{B2} and/or R^{B3} which may be the same or different; preferably phenyl or naphthalenyl, in particular phenyl, which may be unsubstituted or substituted with substituents R^{B1} and or R^{B2} which may be the same or different;
Ar⁴ is phenyl;
Ar^{X} is an unsubstituted benzo ring;
Ar^{Y} is phenyl;
Hetar¹ is a monocyclic heteroaryl with 5 or 6 ring atoms or a bicyclic heteroaryl with 9 or 10 ring atoms wherein 1, 2 or 3 of said ring atoms is/are a hetero atom(s) selected from N, O and/or S and the remaining are carbon atoms, wherein that heteroaryl may be unsubstituted or substituted with substituents R^{B1}, R^{B2} and/or R^{B3} which may be the same or different; preferably the heteroaryl is unsubstituted or substituted with substituents R^{B1} and/or R^{B2} which may be the same or different;
Hetar⁴ is a monocyclic heteroaryl with 5 or 6 ring atoms wherein 1, 2, 3, 4, 5 of said ring atoms is/are a hetero atom(s) selected from N, O and/or S and the remaining are carbon atoms; preferably a monocyclic heteroaryl with 5 ring atoms wherein 1 of said ring atoms is N and the remaining are carbon atoms or 1 of said ring atoms is N and 1 of said ring atoms is S and the remaining are carbon atoms;
Cyc¹ is a saturated or partially unsaturated, mono- or bicyclic carbocycle with 3, 4, 5, 6, 7 or 8 ring carbon atoms, wherein that carbocycle may be unsubstituted or substituted with R^{B8} and/or R^{B9} which may be the same or different; and wherein that carbocycle may optionally be fused to Ar^{X} via 2 adjacent ring atoms of said Ar^{X} and wherein that fused carbocycle may be unsubstituted or substituted with R^{C1} and/or R^{C2} which may be the same or different;
Cyc⁴ is cyclopropyl, cyclobutyl, cyclopentyl, each of which may be unsubstituted or mono-substituted with R^{D6} or di-substituted with independently from each other R^{D6} and R^{D7};
Hetcyc¹ is a saturated or partially unsaturated, monocyclic heterocycle with 5 or 6 ring atoms wherein 1 or 2 of said ring atoms is/are a hetero atom(s) selected from N, O and/or S and the remaining are carbon atoms, wherein that heterocycle may be unsubstituted or substituted with R^{B8} and/or R^{B9} which may be the same or different, wherein, if one of the heteroatoms is S, then that heterocycle may also be substituted with R^{B8}, R^{B9}, R^{B10} and R^{B11}; preferably a saturated monocyclic heterocycle with 5 or 6 ring atoms wherein 1 of said ring atoms is a hetero atom selected from O and S and the remaining are carbon atoms, wherein that heterocycle may be unsubstituted or substituted with R^{B8} and/or R^{B9} which may be the same or different, wherein, if one of the heteroatoms is S, then that heterocycle may also be substituted with R^{B8}, R^{B9}, R^{B10} and R^{B11};
Hetcyc⁴ is pyrrolidinyl, piperidinyl, each of which may unsubstituted or mono-substituted with R^{D6} or di-substituted with independently from each other R^{D6} and R^{D7};
L¹ is a divalent radical selected from the group consisting of —S(═O)₂—, un-substituted or substituted, straight-chain or branched C₁₋₆-alkylene or C₂₋₆-alkenylene, in both of which one of the carbon units of the alkylene or alkenylene chain may be replaced by —O—; preferably selected from the group consisting of —S(═O)₂—, —CH₂—, —CH₂—CH₂—, —CH₂—CH₂—C(CH₃)H—, —CH₂—CH₂—C(CH₃)₂—, —CH₂—CH₂—O—CH₂—, —CH₂—CH═CH—;
L² is a divalent radical selected from the group consisting of un-substituted or substituted, straight-chain or branched C₁₋₆-alkylene or C₂₋₆-alkenylene, in both of which one of the carbon units of the alkylene or alkenylene chain may be replaced by —O—; preferably selected from the group consisting of —CH₂—, —CH₂—CH₂—;
R^{B1}, R^{B2}, R^{B3} represent independently from each other straight-chain or branched C₁₋₆-alkyl, which C₁₋₆-alkyl may be unsubstituted or monosubstituted with —CN or substituted with 1, 2 or 3 halogen, straight-chain or branched C₁₋₄-alkoxy, which C₁₋₄-alkoxy may be unsubstituted or substituted with 1, 2 or 3 halogen, —O—CH₂—C≡CH, straight-chain or branched —S—C₁₋₄-alkyl, which —S—C₁₋₄-alkyl may be unsubstituted or substituted with 1, 2 or 3 halogen, F, Cl, Br, —CN, —S(═O)—C₁₋₃-alkyl, S(═O)₂—C₁₋₃-alkyl, —N(C₁₋₃-alkyl)₂, Ar⁴, —CH₂—Ar⁴, Hetar⁴, Cyc⁴, Hetcyc⁴; or two adjacent R^{B1}, R^{B2} and/or R^{B3} form together a divalent —C₃₋₄-alkylene radical in which one of the alkylene carbon units may be replaced by a carbonyl unit (—C(═O)—), or a divalent —O—C₂₋₃-alkylene radical;
R^{B8}, R^{B9} represent independently from each other F, C₁₋₂-alkyl, which C₁₋₂-alkyl may be unsubstituted or substituted with 1, 2 or 3 F, C₁₋₂-alkoxy, Ar^{Y}; or
R^{B8} and R^{B9} are attached to the same carbon atom of said carbocycle Cyc¹ or said heterocycle Hetcyc¹ and form a divalent oxo (═O) group; or
R^{B8} and R^{B9} and R^{B10} and R^{B11} are attached to the same sulfur atom of said heterocycle and form two divalent oxo (═O) groups thereby forming an —S(═O)₂— moiety;
R^{C1} and R^{C2} represent independently from each other C₁₋₆-alkyl which may be independently from each other be substituted with 1, 2, or 3 F atoms;
R^{D6}, R^{D7}, represent independently from each other C₁₋₆-alkyl which may be substituted with 1, 2, or 3 F atoms or 1 hydroxy group; or hydroxy;
halogen is F, Cl, Br;
and the remaining radicals and residues are as defined for formula I above or for any of the further particular embodiments described herein above or below.

In a particular embodiment. PE3a, of PE3, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ represents Ar¹, Hetar¹, Cyc¹, L¹-Ar¹, L²-Cyc¹, un-substituted or substituted, straight-chain or branched C₁₋₆-alkyl, wherein said C₁₋₆-alkyl is straight-chain or branched and unsubstituted or substituted with 1, 2 or 3 halogen;
Ar¹ is phenyl monosubstituted with R^{B1};
Hetar¹ is pyridyl, in particular, pyrid-2-yl, monosubstituted with R^{B1};
Cyc¹ is a saturated monocyclic carbocycle with 3, 4, 5 or 6 ring carbon atoms, wherein that carbocycle is monosubstituted with R^{B8}; in particular Cyc¹ is a cyclobutane ring;
L¹, L² are independently from each other —CH₂—;
R^{B1}, R^{B8} represent independently from each other C₁₋₂-alkyl substituted with 1, 2 or 3 F atoms, in particular with 3 F atoms; C₁₋₂-alkoxy substituted with 1, 2 or 3 F atoms, in particular with 3 F atoms; and the remaining radicals and residues are as defined for formula I above or for any of the further particular embodiments described herein above or below.

In still another particular embodiment, PE3b, of PE3, which is also a particular embodiment of PE3a, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ represents trifluoromethylphenyl, in particular 4-trifluoromethylphenyl; difluoromethylphenyl, in particular 4-difluoromethylphenyl; difluoromethoxyphenyl, in particular 4-difluoromethoxyphenyl; trifluoro-methoxyphenyl, in particular 4-trifluoromethoxyphenyl; trifluoro-methylsulfanylphenyl, in particular 4-trifluoromethylsulfanylphenyl; trifluoromethylpyridyl, in particular 4-trifluoromethylpyridyl, 4-trifluoromethylpyrid-2-yl; trifluoromethoxypyridyl, in particular 4-trifluoromethoxypyrid-2-yl; difluoromethoxypyridyl, in particular 4-difluoromethoxypyrid-2-yl; trifluoromethylcyclobutylmethyl, in particular 3-(trifluoromethyl)cyclobutylmethyl; 4,4,4-trifluoro-3,3-dimethylbutyl;
and the remaining radicals and residues are as defined for formula I above or for any of the further particular embodiments described herein above or below.

In yet another particular embodiment, PE3c, of PE3, which is also a particular embodiment of PE3a or PE3b, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ represents 4-trifluoromethylphenyl or 4-trifluoromethoxyphenyl; in particular 4-trifluoromethylphenyl;
and the remaining radicals and residues are as defined for formula I above or for any of the further particular embodiments described herein above or below.

In another particular embodiment of the invention, PE4, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—OR^{2a} or Hetcyc^{X};
and the remaining radicals and residues are as defined for formula I above or for any of the further particular embodiments described herein above or below. In particular embodiment PE4 the substituent R² is a carboxylic acid radical or the salt of a carboxylic acid radical or the ester of a carboxylic acid radical or a heterocyclic radical which is optionally a carboxylic acid bioisostere.

In a particular embodiment, PE4a, of PE4, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—OR^{2a};
and the remaining radicals and residues are as defined for formula I above or for any of the further particular embodiments described herein above or below.

In a particular embodiment, PE4aa, of PE4a
R^{2a} represents H, straight-chain or branched, unsubstituted or substituted C₁₋₄-alkyl, in particular methyl, ethyl, n-propyl, 2-propyl, n-butyl, 2-butyl, tert.-butyl; or Cat;
Cat represents a monovalent cation selected from the group consisting of lithium (Li), sodium (Na) and potassium (K);
and the remaining radicals and residues are as defined for formula I above or for any of the further particular embodiments described herein above or below.

In yet another particular embodiment, PE4b, of PE4, which is also a particular embodiment of PE4a or PE4aa, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—OR^{2a};
R^{2a} represents H or Na: in particular H.
and the remaining radicals and residues are as defined for formula I above or for any of the further particular embodiments described herein above or below.

In still another particular embodiment, PE4c, of PE4, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents Hetcyc^{X}.
Hetcyc^{X} represents 1H-1,2,3,4-tetrazol-5-yl, 2H-1,2,3,4-tetrazol-5-yl, 2-methyl-2H-1,2,3,4-tetrazol-5-yl, 5-oxo-2,5-dihydro-1,2,4-oxadiazol-3-yl (2H-1,2,4-oxadiazol-5-on-3-yl), 5-oxo-4,5-dihydro-1,2,4-oxadiazol-3-yl (4H-1,2,4-oxadiazol-5-on-3-yl), 3-bromo-4,5-dihydro-1,2-oxazol-5-yl, 3-chloro-4,5-dihydro-1,2-oxazol-5-yl, 3-(1H-1,2,3-triazol-1-yl)-4,5-dihydro-1,2-oxazol-5-yl, 3-(2H-1,2,3-triazol-2-yl)-4,5-dihydro-1,2-oxazol-5-yl, 3-(pyrimidin-5-yloxy)-4,5-dihydro-1,2-oxazol-5-yl, 3-hydroxy-oxetan-3-yl, 5-hydroxy-4H-pyran-4-on-2-yl, 3,3-difluoropyrrolidin-2-on-4-yl, 3,3-difluoropyrrolidin-2-on-5-yl, 3,3-difluoro-2,3-dihydro-1H-pyrrol-2-on-4-yl, 3,3-difluoro-2,3-dihydro-1H-pyrrol-2-on-5-yl;
and the remaining radicals and residues are as defined for formula I above or for any of the further particular embodiments described herein above or below.

In another particular embodiment of the present invention, PE5, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
and the remaining radicals and residues are as defined for formula I above or for any of the further particular embodiments described herein above or below. In particular embodiment PE5 the substituent R² is an amide.

In one particular embodiment, PE5a, of PE5, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
R^{2b}, R^{2c} both represent H;
and the remaining radicals and residues are as defined for formula I above or for any of the further particular embodiments described herein above or below.

In another particular embodiment, PE5b, of PE5, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
one of R^{2b} and R^{2c} represents H, while the other of R^{2b} and R^{2c} represents Cyc^{2b}, Hetcyc^{2b}, straight-chain or branched C₁₋₁₀-alkyl which may be unsubstituted or substituted with R^{E1}, R^{E2}, R^{E3}, R^{E4} and/or R^{E5} which may be the same or different wherein in that C₁₋₁₀-alkyl 1 or 2 non-adjacent and non-terminal methylene moieties may be replaced by independently from each other —O— and/or —S— and/or —NH— and/or —N(C₁₋₄-alkyl)-;
Cyc^{2b} is a saturated monocyclic carbocycle with 3, 4, 5, 6 or 7 ring carbon atoms, wherein that carbocycle may be unsubstituted or substituted independently from each other with R^{D6}, R^{D7}, R^{D8}, R^{D9} and/or R^{D10} wherein that carbocycle may optionally be fused to Ar^{Z} or Hetar^{Z} via 2 adjacent ring atoms and wherein that fused carbocycle may optionally further be substituted with independently from each other R^{C1}, R^{C2} and/or R^{C3};
Hetcyc^{2b} is a saturated monocyclic heterocycle with 5 or 6 ring atoms wherein 1 or 2 of said ring atoms is/are a hetero atom(s) selected from N, O and/or S and the remaining are carbon atoms, wherein that heterocycle may be unsubstituted or substituted independently from each other with R^{D6}, R^{D7}, R^{D8}, R^{D9} and/or R^{D10} wherein that heterocycle may optionally be fused to Ar^{Z} or Hetar^{Z} and wherein that fused heterocycle may optionally further be substituted with independently from each other R^{C1}, R^{C2} and/or R^{C3};
R^{E1}, R^{E2}, R^{E3}, R^{E4} and/or R^{E5} represent independently from each other halogen, in particular F; —NR^{Ea}R^{Eb} OR^{Ec}, Ar^{E}, Hetar^{E}, Cyc^{E}, Hetcyc^{E};
Ar^{E} is a mono- or bicyclic aryl with 6 or 10 ring carbon atoms, wherein that aryl may be unsubstituted or substituted with substituents R^{F1}, R^{F2} and/or R^{F3} which may be the same or different; in particular phenyl or naphthalenyl;
Ar^{Z} is benzo;
Hetar^{E} is a monocyclic heteroaryl with 5 or 6 ring atoms or a bicyclic heteroaryl with 9 or 10 ring atoms wherein 1, 2, 3, or 4 of said ring atoms is/are a hetero atom(s) selected from N, O and/or S and the remaining are carbon atoms, wherein that heteroaryl may be unsubstituted or substituted with substituents R^{F1}, R^{F2} and/or R^{F3} which may be the same or different; in particular imidazolyl, 1H-imidazol-1-yl, 1H-imidazol-2-yl, each of which unsubstituted or monosubstituted with C₁₋₄-alkyl; pyridyl, pyrid-2-yl, pyrid-3-yl, pyrid-4-yl, each of which may be unsubstituted or monosubstituted with —F; pyrimidinyl, pyrimidin-2-yl, pyrimidin-3-yl, pyrimidin-4-yl; pyrazinyl, pyrazin-2-yl; pyridazinyl, pyridazin-3-yl; furanyl, pyrrolyl, pyrazolyl, oxazolyl, isoxazolyl; oxadiazolyl, triazolyl, thiazolyl, isothiazolyl;
Hetar^{Y1} is a 5 or 6 membered monocyclic heteroaryl wherein 1, 2, 3, 4 ring atoms are hetero atoms selected from N, O and/or S and the remaining are carbon atoms, wherein that heteroaryl may be unsubstituted or substituted with F, C₁₋₄-alkyl which may optionally be substituted with OH; in particular pyrrolyl, thiophenyl, pyrazolyl, methylpyrazolyl, imidazolyl, methylimidazolyl, triazolyl, oxadiazolyl, methyloxadiazolyl, pyrdinyl, fluoropyrdinyl, methylpyridinyl, pyrimidinyl, methylpyrimidinyl;
Hetar^{Y2} is a 5 or 6 membered monocyclic heteroaryl wherein 1, 2, 3, 4 ring atoms are hetero atoms selected from N, O and/or S and the remaining are carbon atoms, wherein that heteroaryl may be unsubstituted or substituted with halogen, C₁₋₄-alkyl which may optionally be substituted with OH; in particular pyrrolyl, furanyl, thiophenyl, pyrazolyl, imidazolyl, triazolyl, oxazolyl, hydroxymethyloxazolyl, pyrimidinyl;
Hetar^{Z} is pyrrole, N-methyl-pyrrole, pyrazole, imidazole, triazole;
Cyc^{E} is a saturated or partially unsaturated, mono- or bicyclic carbocycle with 3, 4, 5, 6, 7 or 8 ring carbon atoms, wherein that carbocycle may be unsubstituted or substituted with R^{G1} and/or R^{G2} which may be the same or different: in particular, a saturated monocyclic carbocycle with 3, 4, 5, or 6 ring carbon atoms, wherein that carbocycle may be unsubstituted or substituted with R^{G1} and/or R^{G2} which may be the same or different;
Cyc^{Y1} is a saturated or partially unsaturated monocyclic carbocycle with 3, 4, 5, 6 or 7 ring carbon atoms, wherein that carbocycle may be unsubstituted or substituted with halogen, OH, C₁₋₄-alkyl, in particular cyclopropyl, cyclohexenyl;
Hetcyc^{E} is a saturated or partially unsaturated, monocyclic heterocycle with 4, 5 or 6 ring atoms wherein 1 or 2 of said ring atoms is/are a hetero atom(s) selected from N, O and/or S and the remaining are carbon atoms, wherein that heterocycle may be unsubstituted or substituted with R^{G1} and/or R^{G2} which may be the same or different; in particular a saturated monocyclic heterocycle with 5 or 6 ring atoms wherein 1 or 2 of said ring atoms is/are a hetero atom(s) selected from N and/or O and the remaining are carbon atoms, wherein that heterocycle may be unsubstituted or substituted with R^{G1} and/or R^{G2}; preferably tetrahydrofuranyl, tetrahydrofuran-2-yl, tetrahydrofuran-3-yl each of which may be unsubstituted or monosubstituted with —OH; pyrrolindinyl, pyrrolindin-1-yl, pyrrolindin-2-yl, pyrrolindin-3-yl, each of which may be unsubstituted or monosubstituted with —OH; piperidinyl, piperidin-1-yl, piperidin-2-yl, piperidin-3-yl, piperidin-4-yl, each of which may be unsubstituted or monosubstituted with —OH; morpholinyl, morpholin-1-yl, morpholin-2-yl, each of which may be unsubstituted or mono-substituted with methyl; 1,4-dioxanyl; dihydropyranyl, tetrahydropyranyl, tetrahydropyran-3-yl;
Hetcyc^{Y1} is a saturated or partially unsaturated monocyclic heterocycle with 5 or 6 ring atoms wherein 1 or 2 of said ring atoms are heteroatoms selected from N, O, and/or S and the remaining are carbon atoms; in particular tetrahydrofuranyl;
Hetcyc^{Y2} is a saturated or partially unsaturated monocyclic heterocycle with 5 or 6 ring atoms wherein 1 or 2 of said ring atoms are heteroatoms selected from N, O, and/or S and the remaining are carbon atoms; in particular tetrahydrofuranyl, morpholinyl, tetrahydropyranyl;
R^{C1}, R^{C2}, R^{C3} represent independently from each other C₁₋₄-alkyl;
R^{D6}, R^{D7}, R^{D8}, R^{D9}, R^{D10} represent independently from each other halogen, in particular F; hydroxy; C₁₋₄-alkyl optionally substituted with —OH and/or halogen, in particular methyl, hydroxymethyl, 2-fluorethyl; —O—C₁₋₄-alkyl, in particular methoxy, ethoxy; Hetar^{Y1}, —CH₂-Hetar^{Y1}, Cyc^{Y1}, Hetcyc^{Y1}, —CH₂-Hetcyc^{Y1};
   and/or two of R^{D6}, R^{D7}, R^{D8}, R^{D9}, R^{D10} which are attached to the same ring atom of that carbocycle or heterocycle form a divalent C₂₋₆-alkylene radical wherein optionally one or two non-adjacent carbon units of that alkylene radical may be replaced by independently from each other O, NH, N—C₁₋₄-alkyl, and wherein that alkylene radical may optionally be substituted with OH, C₁₋₄-alkyl or —O—C₁₋₄-alkyl, in particular —(CH₂)₃—, —CH₂—CH(OC₂H₅)—CH₂—, —(CH₂)₂—O—(CH₂)₂—;
   and/or two of R^{D6}, R^{D7}, R^{D8}, R^{D9}, R^{D10} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl, in particular —CH₂—, —(CH₂)₃—, —O—(CH₂)₂—, —O—(CH₂)₃—;
R^{Ea}, R^{Eb} represent independently from each other H, C₁₋₄-alkyl, —C(═O)—C₁₋₄-alkyl, —C(═O)—OC₁₋₄-alkyl;
R^{Ec} represents H or C₁₋₄-alkyl;
R^{F1}, R^{F2}, R^{F3} represent independently from each other straight-chain or branched C₁₋₆-alkyl, which C₁₋₆-alkyl may be unsubstituted or monosubstituted with —CN, OH, —O—C₁₋₄-alkyl or substituted with 1, 2 or 3 halogen: straight-chain or branched C₁₋₄-alkoxy, which C₁₋₄-alkoxy may be unsubstituted or substituted with 1, 2 or 3 halogen; straight-chain or branched —S—C₁₋₄-alkyl, which —S—C₁₋₄-alkyl may be unsubstituted or substituted with 1, 2 or 3 halogen; C₃₋₇-cycloalkyl optionally substituted with halogen, OH and/or C₁₋₄-alkyl; F, Cl, Br, —CN, —S(═O)—C₁₋₃-alkyl, S(═O)₂—C₁₋₃-alkyl, —NH₂, —NH(C₁₋₃-alkyl), —N(C₁₋₃-alkyl)₂, —OH; in particular methyl, hydroxymethyl, methoxymethyl, F, cyclopropyl, cyclobutyl; preferably only one of R^{F1}, R^{F2} and R^{F3} is present and represents methyl or F;
   and/or two of R^{F1}, R^{F2}, R^{F3} which are attached to two different ring atoms of that aryl or heteroaryl form a divalent C₁₋₆-alkylene radical wherein optionally one or two non-adjacent carbon units of that alkylene radical may be replaced by independently from each other O, NH, N—C₁₋₄-alkyl, in particular —(CH₂)₄—, —CH₂—O—(CH₂)₂—;
R^{G1}, R^{G2} represent independently from each other halogen; hydroxy; unsubstituted or substituted C₁₋₆-aliphatic, in particular C₁₋₄-alkyl optionally substituted with OH; C₁₋₆-aliphatoxy in particular —O—C₁₋₄-alkyl; —C(═O)—O—C₁₋₄-alkyl; Hetar^{Y2}; —CH₂-Hetar^{Y2}; Hetcyc^{Y2}; in particular only one of R^{G1} and R^{G2} is present and represents hydroxy;
   and/or R^{G1} and R^{G2} which are attached to the same ring atom of that carbocycle or heterocycle form a divalent C₂₋₆-alkylene radical wherein optionally one or two non-adjacent carbon units of that alkylene radical may be replaced by independently from each other O, NH, N—C₁₋₄-alkyl, and wherein that alkylene radical may optionally be substituted with OH, C₁₋₄-alkyl or —O—C₁₋₄-alkyl, in particular —(CH₂)₂—O—CH₂—, —(CH₂)₂—O—(CH₂)₂—;
   and/or R^{G1} and R^{G2} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl, in particular —CH₂—;
and the remaining radicals and residues are as defined for formula I above or for any of the further particular embodiments described herein above or below.

In a particular embodiment, PE5ba, of PE5b, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein one of R^{2b} and R^{2c} represents H, while the other of R^{2b} and R^{2c} represents
Cyc^{2b}, Hetcyc^{2b}, straight-chain or branched C₁₋₈-alkyl which may be unsubstituted or substituted with R^{E1}, R^{E2}, R^{E3}, R^{E4} and/or R^{E5} which may be the same or different;
Cyc^{2b} is cyclopropyl, cyclobutyl or 1-hydroxymethyl-cyclobutyl;
Hetcyc^{2b} is tetrahydrofuranyl or hydroxytetrahydrofuranyl;
R^{E1}, R^{E2}, R^{E3}, R^{E4} and/or R^{E5} represent independently from each other F; —NR^{Ea}R^{Eb}, OR^{Ec}, Ar^{E}, Hetar^{E}, Cyc^{E}, Hetcyc^{E};
Ar^{E} is phenyl which may be unsubstituted or substituted with substituents R^{F1}, R^{F2} and/or R^{F3} which may be the same or different;
Hetar^{E} is selected from the group consisting of imidazolyl, 1H-imidazol-1-yl, 1H-imidazol-2-yl, each of which unsubstituted or monosubstituted with C₁₋₄-alkyl; pyridyl, pyrid-2-yl, pyrid-3-yl, pyrid-4-yl, each of which may be unsubstituted or monosubstituted with —F; pyrimidinyl, pyrimidin-2-yl, pyrimidin-3-yl, pyrimidin-4-yl; pyrazinyl, pyrazin-2-yl; pyridazinyl, pyridazin-3-yl; furanyl, pyrrolyl, pyrazolyl, oxazolyl, isoxazolyl; oxadiazolyl, triazolyl, thiazolyl, isothiazolyl;
Cyc^{E} is cyclopropyl or cyclobutyl;
Hetcyc^{E} is selected from the group consisting of tetrahydrofuranyl, tetrahydrofuran-2-yl, tetrahydrofuran-3-yl each of which may be unsubstituted or monosubstituted with —OH; pyrrolindinyl, pyrrolindin-1-yl, pyrrolindin-2-yl, pyrrolindin-3-yl, each of which may be unsubstituted or monosubstituted with —OH; piperidinyl, piperidin-1-yl, piperidin-2-yl, piperidin-3-yl, piperidin-4-yl, each of which may be unsubstituted or monosubstituted with —OH; morpholinyl, morpholin-1-yl, morpholin-2-yl, each of which may be unsubstituted or mono-substituted with methyl; 1,4-dioxanyl; dihydropyranyl, tetrahydropyranyl, tetrahydropyran-3-yl;
R^{Ea}, R^{Eb} both represent H or one represents H and the other represents —C(═O)-methyl or C(═O)—O-tert.-butyl;
R^{Ec} represents H or methyl;
R^{F1}, R^{F2}, R^{F3} represent independently from each other methyl, hydroxymethyl, methoxymethyl, F, cyclopropyl, cyclobutyl; in particular only one of R^{F1}, R^{F2} and R^{F3} is present and represents methyl or F;
and the remaining radicals and residues are as defined for formula I above or for any of the further particular embodiments described herein above or below.

In yet another particular embodiment, PE5baa, of PE5, which is also a particular embodiment of PE5b or PE5ba, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
one of R^{2b} and R^{2c} represents H, while the other of R^{2b} and R^{2c} represents methyl; ethyl; 2-hydroxyethyl; 1-hydroxypropan-2-yl (—CH(CH₃)—CH₂OH), in particular (2R)-1-hydroxypropan-2-yl, (2S)-1-hydroxypropan-2-yl; 2-hydroxypropanyl (2-hydroxypropyl; —CH₂—C(CH₃)—OH), in particular (2S)-2-hydroxypropanyl, (2R)-2-hydroxypropanyl; 1-hydroxy-4-methoxybutan-2-yl (—CH(CH₂OH)—(CH₂)₂OCH₃); 1-hydroxybutan-2-yl (—CH(CH₂OH)—CH₂CH₃), in particular (2S)-1-hydroxybutan-2-yl, (2R)-1-hydroxybutan-2-yl; 1-(hydroxymethyl)cyclopropyl; 2,3-dihydroxypropanyl (—CH₂—C(OH)CH₂OH); pyridinylmethyl, in particular pyridin-2-ylmethyl, pyridin-3-ylmethyl, pyridin-4-ylmethyl; 2-hydroxy-1-pyrdinylethyl, in particular 2-hydroxy-1-(pyridin-2-yl)ethyl, especially (1R)-2-hydroxy-1-(pyridin-2-yl)ethyl, (1 S)-2-hydroxy-1-(pyridin-2-yl)ethyl; 2-hydroxy-1-(1-methyl-1H-pyrazol-3-yl)ethyl.

In another particular embodiment, PE5c, of PE5, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
one of R^{2b} and R^{2c} represents straight-chain or branched alkyl C₁₋₁₀-alkyl optionally substituted with OH or halogen, while the other of R^{2b} and R^{2c} represents Cyc^{2b}, Hetcyc^{2b}, straight-chain or branched C₁₋₁₀-alkyl which may be unsubstituted or substituted with R^{E1}, R^{E2}, R^{E3}, R^{E4} and/or R^{E5} which may be the same or different, wherein in that C₁₋₁₀-alkyl 1 or 2 non-adjacent and non-terminal methylene moieties may be replaced by independently from each other —O—and/or —S— and/or —NH— and/or —N(C₁₋₄-alkyl)-;
Cyc^{2b} is a saturated monocyclic carbocycle with 3, 4, 5, 6 or 7 ring carbon atoms, wherein that carbocycle may be unsubstituted or substituted independently from each other with R^{D6}, R^{D7}, R^{D8}, R^{D9} and/or R^{D10} wherein that carbocycle may optionally be fused to Ar^{Z} or Hetar^{Z} via 2 adjacent ring atoms and wherein that fused carbocycle may optionally further be substituted with independently from each other R^{C1}, R^{C2} and/or R^{C3};
Hetcyc^{2b} is a saturated monocyclic heterocycle with 5 or 6 ring atoms wherein 1 or 2 of said ring atoms is/are a hetero atom(s) selected from N, O and/or S and the remaining are carbon atoms, wherein that heterocycle may be unsubstituted or substituted independently from each other with R^{D6}, R^{D7}, R^{D8}, R^{D9} and/or R^{D10} wherein that heterocycle may optionally be fused to Ar^{Z} or Hetar^{Z} and wherein that fused heterocycle may optionally further be substituted with independently from each other R^{C1}, R^{C2} and/or R^{C3};
R^{E1}, R^{E2}, R^{E3}, R^{E4} and/or R^{E5} represent independently from each other halogen, in particular F; —NR^{Ea}R^{Eb} OR^{Ec}, Ar^{E}, Hetar^{E}, Cyc^{E}, Hetcyc^{E};
Ar^{E} is a mono- or bicyclic aryl with 6 or 10 ring carbon atoms, wherein that aryl may be unsubstituted or substituted with substituents R^{F1}, R^{F2} and/or R^{F3} which may be the same or different; in particular phenyl or naphthalenyl;
Ar^{Z} is benzo;
Hetar^{E} is a monocyclic heteroaryl with 5 or 6 ring atoms or a bicyclic heteroaryl with 9 or 10 ring atoms wherein 1, 2, 3, or 4 of said ring atoms is/are a hetero atom(s) selected from N, O and/or S and the remaining are carbon atoms, wherein that heteroaryl may be unsubstituted or substituted with substituents R^{F1}, R^{F2} and/or R^{F3} which may be the same or different; in particular imidazolyl, 1H-imidazol-1-yl, 1H-imidazol-2-yl, each of which unsubstituted or monosubstituted with C₁₋₄-alkyl; pyridyl, pyrid-2-yl, pyrid-3-yl, pyrid-4-yl, each of which may be unsubstituted or monosubstituted with —F; pyrimidinyl, pyrimidin-2-yl, pyrimidin-3-yl, pyrimidin-4-yl; pyrazinyl, pyrazin-2-yl; pyridazinyl, pyridazin-3-yl; furanyl, pyrrolyl, pyrazolyl, oxazolyl, isoxazolyl; oxadiazolyl, triazolyl, thiazolyl, isothiazolyl;
Hetar^{Y1} is a 5 or 6 membered monocyclic heteroaryl wherein 1, 2, 3, 4 ring atoms are hetero atoms selected from N, O and/or S and the remaining are carbon atoms, wherein that heteroaryl may be unsubstituted or substituted with F, C₁₋₄-alkyl which may optionally be substituted with OH; in particular pyrrolyl, thiophenyl, pyrazolyl, methylpyrazolyl, imidazolyl, methylimidazolyl, triazolyl, oxadiazolyl, methyloxadiazolyl, pyrdinyl, fluoropyrdinyl, methylpyridinyl, pyrimidinyl, methylpyrimidinyl;
Hetar^{Y2} is a 5 or 6 membered monocyclic heteroaryl wherein 1, 2, 3, 4 ring atoms are hetero atoms selected from N, O and/or S and the remaining are carbon atoms, wherein that heteroaryl may be unsubstituted or substituted with halogen, C₁₋₄-alkyl which may optionally be substituted with OH; in particular pyrrolyl, furanyl, thiophenyl, pyrazolyl, imidazolyl, triazolyl, oxazolyl, hydroxymethyloxazolyl, pyrimidinyl;
Hetar^{Z} is pyrrole, N-methyl-pyrrole, pyrazole, imidazole, triazole;
Cyc^{E} is a saturated or partially unsaturated, mono- or bicyclic carbocycle with 3, 4, 5, 6, 7 or 8 ring carbon atoms, wherein that carbocycle may be unsubstituted or substituted with R^{G1} and/or R^{G2} which may be the same or different: in particular, a saturated monocyclic carbocycle with 3, 4, 5, or 6 ring carbon atoms, wherein that carbocycle may be unsubstituted or substituted with R^{G1} and/or R^{G2} which may be the same or different;
Cyc^{Y1} is a saturated or partially unsaturated monocyclic carbocycle with 3, 4, 5, 6 or 7 ring carbon atoms, wherein that carbocycle may be unsubstituted or substituted with halogen, OH, C₁₋₄-alkyl, in particular cyclopropyl, cyclohexenyl;
Hetcyc^{E} is a saturated or partially unsaturated, monocyclic heterocycle with 4, 5 or 6 ring atoms wherein 1 or 2 of said ring atoms is/are a hetero atom(s) selected from N, O and/or S and the remaining are carbon atoms, wherein that heterocycle may be unsubstituted or substituted with R^{G1} and/or R^{G2} which may be the same or different; in particular a saturated monocyclic heterocycle with 5 or 6 ring atoms wherein 1 or 2 of said ring atoms is/are a hetero atom(s) selected from N and/or O and the remaining are carbon atoms, wherein that heterocycle may be unsubstituted or substituted with R^{G1} and/or R^{G2}; preferably tetrahydrofuranyl, tetrahydrofuran-2-yl, tetrahydrofuran-3-yl each of which may be unsubstituted or monosubstituted with —OH; pyrrolindinyl, pyrrolindin-1-yl, pyrrolindin-2-yl, pyrrolindin-3-yl, each of which may be unsubstituted or monosubstituted with —OH; piperidinyl, piperidin-1-yl, piperidin-2-yl, piperidin-3-yl, piperidin-4-yl, each of which may be unsubstituted or monosubstituted with —OH; morpholinyl, morpholin-1-yl, morpholin-2-yl, each of which may be unsubstituted or mono-substituted with methyl; 1,4-dioxanyl; dihydropyranyl, tetrahydropyranyl, tetrahydropyran-3-yl;
Hetcyc^{Y1} is a saturated or partially unsaturated monocyclic heterocycle with 5 or 6 ring atoms wherein 1 or 2 of said ring atoms are heteroatoms selected from N, O, and/or S and the remaining are carbon atoms; in particular tetrahydrofuranyl;
Hetcyc^{Y2} is a saturated or partially unsaturated monocyclic heterocycle with 5 or 6 ring atoms wherein 1 or 2 of said ring atoms are heteroatoms selected from N, O, and/or S and the remaining are carbon atoms; in particular tetrahydrofuranyl, morpholinyl, tetrahydropyranyl;
R^{C1}, R^{C2}, R^{C3} represent independently from each other C₁₋₄-alkyl;
R^{D6}, R^{D7}, R^{D8}, R^{D9}, R^{D10} represent independently from each other halogen, in particular F; hydroxy; C₁₋₄-alkyl optionally substituted with —OH and/or halogen, in particular methyl, hydroxymethyl, 2-fluorethyl; —O—C₁₋₄-alkyl, in particular methoxy, ethoxy; Hetar^{Y1}, —CH₂-Hetar^{Y1}, Cyc^{Y1}, Hetcyc^{Y1}, —CH₂-Hetcyc^{Y1};
   and/or two of R^{D6}, R^{D7}, R^{D8}, R^{D9}, R^{D10} which are attached to the same ring atom of that carbocycle or heterocycle form a divalent C₂₋₆-alkylene radical wherein optionally one or two non-adjacent carbon units of that alkylene radical may be replaced by independently from each other O, NH, N—C₁₋₄-alkyl, and wherein that alkylene radical may optionally be substituted with OH, C₁₋₄-alkyl or —O—C₁₋₄-alkyl, in particular —(CH₂)₃—, —CH₂—CH(OC₂H₅)—CH₂—, —(CH₂)₂—O—(CH₂)₂—;
   and/or two of R^{D6}, R^{D7}, R^{D8}, R^{D9}, R^{D10} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl, in particular —CH₂—, —(CH₂)₃—, —O—(CH₂)₂—, —O—(CH₂)₃—;
R^{Ea}, R^{Eb} represent independently from each other H, C₁₋₄-alkyl, —C(═O)—C₁₋₄-alkyl, —C(═O)—OC₁₋₄-alkyl;
R^{Ec} represents H or C₁₋₄-alkyl;
R^{F1}, R^{F2}, R^{F3} represent independently from each other straight-chain or branched C₁₋₆-alkyl, which C₁₋₆-alkyl may be unsubstituted or monosubstituted with —CN, OH, —O—C₁₋₄-alkyl or substituted with 1, 2 or 3 halogen: straight-chain or branched C₁₋₄-alkoxy, which C₁₋₄-alkoxy may be unsubstituted or substituted with 1, 2 or 3 halogen; straight-chain or branched —S—C₁₋₄-alkyl, which —S—C₁₋₄-alkyl may be unsubstituted or substituted with 1, 2 or 3 halogen; C₃₋₇-cycloalkyl optionally substituted with halogen, OH and/or C₁₋₄-alkyl; F, Cl, Br, —CN, —S(═O)—C₁₋₃-alkyl, S(═O)₂—C₁₋₃-alkyl, —NH₂, —NH(C₁₋₃-alkyl), —N(C₁₋₃-alkyl)₂, —OH; in particular methyl, hydroxymethyl, methoxymethyl, F, cyclopropyl, cyclobutyl; preferably only one of R^{F1}, R^{F2} and R^{F3} is present and represents methyl or F;
   and/or two of R^{F1}, R^{F2}, R^{F3} which are attached to two different ring atoms of that aryl or heteroaryl form a divalent C₁₋₆-alkylene radical wherein optionally one or two non-adjacent carbon units of that alkylene radical may be replaced by independently from each other O, NH, N—C₁₋₄-alkyl, in particular —(CH₂)₄—, —CH₂—O—(CH₂)₂—;
R^{G1}, R^{G2} represent independently from each other halogen; hydroxy; unsubstituted or substituted C₁₋₆-aliphatic, in particular C₁₋₄-alkyl optionally substituted with OH; C₁₋₆-aliphatoxy in particular —O—C₁₋₄-alkyl; —C(═O)—O—C₁₋₄-alkyl; Hetar^{Y2}; —CH₂-Hetar^{Y2}; Hetcyc^{Y2}; in particular only one of R^{G1} and R^{G2} is present and represents hydroxy;
   and/or R^{G1} and R^{G2} which are attached to the same ring atom of that carbocycle or heterocycle form a divalent C₂₋₆-alkylene radical wherein optionally one or two non-adjacent carbon units of that alkylene radical may be replaced by independently from each other O, NH, N—C₁₋₄-alkyl, and wherein that alkylene radical may optionally be substituted with OH, C₁₋₄-alkyl or —O—C₁₋₄-alkyl, in particular —(CH₂)₂—O—CH₂—, —(CH₂)₂—O—(CH₂)₂—;
   and/or R^{G1} and R^{G2} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl, in particular —CH₂—;
and the remaining radicals and residues are as defined for formula I above or for any of the further particular embodiments described herein above or below.

In a particular embodiment, PE5ca, of PE5c, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
one of R^{2b} and R^{2c} represents methyl, ethyl or 2-hydroxyethyl, while the other of R^{2b} and R^{2c} represents Cyc^{2b}, Hetcyc^{2b}, straight-chain or branched C₁₋₈-alkyl which may be unsubstituted or substituted with R^{E1}, R^{E2}, R^{E3}R^{E4} and/or R^{E5} which may be the same or different;
Cyc^{2b} is cyclopropyl, cyclobutyl or 1-hydroxymethyl-cyclobutyl;
Hetcyc^{2b} is tetrahydrofuranyl or hydroxytetrahydrofuranyl;
R^{E1}, R^{E2}, R^{E3}, R^{E4} and/or R^{E5} represent independently from each other F; —NR^{Ea}R^{Eb} OR^{Ec}, Ar^{E}, Hetar^{E}, Cyc^{E}, Hetcyc^{E};
Ar^{E} is phenyl which may be unsubstituted or substituted with substituents R^{F1}, R^{F2} and/or R^{F3} which may be the same or different;
Hetar^{E} is selected from the group consisting of imidazolyl, 1H-imidazol-1-yl, 1H-imidazol-2-yl, each of which unsubstituted or monosubstituted with C₁₋₄-alkyl; pyridyl, pyrid-2-yl, pyrid-3-yl, pyrid-4-yl, each of which may be unsubstituted or monosubstituted with —F; pyrimidinyl, pyrimidin-2-yl, pyrimidin-3-yl, pyrimidin-4-yl; pyrazinyl, pyrazin-2-yl; pyridazinyl, pyridazin-3-yl; furanyl, pyrrolyl, pyrazolyl, oxazolyl, isoxazolyl; oxadiazolyl, triazolyl, thiazolyl, isothiazolyl;
Cyc^{E} is cyclopropyl or cyclobutyl;
Hetcyc^{E} is selected from the group consisting of tetrahydrofuranyl, tetrahydrofuran-2-yl, tetrahydrofuran-3-yl each of which may be unsubstituted or monosubstituted with —OH; pyrrolindinyl, pyrrolindin-1-yl, pyrrolindin-2-yl, pyrrolindin-3-yl, each of which may be unsubstituted or monosubstituted with —OH; piperidinyl, piperidin-1-yl, piperidin-2-yl, piperidin-3-yl, piperidin-4-yl, each of which may be unsubstituted or monosubstituted with —OH; morpholinyl, morpholin-1-yl, morpholin-2-yl, each of which may be unsubstituted or mono-substituted with methyl; 1,4-dioxanyl; dihydropyranyl, tetrahydropyranyl, tetrahydropyran-3-yl;
R^{Ea}, R^{Eb} both represent H or one represents H and the other represents —C(═O)-methyl or C(═O)—O—tert.-butyl;
R^{Ec} represents H or methyl;
R^{F1}, R^{F2}, R^{F3} represent independently from each other methyl, hydroxymethyl, methoxymethyl, F, cyclopropyl, cyclobutyl; in particular only one of R^{F1}, R^{F2} and R^{F3} is present and represents methyl or F;
and the remaining radicals and residues are as defined for formula I above or for any of the further particular embodiments described herein above or below.

In another particular embodiment, PE5d, of PE5, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2e};
R^{2b} and R^{2c} form together with the nitrogen atom to which they are attached to a saturated or partially unsaturated heterocycle optionally substituted with independently from each other R^{Y1}, R^{Y2}, R^{Y3}, R^{Y4} and/or R^{Y5}; wherein that heterocycle may optionally be fused with Hetar^{Z}; and wherein that heterocycle is selected from the group consisting of: azetidine, pyrrolidine, piperidine, piperazine, morpholine;
R^{Y1}, R^{Y2}, R^{Y3}, R^{Y4}, R^{Y5} represent independently from each other halogen, in particular F; —NH₂, —N(H)—C₁₋₄-alkyl, —N(H)—C(═O)—O—C₁₋₄-alkyl, —N(C₁₋₄-alkyl)₂; —OH; C₁₋₄-alkyl optionally substituted with —OH, —O—C₁₋₄-alkyl, —O—C₃₋₇-cycloalkyl, —O—CH₂—C₃₋₇-cycloalkyl, in particular methyl, —CH₂OH, —(CH₂)₂OH, —(CH₂)₃OH, —CH₂OCH₃, —(CH₂)₂OCH₃, cyclopropylmethoxy; —O—C₁₋₄-alkyl, in particular methoxy; Hetar^{Y2}; —CH₂-Hetar^{Y2}; Hetcyc^{Y2}; and/or two of R^{Y1}, R^{Y2}, R^{Y3}, R^{Y4}, R^{Y5} which are attached to the same ring atom of that heterocycle form a divalent C₂₋₆-alkylene radical wherein optionally one or two non-adjacent carbon units of that alkylene radical may be replaced by independently from each other O, NH, N—C₁₋₄-alkyl, in particular —(CH₂)₄—, —(CH₂)₂—O—(CH₂)₂—, —(CH₂)₂—O—(CH₂)₃—; and/or two of R^{Y1}, R^{Y2}, R^{Y3}, R^{Y4}, R^{Y5} which are attached to two different ring atoms of that heterocycle form a divalent C₁₋₆-alkylene radical wherein optionally one or two non-adjacent carbon units of that alkylene radical may be replaced by independently from each other O, NH, N—C₁₋₄-alkyl, in particular —(CH₂)₄—;
Hetar^{Y2} is a 5 or 6 membered monocyclic heteroaryl wherein 1, 2, 3, 4 ring atoms are hetero atoms selected from N, O and/or S and the remaining are carbon atoms, wherein that heteroaryl may be unsubstituted or substituted with halogen, C₁₋₄-alkyl which may optionally be substituted with OH; in particular pyrrolyl, furanyl, thiophenyl, pyrazolyl, imidazolyl, triazolyl, oxazolyl, hydroxymethyloxazolyl, pyrimidinyl;
Hetar^{Z} is pyrrole, N-methyl-pyrrole, pyrazole, imidazole, triazole;
Hetcyc^{Y2} is a saturated or partially unsaturated monocyclic heterocycle with 5 or 6 ring atoms wherein 1 or 2 of said ring atoms are heteroatoms selected from N, O, and/or S and the remaining are carbon atoms; in particular tetrahydrofuranyl, morpholinyl, tetrahydropyranyl;
and the remaining radicals and residues are as defined for formula I above or for any of the further particular embodiments described herein above or below.

In a particular embodiment, PE5da, of PE5d, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R^{2b} and R^{2c} form together with the nitrogen atom to which they are attached to a pyrrolidinyl or piperidinyl ring each of which is unsubstituted or mono-substituted with —OH or di-substituted with independently from each other C₁₋₄-alkyl and/or —OH;
and the remaining radicals and residues are as defined for formula I above or for any of the further particular embodiments described herein above or below.

In a particular embodiment, PE5daa, of PE5da, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R^{2b} and R^{2c} form together with the nitrogen atom to which they are attached to a pyrrolidin-3-ol ring, in particular a (3S)-pyrrolidin-3-ol ring.

In another particular embodiment of the present invention, PE6, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —(CH₂)ₓ—NR^{2d}—C(═O)—R^{2e}, —S—R^{2f}, —S(═O)—R^{2f}, —S(═O)₂—R^{2g}, —S(═O)₂—NR^{2h}R²ⁱ, —S(═O)₂—OH, —S(═O)(═NR^{2j})—OH, —S(═O)(═NR^{2j})—R^{2g}, —S(═O)(═NR^{2k})—NR^{2l}R^{2m}, —(CH₂)_{z}—NR^{2d}—S(═O)₂—R^{2g}, —N═S(═O)—R^{2s}R^{2t}, —C(═O)—N═S(═O)—R^{2s}R^{2t}, —C(═O)—N═S(═N—R^{2u})—R^{2s}R^{2t}; in particular —(CH₂)ₓ—NR^{2d}—C(═O)—R^{2e}, —S(═O)—R^{2f}, —S(═O)₂—R^{2g}, —S(═O)₂—NR^{2h}R²ⁱ, —S(═O)(═NR^{2j})—R^{2g}, —S(═O)(═NR^{2k})—NR^{2l}R^{2m}, —(CH₂)_{z}—NR^{2d}—S(═O)₂—R^{2g}, —N═S(═O)—R^{2s}R^{2t}, —C(═O)—N═S(═O)—R^{2s}R^{2t}, —C(═O)—N═S(═N—R^{2u})—R^{2s}R^{2t}; preferably, —NH—C(═O)—CH₃, —S(═O)—CH₃, —S(═O)₂—CH₃, —S(═O)₂—NH₂, —S(═O)₂—NHCH₃, —S(═O)(═NH)—CH₃, S(═O)(═NH)—N(CH₃)₂, —NH—(S═O)₂—CH₃, —NH—S(═O)₂—CH═CH₂, —CH₂—NH—S(═O)₂—CH═CH₂, —N═S(═O)(CH₃)₂, C(═O)—N═S(═O)(CH₃)₂;
R^{2e} represents H, C₁₋₆-alkyl optionally substituted with —OH or a monocyclic 5- or 6-membered heteroaryl; C₃₋₇-cycloalkyl, monocyclic 5- or 6-membered heteroaryl; in particular H, methyl, hydroxymethyl, methylpyridin-2-yl, methylpyridin-3-yl, methylpyridin-4-yl, cyclopropyl, pyridin-2-yl, pyridin-3-yl, pyridin-4-yl;
R^{2f}, R^{2g} represent independently from each other un-substituted or substituted C₁₋₈-aliphatic; in particular independently from each other C₁₋₄-alkyl or C₂₋₄-alkenyl; preferably independently from each other methyl or —CH═CH₂:
R^{2h}, R²ⁱ represent independently from each other H, un-substituted or substituted C₁₋₈-aliphatic, aryl, heterocyclyl, heteroaryl; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms; in particular independently from each other H or C₁₋₄-alkyl;
R^{2d}, R^{2j}, R^{2k} represent independently from each other H, un-substituted or substituted C₁₋₈-aliphatic; in particular H;
R^{2l}, R^{2m} represent independently from each other H, un-substituted or substituted C₁₋₈-aliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O or S and the remaining are carbon atoms; in particular C₁₋₄-alkyl; preferably methyl;
R^{2s}, R^{2t} represent independently from each other C₁₋₆-alkyl which may optionally be substituted with —OH, O—C₁₋₄-alkyl, NH₂, NHC₁₋₄-alkyl, N(C₁₋₄-alkyl)₂, pyrrolidinyl, piperidinyl, morpholinyl, piperazinyl; in particular methyl, ethyl, 2-hydroxyethyl, 3-hydroxy propyl, 2-aminoethyl, 3-(N,N-dimethylamino)propyl; or form together a divalent C₃₋₄-alkylene radical which may optionally be substituted with —NH₂, —CN, or a divalent C₂₋₅-alkylene radical wherein optionally one of the carbon units of said C₂₋₅-alkylene radical may be replaced by O, NH or N—C₁₋₄-alkyl; in particular —(CH₂)₃—, —CH₂—C(NH₂)H—CH₂—, —CH₂—C(CN)H—CH₂—, —CH₂—C(CH₂—NH—CH₂)—CH₂—, —(CH₂)₄—;
R^{2u} represents hydrogen or C₁₋₄-alkyl;
x represents 0 or 1;
z represents 0 or 1;
and the remaining radicals and residues are as defined for formula I above or for any of the further particular embodiments described herein above or below.

In a particular embodiment, PE6a, of PE6, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² represents —C(═O)—N═S(═O)(CH₃)₂,
and the remaining radicals and residues are as defined for formula I above or for any of the further particular embodiments described herein above or below.

In another particular embodiment of the present invention, PE7, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
Ring A represents a five-membered heteroaromatic ring selected from the group consisting of the following ring moieties:

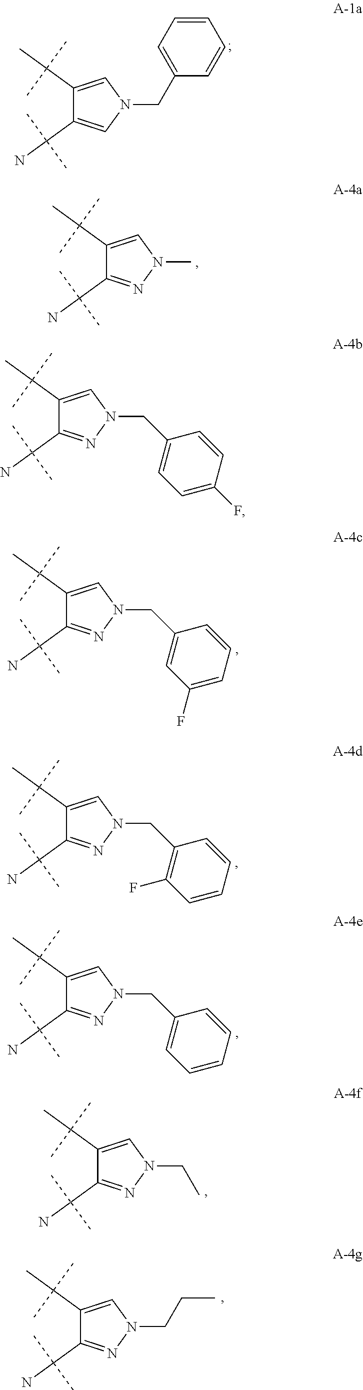

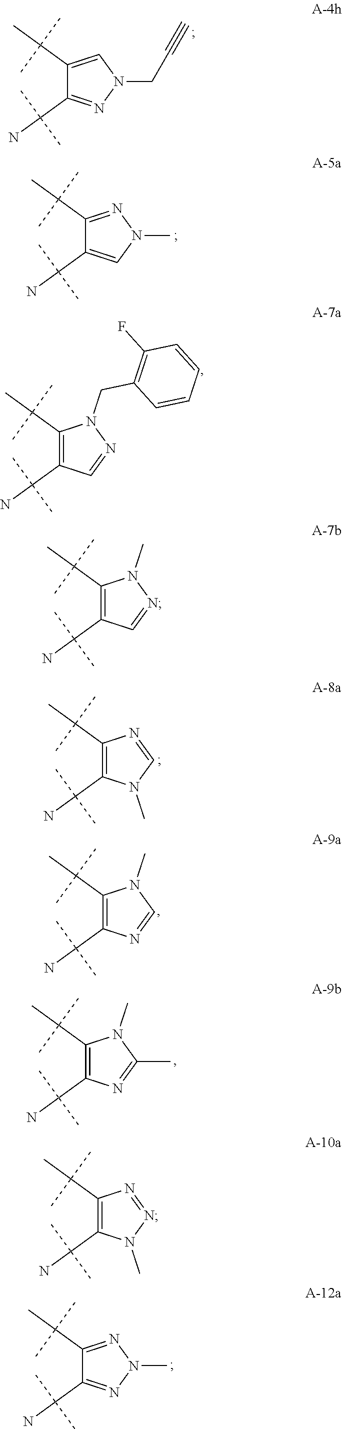

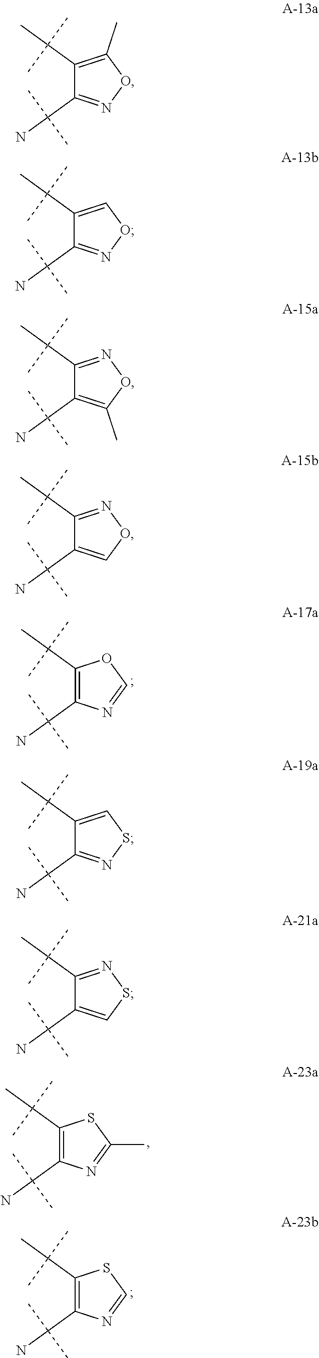

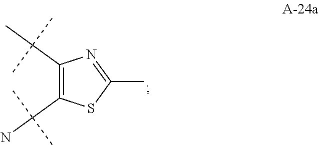

Ring B represents a five-membered heteroaromatic ring selected from the group consisting of the following ring moieties:

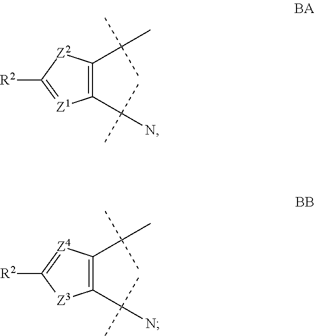

Z¹ is CH or N; and
Z² is S;
or
Z³ is S; and
Z⁴ CH or N;
R¹ represents phenyl, 3-fluorophenyl, 4-fluorophenyl, 4-chlorophenyl, 4-methylphenyl, 4-ethylphenyl, 4-difluoromethylphenyl, 3-trifluoromethyl-phenyl, 4-trifluoromethylphenyl, 4-(1,1-difluorethyl)phenyl, 4-(2,2,2-trifluorethyl)phenyl, 4-(1-trifluoromethylcyclopropyl)-phen-1-yl, 4-cyclopentylphenyl, 4-ethoxyphenyl, 4-difluoromethoxyphenyl, 4-trifluoromethoxyphenyl, 3-(trifluoromethyl)sulfanylphenyl, 4-(trifluoromethyl)sulfanylphenyl, 3-trifluoromethyl-4-methylphenyl, 2-fluoro-4-trifluoromethylphenyl, 2-fluoro-4-trifluoromethoxyphenyl, 3-fluoro-4-(n-propyl)phenyl, 2,3-dimethyl-4-methoxyphenyl, 6-fluoronaphth-2-yl; 5-trifluoromethylfuran-2-yl; 5-trifluoromethylthiophen-2-yl, 2-trifluoromethyl-1,3-thiazol-4-yl, 3-fluoropyridin-2-yl, 6-methylpyridin-3-yl, 6-methoxypyridin-3-yl, 3-ethylpyridin-2-yl, 6-ethylpyridin-3-yl, 4-difluoromethylpyridin-2-yl, 4-trifluoromethylpyridin-2-yl, 4-difluoromethoxypyridin-2-yl, 4-trifluoromethoxypyridin-2-yl, 4-cyanopyridin-2-yl, 5-trifluoromethylpyridin-2-yl, 6-trifluoromethylpyridin-2-yl, 6-trifluoromethylpyridin-3-yl (2-trifluoromethylpyridin-5-yl), 6-trifluoromethoxypyridin-3-yl (2-trifluoromethoxypyridin-5-yl), 5-cyanopyridin-2-yl, 5-cyanomethylpyridin-2-yl, 5-methanesulfonylpyridin-2-yl, 6-methoxypyridin-2-yl, 4-methylpyrimidin-2-yl, 4-ethylpyrimidin-2-yl, 4-methylsulfanylpyrimidin-2-yl, 5-cyclopropylpyrimidin-2-yl, 5-ethylpyrimidin-2-yl, 5-difluoromethyl-pyrimidin-2-yl, 5-trifluoromethylpyrimidin-2-yl, 5-cyanopyrimidin-2-yl, 5-cyano-3-fluoropyridin-2-yl, 5-cyano-6-methylpyridin-2-yl, 3-fluoro-5-(tri-fluoromethyl)pyridin-2-yl, 5-oxo-5H,6H,7H-cyclopenta[b]pyridin-2-yl, 5,6,7,8-tetrahydroquinolin-2-yl, 5-oxo-5,6,7,8-tetrahydroquinolin-2-yl, 5H,6H,7H-cyclopenta[b]pyridin-2-yl, quinolin-2-yl, isoquinolin-3-yl, 6-methylquinolin-2-yl, 8-methoxyquinolin-4-yl, furo[3,2-b]pyridin-5-yl, quinazolin-2-yl, 6-fluoroquinazolin-2-yl, 1,5-naphthyridin-2-yl; 3-methylcyclobutyl, cyclopentyl, 3-methylcyclopentyl, 3,3-dimethylcyclopentyl, 3-trifluoromethyl-bicyclo[1.1.1]petan-1-yl, cyclohexyl, 4-methylcyclohexyl, 4-(trifluoromethyl)cyclohexyl, 4,4-difluorocyclohexyl, cyclohex-1-enyl, 2-oxocycloheptyl, 6,6-difluorospiro[3.3]heptan-2-yl, 1H-inden-2-yl; 4-benzenesulfonyl (phenylsulfonyl), 3-methylphenylsulfonyl, benzyl, 2-ethoxyphenylmethyl, 3-chlorophenylmethyl, 3-fluorophenylmethyl, 4-chlorophenylmethyl, 3-(pyrrolidine-1-yl)phenylmethyl, 3-methylphenylmethyl, 4-methylphenylmethyl, 3-ethylphenylmethyl, 3-(propan-2-yl)phenylmethyl, 3-tert-butylphenylmethyl, 3-(difluoro-methoxy)phenylmethyl, 2-(difluoromethyl)phenylmethyl, 3-(difluoromethyl)phenylmethyl, 3-(trifluoromethyl)phenylmethyl, 4-(trifluoromethyl)phenyl]methyl, 2-(prop-2-yn-1-yloxy)phenylmethyl, 3-(1,3-thiazol-2-yl)phenylmethyl, 3-(trifluoromethyl)sulfanylphenylmethyl, 3-methanesulfonylphenylmethyl, 3-(dimethylamino)phenylmethyl, 3-(pyrrol-1-yl)phenylmethyl, 2-methyl-3-methoxyphenylmethyl, 3-trifluoromethyl-5-methylphenylmethyl, 2-methyl-3-(trifluoromethyl)phenylmethyl, 3-trifluoromethyl-4-fluorophenylmethyl, 2-fluoro-5-(trifluoromethoxy)phenylmethyl, 2-methoxy-3-trifluoro-methoxyphenylmethyl, 2-fluoro-3-methoxyphenylmethyl, 2-fluoro-3-(trifluoromethyl)phenyl]methyl, 2-fluor-3-fluoromethoxyphenylmethyl, 2-trifluoromethoxy-5-fluorophenylmethyl, 2-fluor-5-chlor-phenylmethyl, 3-fluoro-5-methylphenyl)methyl, 3,5-difluorophenylmethyl, 5-fluoro-2-(trifluoromethyl)phenylmethyl, 3-fluoro-5-(trifluoromethyl)phenylmethyl, 2-chloro-3-(trifluoromethyl)phenylmethyl, naphthalin-1-ylmethyl, 5,6,7,8-tetrahydronaphthalen-1-ylmethyl, 2,3-dihydro-1-benzofuran-7-ylmethyl, 3,4-dihydro-2H-1-benzopyran-8-ylmethyl, 2-phenylethyl, 2-(2-methylphenyl)ethyl, 2-(2-methoxyphenyl)ethyl, 2-(3-methoxyphenyl)ethyl, 2-(4-methoxyphenyl)ethyl, 2-(2-fluorophenyl)-ethyl, 2-(3-fluorophenyl)-ethyl, 2-(4-fluorophenyl)-ethyl, 2-(2-chlorophenyl)-ethyl, 2-(4-chlorophenyl)-ethyl, 2-(4-bromophenyl)-ethyl, 2-[4-(trifluoromethyl)phenyl]ethyl, 2-(2,4-difluorophenyl)ethyl, 2-(difluoromethoxy)-5-fluorophenylmethyl, 2-phenylpropyl, 3-phenylpropyl, 3-methyl-3-phenylbutyl, 2-(benzyloxy)ethyl; 5-ethylfuran-2-ylmethyl, 5-(trifluoromethyl)furan-2-ylmethyl, 4-(propan-2-yl)-1,3-thiazol-2-ylmethyl, 2-methyl-1,3-thiazol-4-ylmethyl, 2-trifluoromethyl-1,3-thiazol-4-ylmethyl, 1-ethylpyrazol-5-ylmethyl, 1-(2-propyl)pyrazol-5-ylmethyl, 1-ethylimidazol-5-ylmethyl, 1-ethylimidazol-2-ylmethyl, 1-propylimidazol-2-ylmethyl, 1-benzylimidazol-2-yl)methyl, 1-(2-methylpropyl)-1H-imidazol-5-ylmethyl, 5-tert-butyl-1,3-oxazol-2-ylmethyl, 3-fluoropyridin-2-ylmethyl, 2-methylpyridin-4-ylmethyl, 4-trifluoromethylpyridin-2-yl, 4-trifluoromethylpyridin-2-ylmethyl, 6-(fluoro-methyl)pyridin-2-ylmethyl, 6-trifluoromethylpyridin-2-yl, 2-(trifluoro-methyl)pyridin-4-ylmethyl, 4-methylpyrimidin-2-ylmethyl, 4-trifluoromethylpyridin-2-ylmethyl, 4-trifluoromethylpyridin-2-ylmethyl, 6-(fluoromethyl)pyridin-2-ylmethyl, 6-trifluoromethylpyridin-2-ylmethyl, 2-(trifluoromethyl)pyridin-4-ylmethyl, 4-methylpyrimidin-2-ylmethyl, 2-(thiophen-3-yl)ethyl, 5-trifluoromethylthiophen-2-ylmethyl, 1-methyl-1H-indol-6-yl)methyl, 1-benzofuran-3-ylmethyl, 1-benzothiophen-3-ylmethyl, 4H,5H,6H-pyrrolo[1,2-b]pyrazol-3-ylmethyl, pyrazolo[1,5-a]pyridin-7-ylmethyl, pyrazolo[1,5-a]pyridin-3-ylmethyl, imidazo[1,2-a]pyridin-3-ylmethyl, 6-methylimidazo[1,2-a]pyridin-3-ylmethyl, imidazo[1,2-a]pyridin-5-ylmethyl, imidazo[1,5-a]pyridin-1-ylmethyl, imidazo[1,5-a]pyridin-3-ylmethyl, imidazo[1,5-a]pyridin-5-ylmethyl, pyrazolo[1,5-c]pyrimidin-3-ylmethyl, 3-(furan-2-yl)prop-2-en-1-yl; 3-trifluormethylcyclobutylmethyl, 3-fluoro-3-phenylcyclobutylmethyl, cyclohexylmethyl, 4-methylcyclohexylmethyl, 4-trifluoromethylcyclohexylmethyl, 4-methoxycyclohexylmethyl, 4,4-dimethylcyclohexylmethyl, 4,4-difluorocyclohexylmethyl, 3-trifluoromethyl-bicyclo[1.1.1]pentan-1-ylmethyl, bicyclo[2.2.1]heptan-2-ylmethyl, bicyclo[2.2.2]octan-2-ylmethyl, bicyclo[2.2.1]hept-5-en-2-ylmethyl, 6,6-dimethylbicyclo[3.1.1]hept-2-en-2-yl]methyl; 3,3-dimethyltetrahydrofuran-2-ylmethyl, 1,1-dioxothian-4-ylmethyl, 2-(thian-4-yl)ethyl; 2,2-dimethyl-4,4,4-trifluoropentyl, 4,4,4-trifluorobutyl, 4,4,4-trifluoro-3-methylbutyl, 4,4,4-trifluoro-3,3-dimethylbutyl, 3,3,3-trifluoroprop-1-yn-1-yl; and
R² represents —C(═O)—OH, —C(═O)—ONa, —C(═O)—OCH₃, —C(═O)—NH₂, —C(═O)—NH—CH₃, —C(═O)—NHCH₂CH₃, —C(═O)—NH(CH₂)₂CH₃, —C(═O)—N(H)-cyclopropyl, —C(═O)—N(H)-(1-hydroxymethyl)cyclobutan-1-yl, —C(═O)—N(H)—CH₂CH₂—OH, —C(═O)—N(H)—CH₂CH₂—OCH₃, N(H)—CH₂CH(CF₃)—OH, —C(═O)—N(H)—CH(CH₃)CH₂—OH, —C(═O)—N(H)—CH₂CH(CH₃)—OH, —C(═O)—N(H)—CH₂—C(H)(OH)—CH₃, —C(═O)—N(H)—CH₂C(CH₃)₂OH, —C(═O)—N(H)—C(H)(CH₃)—CH₂OH, —C(═O)—N(H)—CH(CH₂CH₃)CH₂—OH, —C(═O)—N(H)—CH(CH(CH₃)₂)CH₂—OH, —C(═O)—N(H)—CH₂C(CH₃)₂OH, —C(═O)—N(H)—CH(OH)CH₂—OH, —C(═O)—N(H)—C(H)(CH₂CH₃)—CH₂OH, —C(═O)—N(H)—C(H)(CH₂OH)—CH₂CH₂—O—CH₃, —C(═O)—N(H)—C(CH₃)₂CH₂CH₂OH, —C(═O)—N(H)—C(H)(CH₂OH)-phenyl, —C(═O)—N(H)—CH(CH(CH₃)—OH)-phenyl, —C(═O)—N(H)—C(CH₃)(CH₂OH)-phenyl, —C(═O)—N(H)—C(H)(CH(OH)CH₃)-phenyl, —C(═O)—N(H)—CH(CH₂CH₂OH)-1,3-thiazol-5-yl, —C(═O)—N(H)—CH₂-1H-1-methylimidazol-2-yl, —C(═O)—N(H)—(CH₂)₂-1H-imidazol-1-yl, —C(═O)—N(H)—CH₂-pyridin-2-yl, —C(═O)—N(H)—CH₂-pyridin-3-yl, —C(═O)—N(H)—CH₂-pyridin-4-yl, —C(═O)—N(H)—C(H)(CH₂OH)-pyridin-2-yl, —C(═O)—N(H)—CH₂-1,3-pyrimidin-2-yl, —C(═O)—N(H)—CH₂-1,3-pyrimidin-4-yl, —C(═O)—N(H)—CH₂-pyridazin-2-yl, —C(═O)—NH—C(CH₂OH)-cyclobutyl, —C(═O)—N(H)-cyclopropyl, —C(═O)—N(H)-(1-hydroxymethyl)cyclobutan-1-yl, —C(═O)—NH—CH₂-azetidin-3-yl, —C(═O)—N(H)-(4-hydroxy-tetrahydrofuran-3-yl), —(C═O)—NH—CH₂CH₂-morpholin-4-yl, —C(═O)-3-hydroxy-pyrrolidin-1-yl, —C(═O)-3-hydroxy-piperidin-1-yl, —NH—C(═O)—CH═CH₂, —NH—C(═O)—CF═CH₂, —NH—C(═O)—CH₂Cl, —NH—C(═O)—C═CH, —CH₂—NH—C(═O)—CH═CH₂, —CH₂—NH—C(═O)—CH₂Cl, —CH₂—NH—C(═O)—C═CH, —S(═O)—CH₃, —S(═O)₂—CH₃, —S(═O)₂—OH, —S(═O)₂—NH₂, —S(═O)₂—NHCH₃, —S(═O)(═NH)—N(CH₃)₂, —S(═O)(═N—CH₃)—N(CH₃)₂, —S(═O)(═N—CH₃)—OH, —S(═O)(═NH)—CH₃, —C(═O)—N═S(═O)—(CH₃)₂, —C(═O)—N═S(═O)—(CH₃)(CH₂CH₂CH₂OH), —P(═O)(OH)₂, F, —CN.

In a particular embodiment, PE7a, of PE7, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein

Ring A represents

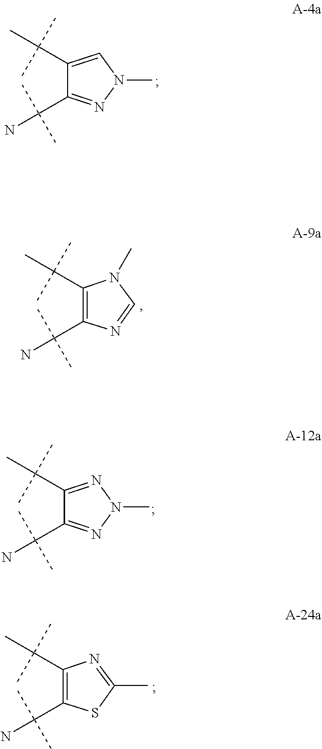

Ring B represents either

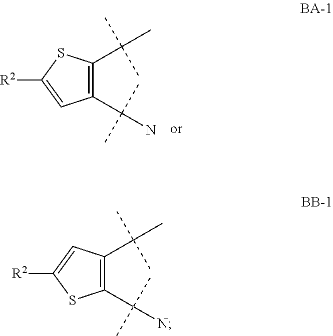

R¹ represents 4-trifluoromethylphenyl; 4-difluoromethoxyphenyl; 4-trifluoromethoxyphenyl; 3-(trifluoromethyl)cyclobutylmethyl; 4,4,4-trifluoro-3,3-dimethylbutyl;
R² represents —C(═O)—OH, —C(═O)—ONa, —C(═O)—OCH₃, —C(═O)—NH₂, —C(═O)—NH—CH₃, —C(═O)—NHCH₂CH₃, —C(═O)—NH(CH₂)₂CH₃, —C(═O)—N(H)-cyclopropyl, —C(═O)—N(H)-(1-hydroxymethyl)cyclobutan-1-yl, —C(═O)—N(H)—CH₂CH₂—OH, —C(═O)—N(H)—CH₂CH₂—OCH₃, —C(═O)—N(H)—CH(CH₃)CH₂—OH, —C(═O)—N(H)—CH₂CH(CH₃)—OH, —C(═O)—N(H)—CH₂—C(H)(OH)—CH₃, —C(═O)—N(H)—CH₂C(CH₃)₂OH, —C(═O)—N(H)—C(H)(CH₃)—CH₂OH, —C(═O)—N(H)—CH(CH₂CH₃)CH₂—OH, —C(═O)—N(H)—CH(CH(CH₃)₂)CH₂—OH, —C(═O)—N(H)—CH₂C(CH₃)₂OH, —C(═O)—N(H)—CH(OH)CH₂—OH, —C(═O)—N(H)—C(H)(CH₂CH₃)—CH₂OH, —C(═O)—N(H)—C(H)(CH₂OH)—CH₂CH₂—O—CH₃, —C(═O)—N(H)—C(CH₃)₂CH₂CH₂OH, —C(═O)—N(H)—C(H)(CH₂OH)-phenyl, —C(═O)—N(H)—CH(CH(CH₃)—OH)-phenyl, —C(═O)—N(H)—C(CH₃)(CH₂OH)-phenyl, —C(═O)—N(H)—C(H)(CH(OH)CH₃)-phenyl, —C(═O)—N(H)—CH(CH₂CH₂OH)-1,3-thiazol-5-yl, —C(═O)—N(H)—CH₂-1H-1-methylimidazol-2-yl, —C(═O)—N(H)—(CH₂)₂-1H-imidazol-1-yl, —C(═O)—N(H)—CH₂-pyridin-2-yl, —C(═O)—N(H)—CH₂-pyridin-3-yl, —C(═O)—N(H)—CH₂-pyridin-4-yl, —C(═O)—N(H)—C(H)(CH₂OH)-pyridin-2-yl, —C(═O)—N(H)—CH₂-1,3-pyrimidin-2-yl, —C(═O)—N(H)—CH₂-1,3-pyrimidin-4-yl, —C(═O)—N(H)—CH₂-pyridazin-2-yl, —C(═O)—NH—C(CH₂OH)-cyclobutyl, —C(═O)—N(H)-cyclopropyl, —C(═O)—N(H)-(1-hydroxymethyl)cyclobutan-1-yl, —C(═O)—NH—CH₂-azetidin-3-yl, —C(═O)—N(H)-(4-hydroxy-tetrahydrofuran-3-yl), —(C═O)—NH—CH₂CH₂-morpholin-4-yl, —C(═O)-3-hydroxy-pyrrolidin-1-yl, —C(═O)-3-hydroxy-piperidin-1-yl, —C(═O)—N═S(═O)—(CH₃)₂, —C(═O)—N═S(═O)—(CH₃)(CH₂CH₂CH₂OH).

In a particular embodiment, PE7b, of PE7, which is also a particular embodiment of PE7a, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
Ring A represents

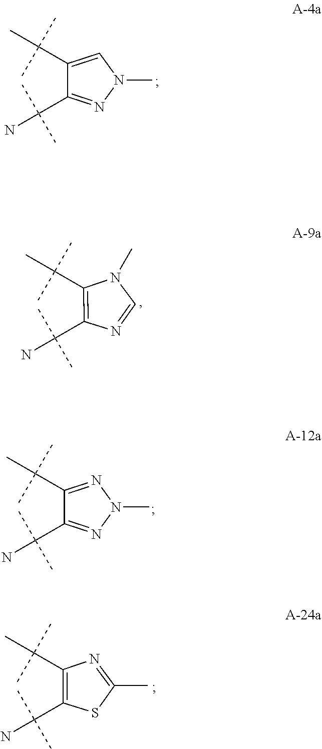

Ring B represents either

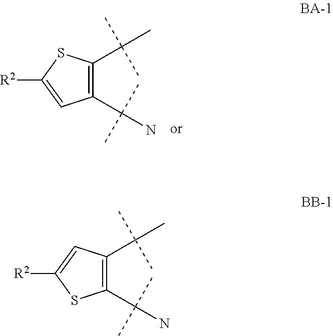

R¹ is 4-trifluoromethylphenyl or 4-trifluoromethoxyphenyl; and
R² is C(═O)—OH or C(═O)—ONa.

In another particular embodiment of the invention, PE8, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ is selected from the group consisting of

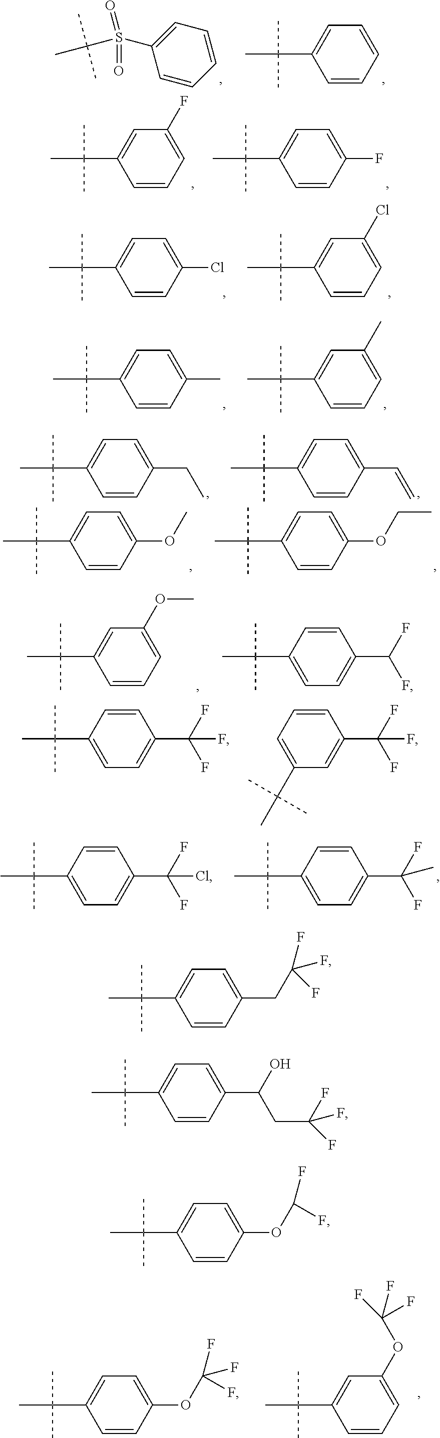

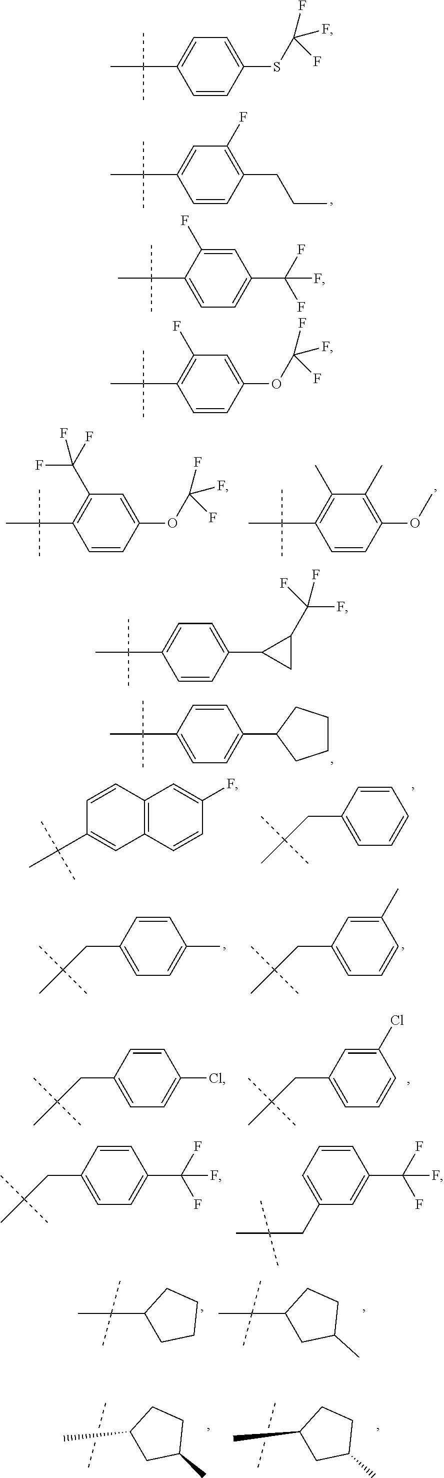

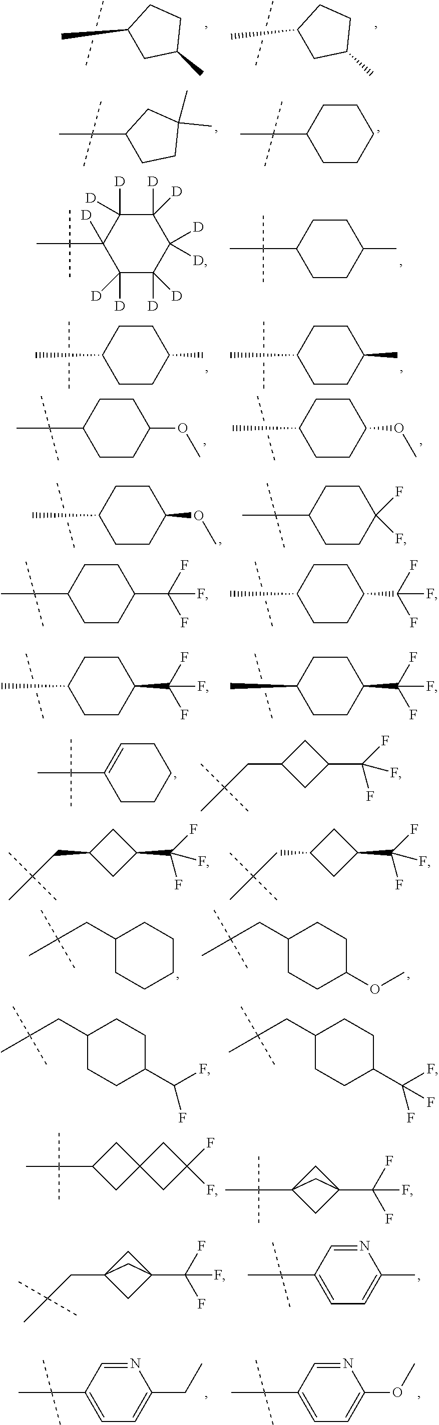

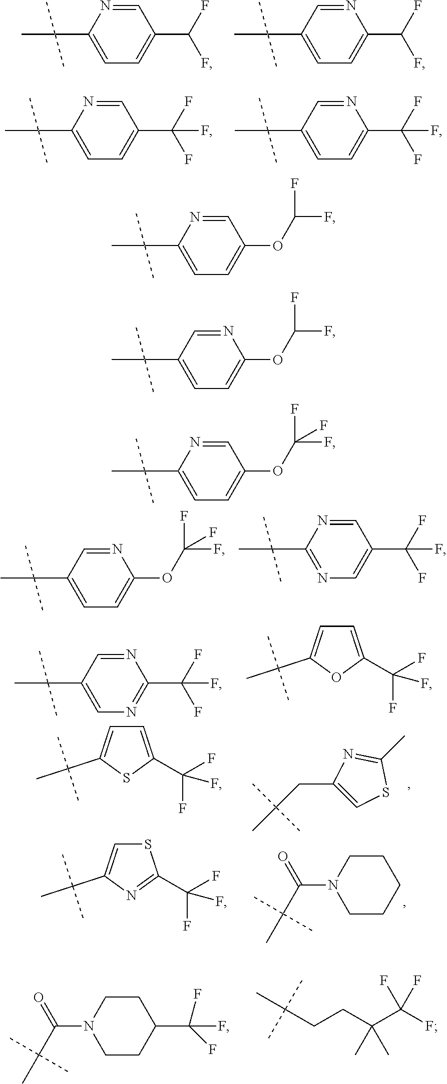

and the remaining radicals and residues are as defined for formula I above or for any of the further particular embodiments described herein above or below.

In a particular embodiment, PE8a, of PE8
R¹ is selected from the group consisting of

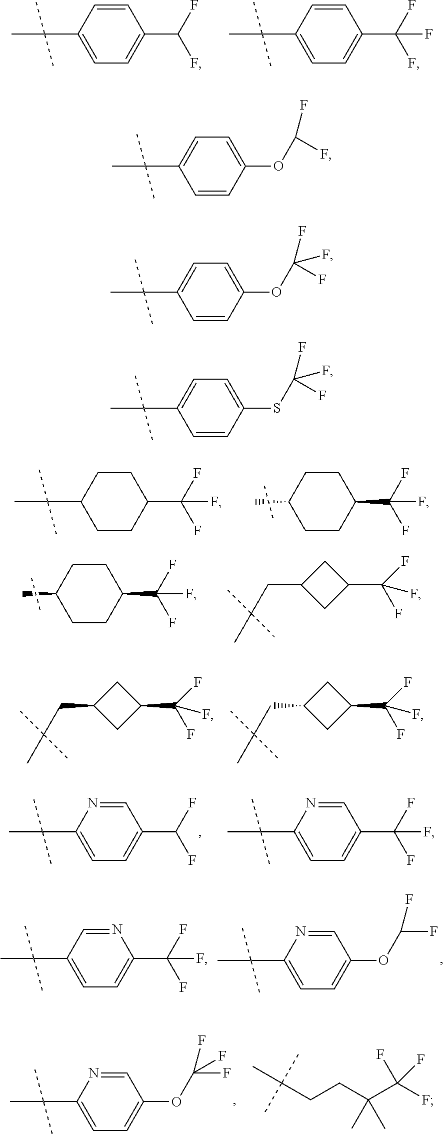

and the remaining radicals and residues are as defined for formula I above or for any of the further particular embodiments described herein above or below. Especially, R¹ is

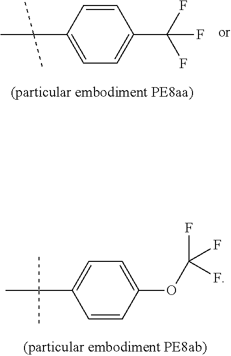

In yet another particular embodiment of the invention, PE9, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² is selected from the group consisting of —COOH, —COONa, —COOCH₃,

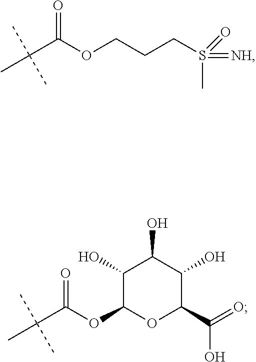

—CN, —F, —Br—; —CH₂CN; B(OH)₂;

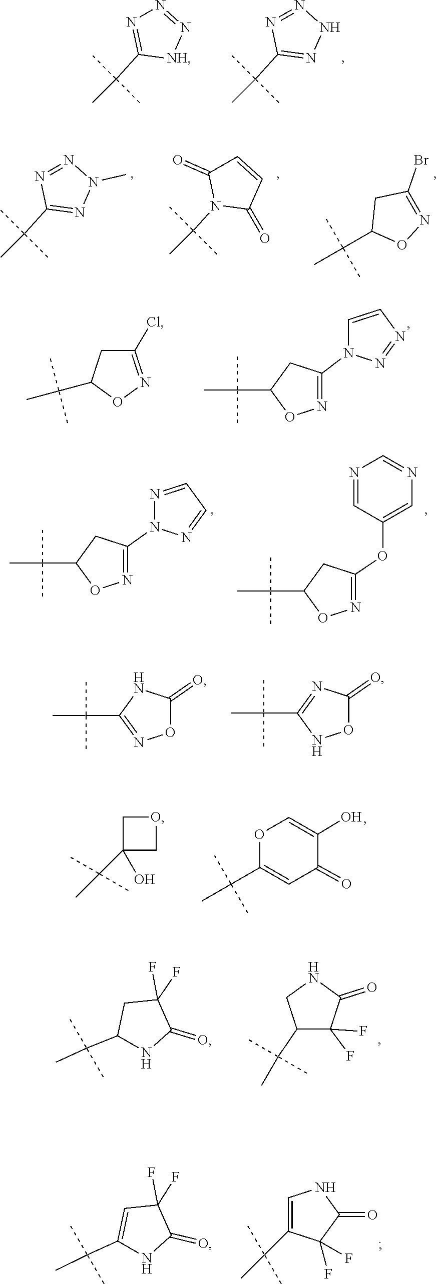

### —C(═O)—NH₂,

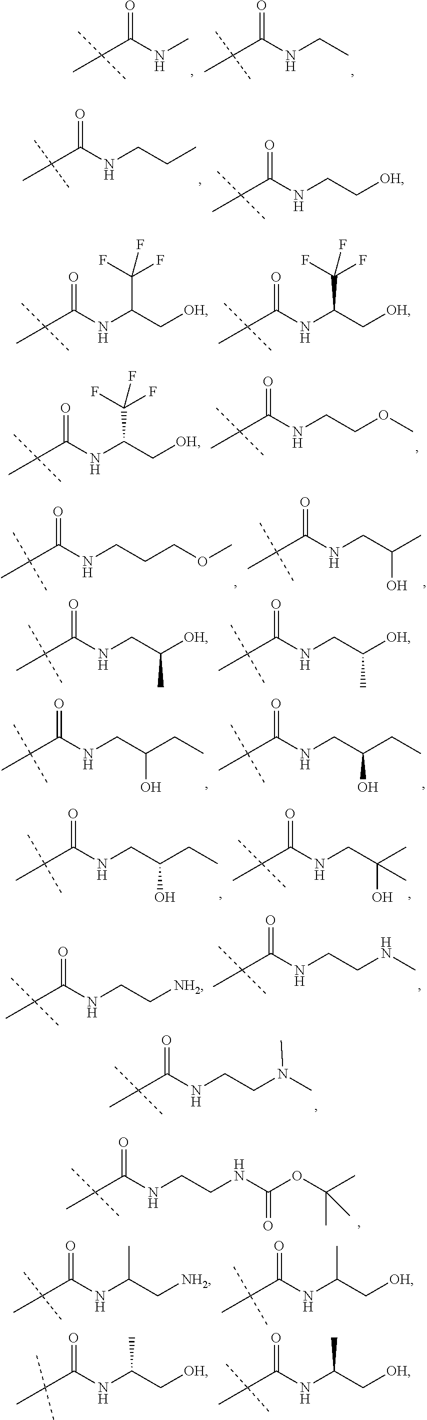

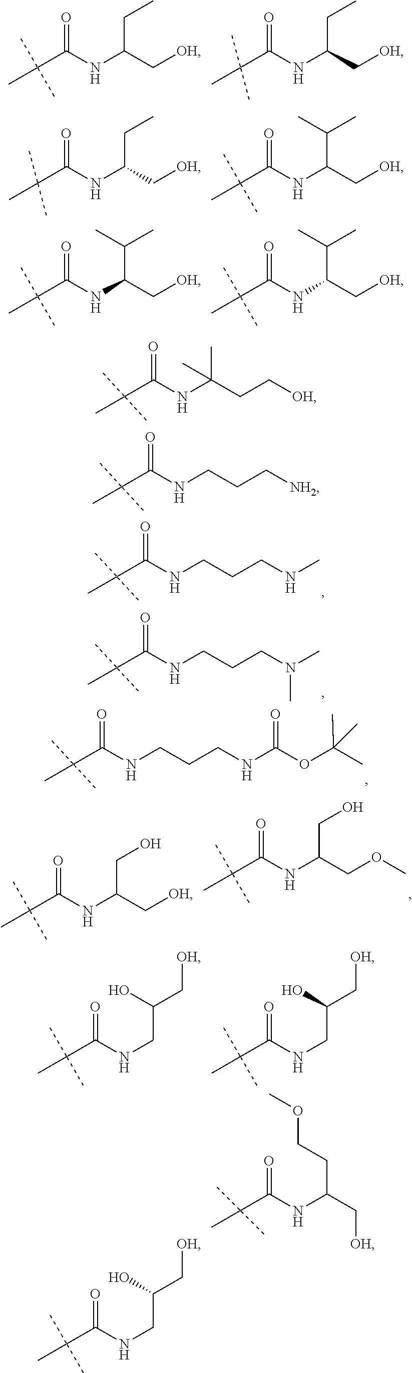

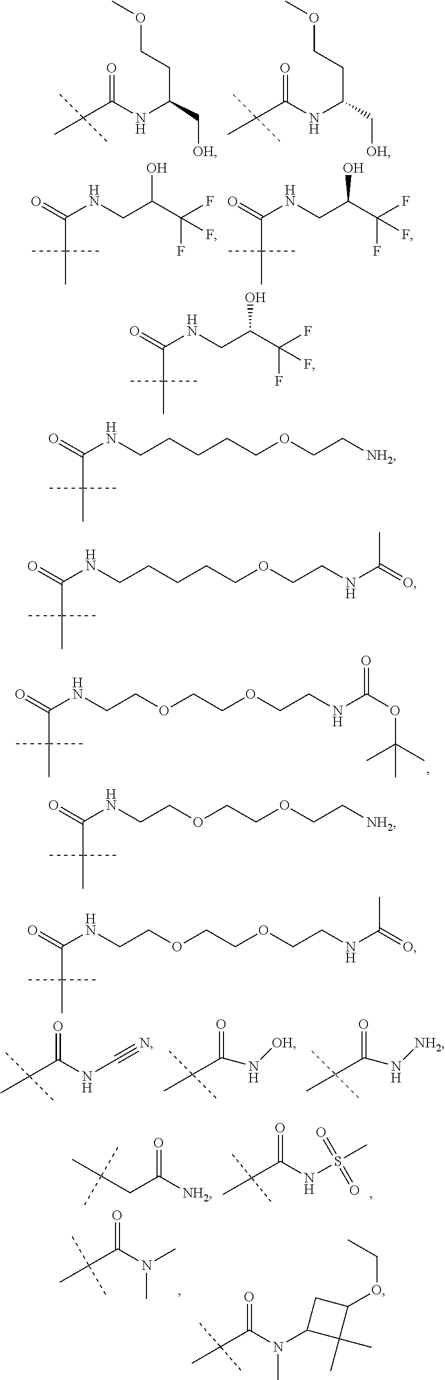

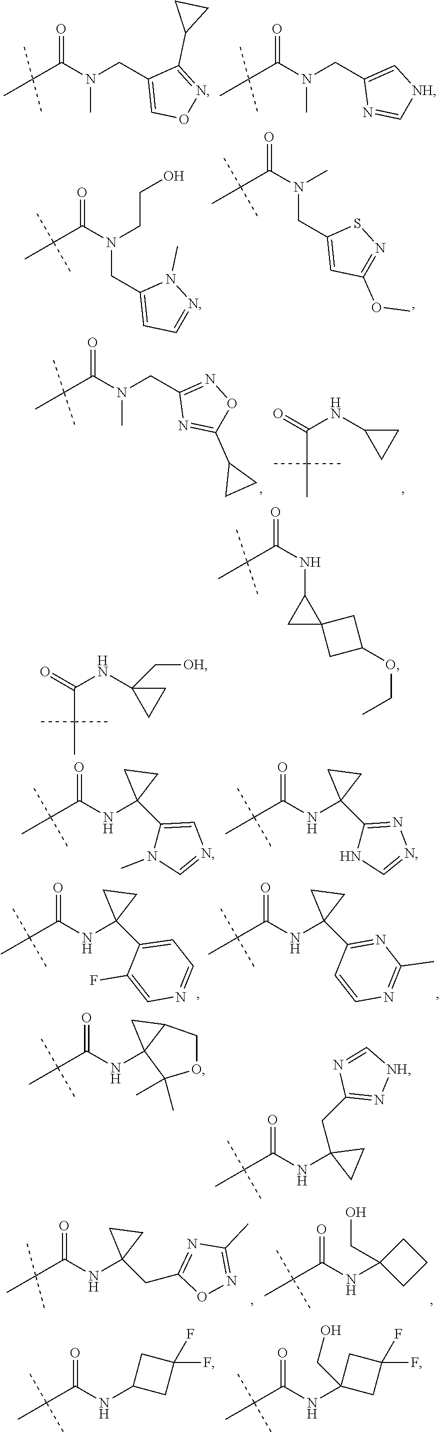

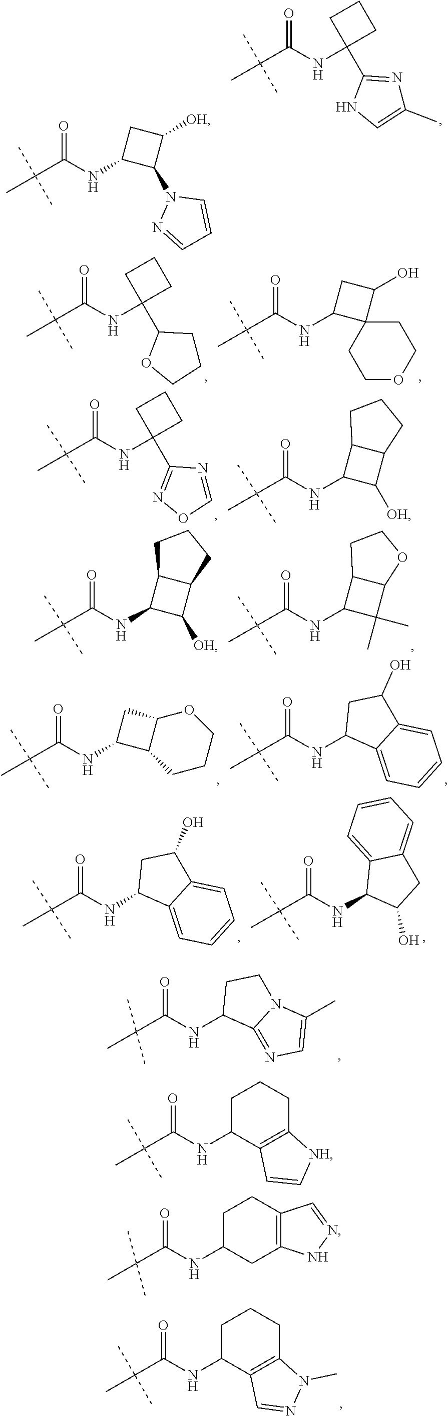

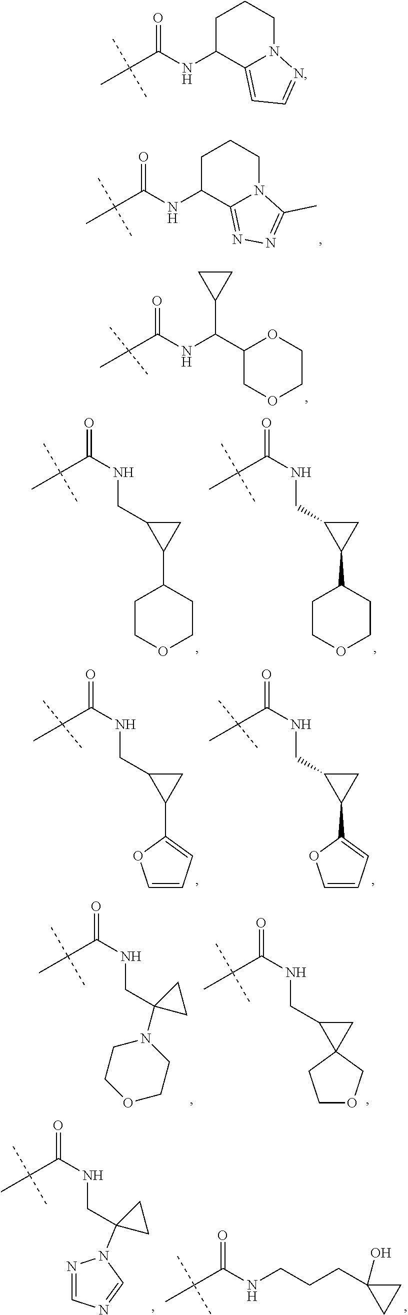

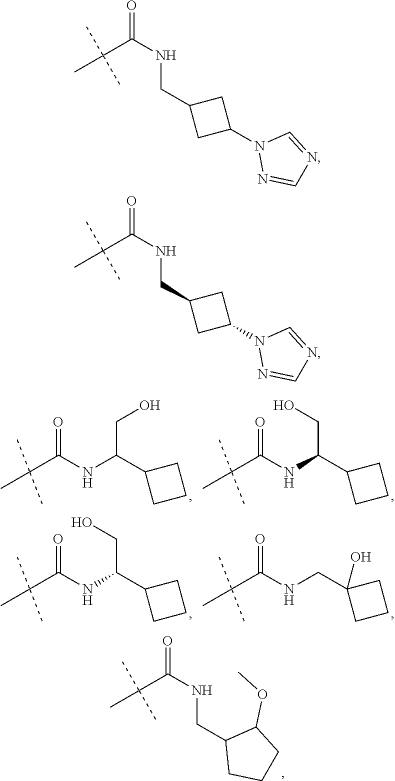

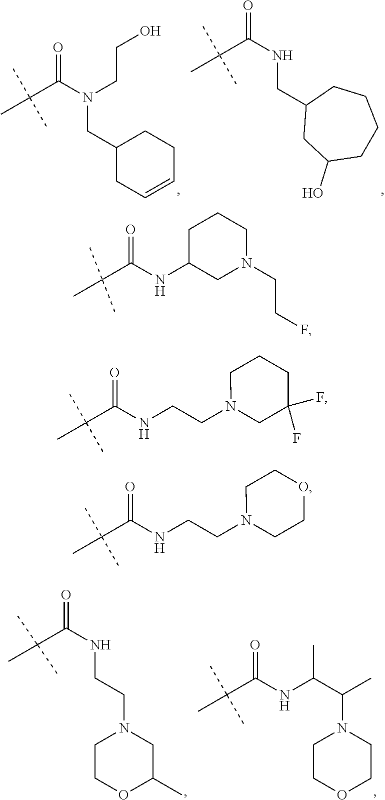

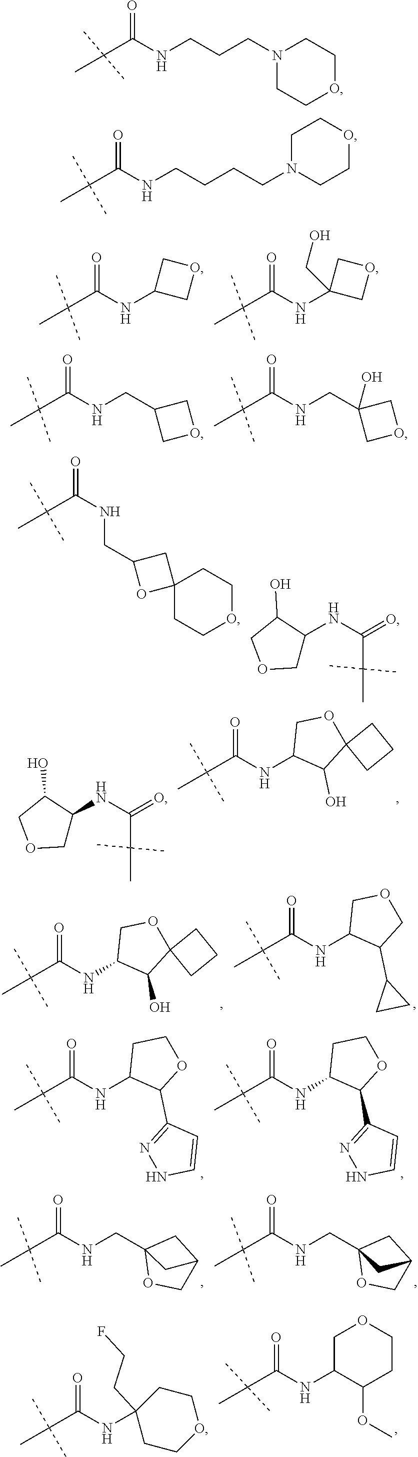

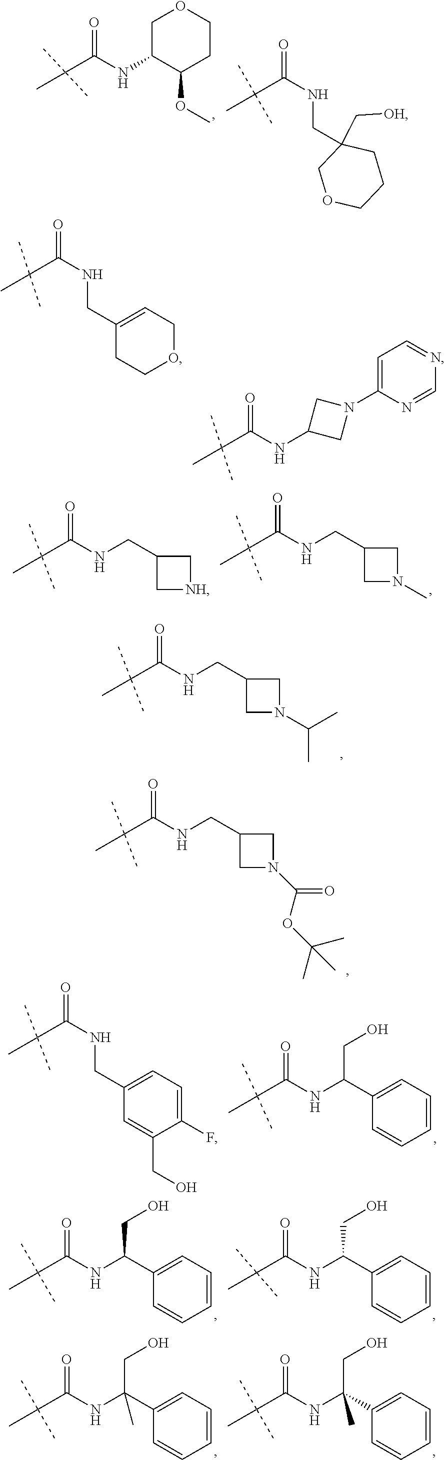

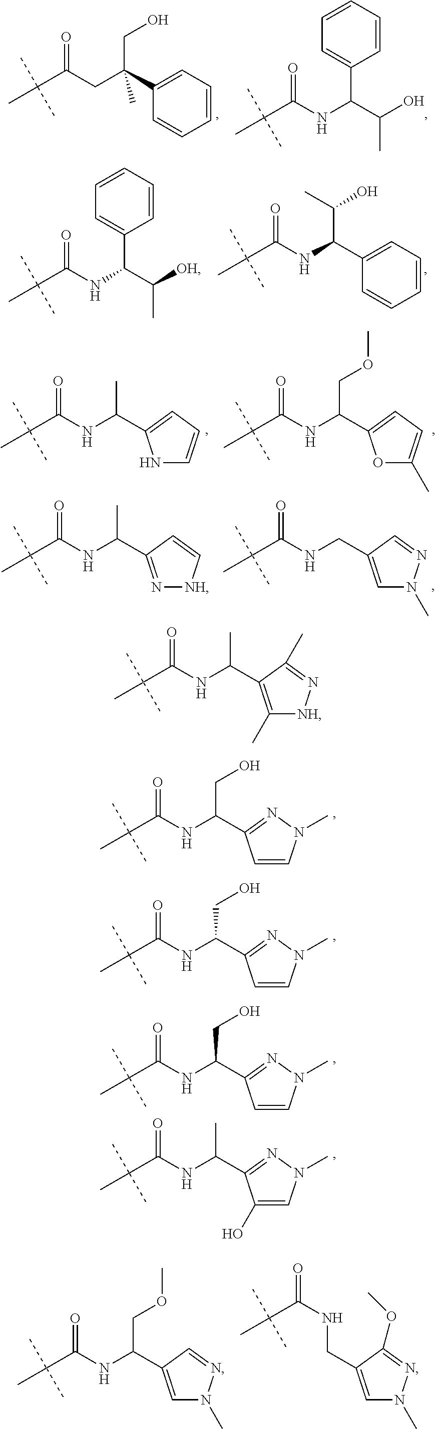

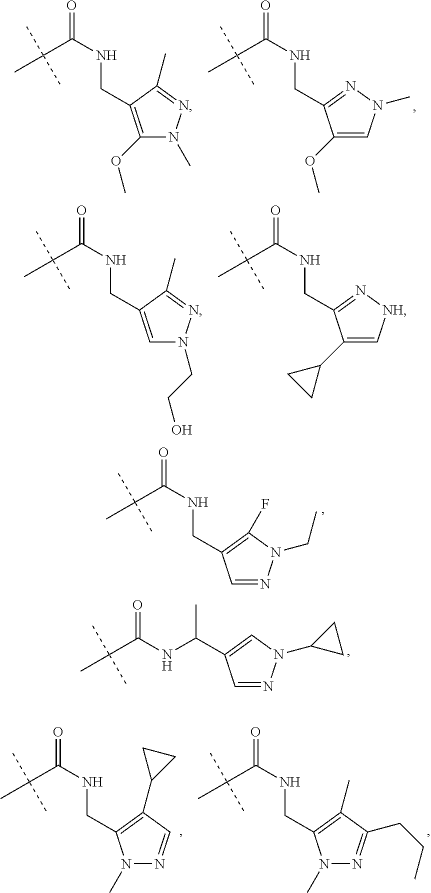

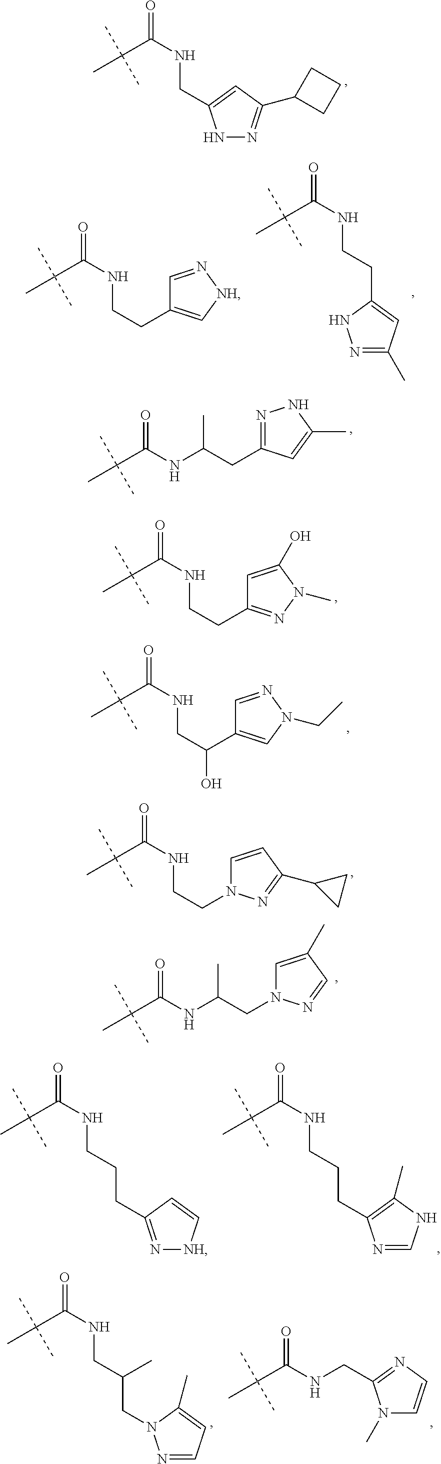

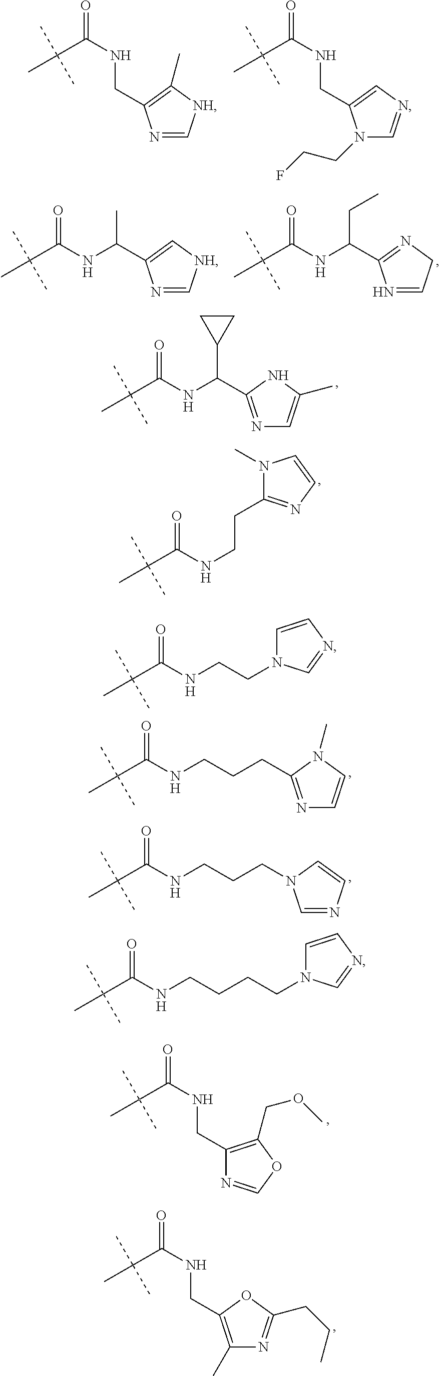

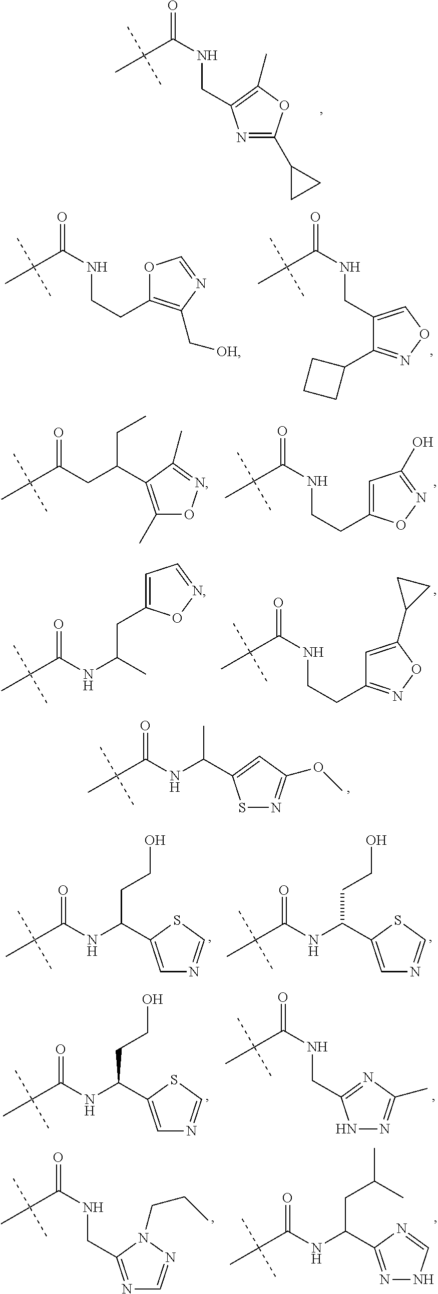

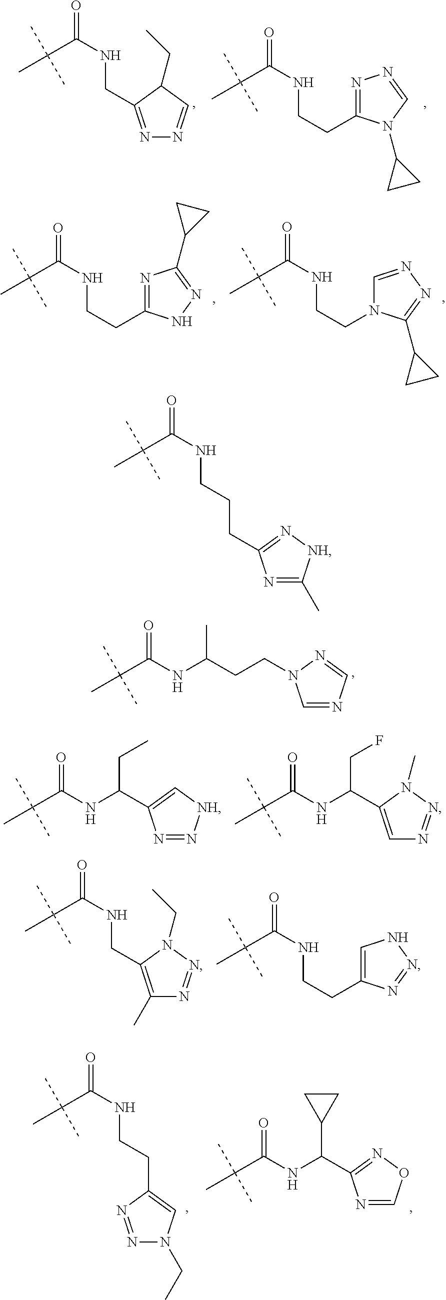

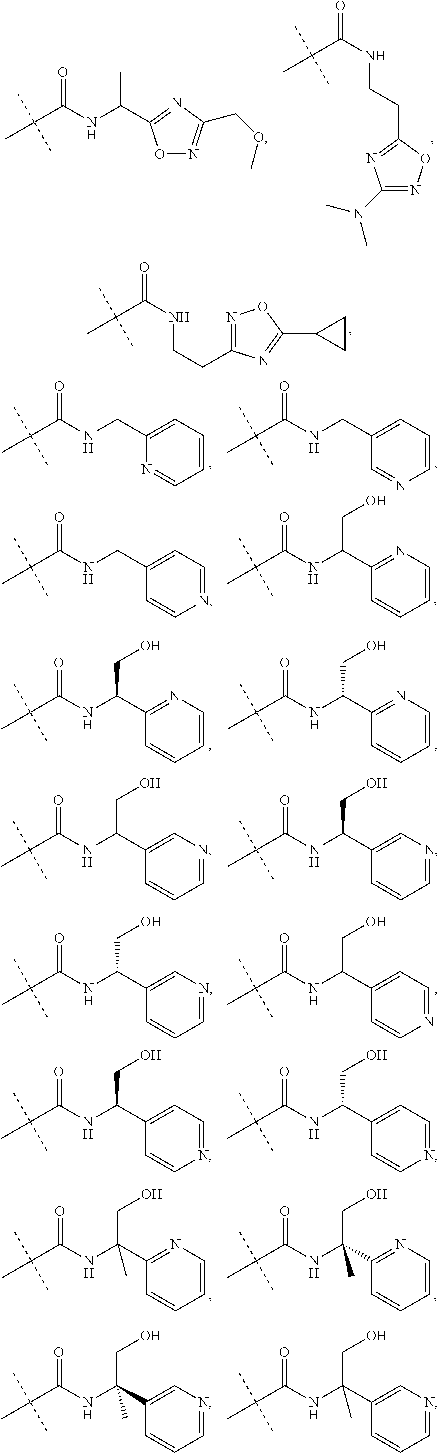

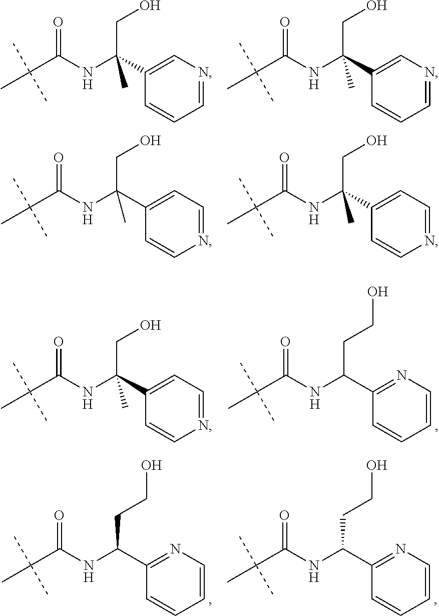

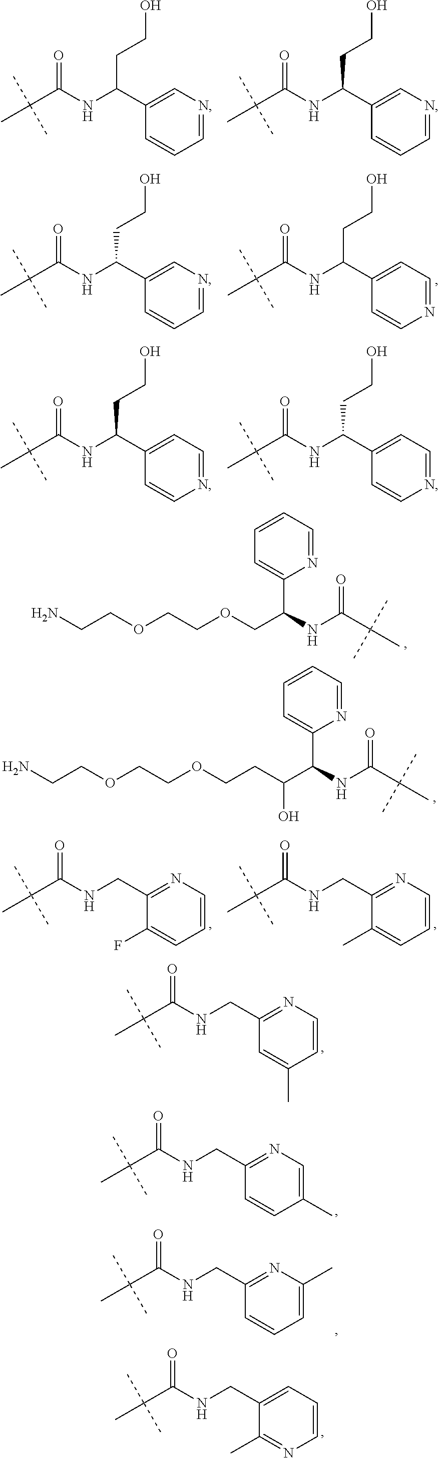

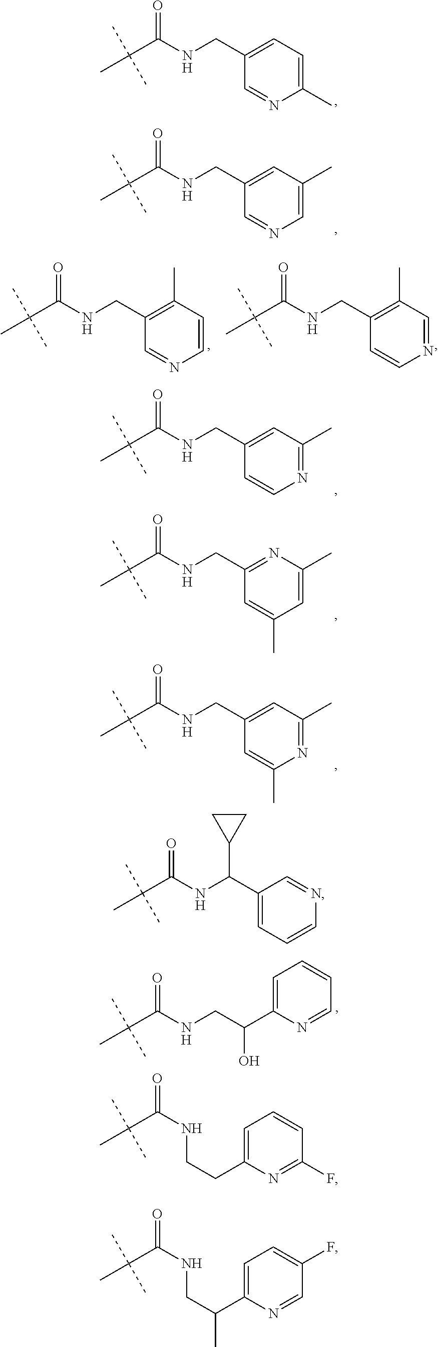

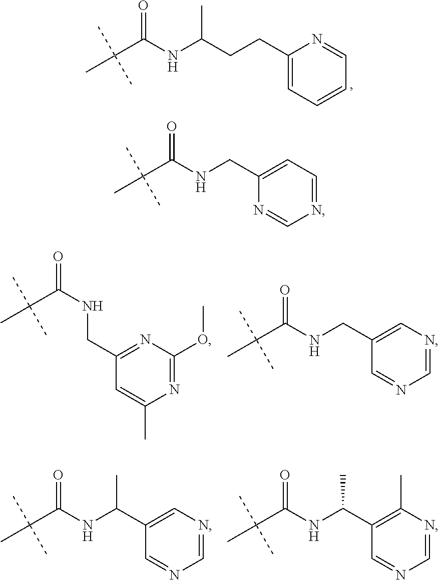

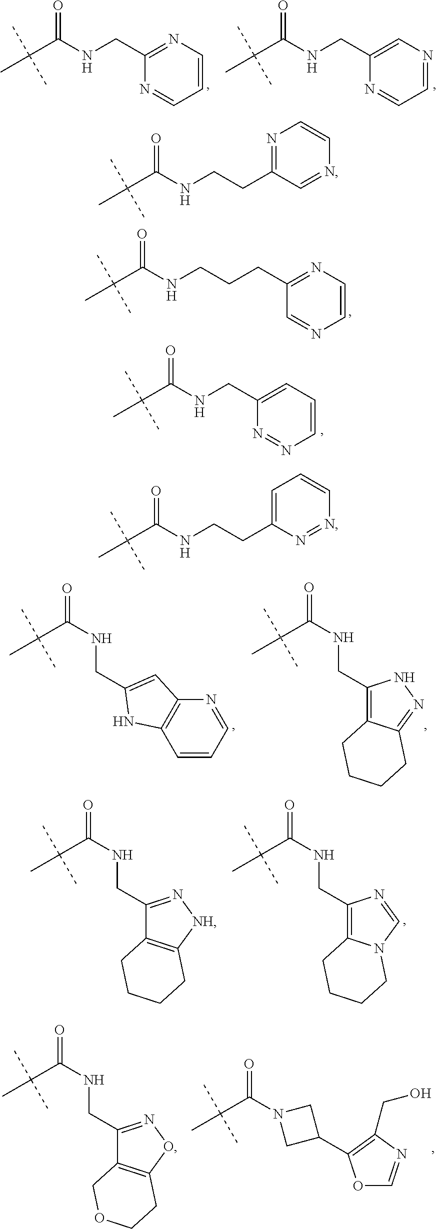

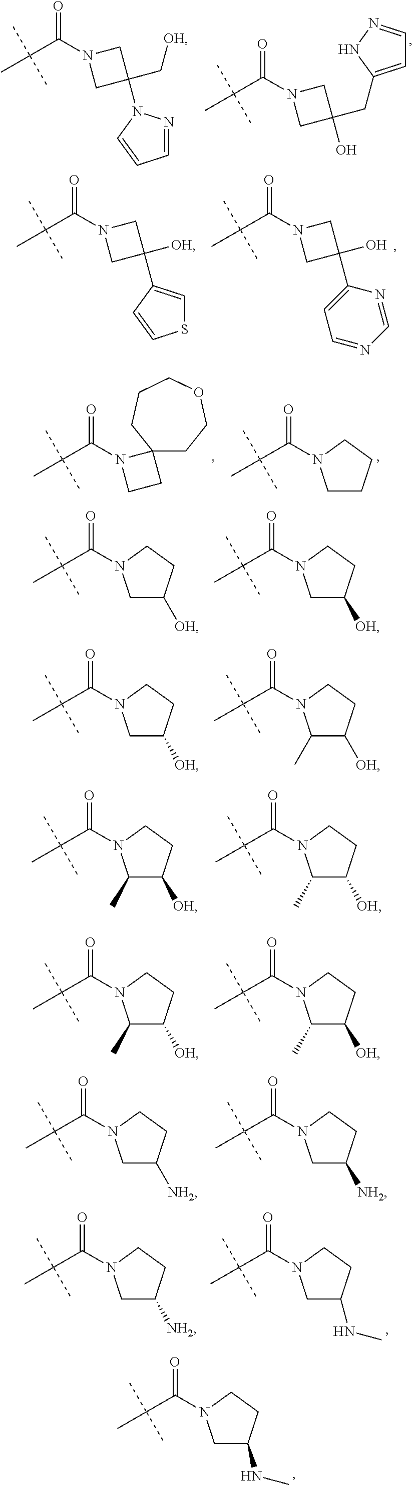

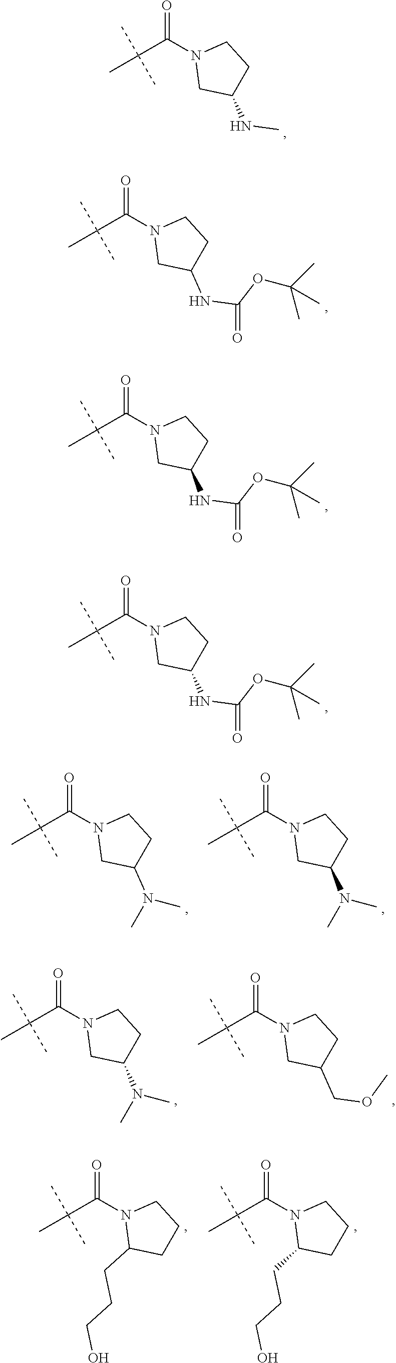

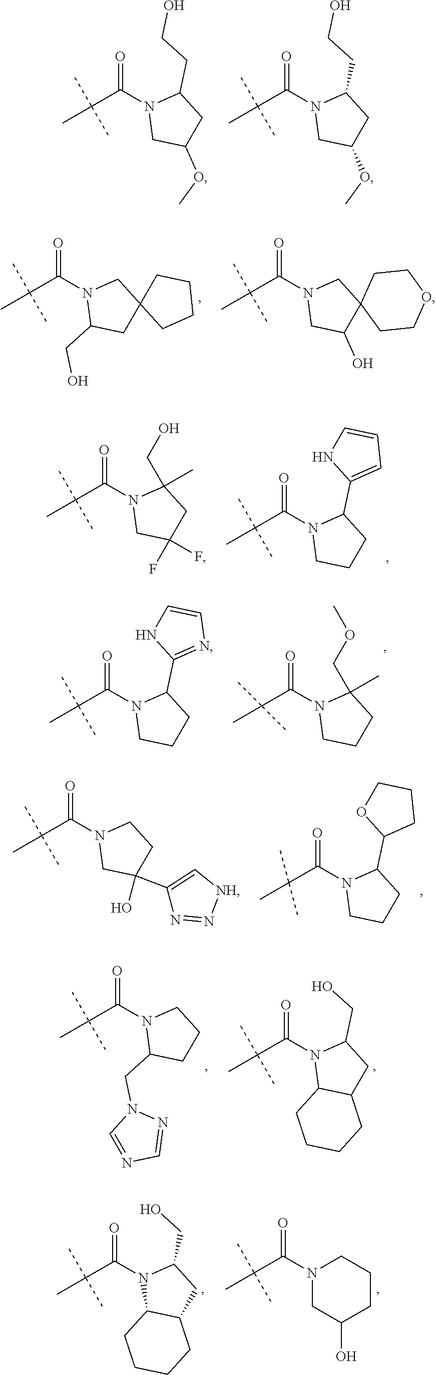

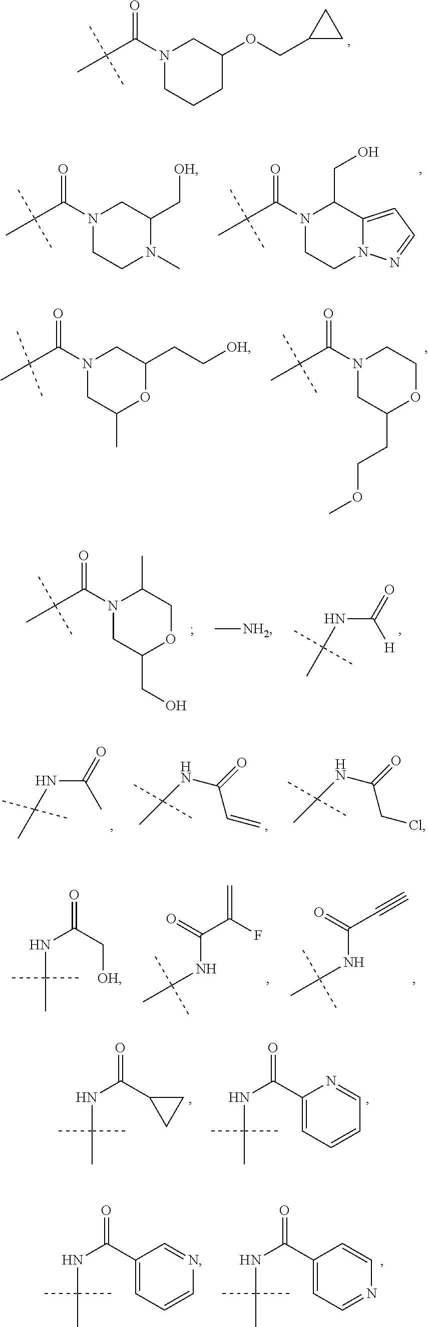

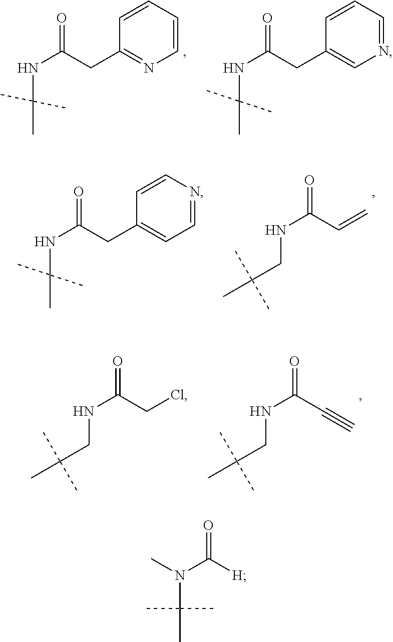

—S—CH₃, —S(═O)—CH₃, —S(═O)₂—CH₃, —S(═O)₂—NH₂,

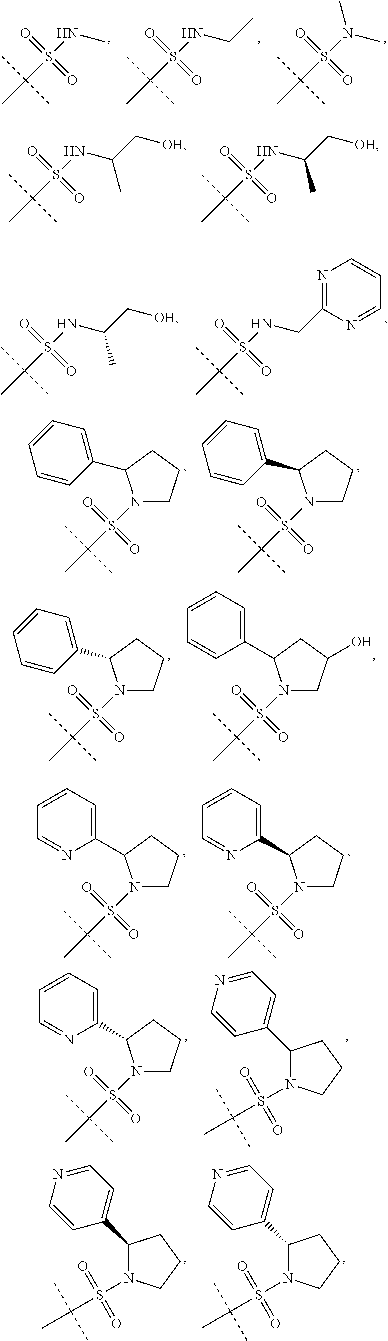

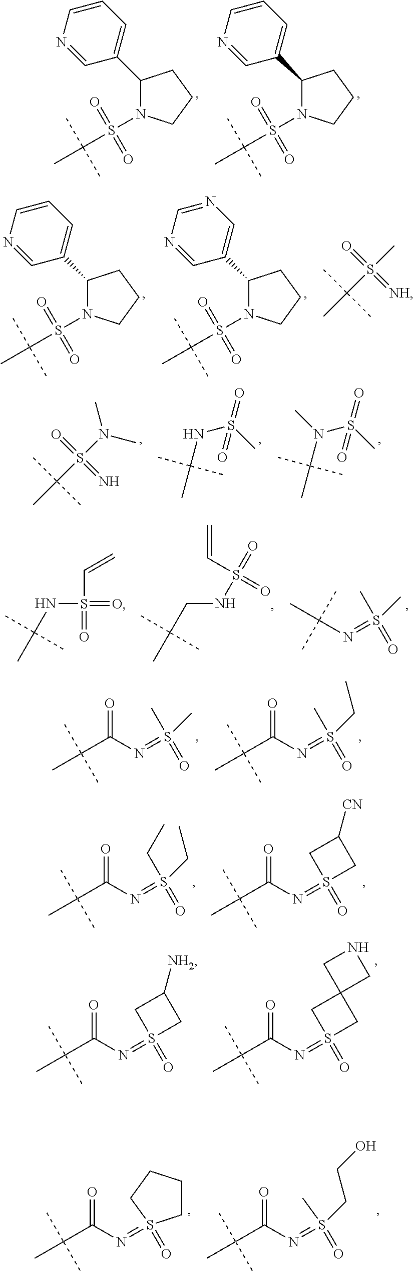

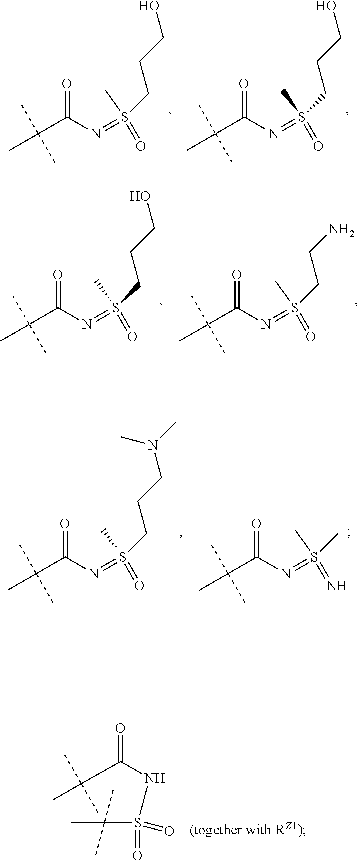

and the remaining radicals and residues are as defined for formula I above or for any of the further particular embodiments described herein above or below.

In a particular embodiment, PE9a, of PE9
the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² is selected from the group consisting of —COOH, —COONa, —COOCH₃,

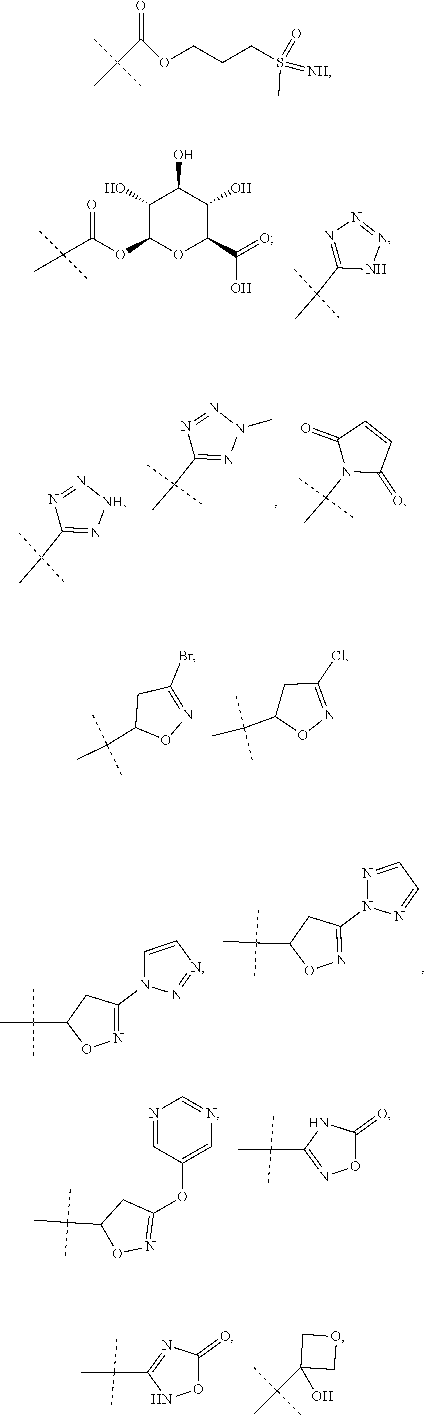

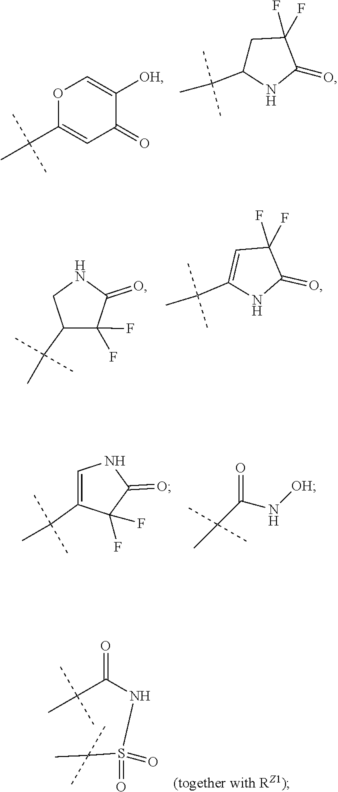

and the remaining radicals and residues are as defined for formula I above or for any of the further particular embodiments described herein above or below.

In a particular embodiment. PE9aa, of PE9a
R² is selected from the group consisting of —COOH and —COONa; in particular —COOH.

In a particular embodiment, PE9b, of PE9
the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² is selected from the group consisting of —C(═O)—NH₂,

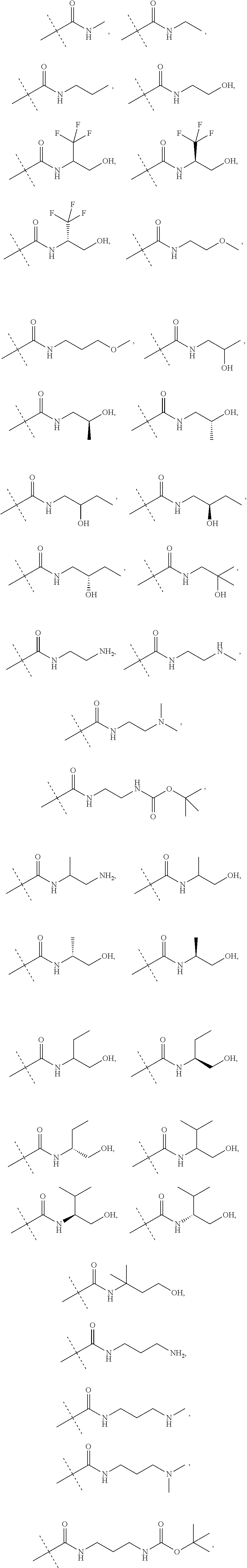

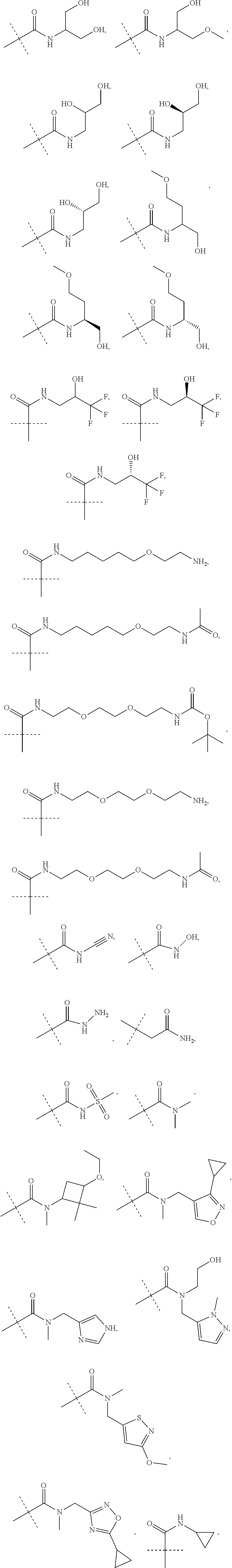

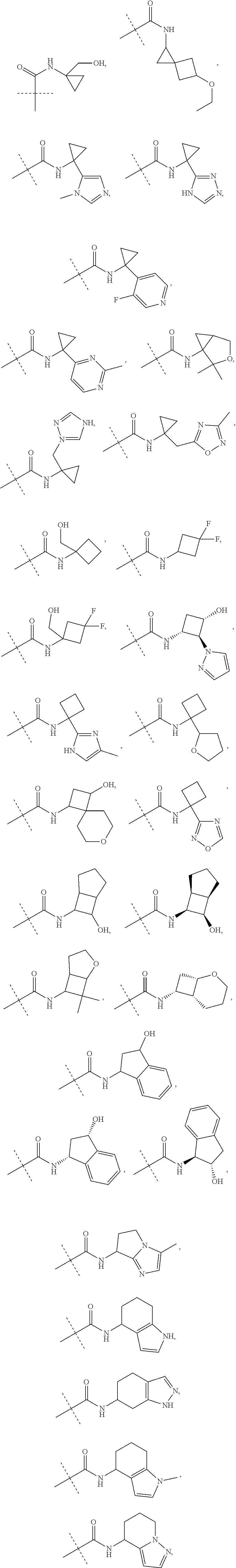

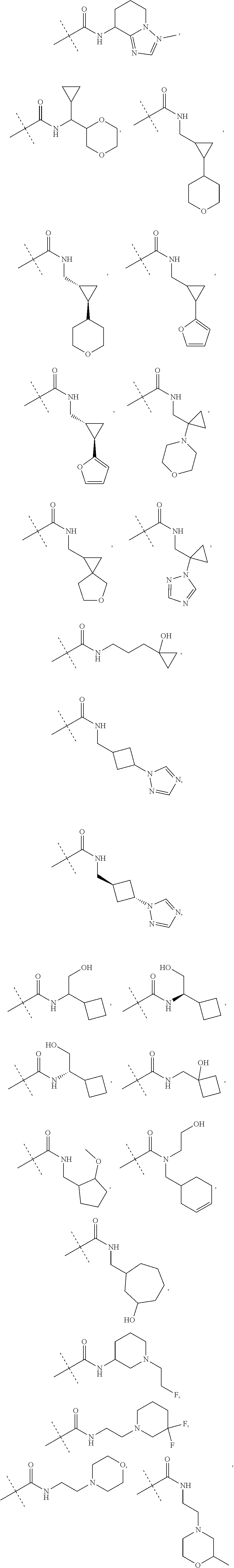

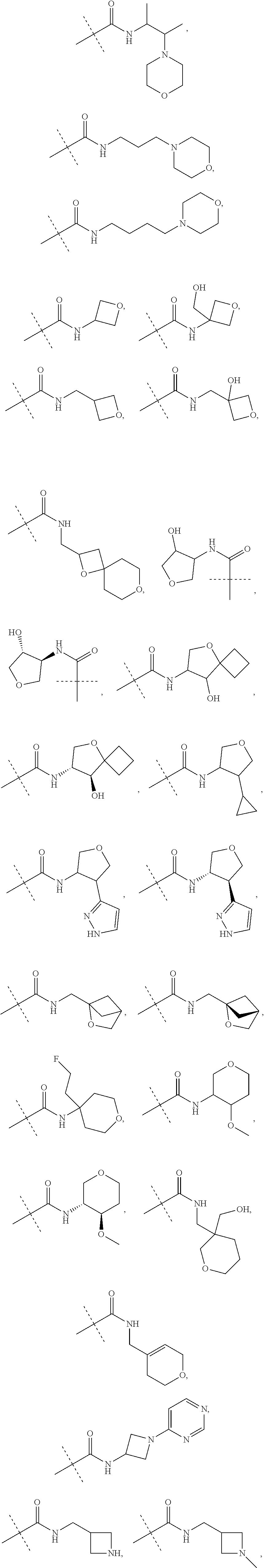

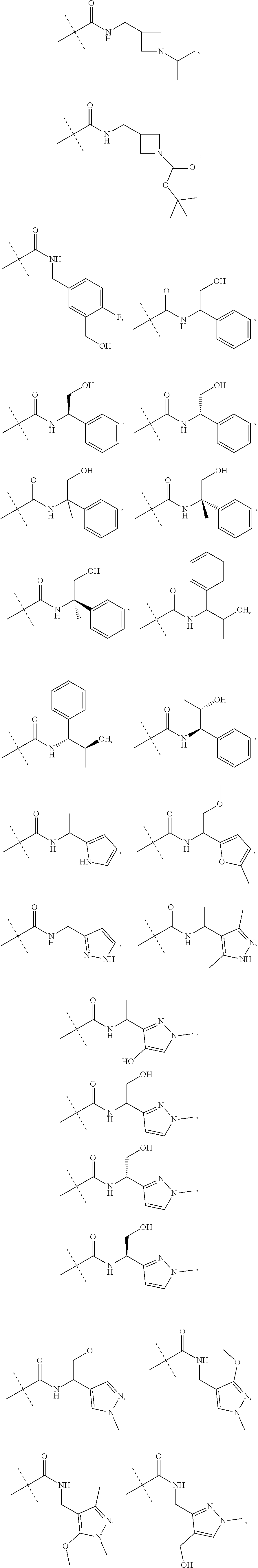

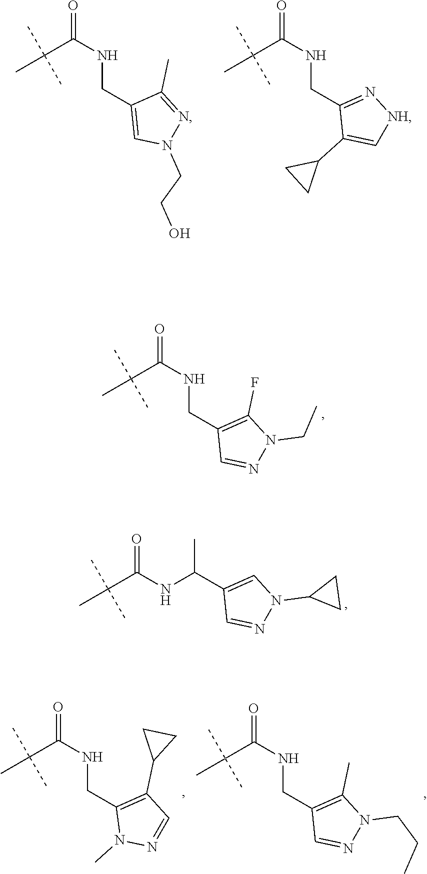

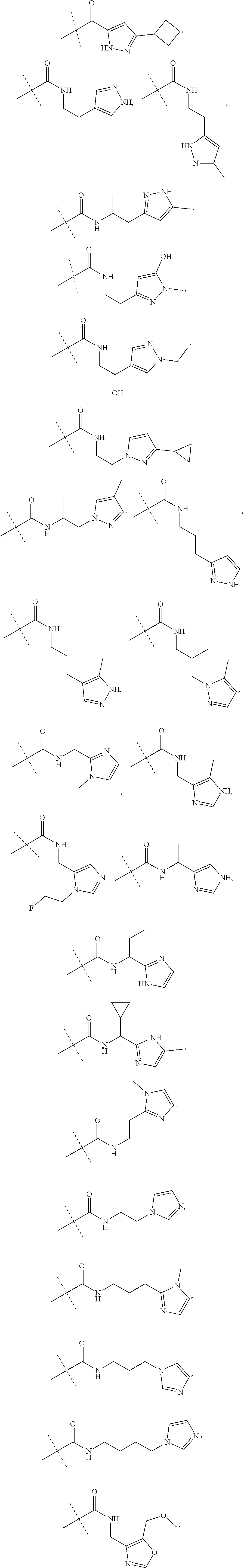

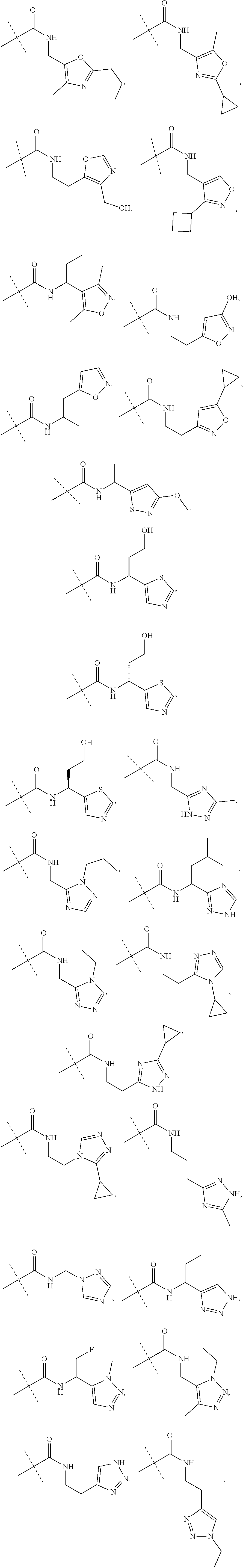

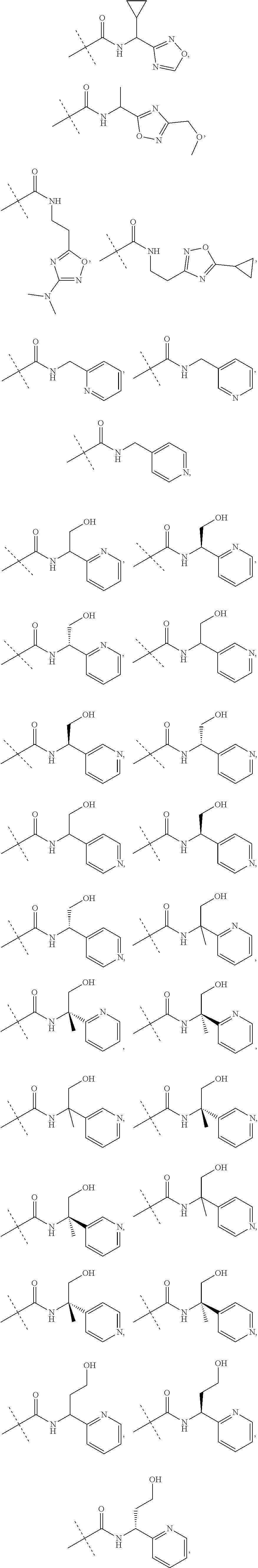

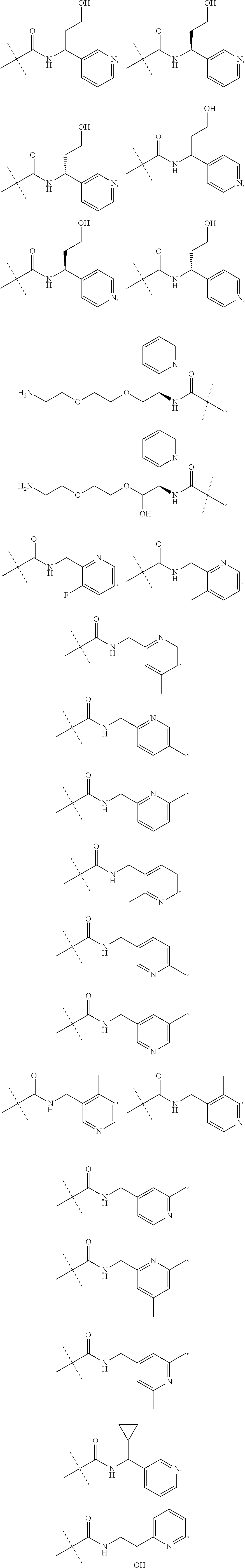

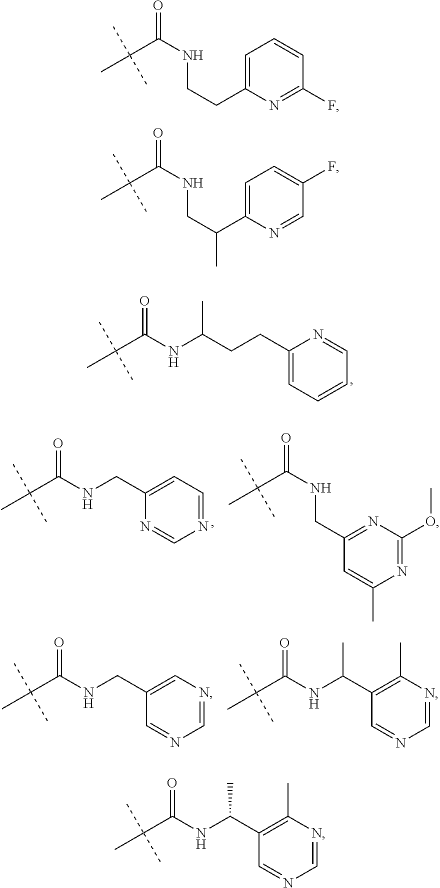

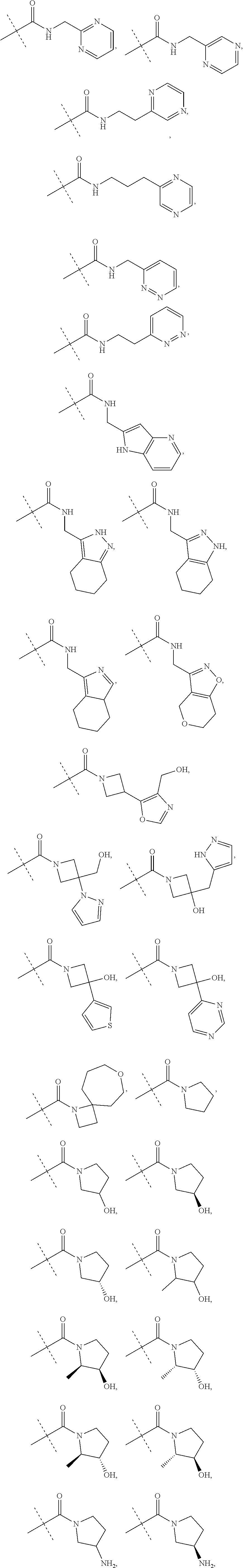

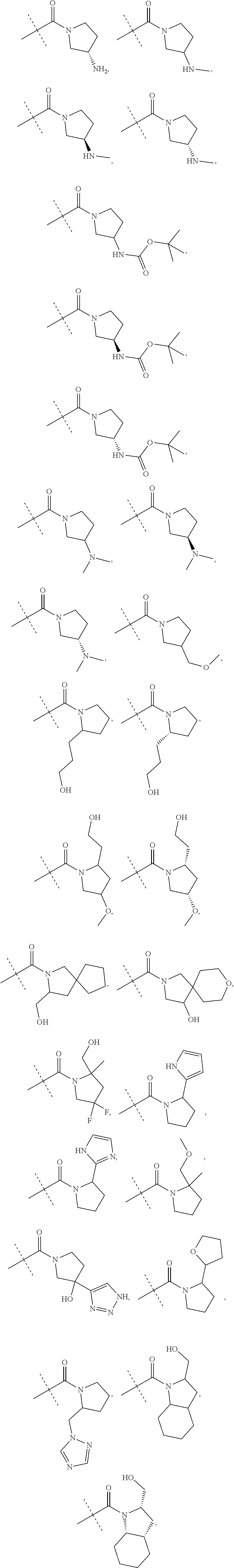

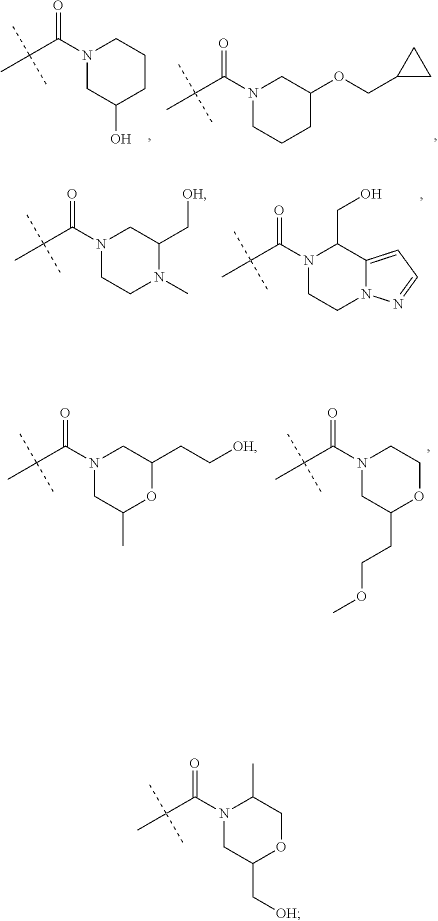

and the remaining radicals and residues are as defined for formula I above or for any of the further particular embodiments described herein above or below.

In a particular embodiment. PE9ba, of PE9b
R² is selected from the group consisting of —C(═O)—NH₂,

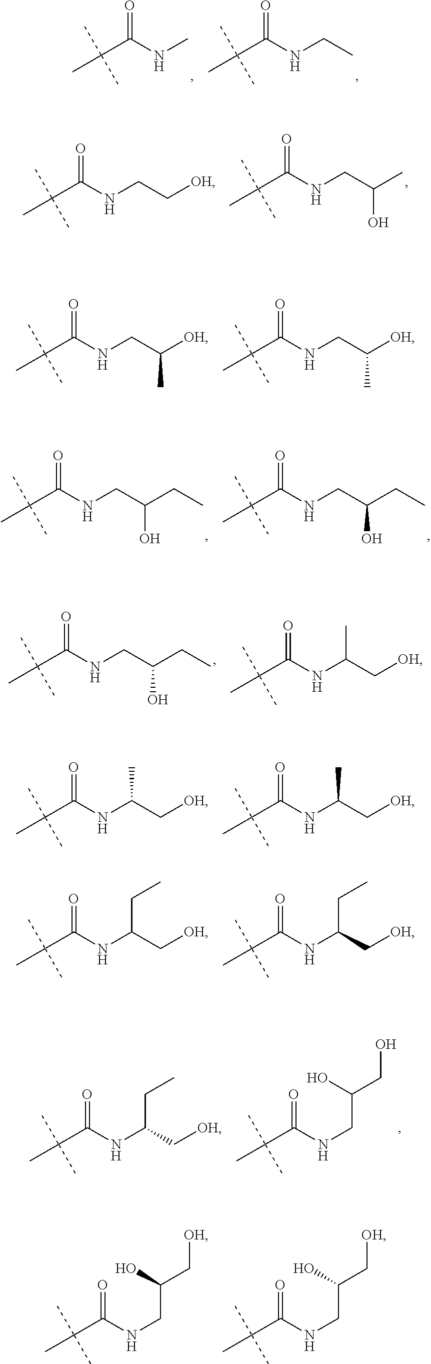

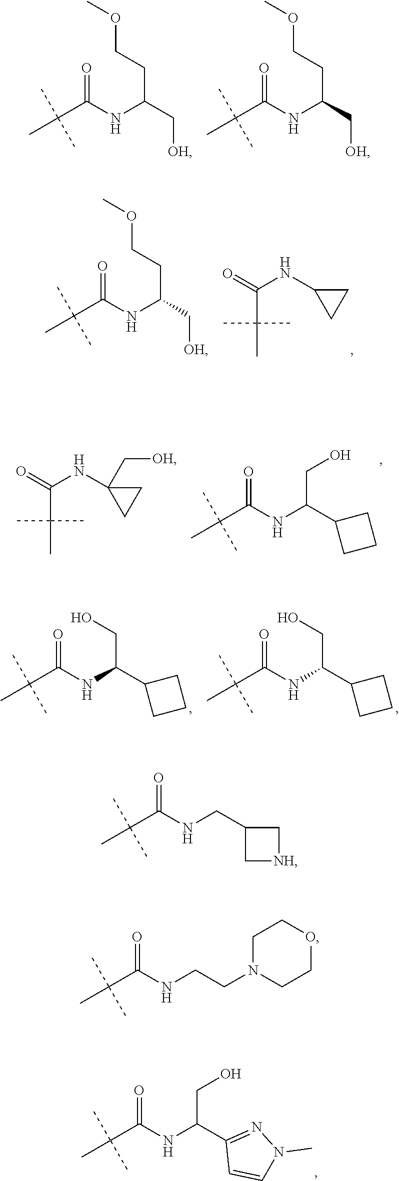

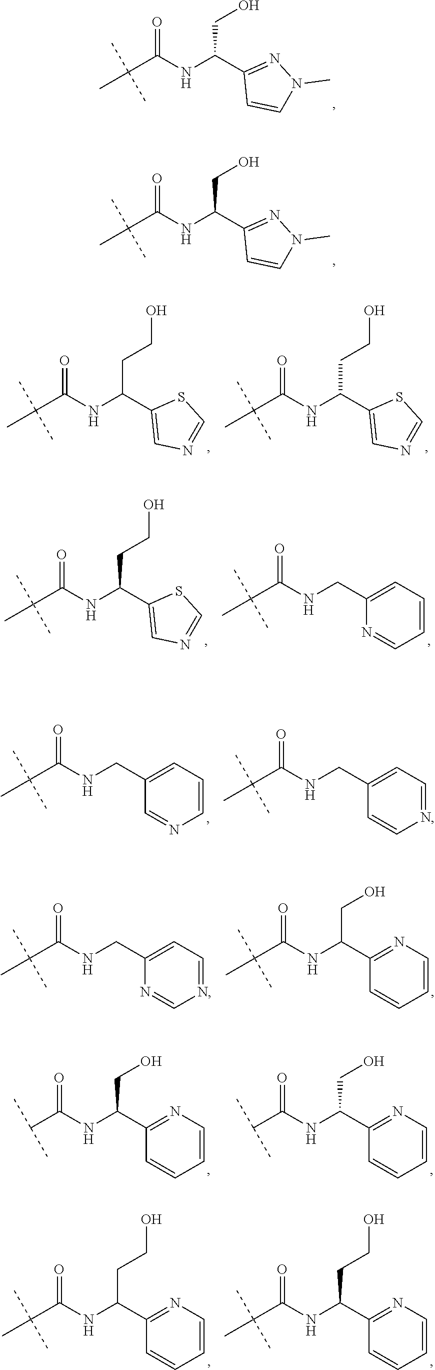

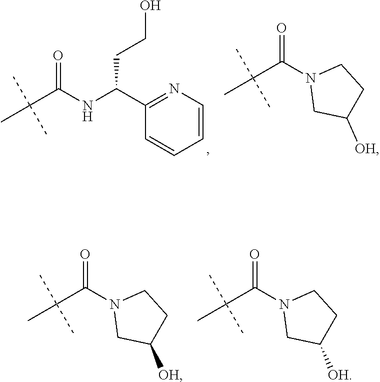

In another particular embodiment, PE9c, of PE9
the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R² is selected from the group consisting of —NH₂,

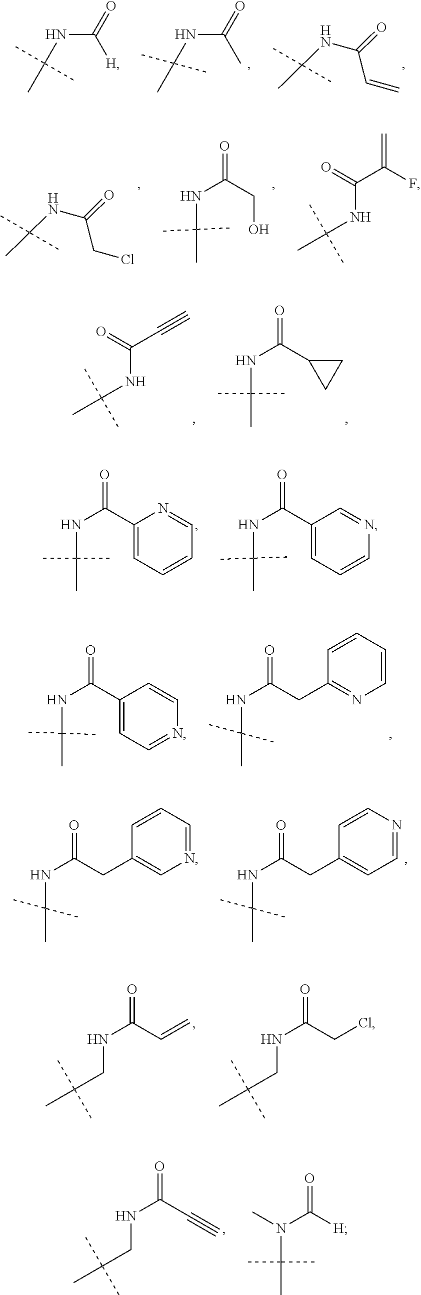

CH₃, —S(═O)—CH₃, —S(═O)₂—CH₃, —S(═O)₂—NH₂,

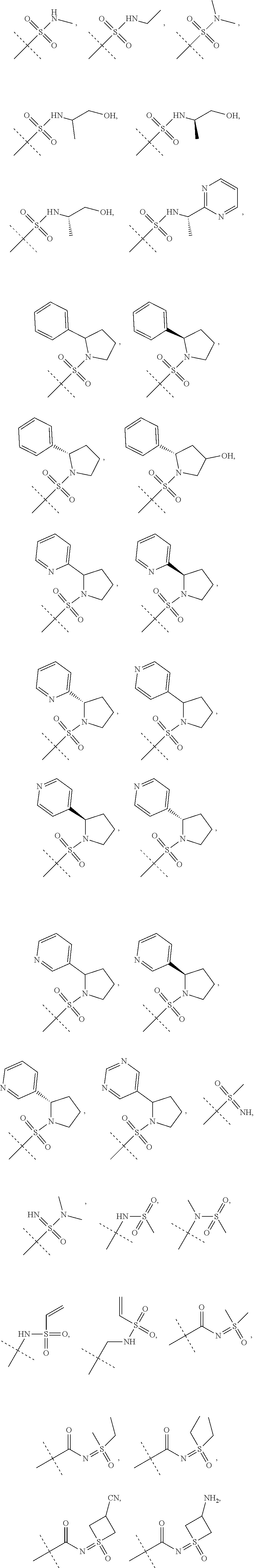

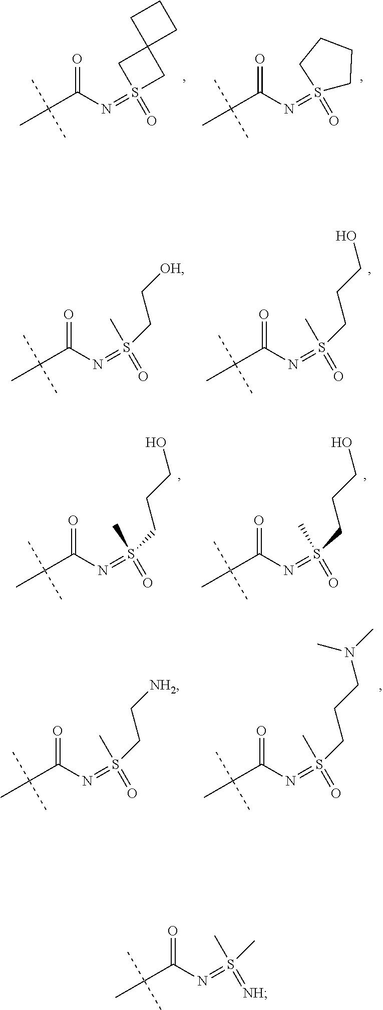

and the remaining radicals and residues are as defined for formula I above or for any of the further particular embodiments described herein above or below.

In a particular embodiment. PE9ca, of PE9c
R² is selected from the group consisting of

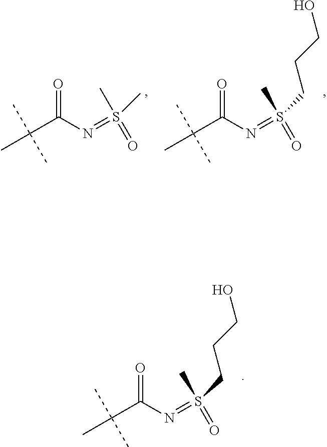

It is understood that in the embodiments PE8, PE8a, PE8aa, PE8ab, PE9, PE9a, PE9aa, PE9b, PE9ba, PE9c, and PE9ca shown above the dotted line

( [symbol] ) is used to indicate the position where the individual radicals R¹ and R², respectively, are attached to the remaining of the molecule, i.e. the compound of formula I.

In still another particular embodiment of the invention, PE10, the compound of the present invention is a tricyclic heterocycle of formula I, or any N-oxide, solvate, tautomer or stereoisomer thereof and/or any pharmaceutically acceptable salt of each of the foregoing, including mixtures thereof in all ratios, wherein
R¹ is selected from the group described for PE8 above; and
R² is selected from the group described for PE9 above;
and the remaining radicals and residues are as defined for formula I above or for any of the further particular embodiments described herein above or below.

It is a particular embodiment, PE10a, of PE10 wherein
R¹ is selected from the group described for PE8a above, especially PE8aa or PE8ab; and
R² is selected from the group described for PE9 above.

It is still another particular embodiment, PE10b, of PE10 wherein
R¹ is selected from the group described for PE8a above, especially PE8aa or PE8ab; and
R² is selected from the group described for PE9a above, especially PE9aa.

It is still another particular embodiment, PE10c, of PE10 wherein
R¹ is selected from the group described for PE8a above, especially PE8aa or PE8ab; and
R² is selected from the group described for PE9b above, especially PE9ba.

It is still another particular embodiment, PE10d, of PE10 wherein
R¹ is selected from the group described for PE8a above, especially PE8aa or PE8ab; and
R² is selected from the group described for PE9c above, especially PE9ca.

It is still another particular embodiment of the invention, PE11, wherein
Ring B is as defined in one of the particular embodiments PE1, PE1a, PE1aa, PE1ab, PE7, PE7a, PE7b; and
R¹ and R² are selected as described for PE10.

In a particular embodiment, PE11a, of PE11, R¹ and R² are selected as described for PE10a. In another particular embodiment, PE11b, of PE11, R¹ and R² are selected as described for PE10b. In yet another particular embodiment, PE11c, of PE11, R¹ and R² are selected as described for PE11c. In still a further particular embodiment, PE11d, of PE11, R¹ and R² are selected as described for PE10d.

It is still another particular embodiment of the invention, PE12, wherein
Ring A is as defined in one of the particular embodiments PE2, PE2a, PE2aa, PE2b, PE2ba, PE2baa, PE7, PE7a, PE7b; and
R¹ and R² are selected as described for PE10.

In a particular embodiment, PE12a, of PE12, R¹ and R² are selected as described for PE10a. In another particular embodiment, PE12b, of PE12, R¹ and R² are selected as described for PE10b. In yet another particular embodiment, PE12c, of PE12, R¹ and R² are selected as described for PE10c. In still a further particular embodiment, PE12d, of PE12, R¹ and R² are selected as described for PE10d.

It is still another particular embodiment of the present invention, PE13, wherein
Ring B is as defined in one of the particular embodiments PE1, PE1a, PE1aa, PE1ab, PE7, PE7a, PE7b;
Ring A is as defined in one of the particular embodiments PE2, PE2a, PE2aa, PE2b, PE2ba, PE2baa, PE7, PE7a, PE7b; and
R¹ and R² are selected as described for PE10; PE10a; PE10b; PE10c; or PE10d.

In a particular embodiment, PE13a, of PE13, R¹ and R² are selected as described for PE10a. In another particular embodiment, PE13b, of PE13, R¹ and R² are selected as described for PE10b. In yet another particular embodiment, PE13c, of PE13, R¹ and R² are selected as described for PE10c. In still a further particular embodiment, PE13d, of PE13, R¹ and R² are selected as described for PE10d.

In still another particular embodiment, PE14, the compound of the present invention is a tricyclic heterocycle selected from the compounds shown in Table 1 and Table 1a below, or any N-oxide, solvate, tautomer or stereoisomer thereof and/or any pharmaceutically acceptable salt of each of the foregoing, including mixtures thereof in all ratios. In yet another particular embodiment, PE14a, of PE14, the compound is selected from Table 1 and Table 1a and is a compound of formula I as described hereinabove and in the claims. It is understood that each single compound depicted in Table 1 and Table 1a as well as any N-oxide, solvate, tautomer or stereoisomer thereof and/or any pharmaceutically acceptable salt of such compound represents a particular embodiment of the present invention. In yet a further particular embodiment, PE14b, of PE14 or PE14a, the compound is selected from Table 1 and Table 1a, is a compound of formula I as described hereinabove and in the claims, and is within Group A in the SK-HEP1 reporter assay and/or within Group A in the NCI-H226 assay as provided in Table 2 below.

As used herein, the following definitions shall apply unless otherwise indicated or defined specifically elsewhere in the description and/or the claims for specific substituents, radicals, residues, groups or moieties.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon or tricyclic hydrocarbon that is completely saturated or that contains one or more units of unsaturation, such as one or more C═C double bond(s) and/or C≡C triple bond(s), but which is not aromatic (also referred to herein as “carbocycle”, “cycloaliphatic” or “cycloalkyl”), that has—in general and if not defined otherwise in this specification or the accompanied claims—a single point of attachment to the rest of the molecule. Unless otherwise specified, aliphatic groups contain 1 to 10 (i.e., 1, 2, 3, 4, 5, 6, 7, 8, 9, or 10) 1 to 8 (i.e., 1, 2, 3, 4, 5, 6, 7, or 8) or 1 to 6 (i.e., 1, 2, 3, 4, 5, or 6) aliphatic carbon atoms (“C₁₋₁₀-aliphatic”, “C₁₋₈-aliphatic” and “C₁₋₆-aliphatic”, respectively). In some embodiments, aliphatic groups contain 1-5 (i.e., 1, 2, 3, 4, or 5) aliphatic carbon atoms (“C₁₋₅-aliphatic”). In other embodiments, aliphatic groups contain 1-4 (i.e., 1, 2, 3, or 4) aliphatic carbon atoms (“C₁₋₄-aliphatic”). In still other embodiments, aliphatic groups contain 1-3 (i.e., 1, 2, or 3) aliphatic carbon atoms (“C₁₋₃-aliphatic”), and in yet other embodiments, aliphatic groups contain 1-2 aliphatic carbon atoms (“C₁₋₂-aliphatic”). In some embodiments, “cycloaliphatic” (“cycloalkyl”) refers to a monocyclic C₃-C₇ hydrocarbon (i.e., a monocyclic hydrocarbon with 3, 4, 5, 6, or 7 ring carbon atoms) or to a bicyclic C₅₋₈ hydrocarbon (i.e. a bicyclic hydrocarbon with 5, 6, 7, or 8 ring carbon atoms) that is completely saturated or that contains one or more units of unsaturation, but which is not aromatic, that has a single point of attachment to the rest of the molecule. In another embodiment the term “cycloaliphatic” or “carbocycle” refers to a monocyclic or bicyclic cycloaliphatic ring system which is fused to an aromatic, heteroaromatic or heterocyclic ring or ring system via 2 adjacent ring atoms of that aromatic, heteroaromatic or heterocyclic ring or ring system; in other words, such carbocycle shares two ring atoms with the ring or ring system to which it is fused thereby having two points of attachment to the rest of the molecule. In another embodiment the term “carbocycle” refers to bicyclic spiro-cycles in which two monocyclic carbocycles are fused to each other via the same single carbon atom. In general, the term “aliphatic” encompasses, to the extent chemically possible, straight-chain, i.e. unbranched, as well as branched hydrocarbon chains, if not defined differently in a particular instance. Also, in general this term encompasses, to the extent chemically possible, unsubstituted and substituted hydrocarbon moieties, if not defined differently in a particular instance. Typical substituents of an aliphatic group include, but are not limited to halogen, in particular F, cyano, hydroxy, alkoxy, unsubstituted or mono- or di-substituted amino, aryl, in particular unsubstituted or substituted phenyl, heteroaryl, in particular unsubstituted or substituted pyridyl or pyrimidinyl, heterocyclyl, in particular unsubstituted or substituted pyrrolidinyl, piperidinyl, piperazinyl or morpholinyl. Suitable aliphatic groups include, but are not limited to, linear or branched, substituted or unsubstituted alkyl, alkenyl, alkynyl, cycloalkyl, cycloalkenyl groups and hybrids thereof as (cycloalkyl)alkyl, (cycloalkenyl)alkyl or (cycloalkyl)alkenyl.

The term “alkyl” usually refers to a saturated aliphatic and acyclic moiety, while the term “alkenyl” usually refers to an unsaturated aliphatic and acyclic moiety with one or more C═C double bonds and the term “alkynyl” usually refers to an aliphatic and acyclic moiety with one or more C≡C triple bonds. It is understood that the term “alkenyl” comprises all forms of isomers, i.e. E-isomers, Z-isomers as well as mixtures thereof (E/Z-isomers). Exemplary aliphatic groups are linear or branched, substituted or unsubstituted C₁₋₁₀-alkyl, C₁₋₈-alkyl, C₁₋₆-alkyl, C₁₋₄-alkyl, C₁₋₃-alkyl, C₁₋₂-alkyl, C₂₋₈-alkenyl, C₂₋₆-alkenyl, C₂₋₈-alkynyl, C₂₋₆-alkynyl groups and hybrids thereof such as (cycloalkyl)alkyl, (cycloalkenyl)alkyl or (cycloalkyl)alkenyl.

In particular, the term “C₁₋₃-alkyl” refers to alkyl groups, i.e. saturated acyclic aliphatic groups, having 1, 2 or 3 carbon atoms. Exemplary C₁₋₃-alkyl groups are methyl, ethyl, propyl and isopropyl. The term “C₁₋₄-alkyl” refers to alkyl groups having 1, 2, 3 or 4 carbon atoms. Exemplary C₁₋₄-alkyl groups are methyl, ethyl, propyl, isopropyl, butyl, isobutyl, and tert-butyl. The term “C₁₋₆-alkyl” refers to alkyl groups having 1, 2, 3, 4, 5 or 6 carbon atoms. Exemplary C₁₋₆-alkyl groups are methyl, ethyl, propyl, isopropyl, butyl, isobutyl, tert-butyl, n-pentyl, 2-pentyl, n-hexyl, and 2-hexyl. The term “C₁₋₈-alkyl” refers to alkyl groups having 1, 2, 3, 4, 5, 6, 7, or 8 carbon atoms. Exemplary C₁₋₈-alkyl groups are methyl, ethyl, propyl, isopropyl, butyl, isobutyl, tert-butyl, n-pentyl, 2-pentyl, n-hexyl, 2-hexyl n-heptyl, 2-heptyl, n-octyl, 2-octyl, and 2,2,4-trimethylpentyl. The term “C₁₋₁₀-alkyl” refers to alkyl groups having 1, 2, 3, 4, 5, 6, 7, 8, 9, or 10 carbon atoms. Exemplary C₁₋₁₀-alkyl groups are methyl, ethyl, propyl, isopropyl, butyl, isobutyl, tert-butyl, n-pentyl, 2-pentyl, n-hexyl, 2-hexyl n-heptyl, 2-heptyl, n-octyl, 2-octyl, 2,2,4-trimethylpentyl, and n-decyl, Each of these alkyl groups may be straight-chain or—except for C₁-alkyl and C₂-alkyl—branched and may be unsubstituted or substituted with 1, 2 or 3 substituents that may be the same or different and may be, if not specified differently elsewhere in this specification and/or the accompanying claims, selected from the group comprising halogen, in particular F, hydroxy, alkoxy, unsubstituted or mono- or di-substituted amino, aryl, in particular unsubstituted or substituted phenyl, heteroaryl, in particular unsubstituted or substituted pyridyl or pyrimidinyl, heterocyclyl, in particular unsubstituted or substituted pyrrolidinyl, piperidinyl, piperazinyl or morpholinyl. Exemplary substituted alkyl groups are difluoromethyl, trifluoromethyl, 2,2,2-trifluoroethyl, hydroxymethyl, 2-hydroxyethyl.

In some instances the C₁₋₃-alkyl, C₁₋₄-alkyl, C₁₋₆-alkyl, C₁₋₈-alkyl, C₁₋₁₀-alkyl groups—both unbranched and branched—may also comprise those residues in which 1 or 2 of non-terminal and non-adjacent —CH₂— (methylene) groups are replaced by —O—, —S— and/or 1 or 2 non-terminal and non-adjacent —CH₂— or —CH— groups are replaced by —NH— or —N—. These replacements yield, for instance, (modified) alkyl groups like —CH₂—CH₂—O—CH₃, —CH₂—CH₂—CH₂—S—CH₃, CH₂—CH₂—NH—CH₂—CH₃, CH₂—CH₂—O—CH₂—CH₂—O—CH₃, CH₂—CH₂—O—CH₂—CH₂—O—CH₂—CH₃, CH₂—CH₂—N(CH₃)—CH₂—CH₃, and the like. Further and/or different replacements of —CH—and —CH₂—groups may be defined for specific alkyl substituents or radicals elsewhere in the description and/or the claims. As described for “unmodified” alkyl groups hereinabove these “modified” alkyl groups may optionally be substituted with 1, 2 or 3 substituents that may be the same or different and may be, if not specified differently elsewhere in this specification and/or the accompanying claims, selected from the group comprising halogen, in particular F, hydroxy, alkoxy, unsubstituted or mono- or di-substituted amino, aryl, in particular unsubstituted or substituted phenyl, heteroaryl, in particular unsubstituted or substituted pyridyl or pyrimidinyl, heterocyclyl, in particular unsubstituted or substituted pyrrolidinyl, piperidinyl, piperazinyl or morpholinyl. Exemplary modified alkyl groups are CH₂—CH₂—O—CH₂—CH₂—O—CH₂—CH₂—NH₂, CH₂—CH₂—O—CH₂—CH₂—O—CH₂—CH₂—NH—C(═O)—CH₃, CH₂—CH₂—O—CH₂—CH₂—O—CH₂—CH₂—NH—C(═O)—OC(CH₃)₃CH₂—CH₂—CH₂—CH₂—CH₂—O—CH₂—CH₂—NH₂, CH₂—CH₂—CH₂—CH₂—CH₂—O—CH₂—CH₂—NH—C(═O)—CH₃, CH₂—CH(OH)—CH₂—CH₂—O—CH₂—CH₂—O—CH₂—CH₂—NH₂, CHR—CH(OH)—CH₂—CH₂—O—CH₂—CH₂—O—CH₂—CH₂—NH₂ wherein “R” denotes another substituent.

The term “C₃₋₇-cycloalkyl” refers to a cycloaliphatic hydrocarbon, as defined above, with 3, 4, 5, 6 or 7 ring carbon atoms. Likewise, the term “C₃₋₆-cycloalkyl” refers to a cycloaliphatic hydrocarbon with 3, 4, 5, or 6 ring carbon atoms. The terms “cycloalkyl”, “C₃₋₇-cycloalkyl” and “C₃₋₆-cycloalkyl” as used herein comprise cyclic hydrocarbons which are saturated or contain one or more units of unsaturation, such as a C═C double bond; such cyclic hydrocarbons having at least one unit of unsaturation may also referred to as “cycloalkenyl” group. C₃₋₇-cycloalkyl groups may be unsubstituted or substituted with—unless specified differently elsewhere in this specification—1, 2 or 3 substituents that may be the same of different and are—unless specified differently elsewhere in this specification—selected from the group comprising C₁₋₆-alkyl, O—C₁₋₆-alkyl (alkoxy), halogen, hydroxy, unsubstituted or mono- or di-substituted amino, aryl, in particular unsubstituted or substituted phenyl. If substituted, C₃₋₇-cycloalkyl comprises all possible stereoisomers. Exemplary C₃₋₇-cycloalkyl groups are cyclopropyl, 2-methyl-cyclopropyl, cyclopropenyl, cyclobutyl, cyclobutenyl, cyclopentyl, cyclopentenyl, cyclohexyl, cyclohexenyl, cycloheptyl, cycloheptenyl. The term “bicyclic C₅₋₈-cycloalkyl” refers to a bicyclic cycloaliphatic hydrocarbon, as defined above, with 5, 6, 7, or 8 ring carbon atoms, it includes spirocyclic ring systems, i.e. ring systems in which the two carbocycles of the bicyclic C₅₋₈-cycloalkyl are attached to each other via the same carbon atom. Bicylic C₅₋₈-cycloalkyl groups may be unsubstituted or substituted with—unless specified differently elsewhere in this specification—1, 2 or 3 substituents that may be the same of different and are—unless specified differently elsewhere in this specification—selected from the group comprising C₁₋₆-alkyl, O—C₁₋₆-alkyl (alkoxy), halogen, hydroxy, unsubstituted or mono- or di-substituted amino. If substituted, bicyclic C₅₋₈-cycloalkyl comprises all possible stereoisomers. Exemplary bicyclic C₅₋₈-cycloalkyl are spiro[3.3]heptanyl, bicyclo[2.2.1]heptan-2-yl, bicyclo[2.2.2]octan-2-yl, bicyclo[2.2.1]hept-5-en-2-ylmethyl, bicyclo[3.1.1]hept-2-en-2-yl.

The term “aliphatoxy” refers to saturated or unsaturated aliphatic groups or substituents as defined above that are connected to another structural moiety via an oxygen atom (—O—). The term “C₁₋₆-aliphatoxy” refers to an aliphatoxy radical with 1, 2, 3, 4, 5, or 6 carbon atoms within the aliphatic group. The term “alkoxy” refers to a particular subgroup of saturated aliphatoxy, i.e. to alkyl substituents and residues that are connected to another structural moiety via an oxygen atom (—O—). Sometimes, it is also referred to as “O-alkyl” and more specifically as “O—C₁₋₂-alkyl”, “O—C₁₋₃-alkyl”, “O—C₁₋₄-alkyl”, “O—C₁₋₆-alkyl”, “O—C₁₋₈-alkyl”. Like the similar alkyl groups, it may be straight-chain or—except for —O—C₁-alkyl and —O—C₂-alkyl—branched and may be unsubstituted or substituted with 1, 2 or 3 substituents that may be the same or different and are, if not specified differently elsewhere in this specification, selected from the group comprising halogen, unsubstituted or mono- or di-substituted amino. Exemplary alkoxy groups are methoxy, fluoromethoxy, difluoromethoxy, trifluoromethoxy, ethoxy, 2,2,2-trifluoroethoxy, n-propoxy, iso-propoxy, n-butoxy, sec-butoxy, tert-butoxy, n-pentoxy.

The term “alkylene” refers to a divalent aliphatic group and in particular a divalent alkyl group. An “alkylene chain” is a polymethylene group, i.e., —(CH₂)ⱼ—, wherein j is a positive integer, preferably 1, 2, 3, 4, 5 or 6. In the context of the present invention “C₁₋₃-alkylene” refers to an alkylene moiety with 1, 2 and 3, respectively, —CH₂—groups; the term “alkylene”, however, not only comprises linear alkylene groups, i.e. “alkylene chains”, but branched alkylene groups as well. The term “C₁₋₆-alkylene” refers to an alkylene moiety that is either linear, i.e. an alkylene chain, or branched and has 1, 2, 3, 4, 5 or 6 carbon atoms. The term “C₂₋₆-alkylene” refers to an alkylene moiety with 2, 3, 4, 5, or 6 carbon atoms, while a “C₃₋₄-alkylene” refers to an alkylene moiety with 3 or 4 carbon atoms and “C₂₋₃-alkylene” refers to an alkylene moiety with 2 or 3 carbon atoms. A substituted alkylene is a group in which one or more methylene hydrogen atoms are replaced by (or with) a substituent. Suitable substituents include those described herein for a substituted alkyl group. In some instances 1 or 2 methylene groups of the alkylene chain may be replaced by, for instance, O, S and/or NH or N—C₁₋₄-alkyl. Exemplary alkylene groups are —CH₂—, —CH₂—CH₂—, —CH₂—CH₂—CH₂—CH₂—, —O—CH₂—CH₂—, —O—CH₂—CH₂—CH₂—, —CH₂—O—CH₂—CH₂—, —O—CH₂—O—, —O—CH₂—CH₂—O—, —O—CH₂—CH₂—CH₂—O—, —CH₂—NH—CH₂—CH₂—, —CH₂—N(CH₃)—CH₂—CH₂—.

The term “alkenylene” refers to a divalent alkenyl group. A substituted alkenylene chain is a polymethylene group containing at least one double bond in which one or more hydrogen atoms are replaced with a substituent. Suitable substituents include those described herein for a substituted aliphatic group. The term “alkenylene” not only refers to straight-chain divalent alkenylene radicals, i.e. an alkenylene chain, but to branched alkenylene groups as well. The term “C₂₋₆-alkenylene” refers to an alkenylene radical having 2, 3, 4, 5, or 6 carbon atoms.

The term “alkynylene” refers to a divalent alkynyl group. A substituted alkynylene chain is a polymethylene group containing at least one triple bond in which one or more hydrogen atoms are replaced with a substituent.

Suitable substituents include those described herein for a substituted aliphatic group.

The term “halogen” means F, Cl, Br, or I.

The term “heteroatom” means one or more of oxygen (O), sulfur (S), or nitrogen (N), including, any oxidized form of nitrogen or sulfur, e.g. N-oxides, sulfoxides and sulfones; the quaternized form of any basic nitrogen or a substitutable nitrogen of a heterocyclic or heteroaromatic ring, for example N (as in 3,4-dihydro-2H-pyrrolyl), NH (as in pyrrolidinyl) or N—SUB with SUB being a suitable substituent (as in N-substituted pyrrolidinyl).

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, that ring members being carbon atoms, wherein at least one ring in the system is aromatic, i.e., it has (4n+2) π (pi) electrons (with n being an integer selected from 0, 1, 2, 3), which electrons are delocalized over the system, and wherein each ring in the system contains three to seven ring members. Preferably, all rings in the aryl system or the entire ring system are aromatic. The term “aryl” is used interchangeably with the term “aryl ring”. In certain embodiments of the present invention, “aryl” refers to an “aromatic ring system”. More specifically, those aromatic ring systems may be mono-, bi- or tricyclic with 5, 6, 7, 8, 9, 10, 11, 12, 13, 14 ring carbon atoms. Even more specifically, those aromatic ring systems may be mono- or bicyclic with 6, 7, 8, 9, 10 ring carbon atoms. Exemplary aryl groups are phenyl, biphenyl, naphthyl, anthracyl and the like, which may be unsubstituted or substituted with one or more identical or different substituents. Also included within the scope of the terms “aryl” or “aromatic ring system”, as they are used herein, is a group in which an aromatic ring is fused to one or more non-aromatic rings, such as indanyl, phthalimidyl, naphthimidyl, phenanthridinyl, or tetrahydronaphthyl, and the like. In the latter case the “aryl” group or substituent is attached to its pendant group via the aromatic part of the ring system.

The term “benzo” refers to a six-membered aromatic ring (with carbon ring atoms) that is fused via two adjacent carbon atoms to another ring, being it a cycloaliphatic, aromatic, heteroaromatic or heterocyclic (heteroaliphatic) ring; as a result a ring system with at least two rings is formed in which the benzo ring shares two common carbon atoms with the other ring to which it is fused. For example, if a benzo ring is fused to a phenyl ring, a napthalene ring system is formed, while fusing a benzo ring to a pyridine provides for either a quinoline or an isoquinoline; fusing a benzo ring to a cyclopentene ring provides an indene ring.

The terms “heteroaryl” and “heteroar-”, used alone or as part of a larger moiety, e.g., “heteroaralkyl”, or “heteroaralkoxy”, refer to groups having 3, 4, 5, 6, 7, 8, 9, 10, 11, 12, 13, 14 ring atoms (which atoms are carbon and hetero atoms), preferably 5, 6, 9 or 10 ring atoms; having 6, 10, or 14 π (pi) electrons shared in a cyclic array; and having, in addition to carbon atoms, 1, 2, 3, 4 or 5 heteroatoms. The term “heteroatom” refers to nitrogen, oxygen, or sulfur, and includes any oxidized form of nitrogen or sulfur, and any quaternized form of a basic nitrogen. In other words, a “heteroaryl” ring or ring system (or a heteroaromatic ring or ring system) may also be described as an aromatic heterocycle. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, furazanyl, pyridyl (pyridinyl), pyridazinyl, pyrimidinyl, pyrazinyl, indolizinyl, purinyl, naphthyridinyl, pteridinyl, and pyrrolopyridinyl, in particular pyrrolo[2,3-b]pyridinyl. The terms “heteroaryl” and “heteroar-”, as used herein, also include groups in which a heteroaromatic ring is fused to one or more aryl, cycloaliphatic, or heterocyclyl rings, where the radical or point of attachment is preferably on the heteroaromatic or, if present, the aryl ring. Nonlimiting examples include indolyl, isoindolyl, benzothienyl (benzothiophenyl), benzofuranyl, dibenzofuranyl, indazolyl, benzimidazolyl, benzothiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, 9H-carbazolyl, dibenzofuranyl and pyrido[2,3-b]-1,4-oxazin-3(4H)-one. For example, an indolyl ring may be attached via one of the ring atoms of the six-membered aryl ring or via one of the ring atoms of the five-membered heteroaryl ring. A heteroaryl group is optionally mono-, bi- or tricyclic. The term “heteroaryl” is used interchangeably with the terms “heteroaryl ring”, “heteroaryl group”, or “heteroaromatic”, any of which terms include rings that are unsubstituted or substituted with one or more identical or different substituents. The term “heteroaralkyl” refers to an alkyl group substituted by a heteroaryl, wherein the alkyl and heteroaryl portions independently are optionally substituted.

A heteroaryl ring can be attached to its pendant group at any of its hetero or carbon ring atoms which attachment results in a stable structure or molecule: any of the ring atoms may be unsubstituted or substituted.

The structures of typical examples of “heteroaryl” substituents as used in the present invention are depicted below:

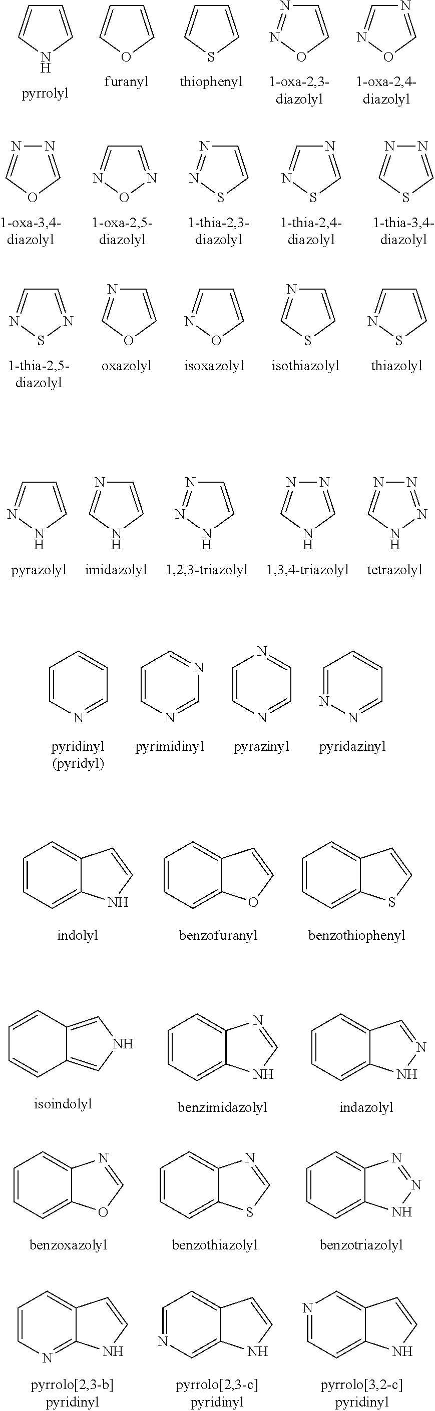

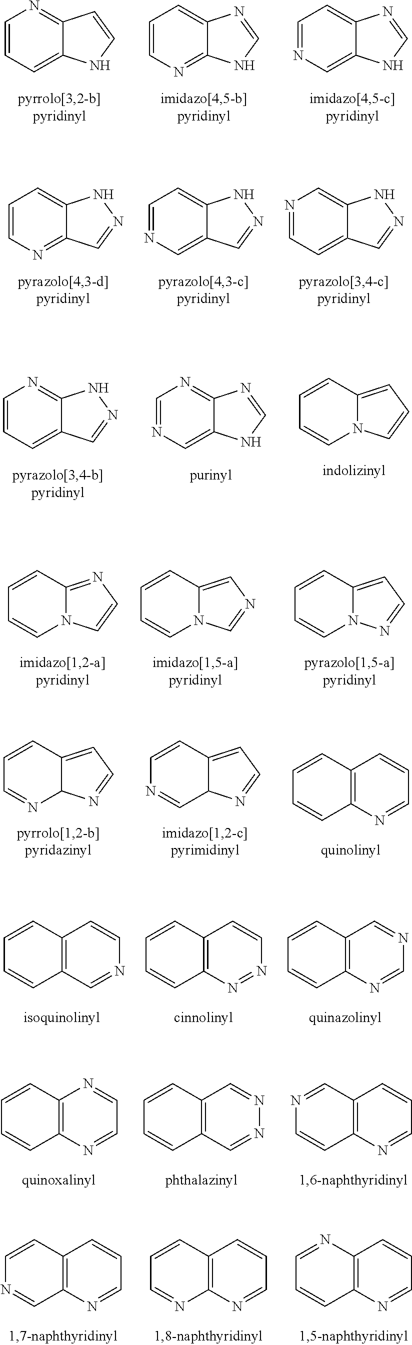

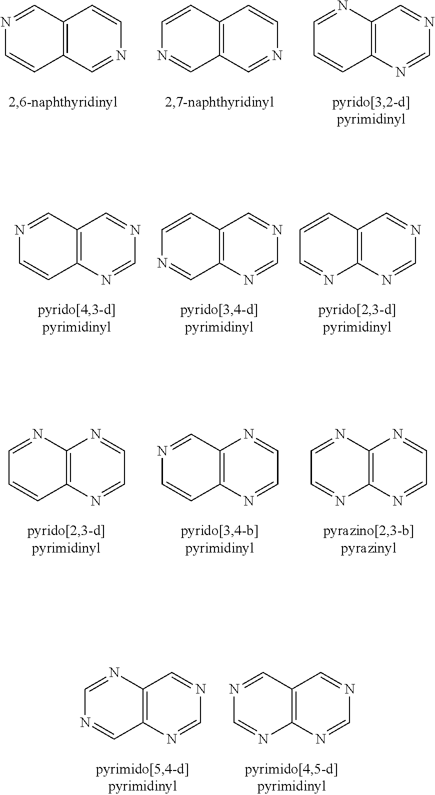

Those heteroaryl substituents can be attached to any pendant group via any of its ring atoms suitable for such an attachment.

As used herein, the terms “heterocycle”, “heterocyclyl”, “heterocyclic radical”, and “heterocyclic ring” are used interchangeably and refer to a stable mono- bi- or tricyclic heterocyclic moiety with 5, 6, 7, 8, 9, 10, 11, 12, 13, 14 ring atoms wherein 1, 2, 3, 4, 5 of said ring atoms are hetero atoms and wherein that heterocyclic moiety is either saturated or partially unsaturated; heterocyclic moieties that are aromatic rings or ring systems are usually referred to as “heteroaryl” moieties as described hereinabove. Preferably, the heterocycle is a stable saturated or partially unsaturated 3-, 4-, 5-, 6-, or 7-membered monocyclic or 7-, 8-, 9-, 10-, or 11-membered bicyclic or 11-, 12-, 13-, or 14-membered tricyclic heterocyclic moiety.

When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen is N (as in 3,4-dihydro-2H-pyrrolyl), NH (as in pyrrolidinyl), or N—SUB with SUB being a suitable substituent (as in N-substituted pyrrolidinyl).

In the context of the term “heterocycle” the term “saturated” refers to a completely saturated heterocyclic system, like pyrrolidinyl, piperidinyl, morpholinyl, piperidinonyl, tetrahydrofuranyl, thianyl, and dioxothianyl. With regard to the term “heterocycle” the term “partially unsaturated” refers to heterocyclic systems (i) that contain one or more units of unsaturation, e.g. a C═C or a C=Heteroatom bond, but that are not aromatic, for instance, tetrahydropyridinyl; or (ii) in which a (saturated or unsaturated but non-aromatic) heterocyclic ring is fused with an aromatic or heteroaromatic ring system, wherein, however, the “partially unsaturated heterocycle” is attached to the rest of the molecule (its pendant group) via one of the ring atoms of the “heterocyclic” part of the system and not via the aromatic or heteroaromatic part. This first class (i) of “partially unsaturated” heterocycles may also be referred to as “non-aromatic partially unsaturated” heterocycles. This second class (ii) of “partially unsaturated” heterocycles may also be referred to as (bicyclic or tricyclic) “partially aromatic” heterocycles indicating that at least one of the rings of that heterocycle is a saturated or unsaturated but non-aromatic heterocycle that is fused with at least one aromatic or heteroaromatic ring system. Typical examples of these “partially aromatic” heterocycles are 1,2,3,4-tetrahydroquinolinyl and 1,2,3,4-tetrahydroisoquinolinyl.

A heterocyclic ring can be attached to its pendant group at any heteroatom or carbon atom that results in a stable structure and any of the ring atoms may be unsubstituted or substituted. Examples of such saturated or partially unsaturated heterocyclic radicals include, without limitation, tetrahydrofuranyl, tetrahydropyranyl, thianyl, dioxothianyl, tetrahydrothiophenyl, pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono-, bi- or tricyclic. The term “heterocyclylalkyl” refers to an alkyl group substituted by a heterocyclyl, wherein the alkyl and heterocyclyl portions independently are unsubstituted or substituted.

The term “bioisostere”, if used alone or in combination with other terms, e.g., “bioisostere radical”, refers to a compound or a group, radical, moiety, substituent and the like, that elicits a similar biological effect as another compound, group, radical, moiety or substituent though they are structurally different to each other. In a broader sense, “bioisosteres” can be understood as compounds or groups that possess near-equal molecular shapes and volumes, approximately the same distribution of electrons, and which exhibit similar physical properties. Typical examples for bioisosteres are carboxylic acid bioisosteres which exhibit similar physico-chemical properties as a carboxylic acid group (“carboxylic acid bioisostere”). Such a carboxylic acid bioisostere group or radical may be utilized in place of a carboxylic acid group or radical thereby providing properties similar to those of the carboxylic group but potentially exhibiting some different properties when compared to the carboxylic acid group, for instance, reduced polarity, increased lipophilicity, or enhanced pharmacokinetic properties. Typical examples of carboxylic acid bioisosteres include, without being limited to, —CN, fluoro, amides, sulfonamides, sulfonimides, and several aromatic and non-aromatic heterocycles such as hydroxy-substituted isoxazoles, sulfonamido-substituted oxadiazoles and oxo-oxadiazoles, e.g., 5-oxo-2,5-dihydro-1,2,4-oxadiazol, and in particular tetrazoles, e.g. 1H-1,2,3,4-tetrazole, 2-methyl-2H-1,2,3,4-tetrazole.

The term “unsaturated”, as used herein, means that a moiety or group or substituent has one or more units of unsaturation.

As used herein with reference to any rings, ring systems, ring moieties, and the like, the term “partially unsaturated” refers to a ring moiety that includes at least one double or triple bond. The term “partially unsaturated” is intended to encompass rings having multiple sites of unsaturation. In particular, it encompasses (i) non-saturated (mono-, bi- or tricyclic) ring systems without any aromatic or heteroaromatic moiety or part; and (ii) bi- or tricyclic ring systems in which one of the rings of that system is an aromatic or heteroaromatic ring which is fused with another ring that is neither an aromatic nor a heteroaromatic ring, e.g. tetrahydronaphthyl or tetrahydroquinolinyl. The first class (i) of “partially unsaturated” rings, ring systems, ring moieties may also be referred to as “non-aromatic partially unsaturated” rings, ring systems, ring moieties, while the second class (ii) may be referred to as “partially aromatic” rings, ring systems, ring moieties.

As used herein, the term “bicyclic”, “bicyclic ring” or “bicyclic ring system” refers to any bicyclic ring system, i.e. carbocyclic or heterocyclic, saturated or having one or more units of unsaturation, i.e. being partially unsaturated or aromatic, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. Likewise, the term “tricyclic”, “tricyclic ring” or “tricyclic ring system” refers to any tricyclic ring system, i.e. carbocyclic or heterocyclic, saturated or having one or more units of unsaturation, i.e. being partially unsaturated or aromatic, in which a bicyclic ring system (as defined above) is fused with another, third ring. Thus, the term includes any permissible ring fusion. As used herein, the term “heterotricyclic” is a subset of “tricyclic” that requires that one or more heteroatoms are present in one or both rings of the tricycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a tricyclic group has 10-14 ring members and 0-5 heteroatoms independently selected from nitrogen, oxygen, or sulfur.

As described herein, certain compounds of the invention contain “substituted” or “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure. Unless otherwise indicated, a “substituted” or “optionally substituted” group has a suitable substituent at each substitutable position of the group, and when more than one position in any given structure is substituted with more than one substituent selected from a specified group, the substituent is either the same or different at every position. If a certain group, substituent, moiety or radical is “mono-substituted”, it bears one (1) substituent. If it is “di-substituted”, it bears two (2) substituents, being either the same or different; if it is “tri-substituted”, it bears three (3) substituents, wherein all three are the same or two are the same and the third is different or all three are different from each other. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

If not specified otherwise elsewhere in the specification or the accompanying claims it is understood that each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with one or more R^{∘}; —(CH₂)₀₋₄O(CH₂)₀₋₁Ph which may be substituted with one or more R^{∘}; —CH═CHPh, which may be substituted with one or more R^{∘}; —(CH₂)₀₋₄O(CH₂)₀₋₁-pyridyl which may be substituted with one or more R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}2; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}2; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂. It is understood that “Ph” means phenyl; and that “—(CH₂)₀₋₄” means that there is either no alkylene group if the subscript is “0” (zero) or an alkylene group with 1, 2, 3 or 4 CH₂ units.

Each R^{∘} is independently hydrogen, halogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{∘}₂, —NO₂, —SiR^{∘}₃, —OSiR^{∘}₃, C(O)SR^{●}, —(C₁₋₄ straight or branched alkylene)C(O)OR^{●}, or —SSR^{●}. It is understood that “Ph” means phenyl; “halo” means halogen; and “—(CH₂)₀₋₂” means that there is either no alkylene group if the subscript is “0” (zero) or an alkylene group with 1 or 2 CH₂ units.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, (haloR^{●}), OH, —OR^{●}, —O(haloR^{●}), —CN, —C(O)OH, —C(O)OR^{●}, —NH₂, —NHR^{●}, —NR^{∘}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{∘}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens. It is understood that “Ph” means phenyl; and “halo” means halogen.

The term “solvates” means addition forms of the compounds of the present invention with solvents, preferably pharmaceutically acceptable solvents that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, e.g. a hemi-, mono- or dihydrate. If the solvent is alcohol, the solvate formed is an alcoholate, e.g., a methanolate or ethanolate. If the solvent is an ether, the solvate formed is an etherate, e.g., diethyl etherate.

The term “N-oxides” means such compounds of the present invention that contain an amine oxide moiety, i.e. the oxide of a tertiary amine group.

The compounds of formula I may—also depending on the nature of substituents they may bear—have one or more centers of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers” for the purpose of the present invention, of these compounds. Since the pharmaceutical activity of the racemates or stereoisomers of the compounds according to the invention may differ, it may be desirable to use a specific stereoisomer, e.g. one specific enantiomer or diastereomer. In these cases, a compound according to the present invention obtained as a racemate or even intermediates thereof—may be separated into the stereoisomeric (enantiomeric, diastereoisomeric) compounds by chemical or physical measures known to the person skilled in the art. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes. In the context of the present invention the term “pure enantiomer” usually refers to a relative purity of one enantiomer over the other (its antipode) of equal to or greater than 95%, preferably >98%, more preferably 98.5%, still more preferably 99%.

Thus, for example, the compounds of the invention which have one or more centers of chirality and which occur as racemates or as mixtures of enantiomers or diastereoisomers can be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical.

In the context of the present invention the term “tautomer” refers to compounds of the present invention that may exist in tautomeric forms and show tautomerism; for instance, carbonyl compounds may be present in their keto and/or their enol form and show keto-enol tautomerism. Those tautomers may occur in their individual forms, e.g., the keto or the enol form, or as mixtures thereof and are claimed separately and together as mixtures in any ratio. The same applies for cis/trans isomers, E/Z isomers, conformers and the like.

In one embodiment the compounds of the present invention are in the form of free base or acid—as the case may be —, i.e. in their non-salt (or salt-free) form. In another embodiment the compounds of the present invention are in the form of a pharmaceutically acceptable salt, a pharmaceutically acceptable solvate, or a pharmaceutically acceptable solvate of a pharmaceutically acceptable salt.

The term “pharmaceutically acceptable salts” refers to salts prepared from pharmaceutically acceptable bases or acids, including inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups, such as carboxyl groups, can be present in salt form, and can be used according to the invention, for example, as alkali metal salts, alkaline earth metal salts, aluminium salts or as ammonium salts. More precise examples of such salts include lithium salts, sodium salts, potassium salts, calcium salts, magnesium salts, barium salts or salts with ammonia or organic amines such as, for example, ethylamine, ethanolamine, diethanolamine, triethanolamine, piperdine, N-methylglutamine or amino acids. These salts are readily available, for instance, by reacting the compound having an acidic group with a suitable base, e.g. lithium hydroxide, sodium hydroxide, sodium propoxide, potassium hydroxide, potassium ethoxide, magnesium hydroxide, calcium hydroxide or barium hydroxide. Other base salts of compounds of the present invention include but are not limited to copper(I), copper(II), iron(II), iron (III), manganese(II) and zinc salts. Compounds of the present invention which contain one or more basic groups, e.g. groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid or aspartic acid, and other acids known to the person skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates and glutamates. The stoichiometry of the salts formed from the compounds of the invention may moreover be an integral or non-integral multiple of one.

Compounds of the present invention which contain basic nitrogen-containing groups can be quaternized using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Therefore, the following items are also in accordance with the invention:
(a) all stereoisomers or tautomers of the compounds, including mixtures thereof in all ratios;
(b) pharmaceutically acceptable salts of the compounds and of the items mentioned under (a);
(c) pharmaceutically acceptable solvates of the compounds and of the items mentioned under (a) and (b);
(d) N-oxides of the compounds and of the items mentioned under (a), (b), and (c).

It should be understood that all references to compounds above and below are meant to include these items, in particular pharmaceutically acceptable solvates of the compounds, or pharmaceutically acceptable solvates of their pharmaceutically acceptable salts.

There is furthermore intended that a compound of the present invention includes isotope-labelled forms thereof. An isotope-labelled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the present invention by well-known methods include isotopes of hydrogen, carbon, nitrogen, oxygen, phosphorus, sulfur, fluorine and chlorine, for example ²H (D), ³H, ¹³C, ¹⁴C, ¹⁵N, ¹⁸O, ¹⁷O, ³¹P, ³²P, ³³S, ³⁴S, ³⁵S, ³⁶S, ¹⁸F and ³⁶Cl, respectively. A compound of formula I or a pharmaceutically acceptable salt thereof which contains one or more of the above-mentioned isotopes and/or other isotopes of other atoms is intended to be part of the present invention. An isotope-labelled compound of formula I can be used in a number of beneficial ways. For example, an isotope-labelled compound of the present invention into which, for example, a radioisotope, such as ³H or ¹⁴C, has been incorporated is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of formula I has therapeutic advantages owing to the higher metabolic stability of this isotope-labelled compound. Higher metabolic stability translates directly into an increased in vivo half-life or lower dosages, which under most circumstances would represent a preferred embodiment of the present invention. An isotope-labelled compound of formula I can usually be prepared by carrying out the procedures disclosed in the synthesis schemes and the related description, in the example part and in the preparation part in the present text, replacing a non-isotope-labelled reactant by a readily available isotope-labelled reactant.

Deuterium (²H; D) can also be incorporated into a compound of formula I for the purpose of manipulating the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus cause a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound of the formula I that is susceptible to oxidation, the profile of this compound in vivo can be drastically modified and result in improved pharmacokinetic properties.

When discovering and developing therapeutic agents, the person skilled in the art attempts to optimize pharmacokinetic parameters while retaining desirable in vitro properties. It is reasonable to assume that many compounds with poor pharmacokinetic profiles are susceptible to oxidative metabolism.

In vitro liver microsomal assays currently available provide valuable information on the course of oxidative metabolism of this type, which in turn permits the rational design of deuterated compounds of the formula I with improved stability through resistance to such oxidative meta-bolism. Significant improvements in the pharmacokinetic profiles of compounds of the formula I are thereby obtained, and can be expressed quantitatively in terms of increases in the in vivo half-life (t½), concentration at maximum therapeutic effect (Cₘₐₓ), area under the dose response curve (AUC), and F; and in terms of reduced clearance, dose and materials costs.

The following is intended to illustrate the above: a compound of formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favourable and accurate determination of the extent of the extent to which the improvement in resistance to oxidative metabolism has improved. In this way, it is deter-mined that the half-life of the parent compound can be extended by up to 100% as the result of deuterium-hydrogen exchange of this type.

Deuterium-hydrogen exchange in a compound of the present invention can also be used to achieve a favourable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1995.

Furthermore, the present invention relates to pharmaceutical compositions comprising at least one compound of formula I, or its N-oxides, solvates, tautomers or stereoisomers thereof as well as the pharmaceutically acceptable salts of each of the foregoing, including mixtures thereof in all ratios, as active ingredient, together with a pharmaceutically acceptable carrier.

For the purpose of the present invention the term “pharmaceutical composition” (or “pharmaceutical formulation”) refers to a composition or product comprising one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier. It may further comprise physiologically acceptable excipients, auxiliaries, adjuvants, diluents and/or additional pharmaceutically active substance other than the compounds of the invention.

The pharmaceutical compositions include compositions and pharmaceutical formulations suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient.

They may be conveniently presented in unit dosage form and prepared by any of the methods well-known in the art of pharmacy.

A pharmaceutical composition of the present invention may additionally comprise one or more other compounds as active ingredients (drugs), such as one or more additional compounds of the present invention. In a particular embodiment the pharmaceutical composition further comprises a second active ingredient or its derivatives, prodrugs, solvates, tautomers or stereoisomers thereof as well as the pharmaceutically acceptable salts of each of the foregoing, including mixtures thereof in all ratios, wherein that second active ingredient is other than a compound of formula I; preferably, that second active ingredient is a compound that is useful in the treatment, prevention, suppression and/or amelioration of medicinal conditions or pathologies for which the compounds of the present invention are useful as well and which are listed elsewhere hereinbefore or hereinafter. Such combination of two or more active ingredients or drugs may be safer or more effective than either drug or active ingredient alone, or the combination is safer or more effective than it would be expected based on the additive properties of the individual drugs. Such other drug(s) may be administered, by a route and in an amount commonly used contemporaneously or sequentially with a compound of the invention. When a compound of the invention is used contemporaneously with one or more other drugs or active ingredients, a combination product containing such other drug(s) and the compound of the invention—also referred to as “fixed dose combination”—is preferred. However, combination therapy also includes therapies in which the compound of the present invention and one or more other drugs are administered on different overlapping schedules. It is contemplated that when used in combination with other active ingredients, the compound of the present invention or the other active ingredient or both may be used effectively in lower doses than when each is used alone. Accordingly, the pharmaceutical compositions of the present invention include those that contain one or more other active ingredients, in addition to a compound of the invention.

The compounds of the present invention—or N-oxides, solvates, tautomers or stereoisomers thereof and/or the pharmaceutically acceptable salts of each of the foregoing, including mixtures thereof in all ratios—can be used as medicaments. They have been found to exhibit pharmacological activity by binding to TEAD and/or disrupting and/or inhibiting YAP-TEAD and/or TAZ-TEAD protein-protein interaction. It is assumed that by this activity the compounds of the present invention may prevent or reverse dysfunction of the Hippo pathway. By preventing its dysfunction, the Hippo pathway may be capable of playing its role as a tumor suppressor. Apart from preventing or reversing dysfunction of the Hippo pathway and independent of upstream Hippo regulation, the pharmacological activity of the compounds of the present invention may also be useful in other pathophysiological scenarios where inhibition or disruption of TEAD binding and/or aberrant YAP-TEAD and/or aberrant TAZ-TEAD signaling would be beneficial.

Thus, the compounds of the present invention being TEAD binders and/or inhibitors of YAP-TEAD and/or TAZ-TEAD interaction are useful in particular in the treatment, prevention, suppression and/or amelioration of hyperproliferative disorders and cancer, in particular tumors including solid tumors, of breast cancer, lung cancer, mesothelioma, epithelioid hemangioendothelioma, uveal melanoma, liver cancer, ovarian cancer, squamous cancer, renal cancer, gastric cancer, medulloblastoma, colon cancer, pancreatic cancer, schwannoma, meningioma, glioma, basal cell carcinoma. Without wishing to commit to any specific theory or explanation it may be assumed that the compounds might be able to achieve this by direct effects on the cancer cells and/or indirectly by modulating the response of the immune system against the tumor. Furthermore, the compounds of the present invention may also be useful in the treatment, prevention, suppression and/or amelioration of non-cancerous disorders and diseases, e.g. cardiovascular diseases and fibrosis (like liver fibrosis).

In a particular embodiment the compounds of the present invention are for use in the prevention and/or treatment, especially in the treatment of any of the disorders or diseases listed above, preferably of cancer, in particular tumors including solid tumors, of the specific types of cancer disclosed in the previous paragraph; or of any of the non-cancerous disorders or diseases disclosed in the previous paragraph.

Another particular embodiment of the present invention is a method for preventing and/or treating, preferably treating a disorder or disease selected from the group consisting of hyperproliferative disorders and cancer, in particular tumors including solid tumors, of the specific types of cancer disclosed in the previous paragraphs; or of any of the non-cancerous disorders or diseases disclosed in the previous paragraphs.

Still another particular embodiment of the invention is the use of a compound of the present invention—or derivatives, N-oxides, prodrugs, solvates, tautomers or stereoisomers thereof and/or the pharmaceutically acceptable salts of each of the foregoing, including mixtures thereof in all ratios—for the manufacturing of a medicament, in particular for preventing and/or treating, preferably treating a disorder or disease selected from the group consisting of hyperproliferative disorders and cancer, in particular tumors including solid tumors, of the specific types of cancer disclosed in the previous paragraphs; or of any of the non-cancerous disorders or diseases disclosed in the previous paragraphs.

Preferably, the present invention relates to a compound of the present invention for use in the prevention and/or treatment of a disease—or, alternatively, a method for preventing and/or treating a disease by administering an effective amount of a compound of the present invention: or, in another alternative, a use of a compound of the present invention for the manufacturing of a medicament for the prevention and/or treatment of a disease—wherein that disease is a cancer, in particular tumors including solid tumors, of the specific types of cancer disclosed in the previous paragraphs; and more preferably, wherein administration of the compound is simultaneous, sequential or in alternation with administration of at least one other active drug agent.

The disclosed compounds of the present invention and in particular of formula I can be administered in combination with other known therapeutic agents, including anticancer agents. As used here, the term “anticancer agent” relates to any agent which is administered to a patient with cancer for the purposes of treating the cancer. The anti-cancer treatment defined above may be applied as a monotherapy or may involve, in addition to the herein disclosed compounds of the present invention, conventional surgery or radiotherapy or medicinal therapy. Such medicinal therapy, e.g. a chemotherapy or a targeted therapy, may include one or more, but preferably one, of the following anti-tumor agents:

### Alkylating Agents

such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, evofosfamide, VAL-083^{[4]};

### Platinum Compounds

such as carboplatin, cisplatin, eptaplatin, miriplatine hydrate, oxaliplatin, lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{[1],[3]};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine; fosbretabulin, tesetaxel;

### Antimetabolites

such as asparaginase^{[3]}, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur^{[2],[3]}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;

### Hormones/Antagonists

such as abarelix, abiraterone, bicalutamide, buserelin, calusterone, chlorotrianisene, degarelix, dexamethasone, estradiol, fluocortolone, fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide^{[1],[3]};

### Aromatase Inhibitors

such as aminoglutethimide, anastrozole, exemestane, fadrozole, letrozole, testolactone; formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tepotinib, tipifarnib, tivantinib, tivozanib, trametinib, pimasertib, brivanib alaninate, cediranib, apatinib^{[4]}, cabozantinib S-malatel^{[1],[3]}, ibrutinib^{[1],[3]}, icotinib^{[4]}, buparlisib^{[2]}, cipatinib^{[4]}, cobimetinib^{[1],[3]}, idelalisib^{[1],[3]}, fedratinib^{[1]}, tesevatinib;

### Photosensitizers

such as methoxsalen^{[3]}; porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{[2],[3]}; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{[1],[2],[3]}, onartuzumabl^{[1],[3]}, racotumomab^{[1]}, tabalumab^{[1],[3]}, EMD-525797^{[4]}, atezolizumab, durvalumab, pembrolizumab, nivolumab^{[1],[3]};

### Cytokines

such as aldesleukin, interferon alfa2, interferon alfa2a^{[3]}, interferon alfa2b^{[2],[3]}; celmoleukin, tasonermin, teceleukin, oprelvekin^{[1],[3]}, recombinant interferon beta-1^{[4]};

### Dru Conjugates

such as denileukin diftitox, ibritumomab tiuxetan, iobenguane I 123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab^{[1],[3]}, vintafolide^{[1],[3]};

### Vaccines

such as sipuleucel^{[3]}; vitespen^{[3]}, emepepimut-S^{[3]}, oncoVAX^{[4]}, rindopepimut^{[3]}, troVax^{[4]}, MGN-1601^{[4]}, MGN-1703^{[4]};

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleucel^{[3]}, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine^{[4]}, picibanil^{[4]}, reolysin^{[4]}, retaspimycin hydrochloride^{[1],[3]}, trebananib^{[2],[3]}, virulizin^{[4]}, carfilzomib^{[1],[3]}, endostatin^{[4]}, immucothel^{[4]}, belinostat^{[3]};

### PARP Inhibitors

Olaparib, Veliparib.

### MCT1 Inhibitors

AZD3965^{[4]}, BAY-8002^{[4]}.
^{[1]} Prop. INN (Proposed International Nonproprietary Name)
^{[2]} Rec. INN (Recommended International Nonproprietary Names)
^{[3]} USAN (United States Adopted Name)
^{[4]} no INN.

In another aspect of the invention, a set or kit is provided comprising a therapeutically effective amount of at least one compound of the invention and/or at least one pharmaceutical composition as described herein and a therapeutically effective amount of at least one further pharmacologically active substance other than the compounds of the invention. It is preferred that this set or kit comprises separate packs of
a) an effective amount of a compound of formula I, or any of its N-oxides, solvates, tautomers or stereoisomers thereof as well as the pharmaceutically acceptable salts of each of the foregoing, including mixtures thereof in all ratios, and
b) an effective amount of a further active ingredient that further active ingredient not being a compound of formula I.

A further embodiment of the present invention is a process for the manufacture of the pharmaceutical compositions of the present invention, characterized in that one or more compounds according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

The pharmaceutical compositions (formulations) of the present invention may be administered by any means that achieve their intended purpose. For example, administration may be via oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be via the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

Tablets: mixing of active ingredient/s and auxiliaries, compression of said mixture into tablets (direct compression), optionally granulation of part of mixture before compression.

Capsules: mixing of active ingredient/s and auxiliaries to obtain a flowable powder, optionally granulating powder, filling powders/granulate into opened capsules, capping of capsules.

Semi-solids (ointments, gels, creams): dissolving/dispersing active ingredient/s in an aqueous or fatty carrier; subsequent mixing of aqueous/fatty phase with complementary fatty/aqueous phase, homogenization (creams only).

Suppositories (rectal and vaginal): dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms.

Aerosols: dispersing/dissolving active agent/s in a propellant, bottling said mixture into an atomizer.

In general, non-chemical routes for the production of pharmaceutical compositions and/or pharmaceutical preparations comprise processing steps on suitable mechanical means known in the art that transfer one or more compounds of the invention into a dosage form suitable for administration to a patient in need of such a treatment. Usually, the transfer of one or more compounds of the invention into such a dosage form comprises the addition of one or more compounds, selected from the group consisting of carriers, excipients, auxiliaries and pharmaceutical active ingredients other than the compounds of the invention. Suitable processing steps include, but are not limited to combining, milling, mixing, granulating, dissolving, dispersing, homogenizing, casting and/or compressing the respective active and nonactive ingredients. Mechanical means for performing said processing steps are known in the art, for example from Ullmann's Encyclopedia of Industrial Chemistry, 5th Edition. In this respect, active ingredients are preferably at least one compound of the invention and optionally one or more additional compounds other than the compounds of the invention, which show valuable pharmaceutical properties, preferably those pharmaceutical active agents other than the compounds of the invention, which are disclosed herein.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The compounds of the invention may also be lyophilized and the resultant lyophilizates used, for example, for the preparation of injection preparations. The preparations indicated may be sterilized and/or comprise assistants, such as lubricants, preservatives, stabilizers and/or wetting agents, emulsifiers, salts for modifying the osmotic pressure, buffer substances, dyes, flavors and/or a plurality of further active ingredients, for example one or more vitamins.

Suitable excipients are organic or inorganic substances, which are suitable for enteral (for example oral), parenteral or topical administration and do not react with the compounds of the invention, for example water, vegetable oils, benzyl alcohols, alkylene glycols, polyethylene glycols, glycerol triacetate, gelatin, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatin, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The compounds of the invention can also be lyophilized and the lyophilizates obtained can be used, for example, for the production of injection preparations.

Other pharmaceutical preparations, which can be used orally include push-fit capsules made of gelatin, as well as soft, sealed capsules made of gelatin and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400).

Aqueous injection suspensions may contain substances, which increase the viscosity of the suspension, including, for example, sodium carboxymethyl cellulose, sorbitol, and/or dextran, optionally, the suspension may also contain stabilizers.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers.

Possible pharmaceutical preparations, which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules, which consist of a combination of the active compounds with a base. Possible base materials include, for example, liquid triglycerides, polyethylene glycols, or paraffin hydrocarbons.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term also includes within its scope a “therapeutically effective amount” which means any amount which, as compared to a corresponding subject who has not received such amount, results in improved treatment, healing, prevention, or amelioration of a disease, disorder, or side effect, or a decrease in the rate of advancement of a disease or disorder, or of symptoms associated with such disease or disorder; it may also refer to preventing or providing prophylaxis for the disease or disorder in a subject having or at risk for developing a disease disclosed herein. The term also includes within its scope amounts effective to enhance normal physiological function. Said therapeutic effective amount of one or more of the compounds of the invention is known to the skilled artisan or can be easily determined by standard methods known in the art.

“Treating” or “treatment” as used herein, means an alleviation, in whole or in part, of symptoms associated with a disorder or disease, or slowing, or halting of further progression or worsening of those symptoms, or prevention or prophylaxis of the disease or disorder in a subject at risk for developing the disease or disorder.

The compounds of the present invention and the optional additional active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The specific dose for the individual patient, in particular for the individual human patient, depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

The compounds of the present invention can be prepared according to the procedures of the following Schemes and Examples, using appropriate materials, and as further exemplified by the following specific examples. They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

It will be recognized by those skilled in the art that some of the compounds of formula I may serve as starting material for making other compounds of formula I. For instance, a compound of formula I bearing a carboxylic functional group may readily be converted into a related compound of formula I bearing an amide functional group by utilizing appropriate synthetic methods.

Preferably, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water.

The reaction temperature is between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The present invention also refers to a process for manufacturing a compound of formula I in its most general form as well as any of the particular embodiments, PE1, PE1a, PE1aa, PE1ab, PE2, PE2a, PE2aa, PE2b, PE2ba, PE2baa, PE3, PE3a, PE3b, PE3c, PE4, PE4a, PE4aa, PE4b, PE4c, PE5, PE5a, PE5b, PE5ba, PE5baa, PE5c, PE5ca, PE5d, PE5da, PE5daa, PE6, PE6a, PE7, PE7a, PE7b, PE8, PE8a, PE8aa, PE8ab, PE9, PE9a, PE9aa, PE9b, PE9ba, PE9c, PE9ca, PE10, PE10a, PE10b, PE10c, PE10d, PE11, PE11a, PE11b, PE11c, PE11d, PE12, PE12a, PE12b, PE12c, PE12d, PE13, PE13a, PE13b, PE13c, PE13d, PE14 and PE14a described herein, or N-oxides, solvates, tautomers or stereoisomers thereof as well as the pharmaceutically acceptable salts of each of the foregoing, the process being characterized in that
a compound of formula II

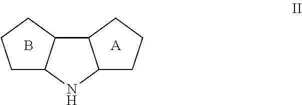

wherein Ring A and Ring B are as defined for the compound of formula I hereinabove or in any of the claims
is
(a) reacted with a compound of formula III

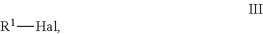

wherein R¹ is as defined for the compound of formula I hereinabove or in any of the claims and Hal represents Cl, Br or I,
in a C—N cross coupling reaction under suitable reaction conditions; to provide
a compound of formula I as defined hereinabove or in any of the claims; and optionally
(b) if in the compound of formula I R² is —C(═O)—OR^{2a} with R^{2a} being unsubstituted or substituted C₁₋₈-aliphatic, then this compound of formula I is subjected to a saponification reaction under suitable conditions to provide the respective compound of formula I with R² being —C(═O)—OH or —C(═O)—OCat; and
optionally
(c) that compound of formula I with R² being —C(═O)—OH or —C(═O)—OCat is reacted with a compound of formula IV

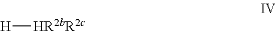

wherein R^{2b} and R^{2c} are as defined for the compound of formula I hereinabove or in any of the claims, under suitable reaction conditions; to provide a compound of formula I with R² being —C(═O)—NR^{2a}R^{2b} wherein R^{2b} and R^{2c} are as defined for the compound of formula I hereinabove or in any of the claims.

As will be understood by the person skilled in the art of organic synthesis compounds of the present invention, in particular compounds of formula I, are readily accessible by various synthetic routes, some of which are exemplified in the accompanying Experimental Part. The skilled artisan will easily recognize which kind of reagents and reactions conditions are to be used and how they are to be applied and adapted in any particular instance—wherever necessary or useful—in order to obtain the compounds of the present invention. Furthermore, some of the compounds of the present invention can readily be synthesized by reacting other compounds of the present invention under suitable conditions, for instance, by converting one particular functional group being present in a compound of the present invention, or a suitable precursor molecule thereof, into another one by applying standard synthetic methods, like reduction, oxidation, addition or substitution reactions; those methods are well known to the skilled person. Likewise, the skilled artisan will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

In the following general synthetic routes that may be utilized to prepare compounds of the present invention are described in more detail in Schemes A and B below:

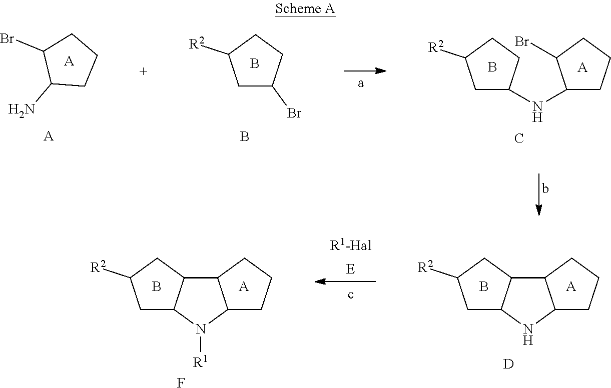

Scheme A above depicts a general route of synthesis for preparing compounds of formula I (here depicted as formula F). Unless specifically defined in a different manner, Ring A, Ring B, R¹ and R² are defined as for the compounds of formula I hereinabove or in the claims. The 1-amino-2-bromo-substituted five-membered heterocycle A is either available from a commercial source or readably available by utilizing synthetic methods and procedures well-known to the skilled person. Similarly, the bromo-substituted starting material B—wherein R² may in particular be a carboxylic acid ester, e.g., —C(═O)O-methyl—is either available from a commercial source or readably available by utilizing synthetic methods and procedures well-known to the skilled person. In reaction step (a) compounds A and B are reacted in a C—N cross-coupling reaction, for instance, under conditions typical for a Hartwig-Buchwald reaction utilizing, e.g., cesium carbonate in a suitable solvent like 1,4-dioxane in the presence of a suitable palladium catalyst like Pd-PEPPSI-IPentCI ([1,3-Bis-(2,6-di-3-pentylphenyl)-imidazol-2-yliden](3-chlorpyridyl)-dichloropalladium(II)) or utilizing potassium carbonate in the presence of BuXPhos (2-Di-tert-butylphosphin-2′,4′,6′-triisopropylbiphenyl)/t-BuXPhos G3 ([(2-Di-tert-butylphosphino-2′,4′,6′-triisopropyl-1,1′-biphenyl)-2-(2′-amino-1,1′-biphenyl)]palladium(II)-methansulfonat), to provide a compound of formula C. The compound of formula C may then be subjected to an intra-molecular C—C cross-coupling reaction utilizing a Palladium catalyst, e.g., Palladium-di-acetate/1,4-Bis-(diphenylphosphino)-butan (DPPB) in a suitable solvent, e.g., dimethylformamide (DMF), under appropriate reaction conditions (heating), thereby yielding compound D (compound of formula II) (reaction step (b)). This tricyclic heterocycle D may then in turn be reacted with R¹-Hal (compound E) (compound of formula III) in another C—N coupling reaction (reaction step (c)) to provide compound F (compound of formula I). Typical reaction conditions are, for instance, if Hal is Br, cesium carbonate in the presence of a suitable palladium catalyst (e.g., Chloro(2-dicyclohexylphosphino-2′,4′,6′-triisopropyl-1,1′-biphenyl)[2-(2′-amino-1,1′-biphenyl)]palladium(II), X-Phos aminobiphenyl palladium chloride, XPhosPd G2), or, if Hal is I (iodine), potassium carbonate in the presence of copper-(I)-iodide (CuI) and 1,2-dimethylethylendiamine (DMEDA) in a suitable solvent like 1,4-dioxane. If R² in compound B is chosen to be a carboxylic acid ester group, the synthesis would provide a compound F with that carboxylic acid ester group as R². Saponification of this ester group, e.g., by reacting with a base like lithium hydroxide, would provide the respective carboxylic acid (R²=—COOH) or carboxylic acid salt (R²=—COOCat). Such carboxylic acid of formula I (or F) may then be converted into further compounds of formula I with a different functional group R²; for instance, carboxamides (R²=—C(═O)—NR^{2b}R^{2c}) are readably available by applying typical amidation reaction conditions. Alternatively, compounds of formula I with other substituents R² may also be prepared by utilizing suitably substituted compounds of formula B as starting material.

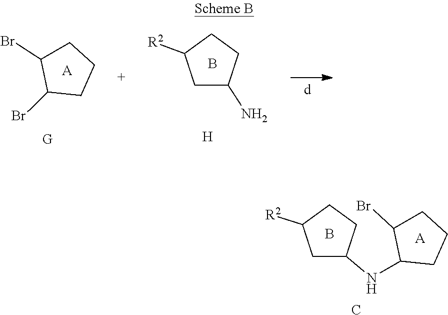

Intermediate compound C having a Ring A which is symmetrical, like, for instance, the triazole ring A-24, may be prepared by utilizing an alternative synthetic approach (see Scheme B): In reaction step (d) the amino-substituted compound H and the dibromo-substituted compound G are reacted in a C—N cross-coupling reaction, for instance, under conditions typical for a Hartwig-Buchwald reaction utilizing, e.g., cesium carbonate in a suitable solvent like 1,4-dioxane in the presence of a suitable palladium catalyst like Pd-PEPPSI-IPentCl ([1,3-Bis-(2,6-di-3-pentylphenyl)-imidazol-2-yliden](3-chlorpyridyl)-dichloropalladium(II)) or utilizing potassium carbonate in the presence of BuXPhos (2-Di-tert-butylphosphin-2′,4′,6′-triisopropyl-biphenyl)/t-BuXPhos G3 ([(2-Di-tert-butylphosphino-2′,4′,6′-triisopropyl-1,1′-biphenyl)-2-(2′-amino-1,1′-biphenyl)]palladium(II)-methansulfonat), to provide a compound of formula C.

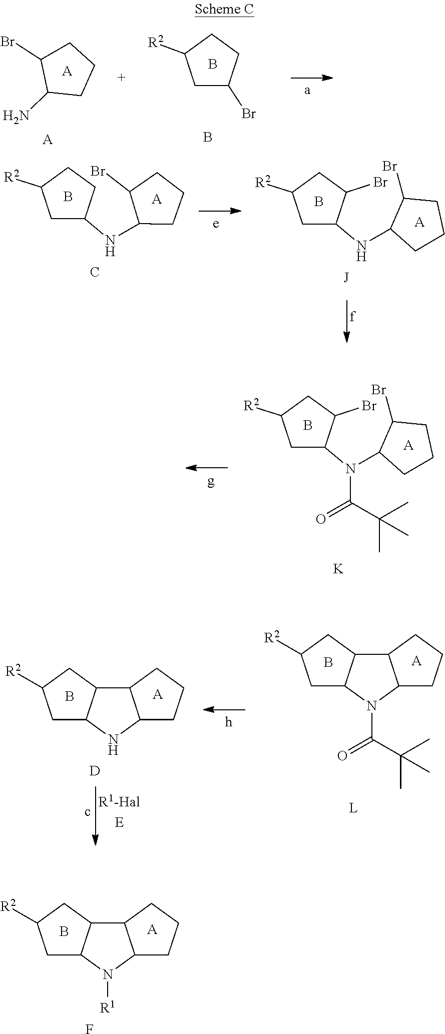

Scheme C above depicts a general route of synthesis for preparing compounds of formula I (here depicted as formula F). Unless specifically defined in a different manner, Ring A, Ring B, R¹ and R² are defined as for the compounds of formula I hereinabove or in the claims. The 1-amino-2-bromo-substituted five-membered heterocycle A is either available from a commercial source or readily available by utilizing synthetic methods and procedures well-known to the skilled person. Similarly, the bromo-substituted starting material B—wherein R² may in particular be a carboxylic acid ester, e.g., —C(═O)O-methyl—is either available from a commercial source or readily available by utilizing synthetic methods and procedures well-known to the skilled person. In reaction step (a) compounds A and B are reacted in a C—N cross-coupling reaction, for instance, under conditions typical for a Hartwig-Buchwald reaction utilizing, e.g., cesium carbonate in a suitable solvent like DMAc in the presence of a suitable palladium catalyst like XantPhos Pd G3 ([(4,5-bis(diphenylphosphino)-9,9-dimethylxanthene)-2-(2′-amino-1,1′-biphenyl)]palladium(II) methanesulfonate) to provide a compound of formula C. The compound of formula C may then be brominated under typical conditions such as NBS in a suitable solvent such as acetonitrile to give compound of formula J (reaction step (e)). Dibrominated compound J may be then protected with a suitable protecting group, e.g. pivaloyl, using standard conditions like reaction with pivaloyl chloride in the presence of a base (e.g. DIPEA) and a catalyst (e.g. DMAP) in a suitable solvent such as DCM, to give intermediate K. Compound with formula K may then be subjected to an intra-molecular Stille-Kelly cross-coupling ring closure utilizing CuI, hexamethylditin, a palladium catalyst, e.g., bis(tri-tert-butylphosphane) palladium/tri-tert-butylphosphine in a suitable solvent, e.g., dioxane, under appropriate reaction conditions (heating), thereby yielding compound L (reaction step (g)). This tricyclic heterocycle L may then be deprotected under suitable conditions (e.g. LDA) in the presence of a suitable solvent (e.g. THF) to give deprotected intermediate of formula D (reaction step (h)). Compound of formula D in turn be reacted with R¹-Hal (compound E) (compound of formula III) in another C—N coupling reaction (reaction step (c)) to provide compound F (compound of formula I). Typical reaction conditions are, for instance, if Hal is Br, cesium carbonate in the presence of a suitable palladium catalyst (e.g., Chloro(2-dicyclohexylphosphino-2′,4′,6′-triisopropyl-1,1′-biphenyl)[2-(2′-amino-1,1′-biphenyl)]palladium(II), X-Phos aminobi-phenyl palladium chloride, XPhosPd G2), or, if Hal is I (iodine), potassium carbonate in the presence of copper-(I)-iodide (CuI) and 1,2-dimethylethylendiamine (DMEDA) in a suitable solvent like 1,4-dioxane. If R² in compound B is chosen to be a carboxylic acid ester group, the synthesis would provide a compound F with that carboxylic acid ester group as R². Saponification of this ester group, e.g., by reacting with a base like lithium hydroxide, would provide the respective carboxylic acid (R²=—COOH) or carboxylic acid salt (R²=—COOCat). Such carboxylic acid of formula I (or F) may then be converted into further compounds of formula I with a different functional group R²; for instance, carboxamides (R²=—C(═O)—NR^{2b}R^{2c}) are readily available by applying typical amidation reaction conditions.

Alternatively, compounds of formula I with other substituents R² may also be prepared by utilizing suitably substituted compounds of formula B as starting material.

It is to be noted that—except for instances where it is specifically stated or the context provides for a different meaning—in general the number of a term, i.e. its singular and plural form, is used and can be read interchangeably. For example, the term “compound” in its singular form may also comprise or refer to a plurality of compounds, while the term “compounds” in its plural form may also comprise or refer to a singular compound.

### EXAMPLES AND EXPERIMENTAL PART

The compounds of the present invention can be prepared according to the procedures of the following Schemes and Examples, using appropriate materials and are further exemplified by the following specific examples. The compounds are shown in Table 1 and Table 1a. Analytical data of compounds made according to the following examples are shown in Table 1 and Table 1a, too.

The invention will be illustrated, but not limited, by reference to the specific embodiments described in the following examples. Unless otherwise indicated in the schemes, the variables have the same meaning as described above and in the claims.

Unless otherwise specified, all starting materials are obtained from commercial suppliers and used without further purifications. Unless otherwise specified, all temperatures are expressed in ° C. and all reactions are conducted at room temperature (RT). Compounds are purified by either silica chromatography or preparative HPLC.

### ¹H NMR:

¹H-NMR data is provided in Table 1 and Table 1a below. ¹H NMR spectra were usually acquired on a Bruker Avance DRX 500, Bruker Avance 400, Bruker DPX 300 or a Bruker Avance III 700 MHz (equipped with a TXI cryoprobe) NMR spectrometer under standard conditions using TMS (tetramethylsilane) as internal reference and DMSO-d6 as standard solvents, if not reported otherwise. NS (Number of Scans): 32, SF (Spectrometer Frequency) as indicated. TE (Temperature): 297 K. Chemical shifts (δ) are reported in ppm relative to the TMS signal. ¹H NMR data are reported as follows: chemical shift (multiplicity, coupling constants and number of hydrogens). Multiplicity is abbreviated as follows: s (singlet), d (doublet), t (triplet), q (quartet), m (multiplet), dd (doublet of doublets), tt (triplet of triplets), td (triplet of doublets) br (broad) and coupling constants (J) are reported in Hz.

### LC-MS:

LC-MS data provided in Table 1 and Table 1a are given with mass in m/z. The results can be obtained by one of the methods described below.

Melting point (m.p.) of selected compounds were determined by using a Tianjin Analytical Instrument RY-1.

### Syntheses

### Example 1: N,10-dimethyl-7-[4-(trifluoromethyl)phenyl]-3-thia-7,9,10-triazatricyclo[6.3.0.0^{2,6}]undeca-1(11),2(6),4,8-tetraene-4-carboxamide

### Example 1-1: Synthesis of methyl 4-[(4-bromo-1-methyl-1H-pyrazol-3-yl)amino]thiophene-2-carboxylate

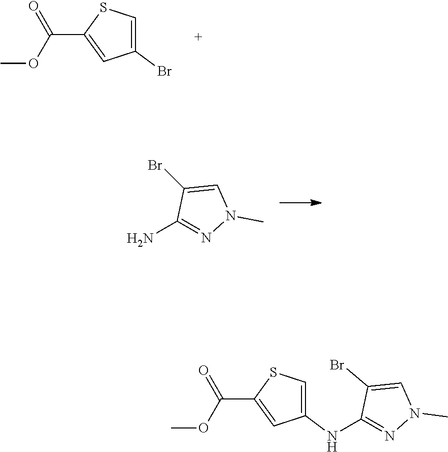

To a solution of methyl 4-bromothiophene-2-carboxylate (20.0 g, 67.9 mmol) and K₂CO₃ (19.8 g, 136 mmol) in dioxane (400 mL) were added t-BuXPhos (3.04 g, 6.79 mmol), 4-bromo-1-methyl-1H-pyrazol-3-amine (15.1 g, 81.5 mmol) and t-BuXPhos G3 ([(2-Di-tert-butylphosphino-2′,4′,6′-triisopropyl-1,1′-biphenyl)-2-(2′-amino-1,1′-biphenyl)]palladium(II) methanesulfonate) (5.68 g, 6.79 mmol) under N₂ atmosphere. The reaction mixture was stirred for 16 h at 120° C. After cooling to room temperature, the reaction was quenched by the addition of water. The resulting mixture was extracted with EtOAc (3×300 mL), the combined organic phases were washed with brine (3×900 mL) and dried over Na₂SO₄. After filtration, the filtrate was concentrated under reduced pressure. The residue was purified by silica gel column chromatography (PE/EtOAc=2:1) to give the desired product (7.00 g, 22.1 mmol, 32%, light yellow solid).

### Example 1-2: Synthesis of methyl 10-methyl-3-thia-7,9,10-triazatricyclo[6.3.0.0^{2,6}]undeca-1(11),2(6),4,8-tetraene-4-carboxylate

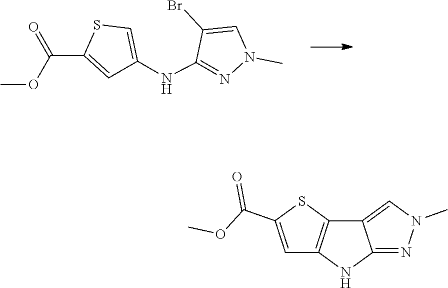

Into a sealed tube were added methyl 4-[(4-bromo-1-methyl-1H-pyrazol-3-yl)amino]thiophene-2-carboxylate (7.00 g, 22.1 mmol), Pd(OAc)₂ (1.05 g, 4.43 mmol) and 1,4-bis(diphenylphosphino)butane (1.99 g, 4.43 mmol) and dissolved in N,N-dimethylacetamide (210 mL). The reaction mixture was irradiated in a microwave for 70 min at 150° C. After cooling to room temperature, the reaction was quenched by the addition of water. The resulting mixture was extracted with EtOAc (3×300 mL), the combined organic phases were washed with brine (3×1000 mL) and dried over Na₂SO₄. After filtration, the filtrate was concentrated under reduced pressure. The residue was purified by silica gel column chromatography (PE/EtOAc=2:1) to give the desired product (805 mg, 3.32 mmol, 15%, green solid).

### Example 1-3: Synthesis of methyl 10-methyl-7-[4-(trifluoromethyl)phenyl]-3-thia-7,9,10-triazatricyclo[6.3.0.0^{2,6}]undeca-1(11),2(6),4,8-tetraene-4-carboxylate

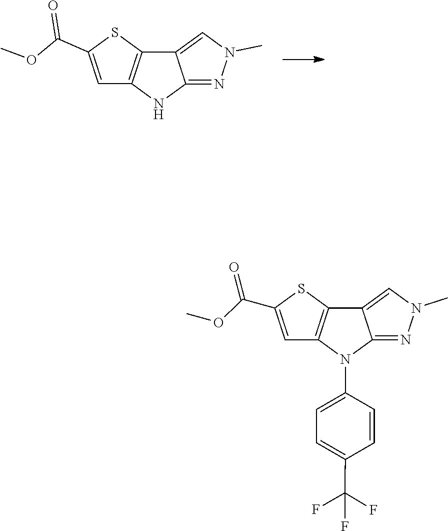

A mixture of methyl 10-methyl-3-thia-7,9,10-triazatricyclo[6.3.0.0^{2,6}]undeca-1(11),2(6),4,8-tetraene-4-carboxylate (375 mg, 1.59 mmol), 4-Iodobenzo-trifluoride 97% (302 μl, 1.99 mmol), Cs₂CO₃ (1.05 g, 3.19 mmol) and 1,2-dimethylethylenediamine (173 μl, 1.59 mmol) was suspended in 1,4-dioxane (7.50 ml). The reaction vial was crimped, put under vacuum, sonicated for 2 minutes and refilled with argon. This procedure was repeated two times, followed by the addition of CuI (152 mg, 0.80 mmol). The vial was crimped and the procedure described above was repeated, followed by stirring at 110° C. for 17 hrs. The reaction mixture was filtered through celite and washed with EtOAc (45 ml) and deionized water (30 ml). The filtrate was concentrated in vacuo to give a mixture of crude methyl 10-methyl-7-[4-(trifluoromethyl)phenyl]-3-thia-7,9,10-triazatricyclo[6.3.0.0^{2,6}]undeca-1(11),2(6),4,8-tetraene-4-carboxylate (605 mg, 1.20 mmol, 76%, beige solid) and 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid (582 mg, 0.39 mmol, 24%, beige solid) which was used as a mixture in the next step without further purification.

### Example 1-4: Synthesis of 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid

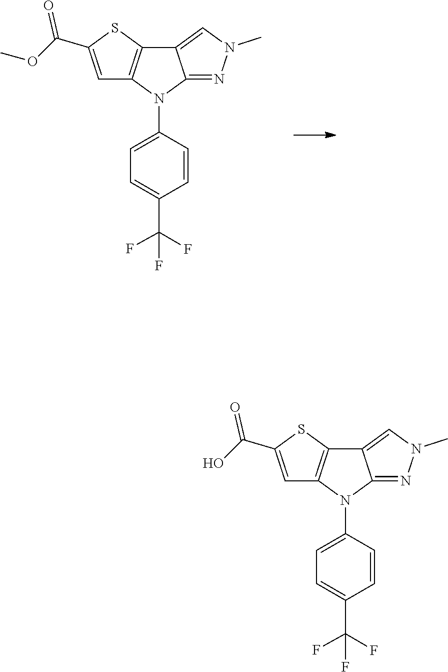

To a solution of methyl 10-methyl-7-[4-(trifluoromethyl)phenyl]-3-thia-7,9,10-triazatricyclo[6.3.0.0^{2,6}]undeca-1(11),2(6),4,8-tetraene-4-carboxylate (47.0 mg, 0.12 mmol) in H₂O (1 mL) and THF (1 mL) was added LiOH (8.00 mg, 0.32 mmol). The reaction was stirred for 16 h at room temperature after which it was acidified to pH 4 with aqueous HCl. The mixture was extracted with EtOAc (3×10 mL), the combined organic phases were dried over Na₂SO₄ and concentrated under reduced pressure. The residue was purified by silica gel column chromatography (CH₂Cl₂/MeOH 10:1) to give 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid (29.1 mg, 0.08 mmol, 63%, off-white solid, m.p. 206-208° C.).

### Example 1-5: Synthesis of N,10-dimethyl-7-[4-(trifluoromethyl)phenyl]-3-thia-7,9,10-triazatricyclo[6.3.0.0^{2,6}]undeca-1(11),2(6),4,8-tetraene-4-carboxamide

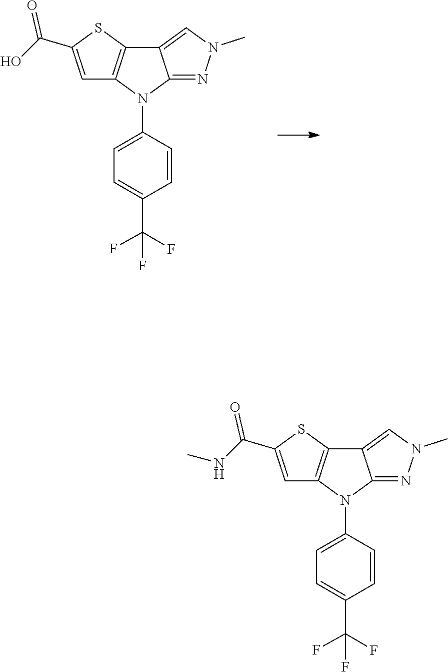

To a solution of 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid (55.0 mg, 0.15 mmol) in DMF (1.10 ml) methylamine (2M in THF, 113 μl, 0.23 mmol), 4-methylmorpholine (83.6 μl, 0.75 mmol), N-(3-Dimethyl-aminopropyl)-N′-ethylcarbodiimide hydro chloride (58.9 mg, 0.30 mmol) and 1-hydroxybenzotriazole hydrate (23.8 mg, 0.15 mmol) were added. The reaction was stirred at room temperature for 15 hrs. The crude product was purified by RP (Reversed Phase) flash chromatography (SunFire C18 5.0 μm 150-30 mm; A: H2O+0.1% TFA B: MeCN+0.1% TFA; 30% B: 0->3.0 min; 30%->68% B: 3.0->19.5 min; Flow: 45 mL/min) to give N,10-dimethyl-7-[4-(trifluoromethyl)phenyl]-3-thia-7,9,10-triazatricyclo[6.3.0.0^{2,6}]undeca-1(11),2(6),4,8-tetraene-4-carboxamide (39.3 mg, 69%, white solid, m.p. 247-249° C.).

### Example 2: 7-[4-(difluoromethoxy)phenyl]-10-methyl-3-thia-7,9,10-triazatricyclo[6.3.0.0^{2,6}]undeca-1(11),2(6),4,8-tetraene-4-carboxylic acid

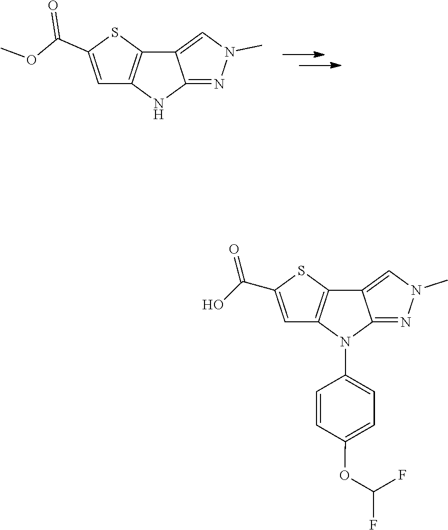

This product was synthesized following the same protocols as described for Example 1-4: 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid.

### Example 3: 10-methyl-7-[4-(trifluoromethoxy)phenyl]-3-thia-7,9,10-triazatricyclo[6.3.0.0^{2,6}]undeca-1(11),2(6),4,8-tetraene-4-carboxylic acid

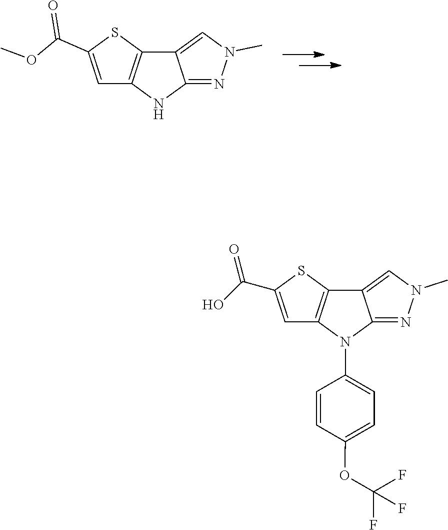

This product was synthesized following the same protocol as described for Example 1-4 utilizing 4-iodo-1-trifluoromethoxyphenyl: 10-methyl-7-[4-(trifluoromethyl)phenyl]-3-thia-7,9,10-triazatricyclo[6.3.0.0^{2,6}]undeca-1(11),2(6),4,8-tetraene-4-carboxylic acid.

### Example 4: 10-methyl-7-(4,4,4-trifluoro-3,3-dimethylbutyl)-3-thia-7,9,10-triazatricyclo[6.3.0.0^{2,6}]undeca-1(11),2(6),4,8-tetraene-4-carboxylic acid

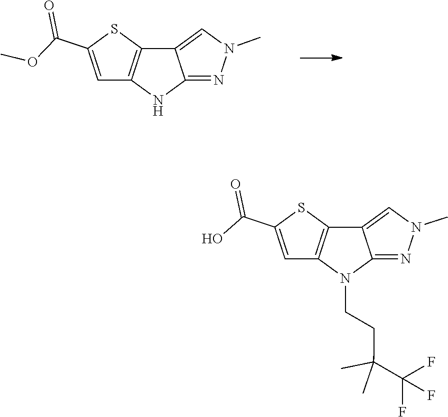

A solution of methyl 10-methyl-3-thia-7,9,10-triazatricyclo[6.3.0.0^{2,6}]undeca-1(11),2(6),4,8-tetraene-4-carboxylate (37.5 mg, 0.16 mmol) in DMF (750 μl) was inertised with argon and cooled down to 0-5° C. with an ice bath. After addition of NaH (60% suspension in paraffin oil, 18.6 mg, 0.46 mmol) the reaction mixture was stirred at 0° C. for 0.25 hrs, followed by the addition of 4-bromo-1,1,1-trifluoro-2,2-dimethylbutane (42.8 mg, 0.19 mmol). The vial was inertised with fresh argon, allowed to warm up to rt and stirred for 17.5 hrs. The reaction was stopped by the addition of water (approx. 1 ml) and the mixture was purified by RP flash chromatography (SunFire C18 5.0 μm 150-30 mm; A: H2O+0.1% TFA B: MeCN+0.1% TFA; 25% B: 0->5.5 min; 25%->60% B: 5.5->27.5 min; Flow: 45 mL/min). The pure fractions were combined, concentrated and the residue was coevaporated with deionized water (2×5 ml) and once with toluene to give 10-methyl-7-(4,4,4-trifluoro-3,3-dimethylbutyl)-3-thia-7,9,10-triazatricyclo[6.3.0.0^{2,6}]undeca-1(11),2(6),4,8-tetraene-4-carboxylic acid (36.9 mg, 0.10 mmol, 66%; pale grey solid).

### Example 5: N,10-dimethyl-7-{[(1s,3s)-3-(trifluoromethyl)cyclobutyl]methyl}-3-thia-7,9,10-triazatricyclo[6.3.0.0^{2,6}]undeca-1(11),2(6),4,8-tetraene-4-carboxamide

### Example 5-1: Synthesis of 10-methyl-7-{[3-(trifluoromethyl)cyclobutyl]-methyl}-3-thia-7,9,10-triazatricyclo[6.3.0.0^{2,6}]undeca-1(11),2(6),4,8-tetraene-4-carboxylic acid

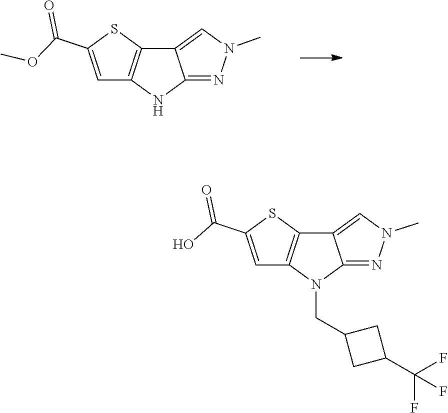

This product was synthesized following the same protocol as described for Example 4: 10-methyl-7-(4,4,4-trifluoro-3,3-dimethylbutyl)-3-thia-7,9,10-tri-azatricyclo[6.3.0.0^{2,6}]undeca-1(11),2(6),4,8-tetraene-4-carboxylic acid.

### Example 5-2: Synthesis of 10-methyl-7-{[(1r,3r)-3-(trifluoromethyl)cyclobutyl]methyl}-3-thia-7,9,10-triazatricyclo[6.3.0.0^{2,6}]undeca-1(11),2(6),4,8-tetraene-4-carboxylic acid and 10-methyl-7-{[(1s,3s)-3-(trifluoromethyl)cyclobutyl]methyl}-3-thia-7,9,10-triazatricyclo[6.3.0.0^{2,6}]undeca-1(11),2(6),4,8-tetraene-4-carboxylic acid

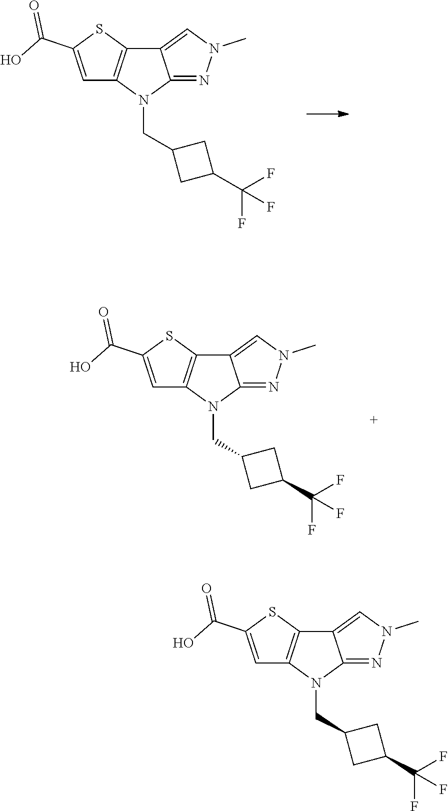 [SMILES: Cn1cc2c3sc(C(=O)O)cc3n(CC3CC(C(F)(F)F)C3)c2n1 ; Cn1cc2c3sc(C(=O)O)cc3n(C[C@H]3C[C@H](C(F)(F)F)C3)c2n1 ; Cn1cc2c3sc(C(=O)O)cc3n(C[C@H]3C[C@@H](C(F)(F)F)C3)c2n1]

The isomeric mixture of 10-methyl-7-{[(1r,3r)-3-(trifluoromethyl)cyclobutyl]-methyl}-3-thia-7,9,10-triazatricyclo[6.3.0.0^{2,6}]undeca-1(11),2(6),4,8-tetraene-4-carboxylic acid and 10-methyl-7-{[(1s,3s)-3-(trifluoromethyl)cyclo-butyl]methyl}-3-thia-7,9,10-triazatricyclo[6.3.0.0^{2,6}]undeca-1(11),2(6),4,8-tetraene-4-carboxylic acid was separated by a second RP flash chromatography (SunFire C18 5.0 μm 150-30 mm; A: H2O+0.1% TFA B: MeCN+0.1% TFA; 25% B: 0->9.0 min; 25%->60% B: 9.0->29.5 min; Flow: 45 mL/min).

### Example 5-3: Synthesis of N,10-dimethyl-7-{[(1s,3s)-3-(trifluoromethyl)-cyclobutyl]methyl}-3-thia-7,9,10-triazatricyclo[6.3.0.0^{2,6}]undeca-1(11),2(6),4,8-tetraene-4-carboxamide

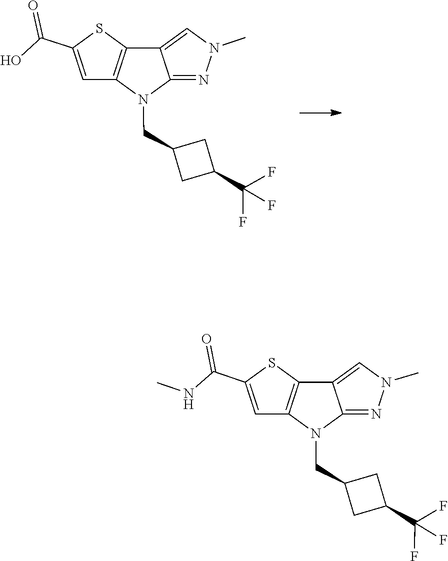 [SMILES: Cn1cc2c3sc(C(=O)O)cc3n(C[C@H]3C[C@@H](C(F)(F)F)C3)c2n1 ; CNC(=O)c1cc2c(s1)c1cn(C)nc1n2C[C@H]1C[C@@H](C(F)(F)F)C1]

This product was synthesized following the same protocol as described for Example 1: Synthesis of N,10-dimethyl-7-[4-(trifluoromethyl)phenyl]-3-thia-7,9,10-triazatricyclo[6.3.0.0^{2,6}]undeca-1(11),2(6),4,8-tetraene-4-carboxamide.

### Example 6: N-[2-hydroxy-1-(pyridin-2-yl)ethyl]-4-methyl-7-[4-(trifluoromethyl)phenyl]-11-thia-3,4,5,7-tetraazatricyclo[6.3.0.0^{2,6}]undeca-1(8),2,5,9-tetraene-10-carboxamide

### Example 6-1: Synthesis of methyl 4-[(5-bromo-2-methyl-2H-1,2,3-triazol-4-yl)amino]thiophene-2-carboxylate

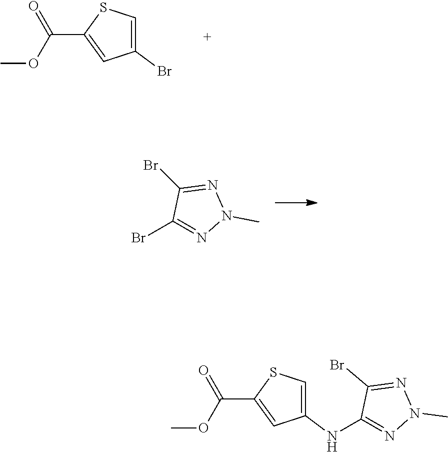

To a solution of methyl 4-aminothiophene-2-carboxylate (1.00 g, 6.04 mmol) and 4,5-dibromo-2-methyl-2H-1,2,3-triazole (1.80 g, 7.25 mmol) in dioxane (10 mL) were added Pd-PEPPSI™-IPentCl 2-methylpyridine ([1,3-Bis(2,6-Di-3-pentylphenyl)imidazol-2-ylidene](3-chloropyridyl)dichloropalladium(II),) (0.50 g, 0.60 mmol) and Cs₂CO₃ (4.10 g, 12.0 mmol). The reaction mixture was stirred for 2 h at 100° C. After cooling to room temperature, the reaction was quenched by the addition of water. The resulting mixture was extracted with EtOAc (3×200 mL), the combined organic phases were washed with brine (3×900 mL) and dried over Na₂SO₄. After filtration, the filtrate was concentrated under reduced pressure. The residue was purified by silica gel column chromatography (PE/EtOAc=85:15) to give the desired product (0.90 g, 2.84 mmol, 43%, yellow solid).

### Example 6-2: Synthesis of methyl 4-methyl-11-thia-3,4,5,7-tetraazatricyclo[6.3.0.0^{2,6}]undeca-1(8),2,5,9-tetraene-10-carboxylate

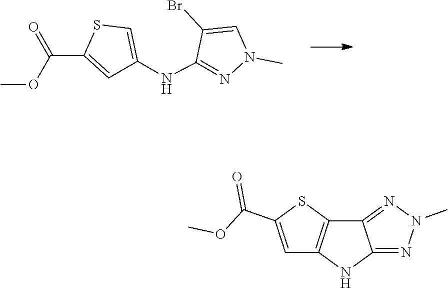

To a stirred mixture of methyl 4-[(5-bromo-2-methyl-2H-1,2,3-triazol-4-yl)amino]thiophene-2-carboxylate (5.88 g, 18.1 mmol) in DMF (100.00 ml) N,N-diisopropylethylamine (6.62 ml, 36.1 mmol) and Bis(tri-tert-butyl-phosphine)palladium(0) (971 mg, 1.81 mmol) were added at room temperature. The reaction was stirred at 100° C. under nitrogen atmosphere for overnight. After cooling down to room temperature, the reaction mixture was diluted with water and the aqueous phase was extracted with EtOAc (3×200 mL). The combined organic layers were washed with saturated NaCl solution, dried over anhydrous Na₂SO₄ and the solvent was removed under reduced pressure. The residue was purified by silica gel column chromatography (PE/EtOAc 75:25) to give methyl 4-methyl-11-thia-3,4,5,7-tetraazatricyclo[6.3.0.0^{2,6}]undeca-1(8),2,5,9-tetraene-10-carboxylate (4.00 g, 8.65 mmol, 48%; light brown solid, purity 51%).

### Example 6-3: Synthesis of methyl 4-methyl-7-[4-(trifluoromethyl)phenyl]-11-thia-3,4,5,7-tetraazatricyclo[6.3.0.0^{2,6}]undeca-1(8),2,5,9-tetraene-10-carboxylate

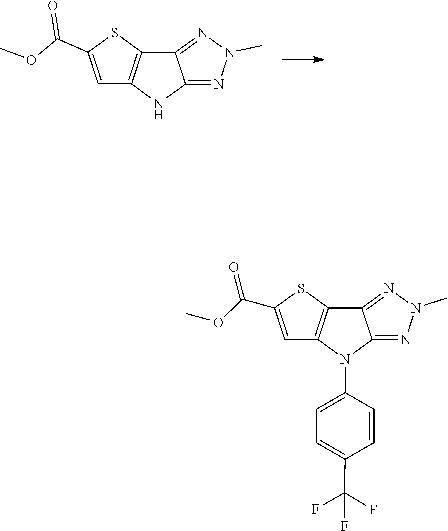

To a stirred mixture of methyl 4-methyl-11-thia-3,4,5,7-tetraaza-tricyclo[6.3.0.0^{2,6}]undeca-1(8),2,5,9-tetraene-10-carboxylate (2.90 g, 6.27 mmol) and 1-iodo-4-(trifluoromethyl)benzene (3.59 g, 12.5 mmol) in 1,4-dioxane (20.0 ml) were added CuI (623.0 mg, 3.11 mmol), N,N′-dimethylethylenediamine (291.0 mg, 3.14 mmol) and K₂CO₃ (1.82 g, 12.5 mmol) at room temperature. The resulting mixture was stirred for overnight at 100° C. under nitrogen atmosphere. After cooling down to room temperature, the reaction was stopped by the addition of water. The resulting mixture was extracted with EtOAc (3×300 mL), the combined organic phases were washed with saturated NaCl solution, dried over Na₂SO₄ and the solvent was removed under reduced pressure. The residue was purified by silica gel column chromatography (PE/EtOAc 80:20) to afford methyl 4-methyl-7-[4-(trifluoromethyl)phenyl]-11-thia-3,4,5,7-tetraazatricyclo[6.3.0.0^{2,6}]undeca-1(8),2,5,9-tetraene-10-carboxylate (2.00 g, 5.14 mmol; 82%, off-white solid).

### Example 6-4: Synthesis of 4-methyl-7-[4-(trifluoromethyl)phenyl]-11-thia-3,4,5,7-tetraazatricyclo[6.3.0.0^{2,6}]undeca-1(8),2,5,9-tetraene-10-carboxylic acid

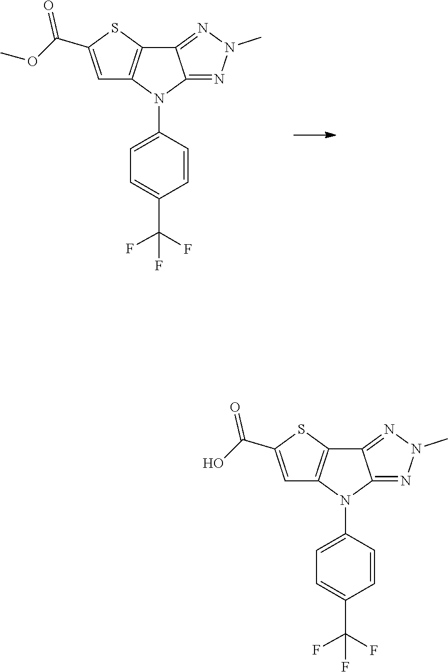

To a solution of methyl 4-methyl-7-[4-(trifluoromethyl)phenyl]-11-thia-3,4,5,7-tetraazatricyclo[6.3.0.0^{2,6}]undeca-1(8),2,5,9-tetraene-10-carboxylate (90.0 mg, 0.23 mmol) in MeOH (5 mL) was added NaOH (28.0 mg, 0.67 mmol) in H₂O (1 mL). The reaction mixture was stirred for 3 h at 60° C. and subsequently acidified to pH 3 with aqueous HCl. The resulting mixture was extracted with EtOAc (3×50 mL), the combined organic phases were dried over Na₂SO₄. After filtration, the filtrate was concentrated under reduced pressure. The residue was purified by preparative HPLC (Column: Halo C18 4.6*100 mm, Solvent A: Water/0.05% TFA, Solvent B: MeCN/0.05% TFA, Flow: 1.2 mL/min, Gradient: 5% B to 100% B in 8.0 min) to give 4-methyl-7-[4-(trifluoromethyl)phenyl]-11-thia-3,4,5,7-tetraazatricyclo[6.3.0.0^{2,6}]undeca-1(8),2,5,9-tetraene-10-carboxylic acid (50.4 mg, 0.14 mmol, 61%, white solid, m.p. 265-267° C.).

### Example 6-5: Synthesis of N-[2-hydroxy-1-(pyridin-2-yl)ethyl]-4-methyl-7-[4-(trifluoromethyl)phenyl]-11-thia-3,4,5,7-tetraazatricyclo[6.3.0.0^{2,6}]undeca-1(8),2,5,9-tetraene-10-carboxamide

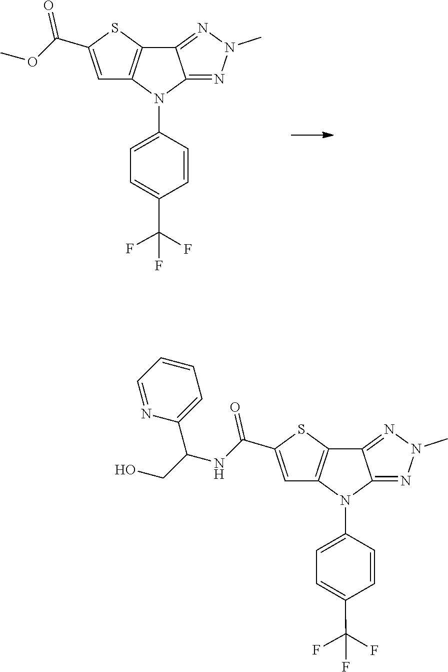

To a solution of 4-methyl-7-[4-(trifluoromethyl)phenyl]-11-thia-3,4,5,7-tetra-azatricyclo[6.3.0.0^{2,6}]undeca-1(8),2,5,9-tetraene-10-carboxylic acid (130 mg, 0.35 mmol) and 2-amino-2-(pyridin-2-yl)ethan-1-ol (102 mg, 0.70 mmol) in DMF (4 mL) were added 1-[Bis(dimethylamin)methylen]-1H-1,2,3-triazol[4,5-b]pyridinium 3-oxid-hexafluorphosphat (210 mg, 0.52 mmol) and DIPEA (238 mg, 1.74 mmol). The reaction was stirred for 12 h at room temperature after which it was stopped by the addition of water. The resulting mixture was extracted with EtOAc (3×50 mL), the combined organic phases were washed with saturated NaCl solution (1×20 mL), dried over MgSO₄ and concentrated under reduced pressure. The residue was purified by preparative HPLC (Column: Halo C18 4.6*100 mm, Solvent A: Water/0.05% TFA, Solvent B: MeCN/0.05% TFA, Flow: 1.2 mL/min, Gradient: 5% B to 95% B in 8 min) to give N-[2-hydroxy-1-(pyridin-2-yl)ethyl]-4-methyl-7-[4-(trifluoromethyl)-phenyl]-11-thia-3,4,5,7-tetraazatricyclo[6.3.0.0^{2,6}]undeca-1(8),2,5,9-tetraene-10-carboxamide (54.2 mg, 0.11 mmol, 32%, white solid, m.p. 220-222° C.).

### Example 7: N-[dimethyl(oxo)-λ⁶-sulfanylidene]-4-methyl-7-[4-(trifluoromethyl)phenyl]-9-thia-4,5,7-triazatricyclo[6.3.0.0^{2,6}]undeca-1(8),2,5,10-tetraene-10-carboxamide

### Example 7-1: Synthesis of methyl 5-[(4-bromo-1-methyl-1H-pyrazol-3-yl)amino]thiophene-2-carboxylate

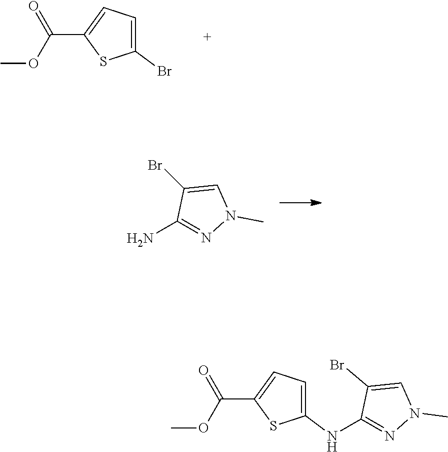

To a suspension of methyl 5-bromothiophene-2-carboxylate (1.00 g, 4.43 mmol), 4-bromo-1-methyl-1H-pyrazol-3-amine (903 mg, 4.88 mmol) and Cs₂CO₃ (2.89 g, 8.87 mmol) in dioxane (20 ml) was added XantPhos Pd G3 ([(4,5-Bis(diphenylphosphino)-9,9-dimethylxanthene)-2-(2′-amino-1,1′-biphenyl)]palladium(II) methanesulfonate) (221 mg, 0.22 mmol). The reaction mixture was stirred for 19 h at 110° C. After cooling to room temperature, the reaction was diluted by the addition of water. The resulting mixture was extracted with EtOAc (3×75 mL), the combined organic phases were washed with brine (3×20 mL) and dried over Na₂SO₄. After filtration, the filtrate was concentrated under reduced pressure. The residue was purified by silica gel column chromatography (cyclohexane/EtOAc=1:1) to give to give methyl 5-[(4-bromo-1-methyl-1H-pyrazol-3-yl)amino]thiophene-2-carboxylate (1.23 g, 2.93 mmol, 66%, dark yellow solid).

### Example 7-2: Synthesis of methyl 4-methyl-9-thia-4,5,7-triazatricyclo[6.3.0.0^{2,6}]undeca-1(8),2,5,10-tetraene-10-carboxylate

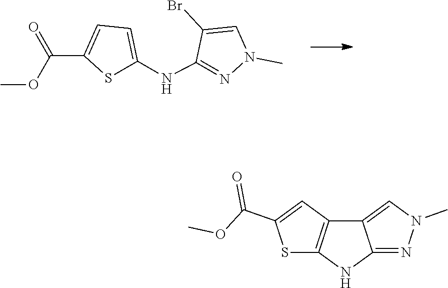

Into a sealed tube were added methyl 5-[(4-bromo-1-methyl-1H-pyrazol-3-yl)amino]thiophene-2-carboxylate (100 mg, 0.29 mmol), potassium 2,2-dimethylpropanoate (58.7 mg, 0.41 mmol) and bis(tri-tert-butyl phosphane)-palladium (30.3 mg, 0.06 mmol) and dissolved in DMF (1.95 ml). The reaction mixture was irradiated for 2 h at 160° C. After cooling to room temperature, the reaction was diluted by the addition of water. The resulting mixture was extracted with EtOAc (3×30 mL), the combined organic phases were washed with brine (2×10 mL) and with water (2×5 ml) and dried over Na₂SO₄. After filtration, the filtrate was concentrated under reduced pressure. The residue was purified by silica gel column chromatography (cyclohexane/EtOAc=1:3) to give methyl 4-methyl-9-thia-4,5,7-triazatricyclo[6.3.0.0^{2,6}]undeca-1(8),2,5,10-tetraene-10-carboxylate (29.0 mg, 0.11 mmol, 18%, deep purple solid).

### Example 7-3: Synthesis of methyl 4-methyl-7-[4-(trifluoromethyl)phenyl]-9-thia-4,5,7-triazatricyclo[6.3.0.0^{2,6}]undeca-1(8),2,5,10-tetraene-10-carboxylate

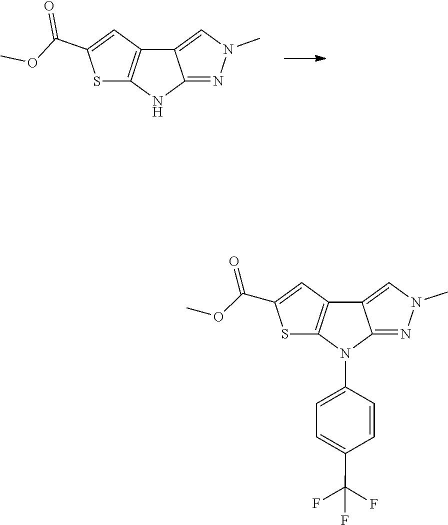

To a solution of methyl 4-methyl-9-thia-4,5,7-triazatricyclo[6.3.0.0^{2,6}]undeca-1(8),2,5,10-tetraene-10-carboxylate (21.7 mg, 0.08 mmol) and 4-Iodobenzo-trifluoride 97% (15.8 μl, 0.10 mmol) in 1,4-dioxane (434 μl) were added Cs₂CO₃ (54.9 mg, 0.17 mmol), NN′-dimethylethylenediamine (9.06 μl, 0.08 mmol) and CuI (7.94 mg, 0.04 mmol). The reaction mixture was stirred for 18 h at 110° C. After cooling to room temperature, the reaction was diluted by the addition of water. The resulting mixture was extracted with EtOAc (3×5 mL), the combined organic phases were washed with brine (1×2 mL) and dried over Na₂SO₄. After filtration, the filtrate was concentrated under reduced pressure to give methyl 4-methyl-7-[4-(trifluoromethyl)phenyl]-9-thia-4,5,7-triazatricyclo[6.3.0.0^{2,6}]undeca-1(8),2,5,10-tetraene-10-carboxylate (32.9 mg, 0.07 mmol, 83%, pale brown solid).

### Example 7-4: Synthesis of 4-methyl-7-[4-(trifluoromethyl)phenyl]-9-thia-4,5,7-triazatricyclo[6.3.0.0^{2,6}]undeca-1(8),2,5,10-tetraene-10-carboxylic acid

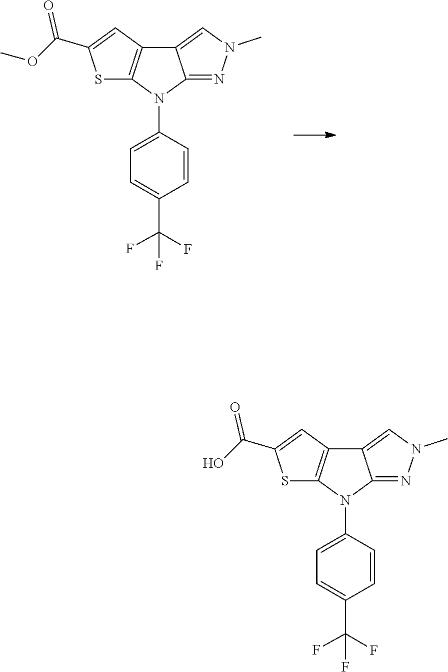

To a solution of methyl 4-methyl-7-[4-(trifluoromethyl)phenyl]-9-thia-4,5,7-triazatricyclo[6.3.0.0^{2,6}]undeca-1(8),2,5,10-tetraene-10-carboxylate (32.9 mg, 0.07 mmol) in THF (2.00 ml) and water (500 μl) LiOH (20.0 mg, 0.82 mmol) was added. The reaction was stirred for 14.5 h at rt after which it was concentrated to dryness and purified by RP flash chromatography (C18 column, H₂O+0.1% TFA/MeCN+0.1% TFA=35%-70%) to give 4-methyl-7-[4-(trifluoromethyl)phenyl]-9-thia-4,5,7-triazatricyclo[6.3.0.0^{2,6}]undeca-1(8),2,5,10-tetraene-10-carboxylic acid (13.5 mg, 0.04 mmol, 53%, pale beige solid).

### Example 7-5: Synthesis of N-[dimethyl(oxo)-λ⁶-sulfanylidene]-4-methyl-7-[4-(trifluoromethyl)phenyl]-9-thia-4,5,7-triazatricyclo[6.3.0.0^{2,6}]undeca-1(8),2,5,10-tetraene-10-carboxamide

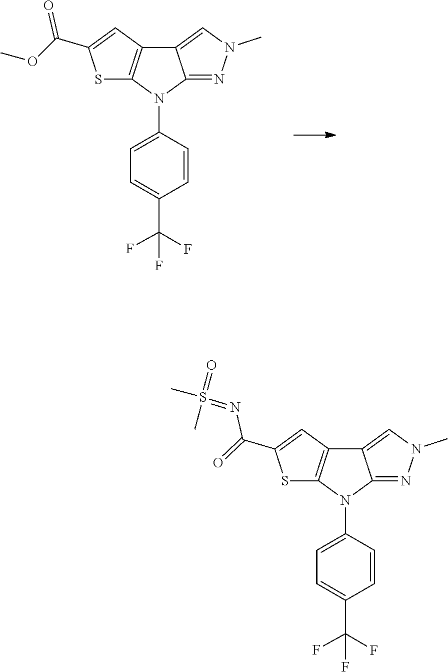

To a solution of 4-methyl-7-[4-(trifluoromethyl)phenyl]-9-thia-4,5,7-triazatricyclo[6.3.0.0^{2,6}]undeca-1(8),2,5,10-tetraene-10-carboxylic acid (19.1 mg, 0.05 mmol), iminodimethyl-λ⁶-sulfanone (10.2 mg, 0.10 mmol) and 4-methylmorpholine (34.8 μl, 0.31 mmol) in DMF (381 μl) were added N-(3-dimethylaminopropyl)-N′-ethylcarbodiimide hydro chloride (20.4 mg, 0.10 mmol) and 1-hydroxybenzotriazole hydrate (8.24 mg, 0.05 mmol). The reaction was stirred at rt for 112 hrs after which the crude product was purified by RP flash chromatography (SunFire C18, A: H2O+0.1% TFA B: MeCN+0.1% TFA, 30% B: 0->4.8 min, 30%->70% B: 4.8->31.2 min). The pure fractions were combined, basified with satiated Na₂CO₃ solution (4 ml) and extraction was carried out with EtOAc (2×10 ml). The combined organic layers were dried over Na₂SO₄, filtered off with suction and concentrated. The solid was dissolved in a mixture of CH₂Cl₂ and EtOH (10 ml/2 ml), filtered through a 4 g silica gel column and washed with a mixture of CH₂Cl₂ and EtOH (50 ml/10 ml). The filtrate was concentrated to give N-[dimethyl(oxo)-λ⁶-sulfanylidene]-4-methyl-7-[4-(trifluoromethyl)phenyl]-9-thia-4,5,7-triazatricyclo[6.3.0.0^{2,6}]undeca-1(8),2,5,10-tetraene-10-carboxamide (14.7 mg, 0.03 mmol, 64%, white solid).

### Example 8: N,3-dimethyl-7-[4-(trifluoromethyl)phenyl]-11-thia-3,5,7-triazatricyclo[6.3.0.0^{2,6}]undeca-1(8),2(6),4,9-tetraene-10-carboxamide

### Example 8-1: 1-methyl-5-(tributylstannyl)-1H-imidazole

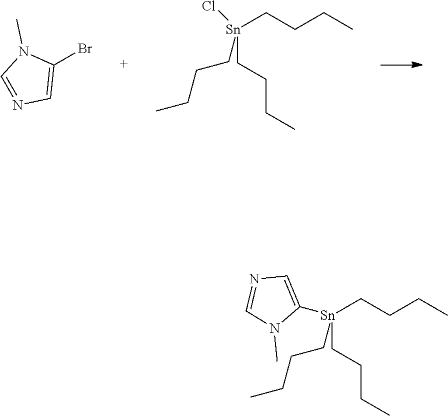

To a mixture of 5-bromo-1-methyl-1H-imidazole (3.00 g, 17.7 mmol) in EtO₂ (30 ml) nBuLi in hexane (7.79 ml, 19.5 mmol) was added dropwise at −78° C. The reaction was stirred for 30 min at −78° C. under nitrogen atmosphere followed by the dropwise addition of tributyl(chloro)stannane (8.73 g, 26.6 mmol). The reaction was stirred for 1 h at −78° C. after which saturated NH₄Cl solution was added at 0° C. The mixture was extracted with EtOAc (3×50 mL) and the combined organic layers were washed with brine and dried over anhydrous Na₂SO₄. After filtration, the filtrate was concentrated under reduced pressure to give 1 methyl-5-(tributylstannyl)-1H-imidazole (6.60 g, 17.5 mmol, 99%, yellow oil).

### Example 8-2: methyl 5-(1-methyl-1H-imidazol-5-yl)-4-nitrothiophene-2-carboxylate

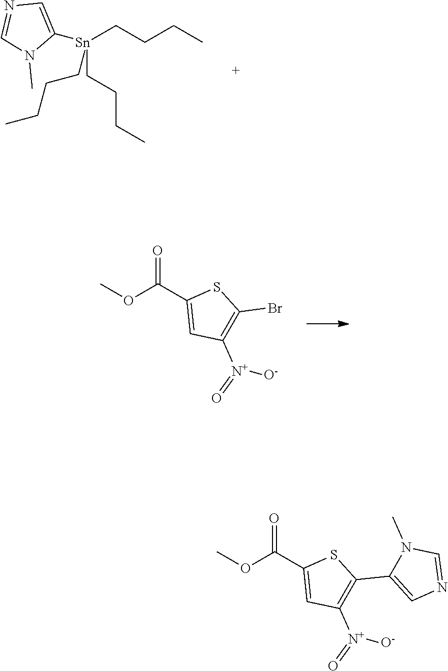 [SMILES: CCC[CH2][Sn]([CH2]CCC)([CH2]CCC)[c]1cncn1C ; COC(=O)c1cc([N+](=O)[O-])c(Br)s1 ; COC(=O)c1cc([N+](=O)[O-])c(-c2cncn2C)s1]

To a stirred mixture of 1-methyl-5-(tributylstannyl)-1H-imidazole (6.30 g, 11.6 mmol) and methyl 5-bromo-4-nitrothiophene-2-carboxylate (3.26 g, 11.6 mmol) in toluene (60 ml) tetrakis(triphenylphosphane) palladium (1.41 g, 1.16 mmol) and CsF (3.72 g, 23.3 mmol) were added in portions at room temperature. The reaction was stirred for overnight under nitrogen atmosphere at 100° C. after which it was allowed to cool down to room temperature. The mixture was extracted with EtOAc (3×50 mL), the combined organic layers were washed with brine, dried over anhydrous Na₂SO₄ and the solvent was removed under reduced pressure. The residue was purified by silica gel column chromatography (CH₂Cl₂/EtOAc 53:47) to give methyl 5-(1-methyl-1H-imidazol-5-yl)-4-nitrothiophene-2-carboxylate (2.40 g, 8.65 mmol, 74%, yellow oil).

### Example 8-3: methyl 3-methyl-11-thia-3,5,7-triazatricyclo[6.3.0.0^{2,6}]undeca-1(8),2(6),4,9-tetraene-10-carboxylate

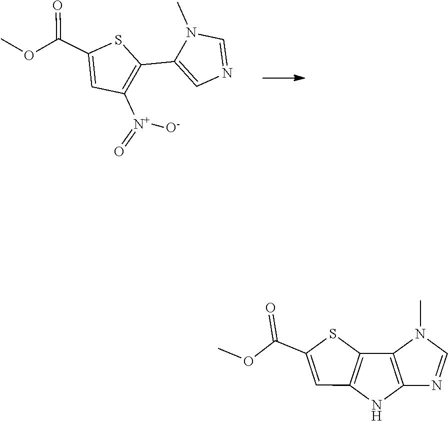 [SMILES: COC(=O)c1cc([N+](=O)[O-])c(-c2cncn2C)s1 ; COC(=O)c1cc2[nH]c3ncn(C)c3c2s1]

To a mixture of methyl 5-(1-methyl-1H-imidazol-5-yl)-4-nitrothiophene-2-carboxylate (600 mg, 2.23 mmol) in 12-dichlorobenzene (6.00 ml) [2-(diphenylphosphanyl)ethyl]diphenylphosphane (1.12 g, 2.68 mmol) was added in portions at room temperature. The reaction was stirred for overnight at 160° C. under nitrogen atmosphere after which it was allowed to cool down to room temperature. The mixture was extracted with EtOAc (3×50 mL) and the combined organic layers were washed with brine, dried over anhydrous Na₂SO₄ and concentrated under reduced pressure. The residue was purified by silica gel column chromatography (PE/EtOAc 45:55) to give methyl 3-methyl-11-thia-3,5,7-triazatricyclo[6.3.0.0^{2,6}]undeca-1(8),2(6),4,9-tetraene-10-carboxylate (350 mg, 1.43 mmol; 64%, brown yellow solid).

### Example 8-4: methyl 3-methyl-7-[4-(trifluoromethyl)phenyl]-11-thia-3,5,7-triazatricyclo[6.3.0.0^{2,6}]undeca-1(8),2(6),4,9-tetraene-10-carboxylate

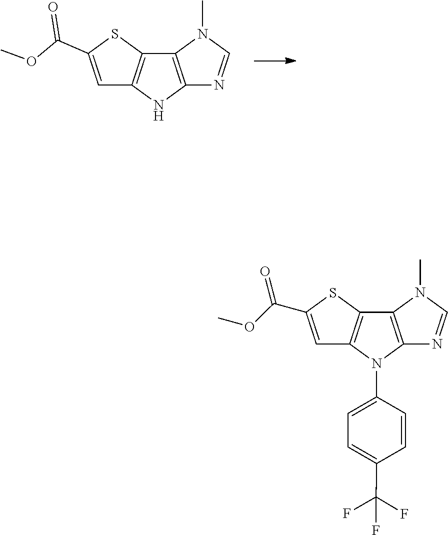 [SMILES: COC(=O)c1cc2[nH]c3ncn(C)c3c2s1 ; COC(=O)c1cc2c(s1)c1c(ncn1C)n2-c1ccc(C(F)(F)F)cc1]

To a mixture of methyl 3-methyl-11-thia-3,5,7-triazatricyclo[6.3.0.0^{2,6}]undeca-1(8),2(6),4,9-tetraene-10-carboxylate (70.0 mg, 0.26 mmol) and 1-iodo-4-(trifluoromethyl)benzene (88.0 mg, 0.31 mmol) in 1,4-dioxane (2.00 ml) CuI (26.0 mg, 0.13 mmol), N,N′-dimethylethylenediamine (12.0 mg, 0.13 mmol) and K₂CO₃ (75.0 mg, 0.52 mmol) were added at room temperature. The reaction was stirred at 100° C. under nitrogen atmosphere for overnight after which it was allowed to cool down to room temperature. The mixture was extracted with EtOAc (3×20 mL) and the combined organic layers were washed with brine, dried over anhydrous Na₂SO₄ and concentrated under reduced pressure. The residue was purified by silica gel column chromatography (PE/EtOAc 50:50) to give methyl 3-methyl-7-[4-(trifluoromethyl)-phenyl]-11-thia-3,5,7-triazatricyclo[6.3.0.0^{2,6}]undeca-1(8),2(6),4,9-tetraene-10-carboxylate (40.0 mg, 0.10 mmol, 40.%, yellow solid).

### Example 8-5: 3-methyl-7-[4-(trifluoromethyl)phenyl]-11-thia-3,5,7-triazatricyclo[6.3.0.0^{2,6}]undeca-1(8),2(6),4,9-tetraene-10-carboxylic acid

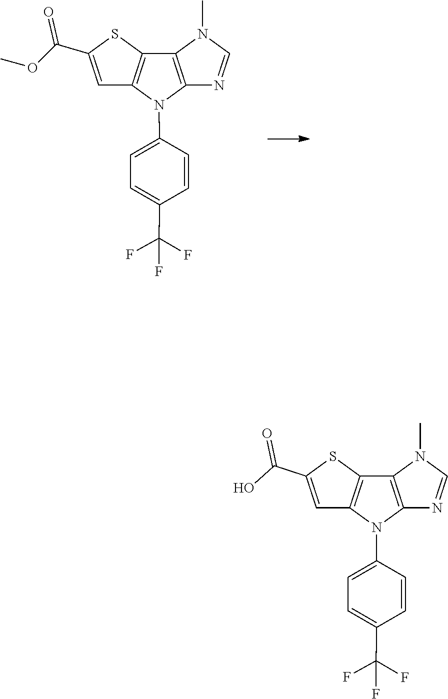 [SMILES: COC(=O)c1cc2c(s1)c1c(ncn1C)n2-c1ccc(C(F)(F)F)cc1 ; Cn1cnc2c1c1sc(C(=O)O)cc1n2-c1ccc(C(F)(F)F)cc1]

To a mixture of methyl 3-methyl-7-[4-(trifluoromethyl)phenyl]-11-thia-3,5,7-triazatricyclo[6.3.0.0^{2,6}]undeca-1(8),2(6),4,9-tetraene-10-carboxylate (130 mg, 0.34 mmol) in methanol (2.00 ml) sodium hydroxide (42.0 mg, 1.00 mmol) dissolved in water (0.40 ml) was added dropwise at room temperature. The resulting mixture was stirred for 3 h at 60° C. under nitrogen atmosphere after which it was allowed to cool down to room temperature. The mixture was diluted with water and acidified to pH=2-3 with aq. HCl. The mixture was extracted with EtOAc (3×50 mL) and the combined organic layers were washed with brine, dried over anhydrous Na₂SO₄ and concentrated under reduced pressure to afford 3-methyl-7-[4-(trifluoromethyl)phenyl]-11-thia-3,5,7-triazatricyclo[6.3.0.0^{2,6}]undeca-1(8),2(6),4,9-tetraene-10-carboxylic acid (120.0 mg, 0.32 mmol, 95%, yellow solid).

### Example 8-6: N,3-dimethyl-7-[4-(trifluoromethyl)phenyl]-11-thia-3,5,7-triazatricyclo[6.3.0.0^{2,6}]undeca-1(8),2(6),4,9-tetraene-10-carboxamide

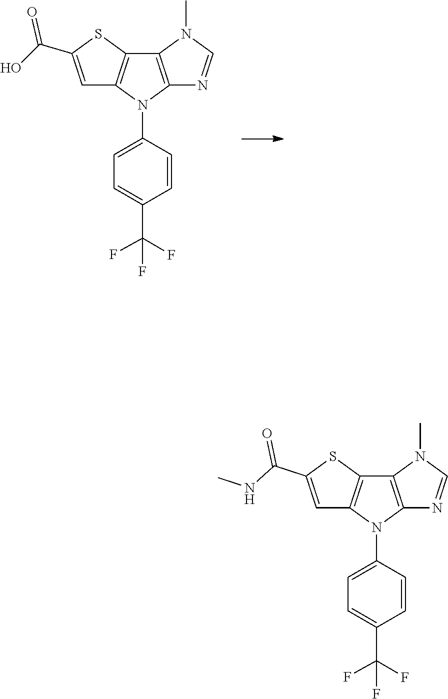 [SMILES: Cn1cnc2c1c1sc(C(=O)O)cc1n2-c1ccc(C(F)(F)F)cc1 ; CNC(=O)c1cc2c(s1)c1c(ncn1C)n2-c1ccc(C(F)(F)F)cc1]

To a solution of 3-methyl-7-[4-(trifluoromethyl)phenyl]-11-thia-3,5,7-triazatricyclo[6.3.0.0^{2,6}]undeca-1(8),2(6),4,9-tetraene-10-carboxylic acid (110.00 mg, 0.29 mmol) in DMF (1.00 ml) HATU (176.0 mg, 0.44 mmol), N,N-Diisopropylethylamine (0.16 ml, 0.88 mmol) and NH₄Cl (33.0 mg, 0.44 mmol) were added at room temperature. The reaction was stirred overnight at room temperature under nitrogen atmosphere after which it was diluted with water. The mixture was acidified to pH=2-3 with aq. HCl and extracted with EtOAc (3×50 mL). The combined organic layers were washed with brine, dried over anhydrous Na₂SO₄ and concentrated under reduced pressure. The crude product was purified by preparative HPLC (Column: C18 4.6*100 mm, mobile phase A: Water/0.05% TFA, mobile phase B: MeCN/0.05% TFA, Flow rate: 1.5 mL/min, Gradient: 5% to 100% in 8.0 min) to give N,3-dimethyl-7-[4-(trifluoromethyl)phenyl]-11-thia-3,5,7-triazatricyclo[6.3.0.0^{2,6}]undeca-1(8),2(6),4,9-tetraene-10-carboxamide (50.2 mg, 0.13 mmol, 45%; pink solid).

### Example 9: Example 24: N,4-dimethyl-7-[4-(trifluoromethyl)phenyl]-5,11-dithia-3,7-diazatricyclo[6.3.0.0^{2,6}]undeca-1(8),2(6),3,9-tetraene-10-carboxamide

### Example 9-1: Synthesis of 2-methyl-4-(tributylstannyl)-1,3-thiazole

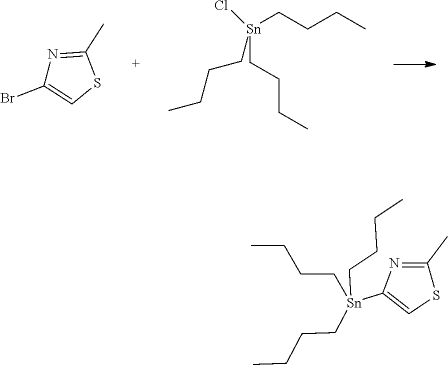

To a mixture of 4-bromo-2-methyl-1,3-thiazole (2.00 g, 10.7 mmol) in ethyl ether (20 ml) n-BuLi in hexane (4.70 ml, 11.7 mmol) was added dropwise at −78° C. The reaction was stirred for 30 min at −78° C. under nitrogen atmosphere after which tributyl(chloro)stannane (7.02 g, 21.3 mmol) was added dropwise. The reaction was stirred for 1 h at −78° C. followed by the addition of saturated Na₂CO₃ solution at 0° C. The mixture was extracted with EtOAc (3×200 mL) and the combined organic layers were washed with brine, dried over anhydrous Na₂SO₄ and concentrated under reduced pressure. The residue was purified by silica gel column chromatography (PE/EtOAc 95:5) to give 2-methyl-4-(tributylstannyl)-1,3-thiazole (4.00 g, 9.72 mmol, 91%, light yellow oil).

### Example 9-2: Synthesis of methyl 5-(2-methyl-1,3-thiazol-4-yl)-4-nitrothiophene-2-carboxylate

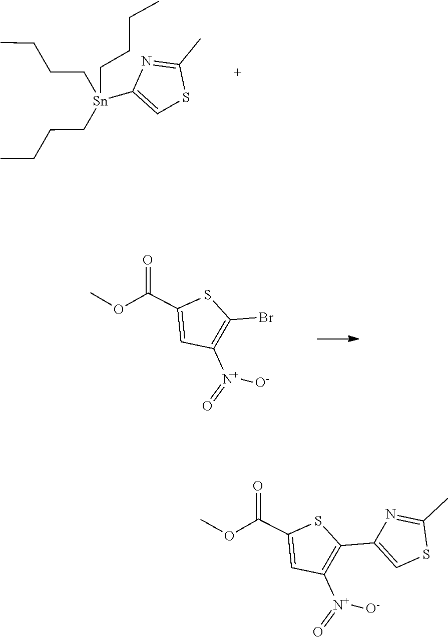

To a mixture of 2-methyl-4-(tributylstannyl)-1,3-thiazole (4.00 g, 9.72 mmol) and methyl 5-bromo-4-nitrothiophene-2-carboxylate (2.72 g, 9.72 mmol) in toluene (40.00 ml) tetrakis(triphenylphosphane)palladium (1.18 g, 0.97 mmol) and CsF (3.11 g, 19.44 mmol) were added in portions at room temperature. The reaction was stirred at 100° C. under nitrogen atmosphere for overnight after which it was allowed to cool down to room temperature. The mixture was diluted with water and extracted with EtOAc (3×200 mL). The combined organic layers were washed with brine, dried over anhydrous Na₂SO₄ and concentrated under reduced pressure. The residue was purified by silica gel column chromatography (PE/EtOAc 85:15) followed by purification with RP flash chromatography (column: C18; mobile phase A: water, mobile phase B: MeCN, 70% to 80% gradient in 20 min) to give methyl 5-(2-methyl-1,3-thiazol-4-yl)-4-nitrothiophene-2-carboxylate (2.00 g, 6.78 mmol, 70%, yellow solid).

### Example 9-3: Synthesis of methyl 4-methyl-5,11-dithia-3,7-diazatricyclo[6.3.0.0^{2,6}]undeca-1(8),2(6),3,9-tetraene-10-carboxylate

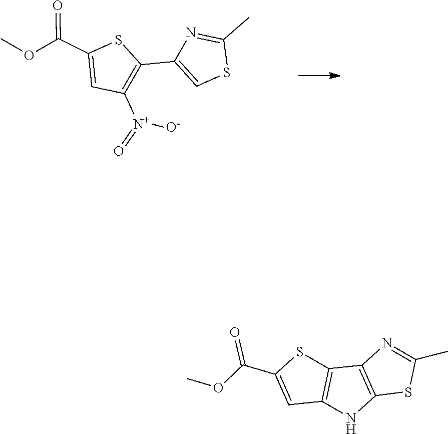

To a mixture of methyl 5-(2-methyl-1,3-thiazol-4-yl)-4-nitrothiophene-2-carboxylate (340 mg, 1.14 mmol) in 1,2-dichlorobenzene (10 ml) [2-(diphenylphosphanyl)ethyl]diphenylphosphane (524 mg, 1.25 mmol) was added in portions at room temperature. The reaction was stirred at 160° C. under nitrogen atmosphere for overnight after which it was allowed to cool down to room temperature and diluted with water. The mixture was extracted with EtOAc (3×50 mL) and the combined organic layers were washed with brine, dried over anhydrous Na₂SO₄ and concentrated under reduced pressure. The residue was purified by silica gel column chromatography (PE/EtOAc 95:5) to give methyl 4-methyl-5,11-dithia-3,7-diaza-tricyclo[6.3.0.0^{2,6}]undeca-1(8),2(6),3,9-tetraene-10-carboxylate (150 mg, 0.59 mmol, 52%, yellow brown solid).

### Example 9-4: Synthesis of methyl 4-methyl-7-[4-(trifluoromethyl)phenyl]-5,11-dithia-3,7-diazatricyclo[6.3.0.0^{2,6}]undeca-1(8),2(6),3,9-tetraene-10-carboxylate

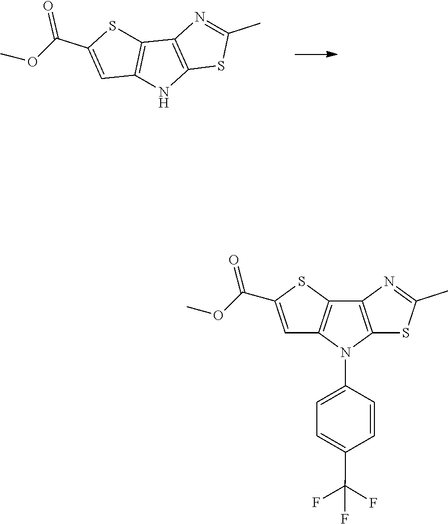

To a mixture of methyl 4-methyl-5,11-dithia-3,7-diazatricyclo[6.3.0.0^{2,6}]n-deca-1(8),2(6),3,9-tetraene-10-carboxylate (110 mg, 0.43 mmol) and 1-iodo-4-(trifluoromethyl)benzene (185 mg, 0.65 mmol) in dioxane (5.00 ml) EPhos Pd G4 (42.00 ma: 0.04 mmol) and Cs₂CO₃ (295 mg. 0.86 mmol) were added in portions at room temperature. The reaction was stirred at 100° C. under nitrogen atmosphere for overnight after which it was allowed to cool down to room temperature and diluted with water. The mixture was extracted with EtOAc (3×100 mL) and the combined organic layers were washed with brine, dried over anhydrous Na₂SO₄ and concentrated under reduced pressure. The residue was purified by silica gel column chromatography (PE/EtOAc 82:18) to give methyl 4-methyl-7-[4-(trifluoromethyl)phenyl]-5,11-dithia-3,7-diazatricyclo[6.3.0.0^{2,6}]undeca-1(8),2(6),3,9-tetraene-10-carboxylate (170 mg, 0.42 mmol, 98%, light yellow solid).

### Example 9-5: Synthesis of 3-methyl-7-[4-(trifluoromethyl)phenyl]-11-thia-3,5,7-triazatricyclo[6.3.0.0^{2,6}]undeca-1(8),2(6),4,9-tetraene-10-carboxylic acid

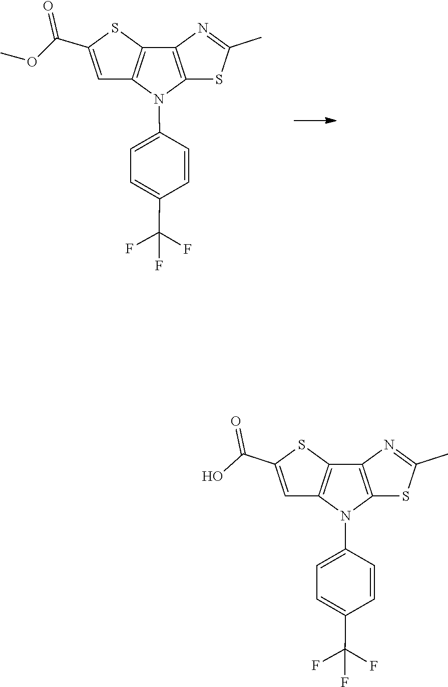

To a mixture of methyl 4-methyl-7-[4-(trifluoromethyl)phenyl]-5,11-dithia-3,7-diazatricyclo[6.3.0.0^{2,6}]undeca-1(8),2(6),3,9-tetraene-10-carboxylate (50.00 mg; 0.12 mmol) in MeOH (2.00 ml) a solution of NaOH (15.00 mg; 0.36 mmol) in water (0.40 ml) was added dropwise at room temperature. The reaction was stirred at 60° C. under nitrogen atmosphere for overnight after which it was allowed to cool down to room temperature and diluted with water at 0° C. The mixture was acidified to pH 2-3 with aq. HCl and extracted with EtOAc (3×50 mL). The combined organic layers were washed with brine, dried over anhydrous MgSO₄ and concentrated under reduced pressure to afford 4-methyl-7-[4-(trifluoromethyl)phenyl]-5,11-dithia-3,7-diazatricyclo[6.3.0.0^{2,6}]undeca-1(8),2(6),3,9-tetraene-10-carboxylic acid (50.0 mg, 0.08 mmol, 68%, yellow solid).

### Example 9-6: Synthesis of N,4-dimethyl-7-[4-(trifluoromethyl)phenyl]-5,11-dithia-3,7-diazatricyclo[6.3.0.0^{2,6}]undeca-1(8),2(6),3,9-tetraene-10-carboxamide

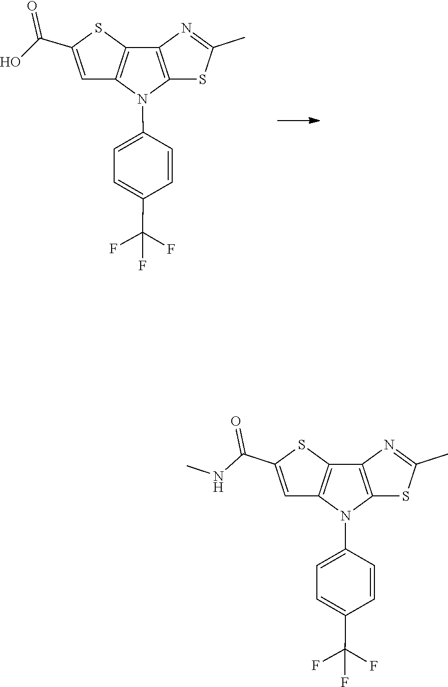

To a mixture of 4-methyl-7-[4-(trifluoromethyl)phenyl]-5,11-dithia-3,7-diazatricyclo[6.3.0.0^{2,6}]undeca-1(8),2(6),3,9-tetraene-10-carboxylic acid (70.0 mg, 0.16 mmol) and methylammonium chloride (24.0 mg, 0.32 mmol) in DMF (5.00 ml) were added HATU (98.0 mg, 0.24 mmol) and N,N-diisopropylethylamine (0.15 ml, 0.82 mmol) at room temperature. The reaction was stirred overnight at room temperature. The mixture was extracted with EtOAc (3×50 mL) and the combined organic layers were washed with brine, dried over anhydrous MgSO₄ and concentrated under reduced pressure. The residue was purified by preparative HPLC (column: Halo C18 4.6*100 mm, phase A: Water/0.05% TFA, phase B: MeCN/0.05% TFA, flow: 1.2 mL/min, Gradient: 5% to 95% in 8 min) to afford N,4-dimethyl-7-[4-(trifluoromethyl)phenyl]-5,11-dithia-3,7-diazatricyclo[6.3.0.0^{2,6}]undeca-1(8),2(6),3,9-tetraene-10-carboxamide (53.8 mg, 0.14 mmol, 83%, off-white solid).

### Example 10: N,4-dimethyl-7-[4-(trifluoromethoxy)phenyl]-5,11-dithia-3,7-diazatricyclo[6.3.0.0^{2,6}]undeca-1(8),2(6),3,9-tetraene-10-carboxamide

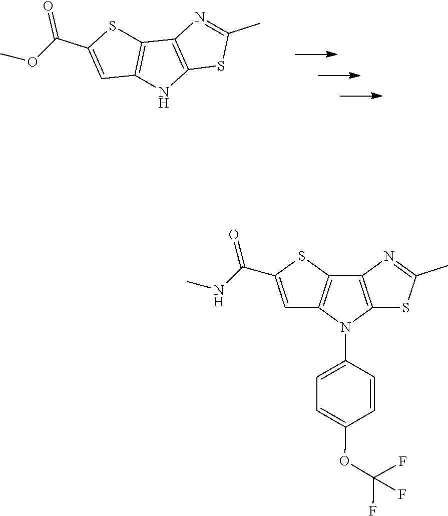

This product was synthesized following the same protocol as described for Example 9: Synthesis of N,4-dimethyl-7-[4-(trifluoromethyl)phenyl]-5,11-di-thia-3,7-diazatricyclo[6.3.0.0^{2,6}]undeca-1(8),2(6),3,9-tetraene-10-carboxamide.

### Example 11: 4-methyl-7-[4-(trifluoromethyl)phenyl]-9-thia-3,4,5,7-tetraazatricyclo[6.3.0.0^{2,6}]undeca-1(8),2,5,10-tetraene-10-carboxylic acid

### Example 11-1: Synthesis of methyl 5-[(5-bromo-2-methyl-2H-1,2,3-triazol-4-yl)amino]thiophene-2-carboxylate

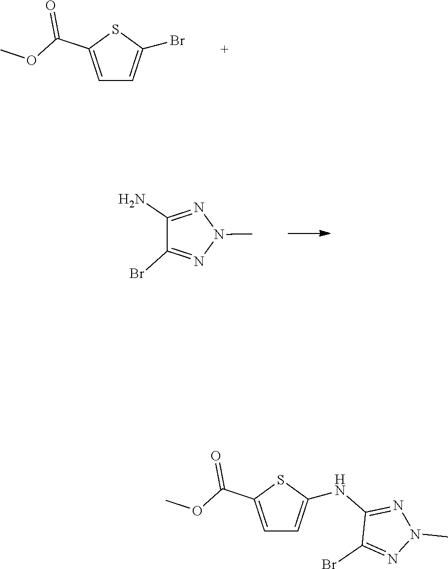

Methyl 5-bromothiophene-2-carboxylate (5.7 g, 25.3 mmol), 5-bromo-2-methyl-2H-1,2,3-triazol-4-amine (5.0 g, 27.8 mmol), Xantphos (2.3 g, 3.8 mmol) and Cs₂CO₃ (12.4 g, 37.9 mmol) were suspended in N,N-dimethyl-acetamide (114 mL). The flask was put under vacuum, sonicated for 2 minutes and refilled with argon three times. XantPhos Pd G3 (2.5 g, 2.5 mmol) was then added. The flask was closed, put under vacuum, sonicated for 2 minutes and refilled with argon three additional times. The reaction mixture was stirred at 110° C. for 4 hours. After cooling to room temperature, the reaction mixture was filtered through celite and washed with DMF (approx. 50 mL). The filtered off solution was poured into deionized water (approx. 1.75 L) and stirred for 0.5 hours with ice cooling to form a greenish precipitate, which was filtered off and washed twice with deionized water. The filtered off solid was dried overnight at 60° C. to give 6.15 g of crude product which was treated with a 3:1 mixture of n-heptane and MTB ether (approx. 40 mL) and stirred vigorously for 3 hours. The solid was filtered to obtain the desired product (5.7 g, 17.2 mmol, 68% yield) as a brown green solid.

### Example 11-2: Synthesis of methyl 4-bromo-5-[(5-bromo-2-methyl-2H-1,2,3-triazol-4-yl)amino]thiophene-2-carboxylate

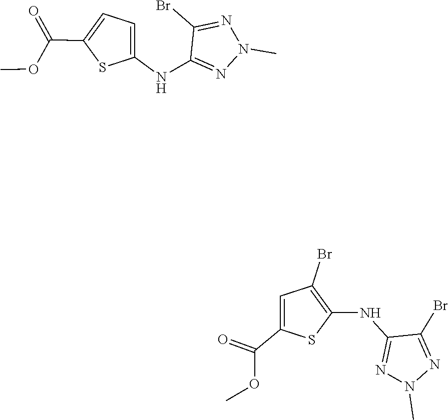

To an ice cooled suspension of methyl 5-[(5-bromo-2-methyl-2H-1,2,3-triazol-4-yl)amino]thiophene-2-carboxylate (1.25 g, 13.76 mmol) in aceto-nitrile, (25 mL)N-bromosuccinimide (697 mg, 3.88 mmol) was added. The reaction mixture was stirred for 1.5 hours at 0-5° C. The reaction mixture was diluted with deionized water and ethyl acetate and the insoluble contents were filtered off. The filtered off solid was washed with additional ethyl acetate and dichloromethane. The aqueous phases were combined with DCM and EtOAc and extracted three times with EtOAc. The combined organic phases were dried over sodium sulfate, filtered off and concentrated under reduced pressure. The residue was purified by silica gel column chromatography (cyclohexane/EtOAc 7:3) to give the desired product (1.35 g, 3.40 mmol, 90% yield) as a blue solid.

### Example 11-3: Synthesis of methyl 4-bromo-5-[N-(5-bromo-2-methyl-2H-1,2,3-triazol-4-yl)-2,2-dimethylpropanamido]thiophene-2-carboxylate

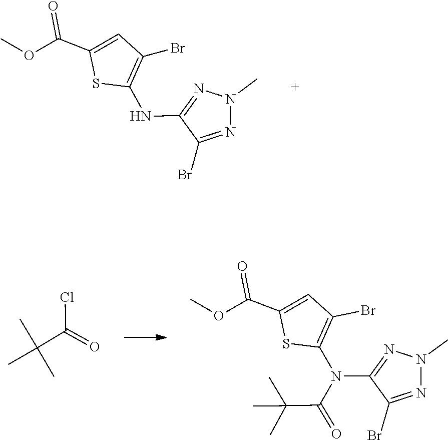

Methyl 4-bromo-5-[(5-bromo-2-methyl-2H-1,2,3-triazol-4-yl)amino]thiophene-2-carboxylate (849 mg, 2.14 mmol) and 4-(dimethylamino)pyridine (53 mg, 0.43 mmol) were dissolved in DCM (17.0 mL) and N-ethyldiisopropyl-amine (515 μL, 3.00 mmol) was added. The reaction mixture was cooled to 0-5° C. and trimethylacetyl chloride (373 μL, 3.00 mmol) was added. The reaction mixture was allowed to warm up to room temperature and stirred for 15 hours. The reaction mixture was neutralized with saturated ammonium chloride solution, diluted with deionized water and extracted with DCM twice. The combined organic phases were washed twice with a mixture of deionized water and saturated ammonium chloride solution, dried over sodium sulfate, filtered and concentrated under reduced pressure. The obtained product was purified by flash column chromatography (cyclohexane/EtOAc, 7:3) to give the desired product (944 mg, 1.94 mmol, 90% yield) as a beige solid.

### Example 11-4: Synthesis of methyl 7-(2,2-dimethylpropanoyl)-4-methyl-9-thia-3,4,5,7-tetraazatricyclo[6.3.0.0^{2,6}]undeca-1(8),2,5,10-tetraene-10-carboxylate

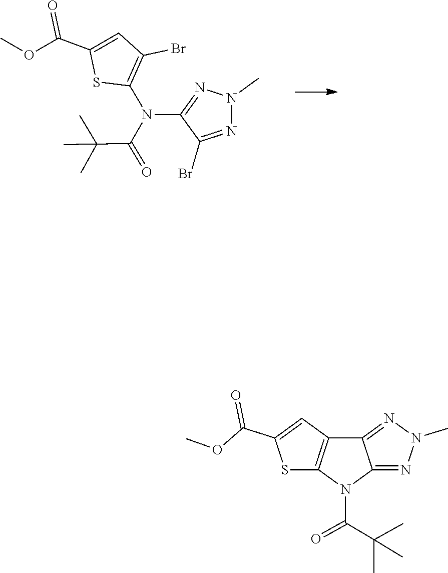

Methyl 4-bromo-5-[N-(5-bromo-2-methyl-2H-1,2,3-triazol-4-yl)-2,2-dimethyl-propanamido]thiophene-2-carboxylate (1.13 g, 2.35 mmol) was dissolved in toluene (22.5 mL). Hexamethylditin (516 μL, 2.46 mmol) and tri-tert-butyl-phosphine (917 μL, 3.71 mmol) were added under argon. The flask was closed, put under vacuum, sonicated for 2 minutes and refilled with argon.

This procedure was repeated two times, followed by addition of copper(I) iodide (232 mg, 1.22 mmol) and bis(tri-tert-butylphosphane) palladium (648 mg, 1.24 mmol) in the glovebox. The flask was then closed, put under vacuum, sonicated for 2 minutes and refilled with argon. The reaction mixture was stirred at 125° C. for 8 hours.

The reaction mixture was filtered over celite, washed with EtOAc and concentrated under vacuum. The obtained solid was triturated with a mixture of n-heptane and MTB ether 4:1 and stirred for 2 hours followed by filtration. Both, the filtered off as well as the filtrate were purified by flash column chromatography (cyclohexane/EtOAc, 8:2) to obtain the desired product (730 mg, 2.21 mmol, 95% yield) as a beige solid.

### Example 11-5: Synthesis of methyl 4-methyl-9-thia-3,4,5,7-tetraazatricyclo[6.3.0.0^{2,6}]undeca-1(8),2,5,10-tetraene-10-carboxylate

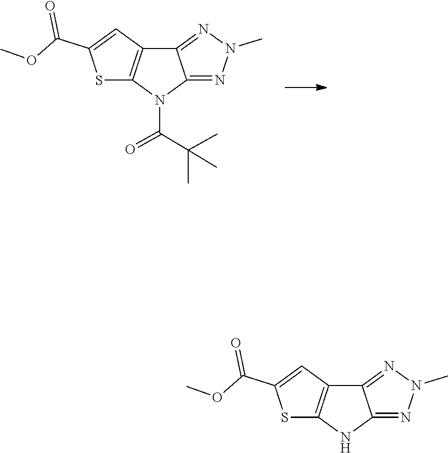

THF (1.7 mL) was cooled to 0° C. and 1.0 mL LDA (1.0 M in THF/hexanes, 1.0 mL, 1.04 mmol) was added. A solution of methyl 7-(2,2-dimethylpropanoyl)-4-methyl-9-thia-3,4,5,7-tetraazatricyclo[6.3.0.0^{2,6}]undeca-1(8),2,5,10-tetraene-10-carboxylate (167 mg, 0.52 mmol) in THF (6.7 mL) was then added dropwise. The reaction was heated to 45° C. and stirred for 2 hours. The reaction mixture was quenched with a saturated ammonium chloride solution, diluted with water and extracted three times with EtOAc. The combined organic phases were dried over sodium sulfate, filtered and concentrated under reduced pressure. The crude product was purified by flash column chromatography (cyclohexane/EtOAc, 60:40 to 100% EtOAc) to obtain the desired product in pure form (105 mg, 0.44 mmol, 80% yield, beige solid).

### Example 11-6: Synthesis of methyl 4-methyl-7-[4-(trifluoromethyl)phenyl]-9-thia-3,4,5,7-tetraazatricyclo[6.3.0.0^{2,6}]undeca-1(8),2,5,10-tetraene-10-carboxylate

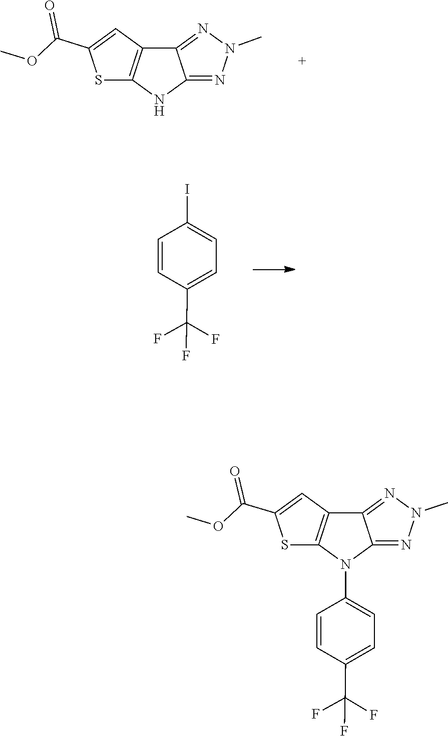

Methyl 4-methyl-9-thia-3,4,5,7-tetraazatricyclo[6.3.0.0^{2,6}]undeca-1(8),2,5,10-tetraene-10-carboxylate (155 mg, 0.53 mmol), 4-iodobenzotrifluoride (101 μL, 0.67 mmol), Cs₂CO₃ (350 mg, 1.06 mmol) and N,N′-dimethylethylenediamine (58 μL, 0.53 mmol) were suspended in 1,4-dioxane (3.1 mL). The vial was put under vacuum, sonicated for 2 minutes and refilled with argon two times, copper(I) iodide (50.7 mg, 0.27 mmol) was added and the vacuum/argon procedure was repeated. The reaction mixture was stirred at 110° C. for 15 hours. The reaction mixture was cooled to room temperature, filtered over celite and washed with EtOAc. The filtrate was concentrated and purified by flash column chromatography (cyclohexane/EtOAc, 8:2 to 100% EtOAc) to give the desired product (131 mg, 0.34 mmol, 40% yield) as pale red solid.

### Example 11-7: Synthesis of 4-methyl-7-[4-(trifluoromethyl)phenyl]-9-thia-3,4,5,7-tetraazatricyclo[6.3.0.0{2,6}]undeca-1(8),2,5,10-tetraene-10-carboxylic acid

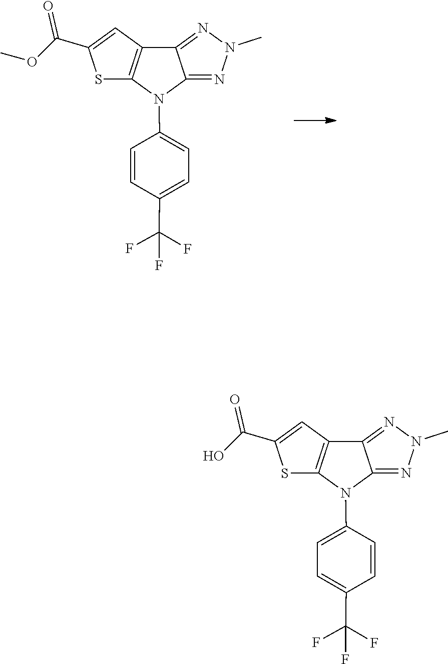

To a suspension of methyl 4-methyl-7-[4-(trifluoromethyl)phenyl]-9-thia-3,4,5,7-tetraazatricyclo[6.3.0.0^{2,6}]undeca-1(8),2,5,10-tetraene-10-carboxy-late (131 mg, 0.34 mmol) in a mixture of THF (4.0 mL) and deionized water (1.0 mL) lithium hydroxide (33.6 mg, 1.4 mmol) was added. The reaction mixture was stirred at 50° C. for 22 hours and at room temperature for 42 hours. The reaction mixture was concentrated, diluted with water, acidified with 1 M aq. HCl to pH 2, and extracted three times with EtOAc. The combined organic phases were dried over sodium sulfate, filtered, concentrated and dried (60° C., 18 h, 0.4 mbar) to afford the desired product (112 mg, 0.30 mmol, 88% yield) as a pale red solid.

### Example 12: N,4-dimethyl-7-[4-(trifluoromethyl)phenyl]-9-thia-3,4,5,7-tetra-azatricyclo[6.3.0.0^{2,6}]undeca-1(8),2,5,10-tetraene-10-carboxamide

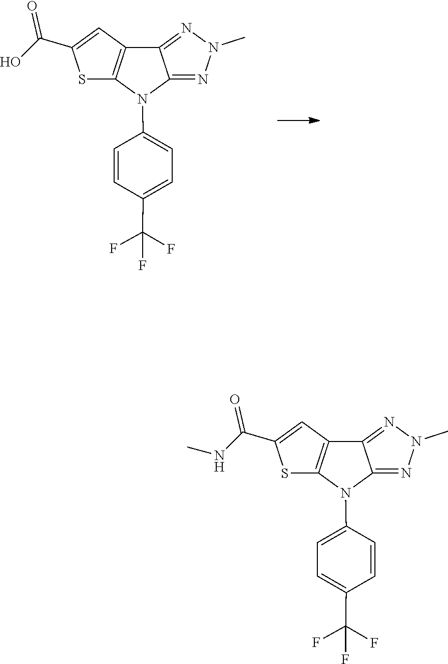

To a solution of 4-methyl-7-[4-(trifluoromethyl)phenyl]-9-thia-3,4,5,7-tetra-azatricyclo[6.3.0.0^{2,6}]undeca-1(8),2,5,10-tetraene-10-carboxylic acid (30.0 mg, 0.08 mmol), methylammonium chloride (11.0 mg, 0.16 mmol) and 4-methylmorpholine (53.5 μL, 0.48 mmol) in DMF (600 μL), EDC·HCl (31.4 mg, 0.16 mmol) and HOBt (12.7 mg, 0.08 mmol) were added. The reaction mixture was stirred at 40° C. for 18 hours. The crude product was purified by by prep HPLC (SunFire C18, A: H2O+0.1% TFA B: MeCN+0.1% TFA, 30% B: 0->4.0 min, 30%->100% B: 4.0->18 min). Pure fractions were combined, basified with saturated NaHO₃ solution and the acetonitrile was evaporated off. The aqueous phase was extracted twice with EtOAc. The combined organic phases were dried over sodium sulfate, filtered and concentrated to give the desired product (24.0 mg, 0.06 mmol, 79% yield) as a white solid.

### Table 1 and Table 1a

Table 1 and Table 1a below show exemplary compounds of the present invention. They have been synthesized as described in the Examples above or similar thereto.

TABLE 1

| Com- | | | |
|---|---|---|---|
| pound | | | |
| No. | | | Conditions and |
| (Example | | | elution time LCMS |
| No.) | Structure & Name | ¹H-NMR | (M + H⁺) |
| 1 (Ex. 1-5) | 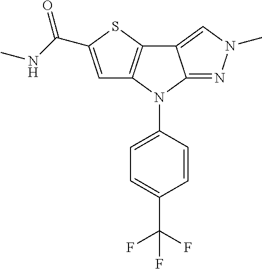 | ¹H NMR (400 MHz, DMSO-d₆) δ 8.56- 8.48 (m, 1H), 8.36- 8.30 (m, 1H), 8.20- 8.12 (m, 2H), 8.12- 8.00 (m, 1H), 7.96- 7.88 (m, 2H), 4.08- 4.01 (m, 3H), 2.83 (d, J = 4.5 Hz, 3H) | Method C, Rₜ = 1.38 min, [M + H]⁺ = 379.0 |
| | N,10-dimethyl-7-[4-(trifluoromethyl)- | | |
| | phenyl]-3-thia-7,9,10-triazatri- | | |
| | cyclo[6.3.0.0^{2,6}]undeca-1(11),2(6),4,8- | | |
| | tetraene-4-carboxamide | | |
| 2 (Ex. 1-4) | 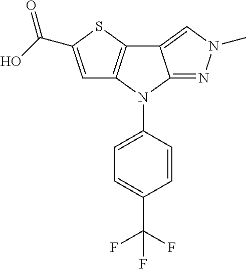 [SMILES: Cn1cc2c3sc(C(=O)O)cc3n(-c3ccc(C(F)(F)F)cc3)c2n1] | ¹H NMR (300 MHz, DMSO-d₆) δ 8.18 (d, J = 8.5 Hz, 2H), 7.99 (d, J = 7.7 Hz, 2H), 7.88 (d, J = 8.7 Hz, 2H), 4.02 (s, 3H). | Method A, Rₜ = 0.83 min, [M + H]⁺ = 366.1 |
| | 10-methyl-7-[4-(trifluoromethyl)phenyl]- | | |
| | 3-thia-7,9,10-triazatricyclo[6.3.0.0^{2,6}]un- | | |
| | deca-1(11),2(6),4,8-tetraene-4- | | |
| | carboxylic acid | | |
| 3 (Ex. 6-4) | 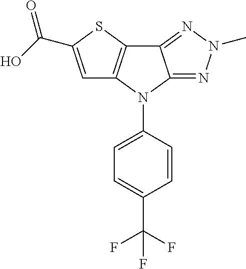 [SMILES: Cn1nc2c3sc(C(=O)O)cc3n(-c3ccc(C(F)(F)F)cc3)c2n1] | ¹H NMR (400 MHz, DMSO-d₆) δ.14 (d, J = 8.5 Hz, 2H), 8.01 (s, 1H), 7.99- 7.91 (m, 2H), 7.25 (s, 1H), 4.35 (s, 3H). | Method B, Rₜ = 1.37 min, [M + H]⁺ = 367.0 |
| | 4-methyl-7-[4-(trifluoromethyl)phenyl]- | | |
| | 11-thia-3,4,5,7-tetraaza- | | |
| | tricyclo[6.3.0.0^{2,6}]undeca-1(8),2,5,9- | | |
| | tetraene-10-carboxylic acid | | |
| 4 (Ex. 6-5) | 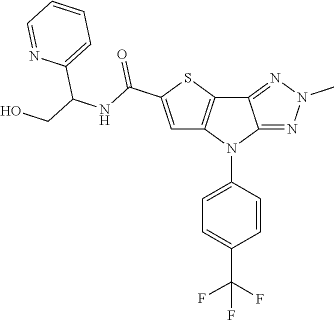 [SMILES: Cn1nc2c3sc(C(=O)NC(CO)c4ccccn4)cc3n(-c3ccc(C(F)(F)F)cc3)c2n1] | ¹H NMR (400 MHz, DMSO-d₆) δ 9.04 (d, J = 8.2 Hz, 1H), 8.74 (s, 1H), 8.57 (d, J = 4.7 Hz, 1H), 8.16 (d, J = 8.5 Hz, 2H), 8.02 (d, J = 8.3 Hz, 2H), 7.83-7.75 (m, 1H), 7.45 (d, J = 8.0 Hz, 1H), 7.35- 7.27 (m, 1H), 5.25- 5.17 (m, 1H), 5.04 (t, J = 5.8 Hz, 1H), 4.37 (s, 3H), 3.94- 3.78 (m, 2H). | Method B, Rₜ = 0.77 min, [M + H]⁺ = 486.9 |
| | N-[2-hydroxy-1-(pyridin-2-yl)ethyl]-4- | | |
| | methyl-7-[4-(trifluoromethyl)phenyl]-11- | | |
| | thia-3,4,5,7-tetraazatri- | | |
| | cyclo[6.3.0.0^{2,6}]undeca-1(8),2,5,9- | | |
| | tetraene-10-carboxamide | | |
| 5 | 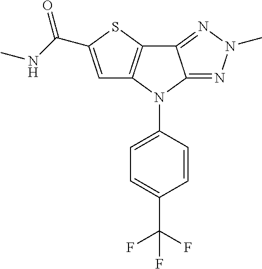 | ¹H NMR (400 MHz, DMSO-d₆) δ 8.72- 8.65 (m, 1H), 8.43 (s, 1H), 8.10 (d, J = 8.4 Hz, 2H), 7.98 (d, J = 8.5 Hz, 2H), 4.36 (s, 3H), 2.85 (d, J = 4.5 Hz, 3H). | Method B, Rₜ = 0.95 min, [M + H]⁺ = 379.8 |
| | N,4-dimethyl-7-[4-(trifluoromethyl)- | | |
| | phenyl]-11-thia-3,4,5,7-tetraazatri- | | |
| | cyclo[6.3.0.0^{2,6}]undeca-1(8),2,5,9- | | |
| | tetraene-10-carboxamide | | |
| 6 | 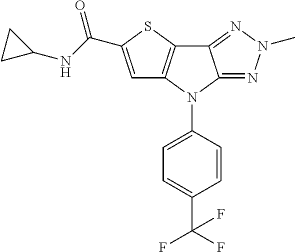 [SMILES: Cn1nc2c3sc(C(=O)NC4CC4)cc3n(-c3ccc(C(F)(F)F)cc3)c2n1] | ¹H NMR (400 MHz, DMSO-d₆) δ 8.70 (d, J = 4.3 Hz, 1H), 8.41 (d, J = 7.3 Hz, 1H), 8.06 (d, J = 8.7 Hz, 2H), 7.98 (d, J = 8.0 Hz, 2H), 4.35 (d, J = 3.6 Hz, 3H), 2.92-2.82 (m, 1H), 0.83-0.71 (m, 2H), 0.66-0.57 (m, 2H). | Method B, Rₜ = 1.00 min, [M + H]⁺ = 406.0 |
| | N-cyclopropyl-4-methyl-7-[4-(trifluoro- | | |
| | methyl)phenyl]-11-thia-3,4,5,7- | | |
| | tetraazatricyclo[6.3.0.0^{2,6}]undeca- | | |
| | 1(8),2,5,9-tetraene-10-carboxamide | | |
| 7 | 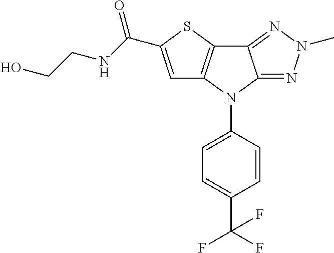 [SMILES: Cn1nc2c3sc(C(=O)NCCO)cc3n(-c3ccc(C(F)(F)F)cc3)c2n1] | ¹H NMR (400 MHz, DMSO-d₆) δ 8.74 (t, J = 5.8 Hz, 1H), 8.50 (s, 1H), 8.07 (d, J = 8.4 Hz, 2H), 7.95 (d, J = 8.4 Hz, 2H), 4.86 (t, J = 5.4 Hz, 1H), 4.34 (s, 3H), 3.62-3.54 (m, 2H), 3.38 (s, 2H). | Method B, Rₜ = 0.85 min, [M + H]⁺ = 410.0 |
| | N-(2-hydroxyethyl)-4-methyl-7-[4- | | |
| | (trifluoromethyl)phenyl]-11-thia-3,4,5,7- | | |
| | tetraazatricyclo[6.3.0.0^{2,6}]undeca- | | |
| | 1(8),2,5,9-tetraene-10-carboxamide | | |
| 8 | 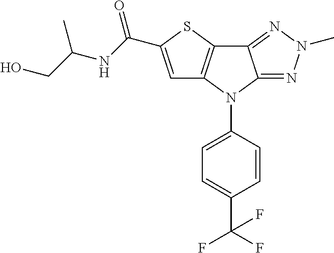 [SMILES: CC(CO)NC(=O)c1cc2c(s1)c1nn(C)nc1n2-c1ccc(C(F)(F)F)cc1] | ¹H NMR (400 MHz, DMSO-d₆) δ 8.50 (s, 1H), 8.39 (d, J = 8.0 Hz, 1H), 8.07 (d, J = 8.4 Hz, 2H), 7.97 (d, J = 8.5 Hz, 2H), 4.84 (t, J = 5.7 Hz, 1H), 4.34 (s, 3H), 4.12-3.98 (m, 1H), 3.57-3.49 (m, 1H), 3.48-3.38 (m, 1H), 1.20 (d, J = 6.8 Hz, 3H). | Method B, Rₜ = 0.90 min, [M + H]⁺ = 423.9 |
| | N-(1-hydroxypropan-2-yl)-4-methyl-7-[4- | | |
| | (trifluoromethyl)phenyl]-11-thia-3,4,5,7- | | |
| | tetraazatricyclo[6.3.0.0^{2,6}]undeca- | | |
| | 1(8),2,5,9-tetraene-10-carboxamide | | |
| 9 | 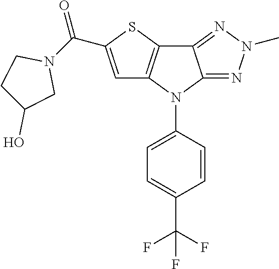 [SMILES: Cn1nc2c3sc(C(=O)N4CCC(O)C4)cc3n(-c3ccc(C(F)(F)F)cc3)c2n1] | ¹H NMR (400 MHz, DMSO-d₆) δ 8.21- 8.06 (m, 3H), 7.94 (s, 2H), 5.09 (d, J = 6.5 Hz, 1H), 4.36 (s, 4H), 4.04 (d, J = 10.1 Hz, 1H), 3.62 (m, 3H), 1.96 (t, J = 34.5 Hz, 2H) | Method B, Rₜ = 0.87 min, [M + H]⁺ = 436.0 |
| | 1-{4-methyl-7-[4-(trifluoromethyl)- | | |
| | phenyl]-11-thia-3,4,5,7-tetraaza- | | |
| | tricyclo[6.3.0.0^{2,6}]undeca-1(8),2,5,9- | | |
| | tetraene-10-carbonyl}pyrrolidin-3-ol | | |
| 10 (Ex. 2) | 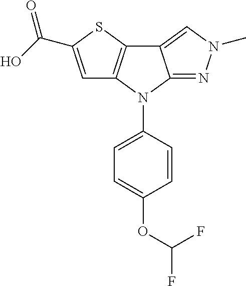 [SMILES: Cn1cc2c3sc(C(=O)O)cc3n(-c3ccc(OC(F)F)cc3)c2n1] | ¹H NMR (400 MHz, DMSO-d₆) δ 13.0 (s, 1H), 8.05 (d, J = 7.0 Hz, 2H), 7.97 (d, J = 9.0 Hz, 2H), 7.38 (d, J = 8.9 Hz, 2H), 7.24 (s, 1H), 4.03 (s, 3H) | Method C, Rₜ = 1.30 min, [M + H]⁺ = 364.0 |
| | 7-[4-(difluoromethoxy)phenyl]-10- | | |
| | methyl-3-thia-7,9,10- | | |
| | triazatricyclo[6.3.0.0^{2,6}]undeca- | | |
| | 1(11),2(6),4,8-tetraene-4-carboxylic acid | | |
| 11 (Ex. 3) | 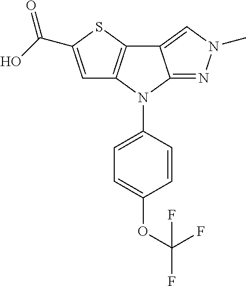 [SMILES: Cn1cc2c3sc(C(=O)O)cc3n(-c3ccc(OC(F)(F)F)cc3)c2n1] | ¹H NMR (700 MHz, DMSO-d₆) δ 13.1 (s, 1H), 8.10 (s, 1H), 8.08-8.05 (m, 3H), 7.56 (d, J = 8.7 Hz, 2H), 4.04 (s, 3H) | Method C, Rₜ = 1.40 min, [M + H]⁺ = 382.0 |
| | 10-methyl-7-[4-(trifluoromethoxy)- | | |
| | phenyl]-3-thia-7,9,10- | | |
| | triazatricyclo[6.3.0.0^{2,6}]undeca- | | |
| | 1(11),2(6),4,8-tetraene-4-carboxylic acid | | |
| 12 | 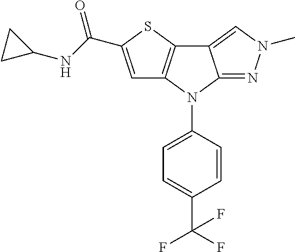 [SMILES: Cn1cc2c3sc(C(=O)NC4CC4)cc3n(-c3ccc(C(F)(F)F)cc3)c2n1] | ¹H NMR (400 MHz, DMSO-d₆) δ 8.52 (d, J = 3.7 Hz, 1H), 8.32 (s, 1H), 8.15 (d, J = 8.5 Hz, 2H), 8.04 (s, 1H), 7.92 (d, J = 8.7 Hz, 2H), 4.04 (s, 3H), 2.86- 2.80 m, 1H), 0.77- 0.68 (m, 2H), 0.65- 0.57 (m, 2H). | Method C, Rₜ = 1.45 min, [M + H]⁺ = 405.1 |
| | N-cyclopropyl-10-methyl-7-[4- | | |
| | (trifluoromethyl)phenyl]-3-thia-7,9,10- | | |
| | triazatricyclo[6.3.0.0^{2,6}]undeca- | | |
| | 1(11),2(6),4,8-tetraene-4-carboxamide | | |
| 13 | 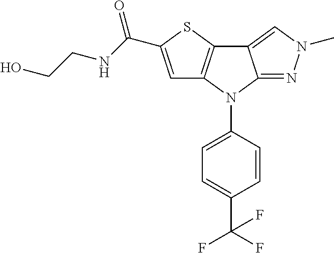 [SMILES: Cn1cc2c3sc(C(=O)NCCO)cc3n(-c3ccc(C(F)(F)F)cc3)c2n1] | ¹H NMR (700 MHz, DMSO-d₆) δ 8.59 (t, J = 5.7 Hz, 1H), 8.44 (s, 1H), 8.18 (d, J = 8.6 Hz, 2H), 8.05 (s, 1H), 7.93 (d, J = 8.6 Hz, 2H), 4.80 (t, J = 5.5 Hz, 1H), 4.04 (s, 3H), 3.54 (q, J = 5.9 Hz, 2H), 3.36 (q, J = 6.0 Hz, 2H). | Method C, Rₜ = 1.30 min, [M + H]⁺ = 409.0 |
| | N-(2-hydroxyethyl)-10-methyl-7-[4- | | |
| | (trifluoromethyl)phenyl]-3-thia-7,9,10- | | |
| | triazatricyclo [6.3.0.0^{2,6}]undeca- | | |
| | 1(11),2(6),4,8-tetraene-4-carboxamide | | |
| 14 | 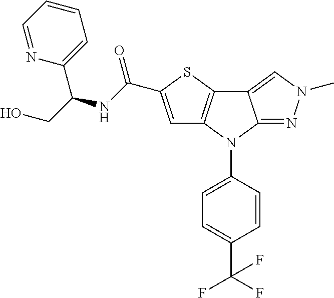 [SMILES: Cn1cc2c3sc(C(=O)N[C@@H](CO)c4ccccn4)cc3n(-c3ccc(C(F)(F)F)cc3)c2n1] | ¹H NMR (400 MHz, DMSO-d₆) δ 8.83 (d, J = 8.1 Hz, 1H), 8.61 (s, 1H), 8.57- 8.55 (m, 1H), 8.21 (d, J = 8.5 Hz, 2H), 8.05 (s, 1H), 7.95 (d, J = 8.6 Hz, 2H), 7.78 (td, J = 7.7, 1.8 Hz, 1H), 7.45 (d, J = 7.9 Hz, 1H), 7.29 (ddd, J = 7.4, 4.8, 0.9 Hz, 1H), 5.21-5.16 (m, 1H), 4.98 (s, 1H), 4.04 (s, 3H), 3.92-3.81 (m, 2H). | Method C, Rₜ = 1.18 min, [M + H]⁺ = 486.1 |
| | N-[(1R)-2-hydroxy-1-(pyridin-2-yl)ethyl]- | | |
| | 10-methyl-7-[4-(trifluoromethyl)phenyl]- | | |
| | 3-thia-7,9,10-triazatricyclo[6.3.0.0^{2,6}]un- | | |
| | deca-1(11),2(6),4,8-tetraene-4- | | |
| | carboxamide | | |
| 15 | 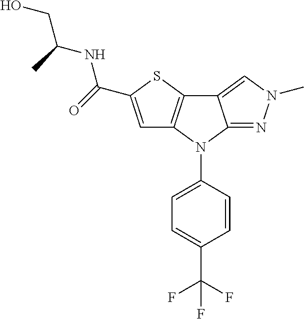 [SMILES: C[C@@H](CO)NC(=O)c1cc2c(s1)c1cn(C)nc1n2-c1ccc(C(F)(F)F)cc1] | ¹H NMR (700 MHz, DMSO-d₆) δ 8.43 (s, 1H), 8.23 (d, J = 8.0 Hz, 1H), 8.18 (d, J = 8.6 Hz, 2H), 8.05 (s, 1H), 7.94 (d, J = 8.6 Hz, 2H), 4.80 (t, J = 5.7 Hz, 1H), 4.04 (s, 3H), 4.04-4.00 (m, 1H) 3.50-3.47 (m, 1H), 3.40-3.37 (m, 1H), 1.18 (d, J = 6.8 Hz, 3H) | Method C, Rₜ = 1.34 min, [M + H]⁺ = 423.0 |
| | N-[(2S)-1-hydroxypropan-2-yl]-10- | | |
| | methyl-7-[4-(trifluoromethyl)phenyl]-3- | | |
| | thia-7,9,10-triazatri- | | |
| | cyclo[6.3.0.0^{2,6}]undeca-1(11),2(6),4,8- | | |
| | tetraene-4-carboxamide | | |
| 16 | 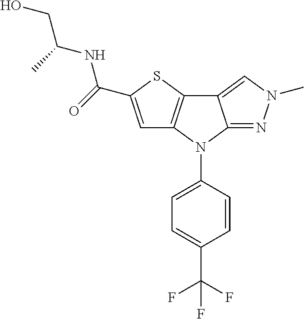 [SMILES: C[C@H](CO)NC(=O)c1cc2c(s1)c1cn(C)nc1n2-c1ccc(C(F)(F)F)cc1] | ¹H NMR (700 MHz, DMSO-d₆) δ 8.43 (s, 1H), 8.23 (d, J = 8.0 Hz, 1H), 8.18 (d, J = 8.6 Hz, 2H), 8.05 (s, 1H), 7.94 (d, J = 8.6 Hz, 2H), 4.80 (t, J = 5.7 Hz, 1H), 4.04 (s, 3H), 4.04-4.00 (m, 1H) 3.50-3.47 (m, 1H), 3.40-3.37 (m, 1H), 1.18 (d, J = 6.8 Hz, 3H) | Method C, Rₜ = 1.34 min, [M + H]⁺ = 423.0 |
| | N-[(2R)-1-hydroxypropan-2-yl]-10- | | |
| | methyl-7-[4-(trifluoromethyl)phenyl]-3- | | |
| | thia-7,9,10-triaza- | | |
| | tricyclo[6.3.0.0^{2,6}]undeca-1(11),2(6),4,8- | | |
| | tetraene-4-carboxamide | | |
| 17 | 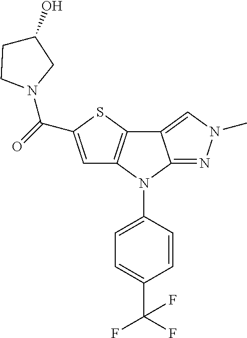 [SMILES: Cn1cc2c3sc(C(=O)N4CC[C@H](O)C4)cc3n(-c3ccc(C(F)(F)F)cc3)c2n1] | ¹H NMR (700 MHz, DMSO-d₆) δ 8.20 (d, J = 7.7 Hz, 2H), 8.06 (s, 1H), 8.04- 7.99 (m, 1H), 7.91 (d, J = 8.6 Hz, 2H), 5.76 (s, 1H), 5.05 (s, 1H), 4.41-4.34 (m, 1H), 4.04-3.97 (m, 4H), 3.73-3.47 (m, 2H), 2.03-1.85 (m, 2H) | Method C, Rₜ = 1.32 min, [M + H]⁺ = 435.0 |
| | (3S)-1-{10-methyl-7-[4-(trifluoro- | | |
| | methyl)phenyl]-3-thia-7,9,10- | | |
| | triazatricyclo[6.3.0.0^{2,6}]undeca- | | |
| | 1(11),2(6),4,8-tetraene-4- | | |
| | carbonyl}pyrrolidin-3-ol | | |
| 18 | 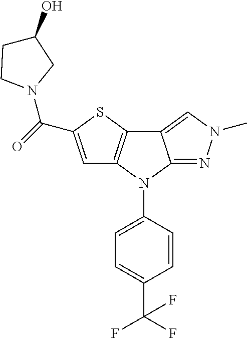 [SMILES: Cn1cc2c3sc(C(=O)N4CC[C@@H](O)C4)cc3n(-c3ccc(C(F)(F)F)cc3)c2n1] | ¹H NMR (700 MHz, DMSO-d₆) δ 8.20 (d, J = 7.7 Hz, 2H), 8.06 (s, 1H), 8.04- 7.99 (m, 1H), 7.91 (d, J = 8.6 Hz, 2H), 5.76 (s, 1H), 5.05 (s, 1H), 4.41-4.34 (m, 1H), 4.04-3.97 (m, 4H), 3.73-3.47 (m, 2H), 2.03-1.85 (m, 2H) | Method C, Rₜ = 1.32 min, [M + H]⁺ = 435.1 |
| | (3R)-1-{10-methyl-7-[4-(trifluoro- | | |
| | methyl)phenyl]-3-thia-7,9,10- | | |
| | triazatricyclo[6.3.0.0^{2,6}]undeca- | | |
| | 1(11),2(6),4,8-tetraene-4- | | |
| | carbonyl}pyrrolidin-3-ol | | |
| 19 (Ex. 7-4) | 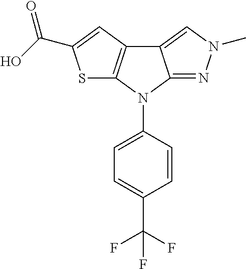 [SMILES: Cn1cc2c3cc(C(=O)O)sc3n(-c3ccc(C(F)(F)F)cc3)c2n1] | 1H NMR (700 MHz, DMSO) δ 13.06 (s, 1H), 8.24 (d, J = 8.6 Hz, 2H), 8.02-8.00 (m, 4H), 4.07 (s, 3H). | Method C, Rₜ = 1.42 min, [M + H]⁺ = 366.0 |
| | 4-methyl-7-[4-(trifluoromethyl)phenyl]- | | |
| | 9-thia-4,5,7-triaza- | | |
| | tricyclo[6.3.0.0^{2,6}]undeca-1(8),2,5,10- | | |
| | tetraene-10-carboxylic acid | | |
| 20 (Ex. 8-5) | 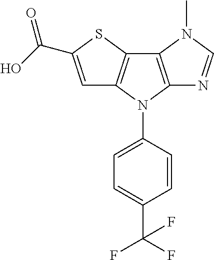 [SMILES: Cn1cnc2c1c1sc(C(=O)O)cc1n2-c1ccc(C(F)(F)F)cc1] | 1H NMR (400 MHz, MeOH-d4) δ 8.08 (d, J = 8.9 Hz, 3H), 7.87 (d, J = 8.5 Hz, 2H), 7.74 (s, 1H), 4.02 (s, 3H) | Method D, Rₜ = 0.90 min, [M + H]⁺ = 366.0 |
| | 3-methyl-7-[4-(trifluoromethyl)phenyl]- | | |
| | 11-thia-3,5,7-triazatri- | | |
| | cyclo[6.3.0.0^{(2,6)}]]undeca-1(8),2(6),4,9- | | |
| | tetraene-10-carboxylic acid | | |
| 21 (Ex. 8-6) | 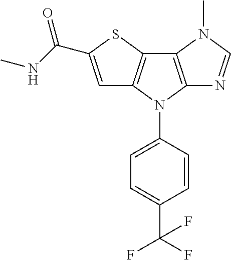 [SMILES: CNC(=O)c1cc2c(s1)c1c(ncn1C)n2-c1ccc(C(F)(F)F)cc1] | 1H NMR (400 MHz, DMSO-d6) δ 8.51 (d, J = 4.8 Hz, 1H), 8.36 (s, 1H), 8.22 (d, J = 8.5 Hz, 2H), 7.94 (d, J = 8.5 Hz, 2H), 7.84 (s, 1H), 3.95 (s, 3H), 2.83 (d, J = 4.5 Hz, 3H) | Method B, Rₜ = 0.83 min, [M + H]⁺ = 379.0 |
| | N,3-dimethyl-7-[4-(trifluoro- | | |
| | methyl)phenyl]-11-thia-3,5,7- | | |
| | triazatricyclo[6.3.0.0^{2,6}]undeca- | | |
| | 1(8),2(6),4,9-tetraene-10-carboxamide | | |
| 22 (Ex. 9-5) | 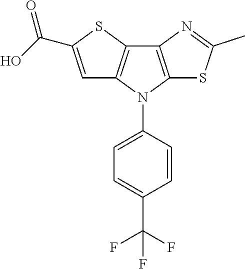 [SMILES: Cc1nc2c3sc(C(=O)O)cc3n(-c3ccc(C(F)(F)F)cc3)c2s1] | 1H NMR (400 MHz, DMSO-d6) δ 8.09- 7.87 (m, 5H), 2.82 (s, 3H) | Method B, Rₜ = 1.14 min, [M + H]⁺ = 383.0 |
| | 4-methyl-7-[4-(trifluoromethyl)phenyl]- | | |
| | 5,11-dithia-3,7-diazatri- | | |
| | cyclo[6.3.0.02,6]undeca-1(8),2(6),3,9- | | |
| | tetraene-10-carboxylic acid | | |
| 23 (Ex. 9-6) | 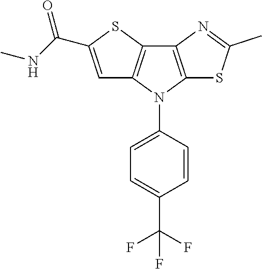 | 1H NMR (400 MHz, DMSO-d6) δ 8.55 (d, J = 5.1 Hz, 1H), 8.23 (s, 1H), 8.03 (d, J = 8.5 Hz, 2H), 7.95 (d, J = 8.4 Hz, 2H), 2.82 (t, J = 2.4 Hz, 6H) | Method E, Rₜ = 1.29 min, [M + H]⁺ = 396.1 |
| | N,4-dimethyl-7-[4-(trifluoro- | | |
| | methyl)phenyl]-5,11-dithia-3,7- | | |
| | diazatricyclo[6.3.0.0^{2,6}]undeca- | | |
| | 1(8),2(6),3,9-tetraene-10-carboxamide | | |
| 24 (Ex. 4) | 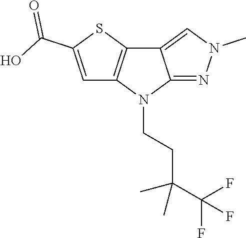 [SMILES: Cn1cc2c3sc(C(=O)O)cc3n(CCC(C)(C)C(F)(F)F)c2n1] | 1H NMR (700 MHz, DMSO) δ 12.85 (s, 1H), 7.91 (s, 1H), 7.90 (s, 1H), 4.23- 4.18 (m, 2H), 3.97 (s, 3H), 2.01-1.97 (m, 2H), 1.18 (s, 6H) | Method C, Rₜ = 1.29 min, [M + H]⁺ = 360.1 |
| | 10-methyl-7-(4,4,4-trifluoro-3,3- | | |
| | dimethylbutyl)-3-thia-7,9,10- | | |
| | triazatricyclo[6.3.0.0^{2,6}]undeca- | | |
| | 1(11),2(6),4,8-tetraene-4-carboxylic acid | | |
| 25 | 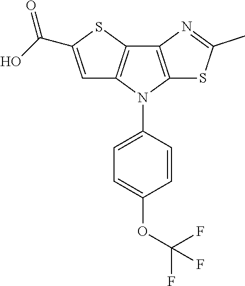 [SMILES: Cc1nc2c3sc(C(=O)O)cc3n(-c3ccc(OC(F)(F)F)cc3)c2s1] | 1H NMR (400 MHz, DMSO-d6) δ 7.88 (d, J = 8.7 Hz, 3H), 7.63 (d, J = 8.5 Hz, 2H), 2.80 (s, 3H). | Method B, Rₜ = 1.15 min, [M + H]⁺ = 399.0 |
| | 4-methyl-7-[4-(trifluoro- | | |
| | methoxy)phenyl]-5,11-dithia-3,7- | | |
| | diazatricyclo[6.3.0.0^{2,6}]undeca- | | |
| | 1(8),2(6),3,9-tetraene-10-carboxylic acid | | |
| 26 (Ex. 10) | 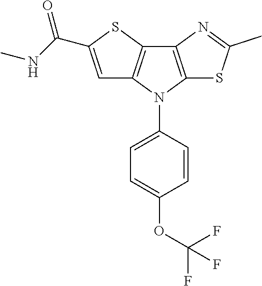 | 1H NMR (400 MHz, DMSO-d6) δ 8.55 (d, J = 5.1 Hz, 1H), 8.23 (s, 1H), 8.03 (d, J = 8.5 Hz, 2H), 7.95 (d, J = 8.4 Hz, 2H), 2.82 (t, J = 2.4 Hz, 6H). | Method E, Rₜ = 1.29 min, [M + H]⁺ = 396.1 |
| | N,4-dimethyl-7-[4-(trifluoro- | | |
| | methoxy)phenyl]-5,11-dithia-3,7- | | |
| | diazatricyclo[6.3.0.0^{2,6}]undeca- | | |
| | 1(8),2(6),3,9-tetraene-10-carboxamide | | |
| 27 | 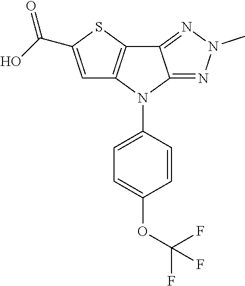 [SMILES: Cn1nc2c3sc(C(=O)O)cc3n(-c3ccc(OC(F)(F)F)cc3)c2n1] | 1H NMR (300 MHz, DMSO-d6) δ 8.07- 7.95 (m, 3H), 7.59 (d, J = 8.5 Hz, 2H), 7.36-7.30 (b, 1H), 4.35 (s, 3H). | Method F, Rₜ = 1.22 min, [M + H]⁺ = 383.1 |
| | 4-methyl-7-[4-(trifluoromethoxy)- | | |
| | phenyl]-11-thia-3,4,5,7-tetraazatri- | | |
| | cyclo[6.3.0.0^{2,6}]undeca-1(8),2,5,9- | | |
| | tetraene-10-carboxylic acid | | |
| 28 | 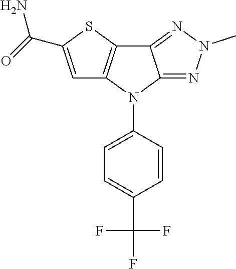 [SMILES: Cn1nc2c3sc(C(N)=O)cc3n(-c3ccc(C(F)(F)F)cc3)c2n1] | 1H NMR (400 MHz, DMSO-d6) δ 8.52 (s, 1H), 8.20 (s, 1H), 8.12 (d, J = 8.5 Hz, 2H), 7.99 (d, J = 8.6 Hz, 2H), 7.66 (s, 1H), 4.37 (s, 3H). | Method G, Rₜ = 1.14 min, [M + H]⁺ = 366.1 |
| | 4-methyl-7-[4-(trifluoromethyl)phenyl]- | | |
| | 11-thia-3,4,5,7-tetraazatri- | | |
| | cyclo[6.3.0.0^{2,6}]undeca-1(8),2,5,9- | | |
| | tetraene-10-carboxamide | | |
| 29 | 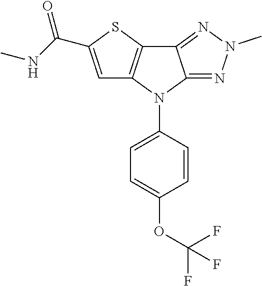 | 1H NMR (300 MHz, DMSO-d6) δ 8.66 (d, J = 4.8 Hz, 1H), 8.34 (s, 1H), 8.02- 7.90 (m, 2H), 7.64 (d, J = 8.6 Hz, 2H), 4.34 (s, 3H), 2.84 (d, J = 4.6 Hz, 3H). | Method G, Rₜ = 1.15 min, [M + H]⁺ = 396.0 |
| | N,4-dimethyl-7-[4-(trifluoro- | | |
| | methoxy)phenyl]-11-thia-3,4,5,7- | | |
| | tetraazatricyclo[6.3.0.0^{2,6}]undeca- | | |
| | 1(8),2,5,9-tetraene-10-carboxamide | | |
| 30 | 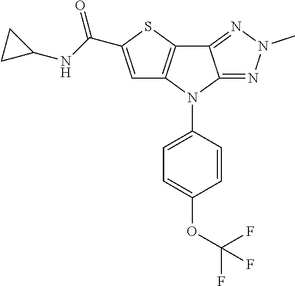 [SMILES: Cn1nc2c3sc(C(=O)NC4CC4)cc3n(-c3ccc(OC(F)(F)F)cc3)c2n1] | 1H NMR (400 MHz, DMSO-d6) δ 8.69 (d, J = 3.9 Hz, 1H), 8.34 (d, J = 2.4 Hz, 1H), 7.96 (m, 2H), 7.65 (d, J = 8.5 Hz, 2H), 4.34 (s, 3H), 2.85 (m, 1H), 0.76 (m, 2H), 0.64-0.56 (m, 2H) | Method F, Rₜ = 1.72 min, [M + H]⁺ = 422.1 |
| | N-cyclopropyl-4-methyl-7-[4-(tri- | | |
| | fluoromethoxy)phenyl]-11-thia-3,4,5,7- | | |
| | tetraazatricyclo[6.3.0.0^{2,6}]deca- | | |
| | 1(8),2,5,9-tetraene-10-carboxamide | | |
| 31 | 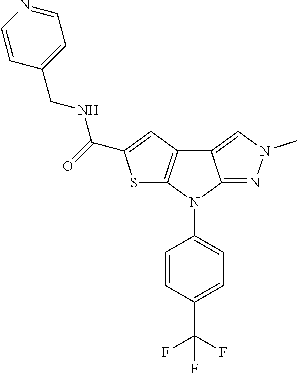 [SMILES: Cn1cc2c3cc(C(=O)NCc4ccncc4)sc3n(-c3ccc(C(F)(F)F)cc3)c2n1] | 1H NMR (700 MHz, DMSO) d 9.20 (t, J = 6.0 Hz, 1H), 8.53 (d, J = 5.1 Hz, 2H), 8.24 (d, J = 8.4 Hz, 2H), 8.14 (s, 1H), 8.06 (s, 1H), 8.02 (d, J = 8.5 Hz, 2H), 7.35 (d, J = 5.2 Hz, 2H), 4.54 (d, J = 6.0 Hz, 2H), 4.07 (s, 3H). | Method C, Rₜ = 1.20 min, [M + H]⁺ = 456.1 |
| | 4-methyl-N-[(pyridin-4-yl)methyl]-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 32 | 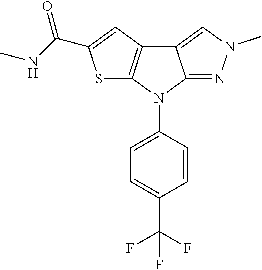 | 1H NMR (700 MHz, DMSO) δ 8.52-8.50 (m, 1H), 8.25-8.21 (m, 2H), 8.04 (s, 1H), 8.02-7.99 (m, 2H), 7.98 (s, 1H), 4.06 (s, 3H), 2.81 (d, J = 4.5 Hz, 3H). | Method C, Rₜ = 1.39 min, [M + H]⁺ = 379.0 |
| | N,4-dimethyl-7-[4-(trifluoro- | | |
| | methyl)phenyl]-9-thia-4,5,7- | | |
| | triazatricyclo[6.3.0.0^{2,6}]undeca- | | |
| | 1(8),2,5,10-tetraene-10-carboxamide | | |
| 33 | 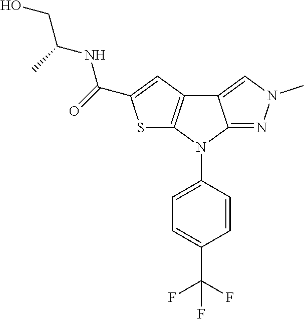 [SMILES: C[C@H](CO)NC(=O)c1cc2c3cn(C)nc3n(-c3ccc(C(F)(F)F)cc3)c2s1] | 1H NMR (700 MHz, DMSO) δ 8.25-8.21 (m, 2H), 8.19 (d, J = 8.0 Hz, 1H), 8.12 (s, 1H), 8.05 (s, 1H), 8.03-7.99 (m, 2H), 4.77-4.75 (m, 1H), 4.06 (s, 3H), 4.04-3.97 (m, 1H), 3.50-3.46 (m, 1H), 3.37-3-34 (m, 1H), 1.16 (d, J = 6.7 Hz, 3H) | Method C, Rₜ = 1.35 min, [M + H]⁺ = 423.0 |
| | N-[(2R)-1-hydroxypropan-2-yl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 34 (Ex. 7-5) | 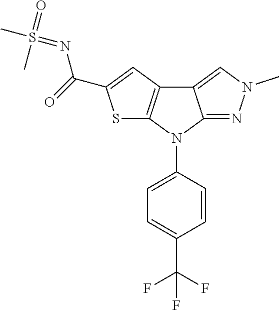 | 1H NMR (700 MHz, DMSO) δ 8.23 (d, J = 8.6 Hz, 2H), 8.02 (d, J = 8.7 Hz, 2H), 7.97 (s, 1H), 7.92 (s, 1H), 4.06 (s, 3H), 3.49 (s, 6H) | Method C, Rₜ = 1.39 min, [M + H]⁺ = 441.0 |
| | N-[dimethyl(oxo)-λ⁶-sulfanylidene]-4- | | |
| | methyl-7-[4-(trifluoromethyl)phenyl]-9- | | |
| | thia-4,5,7-triazatricyclo[6.3.0.0^{2,6}]un- | | |
| | deca-1(8),2,5,10-tetraene-10- | | |
| | carboxamide | | |
| 35 | 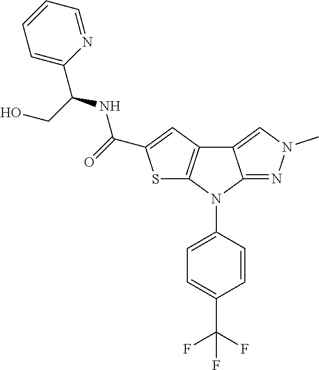 [SMILES: Cn1cc2c3cc(C(=O)N[C@@H](CO)c4ccccn4)sc3n(-c3ccc(C(F)(F)F)cc3)c2n1] | 1H NMR (700 MHz, DMSO) δ 8.79 (d, J = 8.1 Hz, 1H), 8.55- 8.54 (m, 1H), 8.31 (s, 1H), 8.23 (d, J = 8.6 Hz, 2H), 8.07 (s, 1H), 8.01 (d, J = 8.7 Hz, 2H), 7.77-7.76 (m, 1H), 7.47-7.43 (m, 1H), 7.28 (ddd, J = 7.5, 4.8, 1.1 Hz, 1H), 5.17-5.14 (m, 1H), 4.97 (t, J = 5.9 Hz, 1H), 4.07 (s, 3H), 3.88-3-85 (m, 1H), 3.81-3-78 (m, 1H) | Method C, Rₜ = 1.20 min, [M + H]⁺ = 486.0 |
| | N-[(1R)-2-hydroxy-1-(pyridin-2-yl)ethyl]- | | |
| | 4-methyl-7-[4-(trifluoromethyl)phenyl]- | | |
| | 9-thia-4,5,7-triazatricyclo[6.3.0.0^{2,6}]un- | | |
| | deca-1(8),2,5,10-tetraene-10- | | |
| | carboxamide | | |
| 36 (Ex. 5-2) | 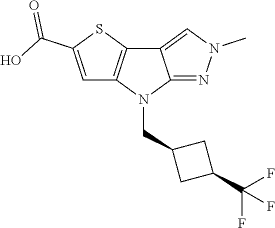 [SMILES: Cn1cc2c3sc(C(=O)O)cc3n(C[C@H]3C[C@@H](C(F)(F)F)C3)c2n1] | 1H NMR (700 MHz, DMSO) δ 12.83 (s, 1H), 7.97 (s, 1H), 7.89 (s, 1H), 4.24 (d, J = 7.7 Hz, 2H), 3.96 (s, 3H), 3.31- 3.28 (m, 1H), 2.93 (q, J = 7.3 Hz, 1H), 2.15 (ddd, J = 12.5, 8.9, 6.6 Hz, 2H), 2.07 (ddd, J = 13.2, 9.3, 5.3 Hz, 2H) | Method C, Rₜ = 1.26 min, [M + H]⁺ = 358.1 |
| | 10-methyl-7-{[(1s,3s)-3- | | |
| | (trifluoromethyl)cyclobutyl]methyl}-3- | | |
| | thia-7,9,10- | | |
| | triazatricyclo[6.3.0.0^{2,6}]undeca- | | |
| | 1(11),2(6),4,8-tetraene-4-carboxylic acid | | |
| 37 (Ex. 5-2) | 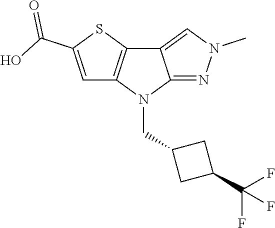 [SMILES: Cn1cc2c3sc(C(=O)O)cc3n(C[C@H]3C[C@H](C(F)(F)F)C3)c2n1] | 1H NMR (700 MHz, DMSO) δ 12.83 (s, 1H), 7.89 (s, 1H), 7.88 (s, 1H), 4.13 (d, J = 6.9 Hz, 2H), 3.96 (s, 3H), 3.04 (dq, J = 18.2, 9.2 Hz, 1H), 2.86 (hept, J = 8.1 Hz, 1H), 2.16-2.09 (m, 2H), 1.92 (qd, J = 9.5, 2.6 Hz, 2H) | Method C, Rₜ = 1.23 min, [M + H]⁺ = 358.1 |
| | 10-methyl-7-{[(1r,3r)-3- | | |
| | (trifluoromethyl)cyclobutyl]methyl}-3- | | |
| | thia-7,9,10-triazatri- | | |
| | cyclo[6.3.0.0^{2,6}]undeca-1(11),2(6),4,8- | | |
| | tetraene-4-carboxylic acid | | |
| 38 | 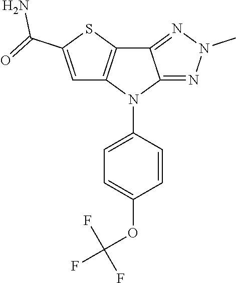 [SMILES: Cn1nc2c3sc(C(N)=O)cc3n(-c3ccc(OC(F)(F)F)cc3)c2n1] | 1H NMR (300 MHz, DMSO-d6) δ 8.43 (s, 1H), 8.18 (s, 1H), 8.03-7.93 (m, 2H), 7.65 (d, J = 8.8 Hz, 3H), 4.34 (s, 3H) | Method G, Rₜ = 1.18 min, [M + H]⁺ = 382.1 |
| | 4-methyl-7-[4-(trifluoromethoxy)- | | |
| | phenyl]-11-thia-3,4,5,7- | | |
| | tetraazatricyclo[6.3.0.0^{2,6}]undeca- | | |
| | 1(8),2,5,9-tetraene-10-carboxamide | | |
| 39 (Ex. 5-3) | 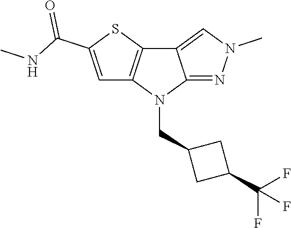 [SMILES: CNC(=O)c1cc2c(s1)c1cn(C)nc1n2C[C@H]1C[C@@H](C(F)(F)F)C1] | 1H NMR (700 MHz, DMSO) δ 8.32-8.30 (m, 1H), 7.85 (s, 1H), 7.83 (s, 1H), 4.17 (d, J = 7.5 Hz, 2H), 3.95 (s, 3H), 3.27-3.22 (m, 1H), 2.96-2.91 (m, 1H), 2.78 (d, J = 4.5 Hz, 3H), 2.20-2.16 (m, 2H), 2.14-2.10 (m, 2H) | Method C, Rₜ = 1.23 min, [M + H]⁺ = 371.0 |
| | N,10-dimethyl-7-{[(1s,3s)-3- | | |
| | (trifluoromethyl)cyclobutyl]methyl}-3- | | |
| | thia-7,9,10-triazatri- | | |
| | cyclo[6.3.0.0^{2,6}]undeca-1(11),2(6),4,8- | | |
| | tetraene-4-carboxamide | | |
| 40 | 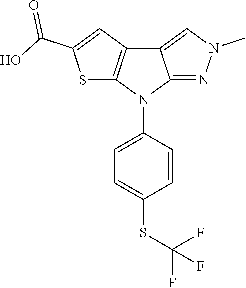 [SMILES: Cn1cc2c3cc(C(=O)O)sc3n(-c3ccc(SC(F)(F)F)cc3)c2n1] | 1H NMR (400 MHz, DMSO) δ 13.00 (s, 1H), 8.23-8.15 (m, 2H), 8.03- 7.95 (m, 4H), 4.06 (s, 3H). | Method C, Rₜ = 1.47 min, [M + H]⁺ = 398.0 |
| | 4-methyl-7-{4-[(trifluoromethyl)- | | |
| | sulfanyl]phenyl}-9-thia-4,5,7- | | |
| | triazatricyclo[6.3.0.0^{2,6}]undeca- | | |
| | 1(8),2,5,10-tetraene-10-carboxylic acid | | |
| 41 | 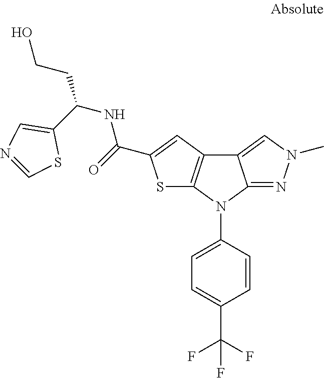 [SMILES: Cn1cc2c3cc(C(=O)N[C@@H](CCO)c4cncs4)sc3n(-c3ccc(C(F)(F)F)cc3)c2n1] | 1H NMR (400 MHz, DMSO) δ 8.98 (d, J = 0.8 Hz, 1H), 8.92 (d, J = 8.2 Hz, 1H), 8.27-8.20 (m, 2H), 8.15 (s, 1H), 8.04 (s, 1H), 8.00 (d, J = 8.7 Hz, 2H), 7.83 (t, J = 0.8 Hz, 1H), 5.52 (q, J = 8.2 Hz, 1H), 4.62 (t, J = 4.9 Hz, 1H), 4.06 (s, 3H), 3.59-3.43 (m, 2H), 2.23- 2.03 (m, 1H). | Method C, Rₜ = 1.32 min, [M + H]⁺ = 506.0 |
| | single enantiomer, absolute | | |
| | configuration has been assigned | | |
| | arbitrarily | | |
| | N-[(1S)-3-hydroxy-1-(1,3-thiazol-5- | | |
| | yl)propyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 42 | 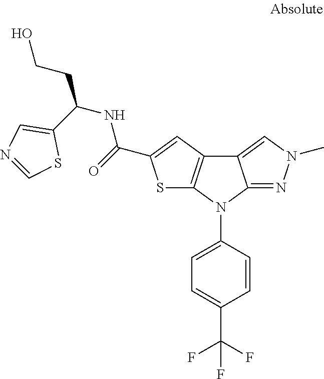 [SMILES: Cn1cc2c3cc(C(=O)N[C@H](CCO)c4cncs4)sc3n(-c3ccc(C(F)(F)F)cc3)c2n1] | 1H NMR (400 MHz, DMSO) δ 8.98 (d, J = 0.8 Hz, 1H), 8.92 (d, J = 8.2 Hz, 1H), 8.27-8.20 (m, 2H), 8.15 (s, 1H), 8.04 (s, 1H), 8.00 (d, J = 8.7 Hz, 2H), 7.83 (t, J = 0.8 Hz, 1H), 5.52 (q, J = 8.2 Hz, 1H), 4.62 (t, J = 4.9 Hz, 1H), 4.06 (s, 3H), 3.59-3.43 (m, 2H), 2.23- 2.03 (m, 1H). | Method C, Rₜ = 1.32 min, [M + H]⁺ = 506.0 |
| | single enantiomer, absolute | | |
| | configuration has been assigned | | |
| | arbitrarily | | |
| | N-[(1R)-3-hydroxy-1-(1,3-thiazol-5- | | |
| | yl)propyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 43 | 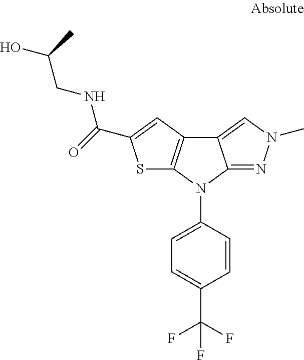 [SMILES: C[C@H](O)CNC(=O)c1cc2c3cn(C)nc3n(-c3ccc(C(F)(F)F)cc3)c2s1] | 1H NMR (400 MHz, DMSO) δ 8.47 (t, J = 5.8 Hz, 1H), 8.27- 8.20 (m, 2H), 8.10 (s, 1H), 8.04 (s, 1H), 8.03-7.96 (m, 2H), 4.75 (d, J = 4.7 Hz, 1H), 4.06 (s, 3H), 3.80 (dt, J = 11.8, 6.0 Hz, 1H), 3.22 (td, J = 6.0, 2.4 Hz, 2H), 1.09 (d, J = 6.2 Hz, 3H). | Method C, Rt = 1.34 min, [M + H]+ = 423.0 |
| | N-[(2S)-2-hydroxypropyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 44 | 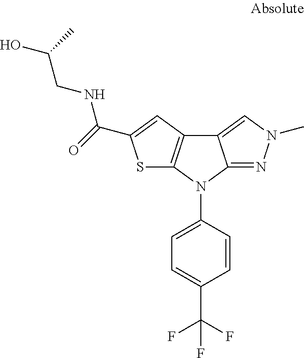 [SMILES: C[C@@H](O)CNC(=O)c1cc2c3cn(C)nc3n(-c3ccc(C(F)(F)F)cc3)c2s1] | 1H NMR (400 MHz, DMSO) δ 8.47 (t, J = 5.8 Hz, 1H), 8.23 (d, J = 8.5 Hz, 2H), 8.10 (s, 1H), 8.04 (s, 1H), 8.02-7.97 (m, 2H), 4.74 (d, J = 4.7 Hz, 1H), 4.06 (s, 3H), 3.80 (dq, J = 11.7, 6.0 Hz, 1H), 3.22 (td, J = 6.0, 2.3 Hz, 2H), 1.09 (d, J = 6.2 Hz, 3H). | Method C, Rt = 1.34 min, [M+H]+ = 423.0 |
| | N-[(2R)-2-hydroxypropyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 45 | 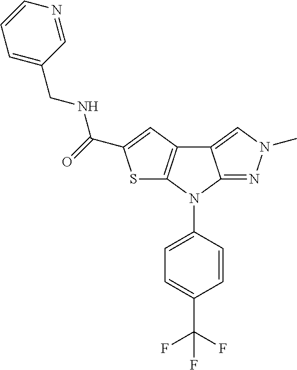 [SMILES: Cn1cc2c3cc(C(=O)NCc4cccnc4)sc3n(-c3ccc(C(F)(F)F)cc3)c2n1] | 1H NMR (400 MHz, DMSO) δ 9.11 (t, J = 5.9 Hz, 1H), 8.61- 8.56 (m, 1H), 8.48 (dd, J = 4.8, 1.7 Hz, 1H), 8.27- 8.20 (m, 2H), 8.10 (s, 1H), 8.04 (s, 1H), 8.01 (d, J = 8.6 Hz, 2H), 7.76 (dt, J = 7.9, 2.0 Hz, 1H), 7.38 (ddd, J = 7.8, 4.8, 0.9 Hz, 1H), 4.53 (d, J = 5.7 Hz, 2H), 4.06 (s, 3H). | Method C, Rt = 1.20 min, [M + H]+ = 456.1 |
| | 4-methyl-N-[(pyridin-3-yl)methyl]-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 46 | 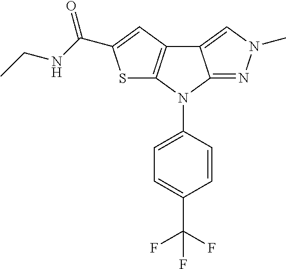 [SMILES: CCNC(=O)c1cc2c3cn(C)nc3n(-c3ccc(C(F)(F)F)cc3)c2s1] | 1H NMR (400 MHz, DMSO) δ 8.50 (t, J = 5.6 Hz, 1H), 8.27- 8.20 (m, 2H), 8.03 (s, 1H), 8.03 (s, 1H), 8.00 (d, J = 8.5 Hz, 2H), 4.06 (s, 3H), 3.36-3.27 (m, 1H), 1.15 (t, J = 7.2 Hz, 3H). | Method C, Rt = 1.42 min, [M + H]+ = 393.1 |
| | N-ethyl-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 47 | 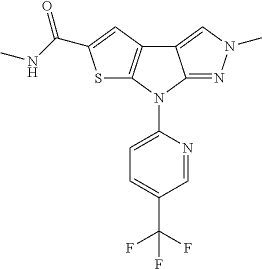 | 1H NMR (400 MHz, DMSO) δ 8.97 (dt, J = 2.0, 1.0 Hz, 1H), 8.46 (dd, J = 9.0, 2.4 Hz, 1H), 8.40 (d, J = 4.6 Hz, 1H), 8.39-8.35 (m, 1H), 8.04 (s, 1H), 7.91 (s, 1H), 4.07 (s, 3H), 2.80 (d, J = 4.5 Hz, 3H). | Method C, Rt = 1.35 min, [M + H]+ = 380.0 |
| | N,4-dimethyl-7-[5- | | |
| | (trifluoromethyl)pyridin-2-yl]-9-thia- | | |
| | 4,5,7-triazatricyclo[6.3.0.0^{2,6}]undeca- | | |
| | 1(8),2,5,10-tetraene-10-carboxamide | | |
| 48 | 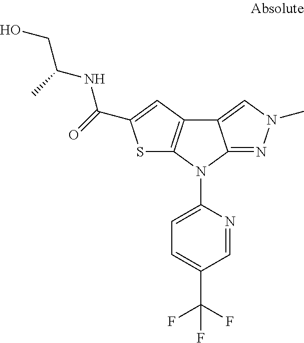 [SMILES: C[C@H](CO)NC(=O)c1cc2c3cn(C)nc3n(-c3ccc(C(F)(F)F)cn3)c2s1] | 1H NMR (400 MHz, DMSO) δ 8.98 (dt, J = 2.0, 0.9 Hz, 1H), 8.46 (dd, J = 8.9, 2.4 Hz, 1H), 8.41- 8.34 (m, 1H), 8.07 (d, J = 8.0 Hz, 1H), 8.05 (s, 2H), 4.71 (t, J = 5.7 Hz, 1H), 4.07 (s, 3H), 4.00 (dt, J = 13.5, 6.6 Hz, 1H), 3.48 (dt, J = 11.1, 5.7 Hz, 1H), 3.36 (dt, J = 10.7, 6.2 Hz, 1H), 1.16 (d, J = 6.8 Hz, 3H). | Method C, Rt = 1.32 min, [M + H]+ = 424.0 |
| | N-[(2R)-1-hydroxypropan-2-yl]-4-methyl- | | |
| | 7-[5-(trifluoromethyl)pyridin-2-yl]-9- | | |
| | thia-4,5,7- | | |
| | triazatricyclo[6.3.0.0^{2,6}]undeca- | | |
| | 1(8),2,5,10-tetraene-10-carboxamide | | |
| 49 | 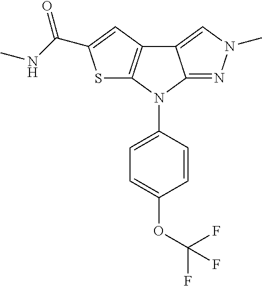 | 1H NMR (400 MHz, DMSO) δ 8.45 (d, J = 4.7 Hz, 1H), 8.17- 8.08 (m, 2H), 8.01 (s, 1H), 7.96 (s, 1H), 7.69-7.60 (m, 2H), 4.04 (s, 3H), 2.81 (d, J = 4.5 Hz, 3H). | Method C, Rt = 1.38 min, [M + H]+ = 395.0 |
| | N,4-dimethyl-7-[4- | | |
| | (trifluoromethoxy)phenyl]-9-thia-4,5,7- | | |
| | triazatricyclo[6.3.0.0^{2,6}]undeca- | | |
| | 1(8),2,5,10-tetraene-10-carboxamide | | |
| 50 | 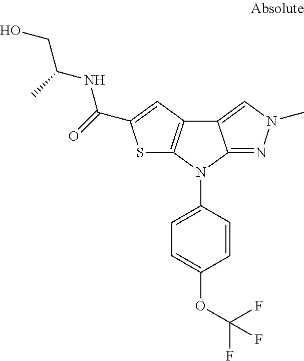 [SMILES: C[C@H](CO)NC(=O)c1cc2c3cn(C)nc3n(-c3ccc(OC(F)(F)F)cc3)c2s1] | 1H NMR (400 MHz, DMSO) δ 8.15- 8.11 (m, 3H), 8.10 (s, 1H), 8.01 (s, 1H), 7.69-7.61 (m, 2H), 4.72 (t, J = 5.8 Hz, 1H), 4.04 (s, 3H), 4.03-3.94 (m, 1H), 3.48 (dt, J = 11.1, 5.7 Hz, 1H), 3.35 (dt, J = 10.5, 6.2 Hz, 1H), 1.16 (d, J = 6.7 Hz, 3H). | Method C, Rt = 1.34 min, [M + H]⁺ = 439.0 |
| | N-[(2R)-1-hydroxypropan-2-yl]-4-methyl- | | |
| | 7-[4-(trifluoromethoxy)phenyl]-9-thia- | | |
| | 4,5,7-triazatricyclo[6.3.0.0^{2,6}]undeca- | | |
| | 1(8),2,5,10-tetraene-10-carboxamide | | |
| 51 | 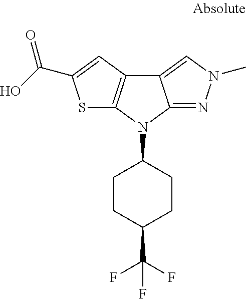 [SMILES: Cn1cc2c3cc(C(=O)O)sc3n([C@H]3CC[C@@H](C(F)(F)F)CC3)c2n1] | 1H NMR (500 MHz, DMSO) δ 12.60 (s, 1H), 7.84 (s, 1H), 7.78 (s, 1H), 4.31 (dq, J = 8.8, 4.2, 3.6 Hz, 1H), 3.95 (s, 3H), 2.57 (dt, J = 10.5, 5.3 Hz, 1H), 2.47 (d, J = 7.0 Hz, 1H), 1.92 (t, J = 4.6 Hz, 1H), 1.88 (dd, J = 10.8, 5.0 Hz, 5H), −4.00 (s, 1H). | Method C, Rt = 1.27 min, [M + H]+ = 372.1 |
| | 4-methyl-7-[(1s,4s)-4- | | |
| | (trifluoromethyl)cyclohexyl]-9-thia-4,5,7- | | |
| | triazatricyclo[6.3.0.0^{2,6}]undeca- | | |
| | 1(8),2,5,10-tetraene-10-carboxylic acid | | |
| 52 | 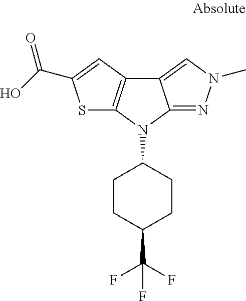 [SMILES: Cn1cc2c3cc(C(=O)O)sc3n([C@H]3CC[C@H](C(F)(F)F)CC3)c2n1] | 1H NMR (500 MHz, DMSO) δ 12.56 (s, 1H), 7.83 (s, 1H), 7.78 (s, 1H), 4.23 (tt, J = 10.7, 5.5 Hz, 1H), 3.95 (s, 3H), 2.45 (td, J = 8.8, 4.2 Hz, 0H), 2.15- 2.07 (m, 4H), 2.03 (dd, J = 15.0, 4.7 Hz, 2H), 1.56 (qd, J = 12.5, 5.4 Hz, 2H). | Method C, Rt = 1.29 min, [M + H]+ = 372.1 |
| | 4-methyl-7-[(1r,4r)-4- | | |
| | (trifluoromethyl)cyclohexyl]-9-thia-4,5,7- | | |
| | triazatricyclo[6.3.0.0^{2,6}]undeca- | | |
| | 1(8),2,5,10-tetraene-10-carboxylic acid | | |
| 53 | 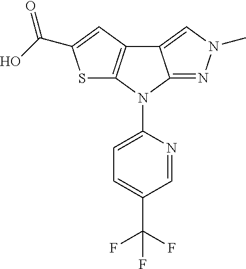 [SMILES: Cn1cc2c3cc(C(=O)O)sc3n(-c3ccc(C(F)(F)F)cn3)c2n1] | 1H NMR (400 MHz, DMSO) δ 8.99 (dt, J = 2.0, 0.9 Hz, 1H), 8.51-8.44 (m, 1H), 8.38 (dt, J = 8.8, 0.8 Hz, 1H), 8.00 (s, 1H), 7.93 (s, 1H), 4.08 (s, 3H). | Method C, Rt = 1.38 min, [M + H]⁺ = 367.0 |
| | 4-methyl-7-[5-(trifluoromethyl)pyridin- | | |
| | 2-yl]-9-thia-4,5,7- | | |
| | triazatricyclo[6.3.0.0^{2,6}]undeca- | | |
| | 1(8),2,5,10-tetraene-10-carboxylic acid | | |
| 54 | 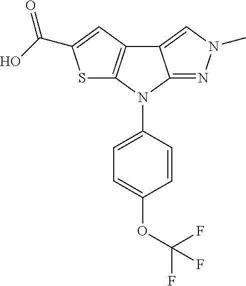 [SMILES: Cn1cc2c3cc(C(=O)O)sc3n(-c3ccc(OC(F)(F)F)cc3)c2n1] | 1H NMR (700 MHz, DMSO) δ 8.14- 8.12 (m, 2H), 7.96 (s, 1H), 7.87 (s, 1H), 7.66 (d, J = 8.7 Hz, 2H), 4.04 (s, 3H). | Method C, Rt = 1.40 min, [M + H]+ = 382.0 |
| | 4-methyl-7-[4- | | |
| | (trifluoromethoxy)phenyl]-9-thia-4,5,7- | | |
| | triazatricyclo[6.3.0.0^{2,6}]undeca- | | |
| | 1(8),2,5,10-tetraene-10-carboxylic acid | | |
| 55 | 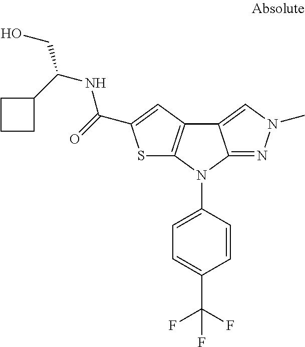 [SMILES: Cn1cc2c3cc(C(=O)N[C@@H](CO)C4CCC4)sc3n(-c3ccc(C(F)(F)F)cc3)c2n1] | 1H NMR (500 MHz, DMSO) δ 8.24 (d, J = 8.6 Hz, 2H), 8.17 (s, 1H), 8.06 (s, 1H), 8.04 (s, 2H), 8.03-7.98 (m, 2H), 4.60 (td, J = 5.7, 1.2 Hz, 1H), 4.06 (s, 3H), 4.02- 3.92 (m, 1H), 3.41 (dq, J = 14.9, 5.5 Hz, 2H), 2.56 (p, J = 7.9 Hz, 1H), 2.01- 1.89 (m, 1H), 1.89- 1.71 (m, 3H). | Method C, Rt = 1.44 min, [M + H]+ = 463.1 |
| | single enantiomer, absolute | | |
| | configuration of the enantiomer has | | |
| | been assigned arbitrarily | | |
| | N-[(1R)-1-cyclobutyl-2-hydroxyethyl]-4- | | |
| | methyl-7-[4-(trifluoromethyl)phenyl]-9- | | |
| | thia-4,5,7- | | |
| | triazatricyclo[6.3.0.0^{2,6}]undeca- | | |
| | 1(8),2,5,10-tetraene-10-carboxamide | | |
| 56 | 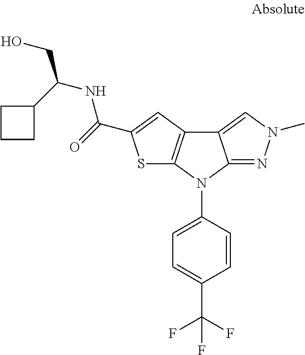 [SMILES: Cn1cc2c3cc(C(=O)N[C@H](CO)C4CCC4)sc3n(-c3ccc(C(F)(F)F)cc3)c2n1] | 1H NMR (500 MHz, DMSO) δ 8.24 (d, J = 8.6 Hz, 2H), 8.17 (s, 1H), 8.07 (d, J = 9.0 Hz, 1H), 8.04 (s, 2H), 8.01 (d, J = 8.7 Hz, 2H), 4.60 (t, J = 5.6 Hz, 1H), 4.06 (s, 3H), 4.02-3.93 (m, 1H), 3.41 (dq, J = 14.8, 5.5 Hz, 2H), 2.57 (p, J = 8.0 Hz, 1H), 2.01-1.88 (m, 1H), 1.87- 1.71 (m, 3H). | Method C, Rt = 1.44 min, [M + H]+ = 463.1 |
| | single enantiomer, absolute | | |
| | configuration of the enantiomer has | | |
| | been assigned arbitrarily | | |
| | N-[(1S)-1-cyclobutyl-2-hydroxyethyl]-4- | | |
| | methyl-7-[4-(trifluoromethyl)phenyl]-9- | | |
| | thia-4,5,7- | | |
| | triazatricyclo[6.3.0.0^{2,6}]undeca- | | |
| | 1(8),2,5,10-tetraene-10-carboxamide | | |
| 57 | 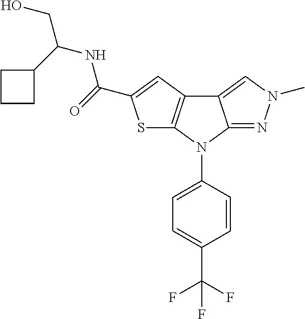 [SMILES: Cn1cc2c3cc(C(=O)NC(CO)C4CCC4)sc3n(-c3ccc(C(F)(F)F)cc3)c2n1] | 1H NMR (500 MHz, DMSO) δ 8.24 (d, J = 8.6 Hz, 2H), 8.17 (s, 1H), 8.06 (s, 1H), 8.04 (s, 2H), 8.01 (d, J = 8.7 Hz, 2H), 4.63-4.57 (m, 1H), 4.06 (s, 3H), 4.02-3.93 (m, 1H), 3.41 (dq, J = 14.9, 5.5 Hz, 2H), 2.57 (p, J = 8.1 Hz, 1H), 2.01-1.89 (m, 1H), 1.89- 1.70 (m, 3H). | Method C, Rt = 1.44 min, [M + H]+ = 463.1 |
| | racemate | | |
| | N-(1-cyclobutyl-2-hydroxyethyl)-4- | | |
| | methyl-7-[4-(trifluoromethyl)phenyl]-9- | | |
| | thia-4,5,7- | | |
| | triazatricyclo[6.3.0.0^{2,6}]undeca- | | |
| | 1(8),2,5,10-tetraene-10-carboxamide | | |
| 58 | 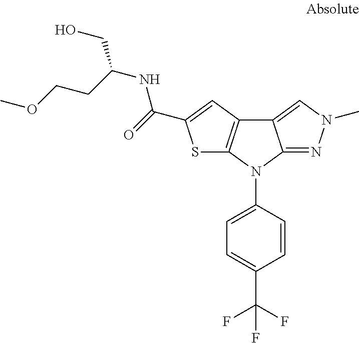 | 1H NMR (500 MHz, DMSO) δ 8.24 (d, J = 8.6 Hz, 2H), 8.21 (d, J = 8.3 Hz, 1H), 8.15 (s, 1H), 8.07 (s, 1H), 8.05-7.99 (m, 2H), 4.82 (t, J = 5.7 Hz, 1H), 4.07 (s, 3H), 4.00 (dq, J = 9.2, 4.5 Hz, 1H), 3.48 (dt, J = 10.8, 5.4 Hz, 1H), 3.44- 3.37 (m, 2H), 3.40- 3.33 (m, 1H), 3.22 (s, 3H), 1.90 (dtd, J = 14.0, 7.3, 4.4 Hz, 1H), 1.70 (ddt, J = 13.7, 9.4, 6.1 Hz, 1H). | Method C, Rt = 1.35 min, [M + H]+ = 467.1 |
| | single enantiomer, absolute | | |
| | configuration of the enantiomer has | | |
| | been assigned arbitrarily | | |
| | N-[(2R)-1-hydroxy-4-methoxybutan-2- | | |
| | yl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 59 | 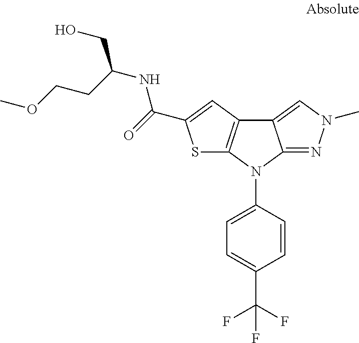 | 1H NMR (500 MHz, DMSO) δ 8.24 (d, J = 8.6 Hz, 2H), 8.21 (d, J = 8.4 Hz, 1H), 8.15 (s, 1H), 8.07 (s, 1H), 8.02 (d, J = 8.7 Hz, 2H), 4.81 (t, J = 5.7 Hz, 1H), 4.07 (s, 3H), 4.00 (td, J = 9.0, 4.4 Hz, 1H), 3.48 (dt, J = 10.8, 5.5 Hz, 1H), 3.44-3.33 (m, 3H), 3.22 (s, 3H), 1.90 (dtd, J = 14.0, 7.3, 4.4 Hz, 1H), 1.71 (dt, J = 16.9, 7.4 Hz, 1H). | Method C, Rt = 1.35 min, [M + H]+ = 467.1 |
| | single enantiomer, absolute | | |
| | configuration of the enantiomer has | | |
| | been assigned arbitrarily | | |
| | N-[(2S)-1-hydroxy-4-methoxybutan-2- | | |
| | yl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 60 | 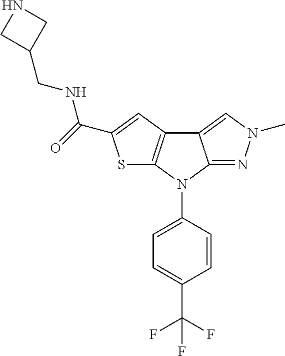 [SMILES: Cn1cc2c3cc(C(=O)NCC4CNC4)sc3n(-c3ccc(C(F)(F)F)cc3)c2n1] | 1H NMR (500 MHz, DMSO) δ 8.80 (d, J = 6.0 Hz, 1H), 8.46 (s, 1H), 8.23 (d, J = 8.6 Hz, 2H), 8.09 (s, 1H), 8.07 (s, 1H), 8.02 (d, J = 8.6 Hz, 2H), 4.06 (s, 3H), 3.86 (t, J = 9.0 Hz, 2H), 3.66 (dd, J = 9.8, 6.8 Hz, 2H), 3.50 (d, J = 12.1 Hz, 1H), 3.00-2.94 (m, 1H). | Method C, Rt = 1.17 min, [M+H]+ = 434.0 |
| | N-[(azetidin-3-yl)methyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 61 | 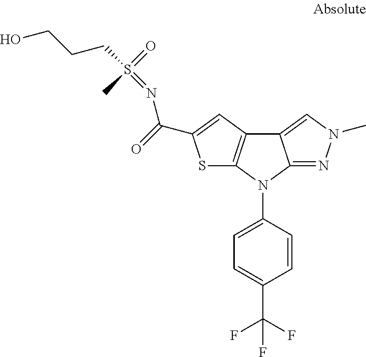 [SMILES: Cn1cc2c3cc(C(=O)N=[S@@](C)(=O)CCCO)sc3n(-c3ccc(C(F)(F)F)cc3)c2n1] | 1H NMR (500 MHz, DMSO) δ 8.23 (d, J = 8.6 Hz, 2H), 8.02 (d, J = 8.7 Hz, 2H), 7.97 (s, 1H), 7.92 (s, 1H), 4.78 (t, J = 5.2 Hz, 1H), 4.06 (s, 3H), 3.71-3.60 (m, 2H), 3.57 (q, J = 5.9 Hz, 2H), 3.48 (s, 3H), 2.04-1.91 (m, 2H). | Method C, Rt = 1.34 min, [M + H]+ = 485.1 |
| | single enantiomer, absolute | | |
| | configuration of the enantiomer has | | |
| | been assigned arbitrarily | | |
| | N-[(S)-(3-hydroxypropyl)(methyl)oxo-λ⁶- | | |
| | sulfanylidene]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 62 | 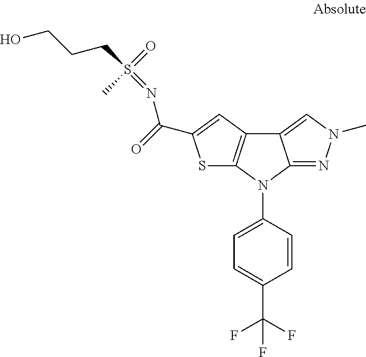 [SMILES: Cn1cc2c3cc(C(=O)N=[S@](C)(=O)CCCO)sc3n(-c3ccc(C(F)(F)F)cc3)c2n1] | 1H NMR (700 MHz, DMSO) δ 8.23 (d, J = 8.6 Hz, 2H), 8.02 (d, J = 8.6 Hz, 2H), 7.97 (s, 1H), 7.92 (s, 1H), 4.79 (t, J = 5.2 Hz, 1H), 4.06 (s, 3H), 3.66 (ddd, J = 14.1, 10.3, 5.7 Hz, 1H), 3.63-3.59 (m, 1H), 3.57 (q, J = 6.1 Hz, 2H), 3.48 (s, 3H), 1.97 (tddd, J = 13.4, 10.2, 7.4, 6.0 Hz, 2H). | Method C, Rt = 1.35 min, [M + H]+ = 485.1 |
| | single enantiomer, absolute | | |
| | configuration of the enantiomer has | | |
| | been assigned arbitrarily | | |
| | N-[(R)-(3-hydroxypropyl)(methyl)oxo-λ⁶- | | |
| | sulfanylidene]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 63 | 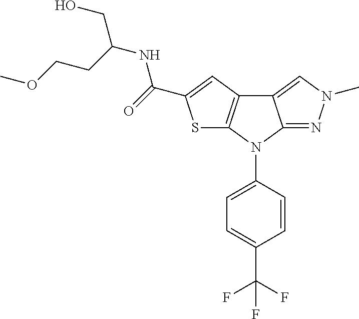 | 1H NMR (700 MHz, DMSO) δ 8.26- 8.22 (m, 2H), 8.17 (d, J = 8.4 Hz, 1H), 8.14 (s, 1H), 8.05 (s, 1H), 8.03-7.99 (m, 2H), 4.76 (t, J = 5.7 Hz, 1H), 4.06 (s, 3H), 4.00 (tt, J = 9.9, 5.0 Hz, 1H), 3.48 (dt, J = 10.9, 5.5 Hz, 1H), 3.44- 3.35 (m, 3H), 3.22 (s, 3H), 1.90 (dtd, J = 14.2, 7.3, 4.5 Hz, 1H), 1.75-1.67 (m, 1H). | Method C, Rt = 1.35 min, [M + H]+ = 467.1 |
| | racemate | | |
| | N-(1-hydroxy-4-methoxybutan-2-yl)-4- | | |
| | methyl-7-[4-(trifluoromethyl)phenyl]-9- | | |
| | thia-4,5,7- | | |
| | triazatricyclo[6.3.0.0^{2,6}]undeca- | | |
| | 1(8),2,5,10-tetraene-10-carboxamide | | |
| 64 | 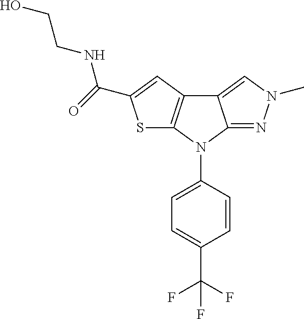 [SMILES: Cn1cc2c3cc(C(=O)NCCO)sc3n(-c3ccc(C(F)(F)F)cc3)c2n1] | 1H NMR (500 MHz, DMSO) δ 8.53 (s, 1H), 8.23 (d, J = 8.6 Hz, 2H), 8.07 (s, 1H), 8.05 (s, 1H), 8.03-7.98 (m, 2H), 4.79-4.73 (m, 1H), 4.06 (s, 3H), 3.53 (q, J = 6.0 Hz, 2H), 3.35 (q, J = 6.0 Hz, 2H). | Method C, Rt = 1.31 min, [M + H]+ = 409.0 |
| | N-(2-hydroxyethyl)-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 65 | 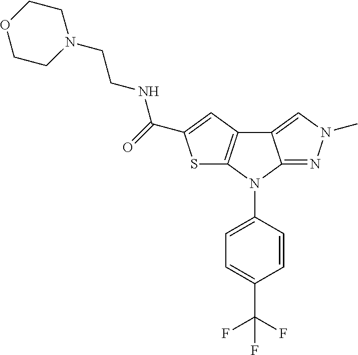 [SMILES: Cn1cc2c3cc(C(=O)NCCN4CCOCC4)sc3n(-c3ccc(C(F)(F)F)cc3)c2n1] | 1H NMR (500 MHz, DMSO) δ 8.48 (t, J = 5.7 Hz, 1H), 8.23 (d, J = 8.6 Hz, 2H), 8.04 (s, 1H), 8.04 (s, 1H), 8.01 (d, J = 8.7 Hz, 2H), 4.06 (s, 3H), 3.58 (t, J = 4.6 Hz, 4H), 3.41 (q, J = 6.5 Hz, 2H), 2.48 (d, J = 6.8 Hz, 2H), 2.43 (t, J = 4.6 Hz, 4H). | Method C, Rt = 1.19 min, [M + H]+ = 478.1 |
| | 4-methyl-N-[2-(morpholin-4-yl)ethyl]-7- | | |
| | [4-(trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 66 | 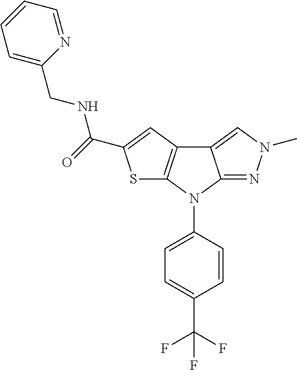 [SMILES: Cn1cc2c3cc(C(=O)NCc4ccccn4)sc3n(-c3ccc(C(F)(F)F)cc3)c2n1] | 1H NMR (500 MHz, DMSO) δ 9.18 (t, J = 6.1 Hz, 1H), 8.53 (ddd, J = 4.8, 1.8, 0.9 Hz, 1H), 8.27- 8.21 (m, 2H), 8.15 (s, 1H), 8.06 (s, 1H), 8.04-7.99 (m, 2H), 7.78 (td, J = 7.7, 1.8 Hz, 1H), 7.38 (dt, J = 7.9, 1.0 Hz, 1H), 7.28 (ddd, J = 7.5, 4.9, 1.2 Hz, 1H), 4.60 (d, J = 5.9 Hz, 2H), 4.06 (s, 3H), −3.99 (s, 0H). | Method C, Rt = 1.21 min, [M + H]+ = 456.0 |
| | 4-methyl-N-[(pyridin-2-yl)methyl]-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 67 | 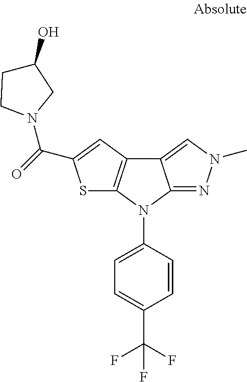 [SMILES: Cn1cc2c3cc(C(=O)N4CC[C@@H](O)C4)sc3n(-c3ccc(C(F)(F)F)cc3)c2n1] | 1H NMR (700 MHz, DMSO) δ 8.24 (d, J = 8.6 Hz, 2H), 8.01 (d, J = 8.7 Hz, 2H), 7.96 (s, 0H), 7.95 (s, 1H), 7.91 (s, 1H), 5.06 (d, J = 38.2 Hz, 1H), 4.38 (d, J = 74.1 Hz, 1H), 4.07 (s, 3H), 3.98 (s, 1H), 3.72 (d, J = 10.0 Hz, 1H), 3.62 (s, 1H), 3.58 (d, J = 11.4 Hz, 0H), 3.50 (s, 0H), 1.96 (t, J = 78.3 Hz, 2H). | Method C, Rt = 1.34 min, [M + H]+ = 435.0 |
| | (3R)-1-{4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10- | | |
| | carbonyl}pyrrolidin-3-ol | | |
| 68 | 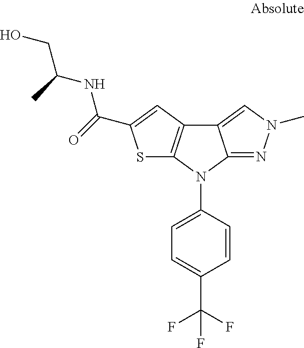 [SMILES: C[C@@H](CO)NC(=O)c1cc2c3cn(C)nc3n(-c3ccc(C(F)(F)F)cc3)c2s1] | 1H NMR (500 MHz, DMSO) δ 8.23 (d, J = 8.6 Hz, 2H), 8.18 (d, J = 8.0 Hz, 1H), 8.12 (s, 1H), 8.04 (s, 1H), 8.03-7.98 (m, 2H), 4.75 (t, J = 5.7 Hz, 1H), 4.06 (s, 3H), 4.04-3.94 (m, 1H), 3.48 (dt, J = 11.0, 5.6 Hz, 1H), 3.40-3.33 (m, 1H), 1.16 (d, J = 6.7 Hz, 3H). | Method C, Rt = 1.34 min, [M + H]+ = 423.0 |
| | N-[(2S)-1-hydroxypropan-2-yl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 69 | 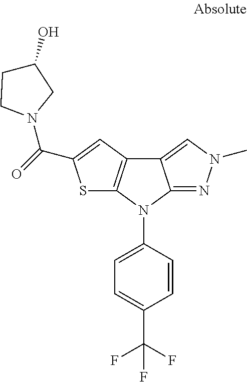 [SMILES: Cn1cc2c3cc(C(=O)N4CC[C@H](O)C4)sc3n(-c3ccc(C(F)(F)F)cc3)c2n1] | 1H NMR (500 MHz, DMSO) δ 8.24 (d, J = 8.6 Hz, 2H), 8.04- 7.98 (m, 2H), 7.95 (s, 1H), 7.91 (s, 1H), 5.05 (d, J = 21.2 Hz, 1H), 4.38 (d, J = 49.5 Hz, 1H), 4.07 (s, 3H), 3.95 (d, J = 25.6 Hz, 2H), 3.82-3.40 (m, 3H), 1.96 (t, J = 54.0 Hz, 2H). | Method C, Rt = 1.34 min, [M + H]+ = 435.1 |
| | (3S)-1-{4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10- | | |
| | carbonyl}pyrrolidin-3-ol | | |
| 70 | 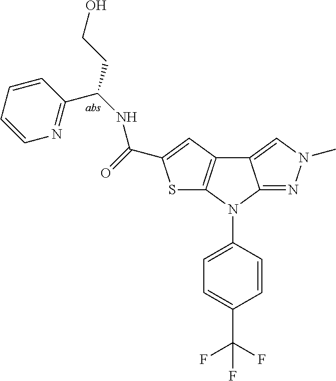 [SMILES: Cn1cc2c3cc(C(=O)N[C@@H](CCO)c4ccccn4)sc3n(-c3ccc(C(F)(F)F)cc3)c2n1] | 1H NMR (500 MHz, DMSO) δ 1.98- 2.07 (m, 1H), 2.07- 2.16 (m, 1H), 3.45-3.58 (m, 2H), 4.07 (s, 3H), 4.58 (t, J = 5.0 Hz, 1H), 5.23 (td, J = 8.7, 5.4 Hz, 1H), 7.26 (ddd, J = 7.5, 4.8, 1.2 Hz, 1H), 7.43 (dt, J = 7.9, 1.1 Hz, 1H), 7.77 (td, J = 7.7, 1.8 Hz, 1H), 7.97-8.03 (m, 2H), 8.06 (s, 1H), 8.20-8.26 (m, 2H), 8.27 (s, 1H), 8.54 (ddd, J = 4.9, 1.8, 0.9 Hz, 1H), 8.83 (d, J = 8.1 Hz, 1H). | Method for chiral separation: SFC; column: YMC Amylose-C; eluent: CO2:2-propanol (60:40), wave length: 240 nm; flow: 5mL/min Rt: 3.87 min |
| | N-[(1S)-3-hydroxy-1-(pyridin-2- | | |
| | yl)propyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| | Absolute configuration assigned | | |
| | arbitrarily | | |
| 71 | 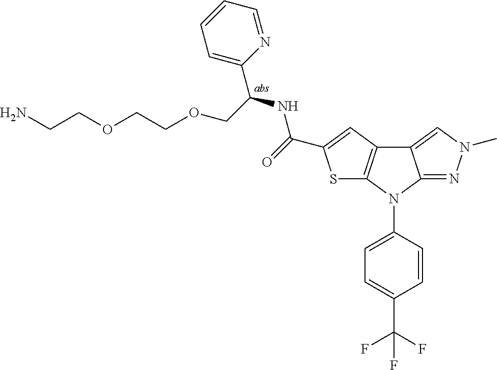 [SMILES: Cn1cc2c3cc(C(=O)N[C@@H](COCCOCCN)c4ccccn4)sc3n(-c3ccc(C(F)(F)F)cc3)c2n1] | | |
| | N-[(1R)-2-[2-(2-aminoethoxy)ethoxy]-1- | | |
| | (pyridin-2-yl)ethyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 72 | 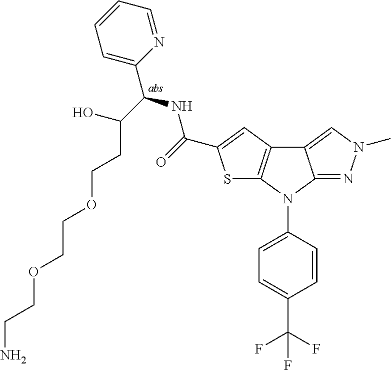 [SMILES: Cn1cc2c3cc(C(=O)N[C@H](c4ccccn4)C(O)CCOCCOCCN)sc3n(-c3ccc(C(F)(F)F)cc3)c2n1] | | |
| | N-[(1R)-4-[2-(2-aminoethoxy)ethoxy]-2- | | |
| | hydroxy-1-(pyridin-2-yl)butyl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 73 | 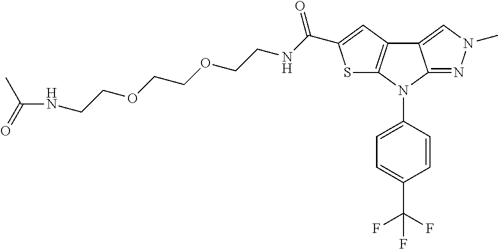 [SMILES: CC(=O)NCCOCCOCCNC(=O)c1cc2c3cn(C)nc3n(-c3ccc(C(F)(F)F)cc3)c2s1] | ¹H NMR (500 MHz, DMSO-d₆) δ 8.60 (t, J = 5.6 Hz, 1H), 8.23 (d, J = 8.6 Hz, 2H), 8.07 (s, 1H), 8.04 (s, 1H), 7.98- 8.02 (m, 2H), 7.86 (t, J = 5.8 Hz, 1H), 4.06 (s, 3H), 3.50- 3.60 (m, 6H), 3.43 (dt, J = 20.8, 5.8 Hz, 4H), 3.18 (q, J = 5.8 Hz, 2H), 1.79 (s, 3H). | Method C, Rₜ = 1.34 min, [M + H]⁺ = 538.1 |
| | N-(2-{2-[2-({4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraen-10-yl}formamido)- | | |
| | ethoxy]ethoxy}ethyl)acetamide | | |
| 74 | 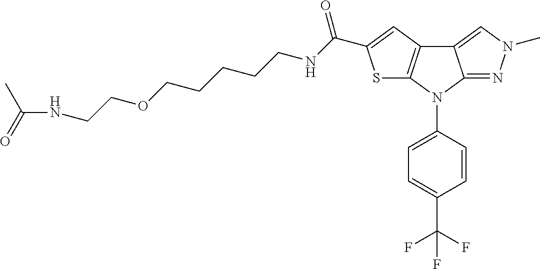 [SMILES: CC(=O)NCCOCCCCCNC(=O)c1cc2c3cn(C)nc3n(-c3ccc(C(F)(F)F)cc3)c2s1] | | |
| | N-(2-{[5-{{4-methyl-7-[4- | | |
| | (trifluoromethyl)phenyl]-9-thia-4,5,7-tri- | | |
| | azatricyclo[6.3.0.0^{2,6}]undeca-1(8),2,5,10- | | |
| | tetraen-10-yl}formamido)pentyl]- | | |
| | oxy}ethyl)acetamide | | |
| 75 | 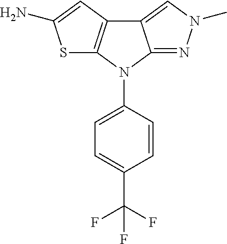 [SMILES: Cn1cc2c3cc(N)sc3n(-c3ccc(C(F)(F)F)cc3)c2n1] | | |
| | 4-methyl-7-[4-(trifluoromethyl)phenyl]- | | |
| | 9-thia-4,5,7-triazatri- | | |
| | cyclo[6.3.0.0^{2,6}]undeca-1(8),2,5,10- | | |
| | tetraen-10-amine | | |
| 76 | 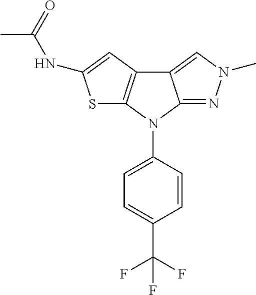 [SMILES: CC(=O)Nc1cc2c3cn(C)nc3n(-c3ccc(C(F)(F)F)cc3)c2s1] | | |
| | N-{4-methyl-7-[4- | | |
| | (trifluoromethyl)phenyl]-9-thia- | | |
| | 4,5,7-triazatricyclo[6.3.0.0^{2,6}]undeca- | | |
| | 1(8),2,5,10-tetraen-10-yl}acetamide | | |
| 77 | 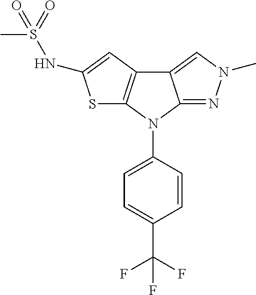 | | |
| | N-{4-methyl-7-[4- | | |
| | (trifluoromethyl)phenyl]-9-thia- | | |
| | 4,5,7-triazatricyclo[6.3.0.0^{2,6}]undeca- | | |
| | 1(8),2,5,10-tetraen-10- | | |
| | yl}methanesulfonamide | | |
| 78 | 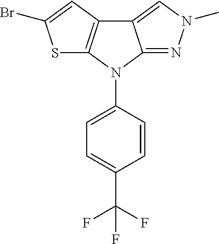 [SMILES: Cn1cc2c3cc(Br)sc3n(-c3ccc(C(F)(F)F)cc3)c2n1] | | |
| | 10-bromo-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene | | |
| 79 | 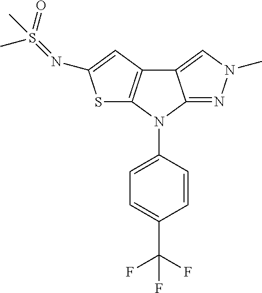 | | |
| | Dimethyl({4-methyl-7-[4- | | |
| | (trifluoromethyl)phenyl]-9- | | |
| | thia-4,5,7- | | |
| | triazatricyclo[6.3.0.0^{2,6}]undeca- | | |
| | 1(8),2,5,10-tetraen-10-yl}imino)- | | |
| | lambda6-sulfanone | | |
| 80 | 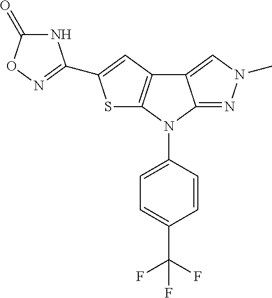 [SMILES: Cn1cc2c3cc(-c4noc(=O)[nH]4)sc3n(-c3ccc(C(F)(F)F)cc3)c2n1] | ¹H NMR (500 MHz, DMSO-d₆) δ 8.21 (d, J = 8.6 Hz, 2H), 7.99 (d, J = 8.6 Hz, 2H), 7.96 (s, 1H), 7.62 (s, 1H), 4.05 (s, 3H). | Method C, Rₜ =1.43 min, [M + H]⁺ = 406.0 |
| | 3-{4-methyl-7-[4- | | |
| | (trifluoromethyl)phenyl]-9-thia- | | |
| | 4,5,7-triazatricyclo[6.3.0.0^{2,6}]undeca- | | |
| | 1(8),2,5,10-tetraen-10-yl}-4,5-dihydro- | | |
| | 1,2,4-oxadiazol-5-one | | |
| 81 | 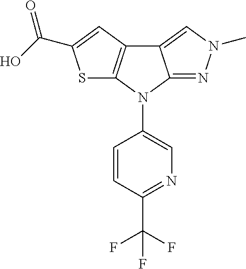 [SMILES: Cn1cc2c3cc(C(=O)O)sc3n(-c3ccc(C(F)(F)F)nc3)c2n1] | 1H NMR (700 MHz, DMSO) δ 13.14 (s, 1H), 9.45 (d, J = 2.7 Hz, 1H), 8.60 (dd, J = 8.6, 2.8 Hz, 1H), 8.18 (d, J = 8.7 Hz, 1H), 8.02 (s, 1H), 8.01 (s, 1H), 4.07 (s, 3H). | Method C: Rt: 1.29 min M + H+: 367.00 |
| | 4-methyl-7-[6-(trifluoromethyl)pyridin- | | |
| | 3-yl]-9-thia-4,5,7- | | |
| | triazatricyclo[6.3.0.0^{2,6}]undeca- | | |
| | 1(8),2,5,10-tetraene-10-carboxylic acid | | |
| 82 | 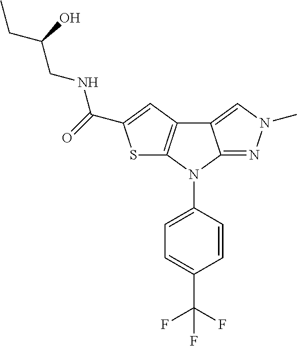 [SMILES: CC[C@@H](O)CNC(=O)c1cc2c3cn(C)nc3n(-c3ccc(C(F)(F)F)cc3)c2s1] | 1H NMR (500 MHz, DMSO) δ 8.46 (t, J = 5.8 Hz, 1H), 8.26- 8.20 (m, 2H), 8.10 (s, 1H), 8.04 (s, 1H), 8.03-7.97 (m, 2H), 4.72 (d, J = 5.2 Hz, 1H), 4.06 (s, 3H), 3.55 (ddt, J = 12.2, 7.2, 5.1 Hz, 1H), 3.35-3.26 (m, 1H), 3.23- 3.15 (m, 1H), 1.55- 1.44 (m, 1H), 1.33 (dt, J = 13.5, 7.3 Hz, 1H), 0.91 (t, J = 7.4 Hz, 3H). | Method C: Rt = 1.38 min M + H+: 437.10 2M + Na: 895.10 |
| | N-[(2R)-2-hydroxybutyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 83 | 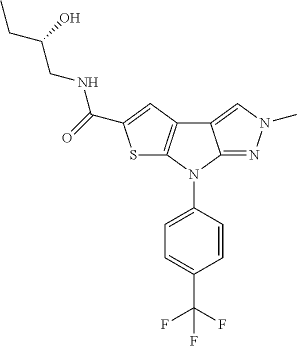 [SMILES: CC[C@H](O)CNC(=O)c1cc2c3cn(C)nc3n(-c3ccc(C(F)(F)F)cc3)c2s1] | 1H NMR (500 MHz, DMSO) δ 8.46 (t, J = 5.8 Hz, 1H), 8.26- 8.21 (m, 2H), 8.10 (s, 1H), 8.04 (s, 1H), 8.03-7.98 (m, 2H), 4.72 (d, J = 5.2 Hz, 1H), 4.06 (s, 3H), 3.54 (ddt, J = 12.3, 7.1, 5.1 Hz, 1H), 3.28 (s, 1H), 3.19 (ddd, J = 12.9, 6.8, 5.5 Hz, 1H), 1.55-1.43 (m, 1H), 1.33 (dt, J = 13.5, 7.3 Hz, 1H), 0.91 (t, J = 7.4 Hz, 3H). | Method C: Rt = 1.38 min M + H+: 437.10 2M + Na: 895.10 |
| | N-[(2S)-2-hydroxybutyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 84 | 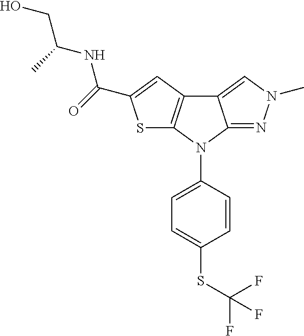 [SMILES: C[C@H](CO)NC(=O)c1cc2c3cn(C)nc3n(-c3ccc(SC(F)(F)F)cc3)c2s1] | 1H NMR (500 MHz, DMSO) δ 8.21- 8.13 (m, 3H), 8.11 (s, 1H), 8.03 (s, 1H), 8.01-7.95 (m, 2H), 4.74 (t, J = 5.7 Hz, 1H), 4.05 (s, 3H), 4.03-3.94 (m, 1H), 3.48 (dt, J = 11.0, 5.6 Hz, 1H), 3.36 (dt, J = 10.6, 6.1 Hz, 1H), 1.16 (d, J = 6.7 Hz, 3H). | Method C: Rt: 1.41 min M + H+: 455.00 2M + Na: 931.00 |
| | N-[(2R)-1-hydroxypropan-2-yl]-4-methyl- | | |
| | 7-{4-[(trifluoromethyl)sulfanyl]phenyl}- | | |
| | 9-thia-4,5,7- | | |
| | triazatricyclo[6.3.0.0^{2,6}]undeca- | | |
| | 1(8),2,5,10-tetraene-10-carboxamide | | |
| 85 | 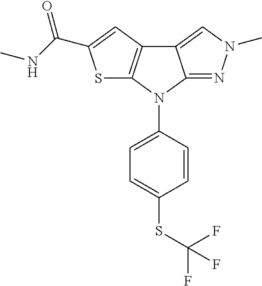 | 1H NMR (500 MHz, DMSO) δ 8.49 (q, J = 4.5 Hz, 1H), 8.21- 8.15 (m, 2H), 8.03 (s, 1H), 8.01- 7.95 (m, 3H), 4.05 (s, 3H), 2.81 (d, J = 4.5 Hz, 3H). | Method C: Rt: 1.44 min M + H+: 411.00 2M + Na: 843.00 |
| | N,4-dimethyl-7-{4-[(trifluoromethyl)- | | |
| | sulfanyl]phenyl}-9-thia-4,5,7- | | |
| | triazatricyclo[6.3.0.0^{2,6}]undeca- | | |
| | 1(8),2,5,10-tetraene-10-carboxamide | | |
| 86 | 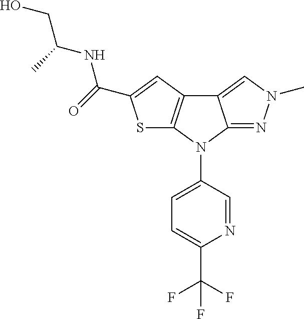 [SMILES: C[C@H](CO)NC(=O)c1cc2c3cn(C)nc3n(-c3ccc(C(F)(F)F)nc3)c2s1] | 1H NMR (500 MHz, DMSO) δ 9.46 (d, J = 2.8 Hz, 1H), 8.61- 8.55 (m, 1H), 8.21 (d, J = 8.0 Hz, 1H), 8.17 (dd, J = 8.7, 0.7 Hz, 1H), 8.13 (s, 1H), 8.07 (s, 1H), 4.75 (t, J = 5.7 Hz, 1H), 4.07 (s, 3H), 4.05-3.96 (m, 1H), 3.48 (dt, J = 11.0, 5.6 Hz, 1H), 3.36 (dt, J = 10.6, 6.1 Hz, 1H), 1.16 (d, J = 6.7 Hz, 3H). | Method C: Rt: 1.24 min M + H+: 424.00 2M + Na: 869.10 |
| | N-[(2R)-1-hydroxypropan-2-yl]-4-methyl- | | |
| | 7-[6-(trifluoromethyl)pyridin-3-yl]-9- | | |
| | thia-4,5,7- | | |
| | triazatricyclo[6.3.0.0^{2,6}]undeca- | | |
| | 1(8),2,5,10-tetraene-10-carboxamide | | |
| 87 | 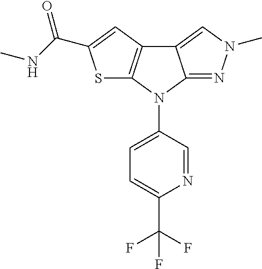 | 1H NMR (500 MHz, DMSO) δ 9.46 (d, J = 2.8 Hz, 1H), 8.57 (dd, J = 8.7, 2.8 Hz, 1H), 8.53 (q, J = 4.5 Hz, 1H), 8.16 (d, J = 8.7 Hz, 1H), 8.06 (s, 1H), 7.99 (s, 1H), 4.06 (s, 3H), 2.82 (d, J = 4.5 Hz, 3H). | Method C: Rt: 1.27 min M + H+: 380.00 2M + Na: 781.00 |
| | N,4-dimethyl-7-[6- | | |
| | (trifluoromethyl)pyridin-3-yl]-9-thia- | | |
| | 4,5,7-triazatricyclo[6.3.0.0^{2,6}]undeca- | | |
| | 1(8),2,5,10-tetraene-10-carboxamide | | |
| 88 | 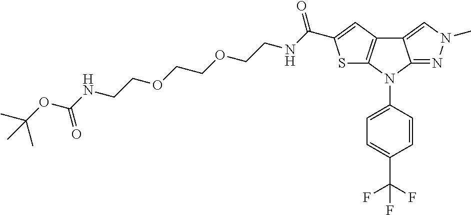 [SMILES: Cn1cc2c3cc(C(=O)NCCOCCOCCNC(=O)OC(C)(C)C)sc3n(-c3ccc(C(F)(F)F)cc3)c2n1] | 1H NMR (700 MHz, DMSO) δ 1.36 (s, 9H), 3.06 (q, J = 6.2 Hz, 2H), 3.38 (h, J = 5.2 Hz, 2H), 3.44 (q, J = 5.8 Hz, 2H), 3.52 (dd, J = 5.8, 3.4 Hz, 2H), 3.54- 3.57 (m, 4H), 4.06 (s, 3H), 6.75 (t, J = 5.9 Hz, 1H), 8.01 (d, J = 8.7 Hz, 2H), 8.04 (s, 1H), 8.07 (s, 1H), 8.23 (d, J = | Method C: Rt: 1.48 min M + H+: 596.20 M − tBu + H+: 540.10 M − BOC + H+: 496.10 M + Na: 618.20 |
| | tert-butyl N-(2-{2-[2-({4-methyl-7-[4- | 8.5 Hz, 2H), 8.61 (t, | |
| | (trifluoromethyl)phenyl]-9-thia-4,5,7- | J = 5.7 Hz, 1H). | |
| | triazatricyclo[6.3.0.0^{2,6}]undeca- | | |
| | 1(8),2,5,10-tetraen-10- | | |
| | yl}formamido)ethoxy]ethoxy}ethyl)carba- | | |
| | mate | | |
| 89 | 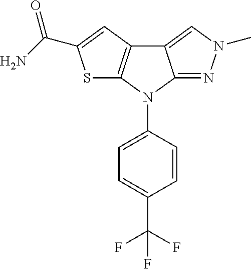 [SMILES: Cn1cc2c3cc(C(N)=O)sc3n(-c3ccc(C(F)(F)F)cc3)c2n1] | 1H NMR (500 MHz, DMSO) δ 4.06 (s, 3H), 7.40 (s, 2H), 8.00 (d, J = 8.7 Hz, 2H), 8.03 (s, 2H), 8.23 (d, J = 8.5 Hz, 2H). | Method C: Rt: 1.32 min M + H+: 365.00 M + Na: 387.00 2M + Na: 751.00 |
| | 4-methyl-7-[4-(trifluoromethyl)phenyl]- | | |
| | 9-thia-4,5,7- | | |
| | triazatricyclo[6.3.0.0^{2,6}]undeca- | | |
| | 1(8),2,5,10-tetraene-10-carboxamide | | |
| 90 | 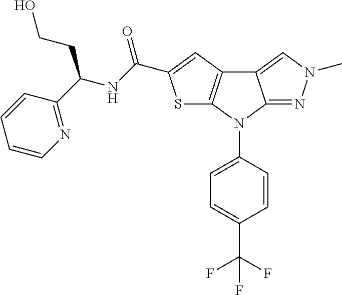 [SMILES: Cn1cc2c3cc(C(=O)N[C@H](CCO)c4ccccn4)sc3n(-c3ccc(C(F)(F)F)cc3)c2n1] | 1H NMR (500 MHz, DMSO) δ 1.98- 2.07 (m, 1H), 2.07- 2.16 (m, 1H), 3.45-3.58 (m, 2H), 4.07 (s, 3H), 4.58 (t, J = 5.0 Hz, 1H), 5.23 (td, J = 8.8, 5.4 Hz, 1H), 7.26 (ddd, J = 7.5, 4.8, 1.2 Hz, 1H), 7.43 (dt, J = 8.0, 1.1 Hz, 1H), 7.77 (td, J = 7.7, 1.8 Hz, 1H), 7.97-8.03 (m, 2H), 8.06 (s, 1H), 8.20-8.25 (m, 2H), 8.27 (s, | Method for chiral separation: SFC; column: YMC Amylose-C; eluent: CO2:2-propanol (60:40), wave length: 240 nm; flow: 5 ml/min Rt: 2.41 min |
| | N-[(1R)-3-hydroxy-1-(pyridin-2- | 1H), 8.54 (ddd, J = | |
| | yl)propyl]-4-methyl-7-[4- | 4.8, 1.8, 0.9 Hz, | |
| | (trifluoromethyl)phenyl]-9-thia-4,5,7- | 1H), 8.83 (d, J = 8.1 | |
| | triazatricyclo[6.3.0.0^{2,6}]undeca- | Hz, 1H). | |
| | 1(8),2,5,10-tetraene-10-carboxamide | | |
| | Absolute configuration assigned | | |
| | arbitrarily | | |

TABLE 1a

| Compound | | | |
|---|---|---|---|
| No. | | | Conditions and |
| (Example | | | elution time LCMS |
| No.) | Structure & Name | ¹H-NMR | (M + H⁺) |
| 91 | 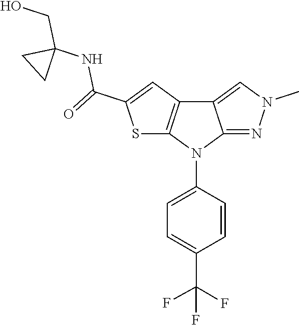 [SMILES: Cn1cc2c3cc(C(=O)NC4(CO)CC4)sc3n(-c3ccc(C(F)(F)F)cc3)c2n1] | ¹H NMR (500 MHz, DMSO-d₆) δ 8.76 (s, 1H), 8.23 (d, J = 8.6 Hz, 2H), 8.08 (s, 1H), 8.04 (s, 1H), 8.02-7.98 (m, 2H), 4.78 (t, J = 5.8 Hz, 1H), 4.05 (s, 3H), 3.54 (d, J = 5.8 Hz, 2H), 0.82-0.75 (m, 2H), 0.75-0.69 (m, 2H). | Method C, Rₜ = 1.34 min, [M + H]⁺ = 435.1 |
| | N-[1-(hydroxymethyl)cyclopropyl]-4- | | |
| | methyl-7-[4-(trifluoromethyl)phenyl]-9- | | |
| | thia-4,5,7-triazatri- | | |
| | cyclo[6.3.0.0^{2,6}]undeca-1(8),2,5,10- | | |
| | tetraene-10-carboxamide | | |
| 92 | 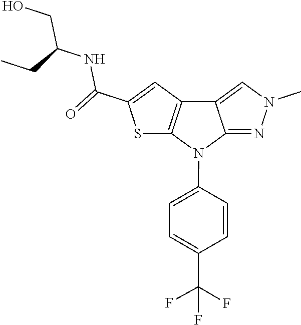 [SMILES: CC[C@@H](CO)NC(=O)c1cc2c3cn(C)nc3n(-c3ccc(C(F)(F)F)cc3)c2s1] | ¹H NMR (500 MHz, DMSO-d₆) δ 8.24 (d, J = 8.6 Hz, 2H), 8.15 (s, 1H), 8.09 (d, J = 8.4 Hz, 1H), 8.04 (s, 1H), 7.98-8.03 (m, 2H), 4.69 (t, J = 5.7 Hz, 1H), 4.06 (s, 3H), 3.84 (ddt, J = 14.3, 8.6, 5.6 Hz, 1H), 3.48 (dt, J = 11.0, 5.6 Hz, 1H), 3.41 (dt, J = 10.8, 6.0 Hz, 1H), 1.68 (dtd, J = 14.8, 7.4, 4.9 Hz, 1H), 1.47 (ddq, J = 14.5, 8.8, 7.4 Hz, 1H), 0.90 (t, J = 7.4 Hz, 3H). | Method C, Rₜ = 1.37 min, [M + H]⁺ = 437.1 |
| | N-[(2S)-1-hydroxybutan-2-yl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 93 | 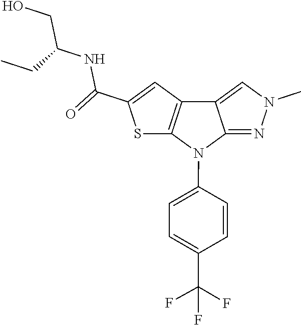 [SMILES: CC[C@H](CO)NC(=O)c1cc2c3cn(C)nc3n(-c3ccc(C(F)(F)F)cc3)c2s1] | ¹H NMR (500 MHz, DMSO-d₆) δ 8.26- 8.21 (m, 2H), 8.15 (s, 1H), 8.09 (d, J = 8.4 Hz, 1H), 8.04 (s, 1H), 8.03-7.98 (m, 2H), 4.69 (t, J = 5.7 Hz, 1H), 4.06 (s, 3H), 3.85 (ddt, J = 14.3, 8.7, 5.6 Hz, 1H), 3.48 (dt, J = 11.0, 5.6 Hz, 1H), 3.41 (dt, J = 10.8, 6.0 Hz, 1H), 1.68 (dtd, J = 14.8, 7.4, 4.9 Hz, 1H), 1.47 (ddt, J = 13.5, 8.8, 7.4 Hz, 1H), 0.90 (t, J = 7.4 Hz, 3H). | Method C, Rₜ = 1.37 min, [M + H]⁺ = 437.1 |
| | N-[(2R)-1-hydroxybutan-2-yl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 94 | 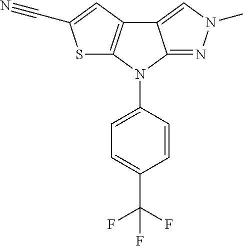 [SMILES: Cn1cc2c3cc(C#N)sc3n(-c3ccc(C(F)(F)F)cc3)c2n1] | ¹H NMR (500 MHz, DMSO-d₆) δ 8.23 (s, 1H), 8.13-8.19 (m, 2H), 8.04 (s, 1H), 7.97-8.03 (m, 2H), 4.06 (s, 3H). | Method C, Rₜ = 1.51 min, [M + H]⁺ = 347.0 |
| | 4-methyl-7-[4-(trifluoromethyl)phenyl]- | | |
| | 9-thia-4,5,7- | | |
| | triazatricyclo[6.3.0.0^{2,6}]undeca- | | |
| | 1(8),2,5,10-tetraene-10-carbonitrile | | |
| 95 | 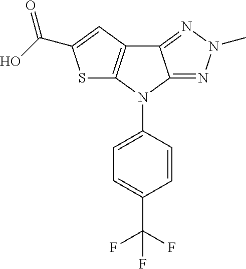 [SMILES: Cn1nc2c3cc(C(=O)O)sc3n(-c3ccc(C(F)(F)F)cc3)c2n1] | ¹H NMR (700 MHz, DMSO-d₆) δ 13.37 (s, 1H), 8.18-8.13 (m, 3H), 8.06 (d, J = 8.5 Hz, 2H), 4.38 (s, 3H). | Method C, Rₜ = 1.40 min, [M + H]⁺ = 367.0 |
| | 4-methyl-7-[4-(trifluoromethyl)phenyl]- | | |
| | 9-thia-3,4,5,7- | | |
| | tetraazatricyclo[6.3.0.0^{2,6}]undeca- | | |
| | 1(8),2,5,10-tetraene-10-carboxylic acid | | |
| 96 | 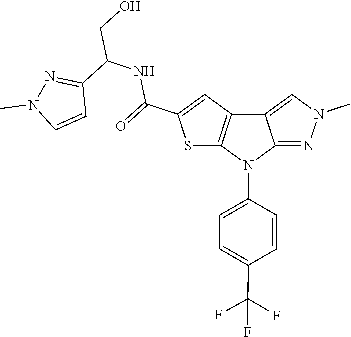 | ¹H NMR (700 MHz, DMSO-d₆) δ 8.60 (d, J = 8.6 Hz, 1H), 8.24 (d, J = 8.6 Hz, 2H), 8.23 (s, 1H), 8.06 (s, 1H), 8.02 (d, J = 8.7 Hz, 2H), 7.59 (d, J = 2.1 Hz, 1H), 6.22 (d, J = 2.2 Hz, 1H), 5.16 (td, J = 8.3, 5.3 Hz, 1H), 4.07 (s, 3H), 3.80 (s, 3H), 3.77 (dd, J = 11.1, 5.3 Hz, 1H), 3.72 (dd, J = 11.1, 8.1 Hz, 1H). | Method H, Rₜ = 0.76 min, [M + H]⁺ = 489.3 |
| | N-[2-hydroxy-1-(1-methyl-1H-pyrazol-3- | | |
| | yl)ethyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 97 | 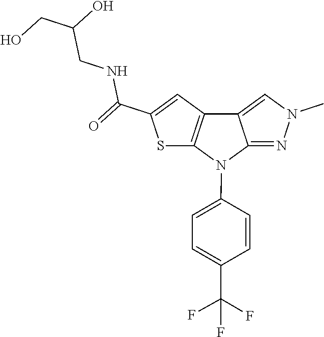 [SMILES: Cn1cc2c3cc(C(=O)NCC(O)CO)sc3n(-c3ccc(C(F)(F)F)cc3)c2n1] | ¹H NMR (700 MHz DMSO-d₆) δ 8.51 (t, J = 5.8 Hz, 1H), 8.24 (d, J = 8.7 Hz, 2H), 8.11 (s, 1H), 8.06 (s, 1H), 8.02 (d, J = 8.8 Hz, 2H), 4.84 (d, J = 5.0 Hz, 1H), 4.59 (t, J = 5.8 Hz, 1H), 4.07 (s, 3H), 3.65 (dq, J = 7.0, 5.2 Hz, 1H), 3.45-3.40 (m, 1H), 3.38 (t, J = 5.7 Hz, 2H), 3.24-3.17 (m, 1H). | Method H, Rₜ = 0.71 min, [M + H]⁺ = 439.3 |
| | N-(2,3-dihydroxypropyl)-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| 98 | 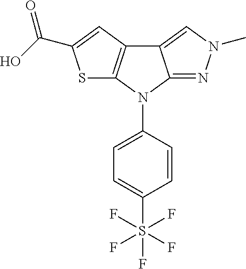 [SMILES: Cn1cc2c3cc(C(=O)O)sc3n(-c3ccc(S(F)(F)(F)(F)F)cc3)c2n1] | ¹H NMR (700 MHz, DMSO-d₆) δ 13.07 (s, 1H), 8.24-8.18 (m, 4H), 8.02 (d, J = 7.8 Hz, 2H), 4.07 (s, 3H). | Method H, Rₜ = 0.88 min, [M + H]⁺ = 424.2 |
| | 4-methyl-7-[4-(pentafluoro-lambda6- | | |
| | sulfanyl)phenyl]-9-thia-4,5,7- | | |
| | triazatricyclo[6.3.0.0^{2,6}]undeca- | | |
| | 1(8),2,5,10-tetraene-10-carboxylic acid | | |
| 99 | 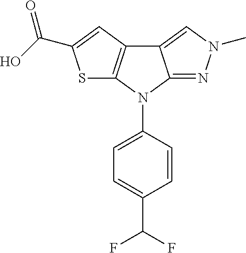 [SMILES: Cn1cc2c3cc(C(=O)O)sc3n(-c3ccc(C(F)F)cc3)c2n1] | ¹H NMR (400 MHz, DMSO-d₆) δ 12.92 (s, 1H), 8.22-8.14 (m, 2H), 8.01 (s, 1H), 7.99 (s, 1H), 7.85 (dt, J = 8.8, 1.2 Hz, 2H), 7.11 (t, J = 56.0 Hz, 1H), 4.07 (s, 3H). | Method H, Rₜ = 0.77 min, [M + H]⁺ = 348.2 |
| | 7-[4-(difluoromethyl)phenyl]-4-methyl- | | |
| | 9-thia-4,5,7- | | |
| | triazatricyclo[6.3.0.0^{2,6}]undeca- | | |
| | 1(8),2,5,10-tetraene-10-carboxylic acid | | |
| 100 | 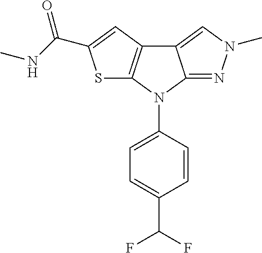 | ¹H NMR (700 MHz, DMSO-d₆) δ 8.49 (q, J = 4.5 Hz, 1H), 8.17 (d, J = 8.5 Hz, 2H), 8.03 (s, 1H), 7.98 (s, 1H), 7.84 (d, J = 8.3 Hz, 2H), 7.11 (t, J = 56.0 Hz, 1H), 4.06 (s, 3H), 2.82 (d, J = 4.5 Hz, 3H). | Method H, Rₜ = 0.74 min, [M + H]⁺ = 361.3 |
| | 7-[4-(difluoromethyl)phenyl]-N,4- | | |
| | dimethyl-9-thia-4,5,7- | | |
| | triazatricyclo[6.3.0.0^{2,6}]undeca- | | |
| | 1(8),2,5,10-tetraene-10-carboxamide | | |
| 101 | 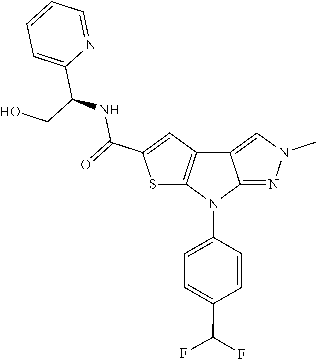 [SMILES: Cn1cc2c3cc(C(=O)N[C@@H](CO)c4ccccn4)sc3n(-c3ccc(C(F)F)cc3)c2n1] | ¹H NMR (700 MHz, DMSO-d₆) δ 8.76 (d, J = 8.1 Hz, 1H), 8.56 (dt, J = 4.7, 1.6 Hz, 1H), 8.30 (s, 1H), 8.19-8.15 (m, 2H), 8.06 (s, 1H), 7.84 (d, J = 8.2 Hz, 2H), 7.78 (td, J = 7.7, 1.8 Hz, 1H), 7.48-7.43 (m, 1H), 7.29 (ddd, J = 7.5, 4.8, 1.1 Hz, 1H), 7.10 (t, J = 56.0 Hz, 1H), 5.16 (td, J = 7.8, 5.2 Hz, 1H), 4.97 (t, J = 5.9 Hz, 1H), 4.07 (s, 3H), 3.87 (dt, J = 11.0, 5.4 Hz, 1H), 3.80 (ddd, J = 11.0, 7.6, 6.2 Hz, 1H). | Method H, Rₜ = 0.67 min, [M + H]⁺ = 468.3 |
| | 7-[4-(difluoromethyl)phenyl]-N-[(1R)-2- | | |
| | hydroxy-1-(pyridin-2-yl)ethyl]-4-methyl- | | |
| | 9-thia-4,5,7- | | |
| | triazatricyclo[6.3.0.0^{2,6}]undeca- | | |
| | 1(8),2,5,10-tetraene-10-carboxamide | | |
| 102 | 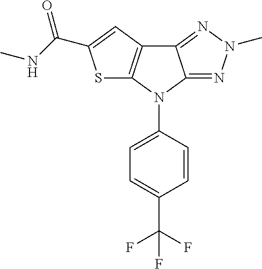 | ¹H NMR (700 MHz, DMSO-d₆) δ 8.65 (q, J = 4.5 Hz, 1H), 8.15- 8.11 (m, 3H), 8.06- 8.02 (m, 2H), 4.37 (s, 3H), 2.84 (d, J = 4.5 Hz, 3H). | Method H, Rₜ = 0.83 min, [M + H]⁺ = 380.3 |
| | N,4-dimethyl-7-[4- | | |
| | (trifluoromethyl)phenyl]-9-thia-3,4,5,7- | | |
| | tetraazatricyclo[6.3.0.0^{2,6}]undeca- | | |
| | 1(8),2,5,10-tetraene-10-carboxamide | | |
| 103 | 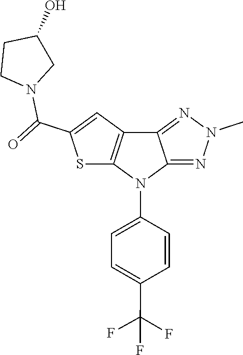 [SMILES: Cn1nc2c3cc(C(=O)N4CC[C@H](O)C4)sc3n(-c3ccc(C(F)(F)F)cc3)c2n1] | ¹H NMR (400 MHz, DMSO-d₆) δ 8.16 (d, J = 8.5 Hz, 2H), 8.07 (s, 2H), 8.05 (d, J = 1.9 Hz, 1H), 5.05 (d, J = 16.1 Hz, 1H), 4.44 (s, 1H), 4.38 (s, 3H), 4.01 (s, 2H), 3.75 (s, 0H), 3.64 (s, 2H), 3.53 (s, 1H), 1.99 (s, 2H). | Method C, Rₜ = 1.33 min, [M + H]⁺ = 436.0 |
| | (3S)-1-{4-methyl-7-[4-(trifluoromethyl)- | | |
| | phenyl]-9-thia-3,4,5,7-tetraazatri- | | |
| | cyclo[6.3.0.0^{2,6}]undeca-1(8),2,5,10- | | |
| | tetraene-10-carbonyl}pyrrolidin-3-ol | | |
| 104 | 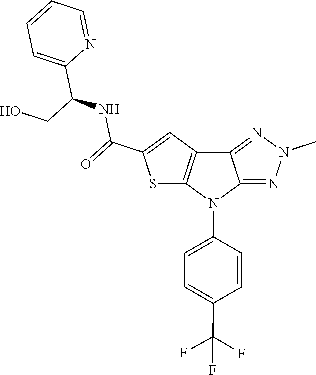 [SMILES: Cn1nc2c3cc(C(=O)N[C@@H](CO)c4ccccn4)sc3n(-c3ccc(C(F)(F)F)cc3)c2n1] | ¹H NMR (700 MHz, DMSO-d₆) δ 8.95 (d, J = 8.2 Hz, 1H), 8.57 (ddd, J = 4.8, 1.8, 0.9 Hz, 1H), 8.47 (s, 1H), 8.17-8.13 (m, 2H), 8.07-8.03 (m, 2H), 7.79 (td, J = 7.7, 1.8 Hz, 1H), 7.47 (dt, J = 8.0, 1.1 Hz, 1H), 7.30 (ddd, J = 7.5, 4.8, 1.1 Hz, 1H), 5.19 (td, J = 7.8, 5.1 Hz, 1H), 5.01 (t, J = 5.8 Hz, 1H), 4.39 (s, 3H), 3.89 (dt, J = 10.9, 5.4 Hz, 1H), 3.82 (ddd, J = 11.0, 7.6, 6.0 Hz, 1H). | Method H, Rₜ = 0.76 min, [M + H]⁺ = 487.3 |
| | N-[(1R)-2-hydroxy-1-(pyridin-2-yl)ethyl]- | | |
| | 4-methyl-7-[4-(trifluoromethyl)phenyl]- | | |
| | 9-thia-3,4,5,7- | | |
| | tetraazatricyclo[6.3.0.0^{2,6}]undeca- | | |
| | 1(8),2,5,10-tetraene-10-carboxamide | | |
| 105 | 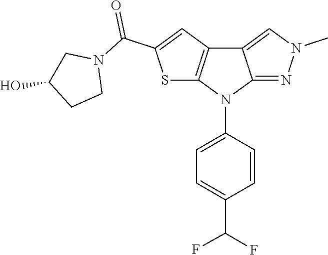 [SMILES: Cn1cc2c3cc(C(=O)N4CC[C@H](O)C4)sc3n(-c3ccc(C(F)F)cc3)c2n1] | ¹H NMR (400 MHz, DMSO-d₆) δ 8.21- 8.14 (m, 2H), 7.94 (s, 1H), 7.88-7.81 (m, 2H), 7.10 (t, J = 56.0 Hz, 1H), 5.04 (s, 1H), 4.42 (s, 1H), 4.35 (s, 0H), 4.07 (s, 3H), 3.98 (s, 2H), 3.64 (s, 3H), 1.99 (s, 2H). | Method H, Rₜ = 0.70 min, [M + H]⁺ = 417.3 |
| | (3S)-1-{7-[4-(difluoromethyl)phenyl]-4- | | |
| | methyl-9-thia-4,5,7-triazatri- | | |
| | cyclo[6.3.0.0^{2,6}]undeca-1(8),2,5,10- | | |
| | tetraene-10-carbonyl}pyrrolidin-3-ol | | |
| 106 | 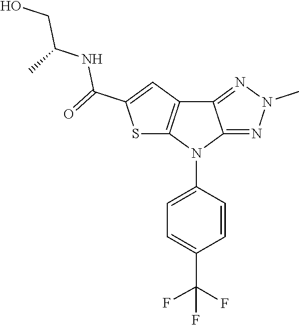 [SMILES: C[C@H](CO)NC(=O)c1cc2c3nn(C)nc3n(-c3ccc(C(F)(F)F)cc3)c2s1] | ¹H NMR (700 MHz, DMSO-d₆) δ 8.34 (d, J = 8.0 Hz, 1H), 8.27 (s, 1H), 8.15 (d, J = 8.6 Hz, 2H), 8.06 (d, J = 8.6 Hz, 2H), 4.80 (t, J = 5.7 Hz, 1H), 4.38 (s, 3H), 4.03 (dq, J = 7.9, 6.2 Hz, 1H), 3.50 (dt, J = 11.0, 5.6 Hz, 1H), 3.39 (dt, J = 10.7, 6.1 Hz, 1H), 1.18 (d, J = 6.7 Hz, 3H). | Method H, Rₜ = 0.79 min, [M + H]⁺ = 424.3 |
| | N-[(2R)-1-hydroxypropan-2-yl]-4-methyl- | | |
| | 7-[4-(trifluoromethyl)phenyl]-9-thia- | | |
| | 3,4,5,7-tetraazatricyclo[6.3.0.0^{2,6}]un- | | |
| | deca-1(8),2,5,10-tetraene-10- | | |
| | carboxamide | | |
| 107 | 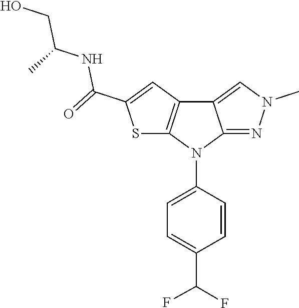 [SMILES: C[C@H](CO)NC(=O)c1cc2c3cn(C)nc3n(-c3ccc(C(F)F)cc3)c2s1] | ¹H NMR (400 MHz, DMSO-d₆) δ 8.20- 8.09 (m, 4H), 8.03 (s, 1H), 7.84 (dt, J = 8.2, 1.3 Hz, 2H), 7.10 (t, J = 56.0 Hz, 1H), 4.73 (t, J = 5.7 Hz, 1H), 4.06 (s, 3H), 4.05-3.94 (m, 1H), 3.49 (dt, J = 11.0, 5.6 Hz, 1H), 3.37 (dt, J = 10.6, 6.1 Hz, 1H), 1.17 (d, J = 6.7 Hz, 3H). | Method C, Rₜ = 0.71 min, [M + H]⁺ = 405.3 |
| | 7-[4-(difluoromethyl)phenyl]-N-[(2R)-1- | | |
| | hydroxypropan-2-yl]-4-methyl-9-thia- | | |
| | 4,5,7-triazatricyclo[6.3.0.0^{2,6}]undeca- | | |
| | 1(8),2,5,10-tetraene-10-carboxamide | | |
| 108 | 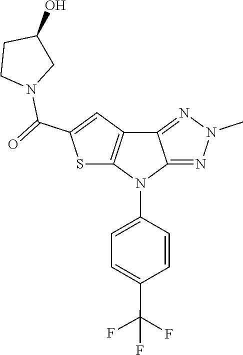 [SMILES: Cn1nc2c3cc(C(=O)N4CC[C@@H](O)C4)sc3n(-c3ccc(C(F)(F)F)cc3)c2n1] | ¹H NMR (400 MHz, DMSO-d₆) δ 8.16 (d, J = 8.4 Hz, 2H), 8.05-7.95 (m, 2H), 4.77 (d, J = 3.8 Hz, 1H), 4.42 (s, 1H), 4.37 (d, J = 3.2 Hz, 3H), 3.84 (s, 3H), 3.65 (d, J = 11.5 Hz, 1H), 2.50 (d, J = 4.0 Hz, 1H), 2.06 (s, 1H), 1.95 (s, 1H). | Method C, Rₜ = 1.33 min, [M + H]⁺ = 436.1 |
| | (3R)-1-{4-methyl-7-[4-(trifluoromethyl)- | | |
| | phenyl]-9-thia-3,4,5,7-tetraazatri- | | |
| | cyclo[6.3.0.0^{2,6}]undeca-1(8),2,5,10- | | |
| | tetraene-10-carbonyl}pyrrolidin-3-ol | | |
| 109 | 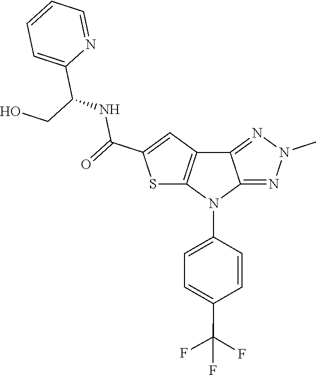 [SMILES: Cn1nc2c3cc(C(=O)N[C@H](CO)c4ccccn4)sc3n(-c3ccc(C(F)(F)F)cc3)c2n1] | ¹H NMR (400 MHz, DMSO-d₆) δ 8.92 (d, J = 8.1 Hz, 1H), 8.57 (ddd, J = 4.8, 1.9, 0.9 Hz, 1H), 8.46 (s, 1H), 8.15 (d, J = 8.6 Hz, 2H), 8.05 (d, J = 8.8 Hz, 2H), 7.79 (td, J = 7.7, 1.8 Hz, 1H), 7.47 (dt, J = 7.8, 1.1 Hz, 1H), 7.30 (ddd, J = 7.5, 4.8, 1.2 Hz, 1H), 5.24-5.14 (m, 1H), 4.98 (t, J = 5.8 Hz, 1H), 4.39 (s, 3H), 3.95-3.77 (m, 2H). | Method H, Rₜ = 0.76 min, [M + H]⁺ = 487.3 |
| | N-[(1S)-2-hydroxy-1-(pyridin-2-yl)ethyl]- | | |
| | 4-methyl-7-[4-(trifluoromethyl)phenyl]- | | |
| | 9-thia-3,4,5,7-tetraazatri- | | |
| | cyclo[6.3.0.0^{2,6}]undeca-1(8),2,5,10- | | |
| | tetraene-10-carboxamide | | |
| 110 | 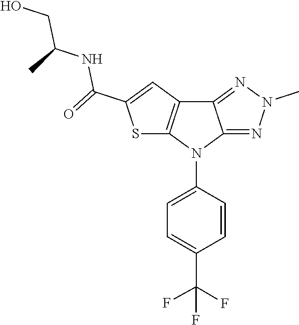 [SMILES: C[C@@H](CO)NC(=O)c1cc2c3nn(C)nc3n(-c3ccc(C(F)(F)F)cc3)c2s1] | ¹H NMR (400 MHz, DMSO-d₆) δ 8.31 (d, J = 8.0 Hz, 1H), 8.26 (s, 1H), 8.15 (d, J = 8.6 Hz, 2H), 8.05 (d, J = 8.7 Hz, 2H), 4.77 (t, J = 5.7 Hz, 1H), 4.38 (s, 3H), 4.04 (dq, J = 13.4, 6.5 Hz, 1H), 3.50 (dt, J = 11.1, 5.6 Hz, 1H), 3.39 (dt, J = 10.7, 6.1 Hz, 1H), 1.19 (d, J = 6.7 Hz, 3H). | Method H, Rₜ = 0.79 min, [M + H]⁺ = 424.3 |
| | N-[(2S)-1-hydroxypropan-2-yl]-4-methyl- | | |
| | 7-[4-(trifluoromethyl)phenyl]-9-thia- | | |
| | 3,4,5,7-tetraazatricyclo[6.3.0.0^{2,6}]un- | | |
| | deca-1(8),2,5,10-tetraene-10- | | |
| | carboxamide | | |
| 111 | 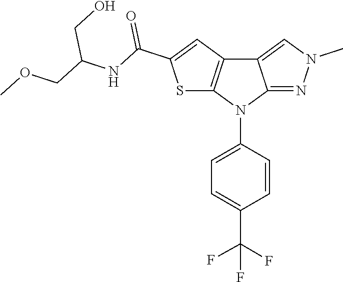 | ¹H NMR (700 MHz, DMSO-d₆) δ 8.26- 8.22 (m, 3H), 8.17 (s, 1H), 8.06 (s, 1H), 8.02 (d, J = 8.5 Hz, 2H), 4.80 (t, J = 5.7 Hz, 1H), 4.12 (dq, J = 7.7, 5.9 Hz, 1H), 4.07 (s, 3H), 3.55- 3.46 (m, 4H), 3.29 (s, 3H). | Method H, Rₜ = 0.77 min, [M + H]⁺ = 453.3 |
| | N-(1-hydroxy-3-methoxypropan-2-yl)-4- | | |
| | methyl-7-[4-(trifluoromethyl)phenyl]-9- | | |
| | thia-4,5,7-triazatricyclo[6.3.0.0^{2,6}]un- | | |
| | deca-1(8),2,5,10-tetraene-10- | | |
| | carboxamide | | |
| 112 | 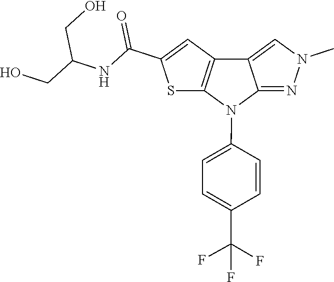 [SMILES: Cn1cc2c3cc(C(=O)NC(CO)CO)sc3n(-c3ccc(C(F)(F)F)cc3)c2n1] | ¹H NMR (700 MHz, DMSO-d₆) δ 8.24 (d, J = 8.6 Hz, 2H), 8.16 (s, 1H), 8.10 (d, J = 8.2 Hz, 1H), 8.06 (s, 1H), 8.02 (d, J = 8.7 Hz, 2H), 4.70 (t, J = 5.7 Hz, 2H), 4.07 (s, 3H), 3.96 (dp, J = 8.1, 5.9 Hz, 1H), 3.55 (hept, J = 5.5 Hz, 4H). | Method H, Rₜ = 0.70 min, [M + H]⁺ = 439.3 |
| | N-(1,3-dihydroxypropan-2-yl)-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| 113 | 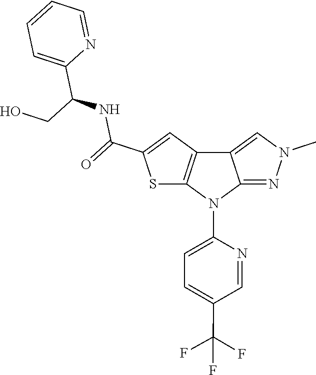 [SMILES: Cn1cc2c3cc(C(=O)N[C@@H](CO)c4ccccn4)sc3n(-c3ccc(C(F)(F)F)cn3)c2n1] | ¹H NMR (700 MHz, DMSO-d₆) δ 9.00 (dd, J = 2.3, 1.2 Hz, 1H), 8.70 (d, J = 8.1 Hz, 1H), 8.55 (dt, J = 4.9, 1.5 Hz, 1H), 8.48 (dd, J = 8.8, 2.4 Hz, 1H), 8.39 (d, J = 8.7 Hz, 1H), 8.24 (s, 1H), 8.09 (s, 1H), 7.78 (td, J = 7.7, 1.9 Hz, 1H), 7.46 (dt, J = 8.1, 1.2 Hz, 1H), 7.29 (ddd, J = 7.4, 4.9, 1.1 Hz, 1H), 5.17 (td, J = 7.7, 5.2 Hz, 1H), 4.96 (t, J = 5.9 Hz, 1H), 4.09 (s, 3H), 3.87 (dt, J = 10.9, 5.4 Hz, 1H), 3.80 (ddd, J = 11.1, 7.6, 6.3 Hz, 1H). | Method H, Rₜ = 0.72 min, [M + H]⁺ = 487.3 |
| | N-[(1R)-2-hydroxy-1-(pyridin-2-yl)ethyl]- | | |
| | 4-methyl-7-[5-(trifluoromethyl)pyridin- | | |
| | 2-yl]-9-thia-4,5,7- | | |
| | triazatricyclo[6.3.0.0^{2,6}]undeca- | | |
| | 1(8),2,5,10-tetraene-10-carboxamide | | |
| 114 | 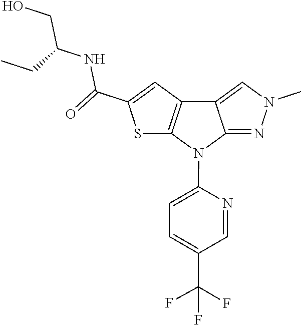 [SMILES: CC[C@H](CO)NC(=O)c1cc2c3cn(C)nc3n(-c3ccc(C(F)(F)F)cn3)c2s1] | ¹H NMR (700 MHz, DMSO-d₆) δ 8.99 (dd, J = 2.3, 1.2 Hz, 1H), 8.47 (dd, J = 8.8, 2.4 Hz, 1H), 8.38 (d, J = 8.7 Hz, 1H), 8.09 (s, 1H), 8.06 (s, 1H), 8.03 (d, J = 8.4 Hz, 1H), 4.70 (t, J = 5.7 Hz, 1H), 4.08 (s, 3H), 3.85 (ddt, J = 14.5, 8.7, 5.7 Hz, 1H), 3.49 (dt, J = 10.9, 5.5 Hz, 1H), 3.41 (dt, J = 10.7, 6.0 Hz, 1H), 1.68 (dtd, J = 14.8, 7.4, 4.9 Hz, 1H), 1.47 (ddt, J = 16.3, 14.6, 7.4 Hz, | Method H, Rₜ = 0.79 min, [M + H]⁺ = 438.3 |
| | N-[(2R)-1-hydroxybutan-2-yl]-4-methyl- | 1H), 0.91 (t, J = 7.4 | |
| | 7-[5-(trifluoromethyl)pyridin-2-yl]-9- | Hz, 3H). | |
| | thia-4,5,7-triazatricyclo[6.3.0.0^{2,6}]un- | | |
| | deca-1(8),2,5,10-tetraene-10- | | |
| | carboxamide | | |
| 115 | 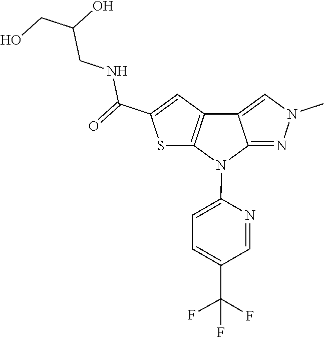 [SMILES: Cn1cc2c3cc(C(=O)NCC(O)CO)sc3n(-c3ccc(C(F)(F)F)cn3)c2n1] | ¹H NMR (700 MHz, DMSO-d₆) δ 8.99 (dt, J = 2.0, 1.0 Hz, 1H), 8.48 (dd, J = 8.8, 2.5 Hz, 1H), 8.43 (t, J = 5.8 Hz, 1H), 8.38 (d, J = 8.7 Hz, 1H), 8.07 (s, 1H), 8.04 (s, 1H), 4.84 (d, J = 4.9 Hz, 1H), 4.59 (t, J = 5.8 Hz, 1H), 4.08 (s, 3H), 3.65 (dq, J = 6.9, 5.3 Hz, 1H), 3.42 (dt, J = 13.4, 5.6 Hz, 1H), 3.37 (t, J = 5.7 Hz, 2H), 3.20 (ddd, J = 13.2, 7.0, 5.6 Hz, 1H). | Method H, Rₜ = 0.70 min, [M + H]⁺ = 440.3 |
| | N-(2,3-dihydroxypropyl)-4-methyl-7-[5- | | |
| | (trifluoromethyl)pyridin-2-yl]-9-thia- | | |
| | 4,5,7-triazatricyclo[6.3.0.0^{2,6}]undeca- | | |
| | 1(8),2,5,10-tetraene-10-carboxamide | | |
| 116 | 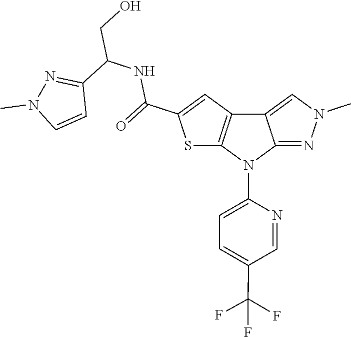 | ¹H NMR (700 MHz, DMSO-d₆) δ 9.00 (dd, J = 2.2, 1.2 Hz, 1H), 8.51 (d, J = 8.7 Hz, 1H), 8.47 (dd, J = 8.8, 2.4 Hz, 1H), 8.38 (d, J = 8.7 Hz, 1H), 8.16 (s, 1H), 8.07 (s, 1H), 7.58 (d, J = 2.1 Hz, 1H), 6.22 (d, J = 2.2 Hz, 1H), 5.16 (td, J = 8.3, 5.3 Hz, 1H), 4.84 (t, J = 5.9 Hz, 1H), 4.08 (s, 3H), 3.80 (s, 3H), 3.78- 3.68 (m, 2H). | Method H, Rₜ = 0.74 min, [M + H]⁺ = 490.3 |
| | N-[2-hydroxy-1-(1-methyl-1H-pyrazol-3- | | |
| | yl)ethyl]-4-methyl-7-[5- | | |
| | (trifluoromethyl)pyridin-2-yl]-9-thia- | | |
| | 4,5,7-triazatricyclo[6.3.0.0^{2,6}]undeca- | | |
| | 1(8),2,5,10-tetraene-10-carboxamide | | |
| 117 | 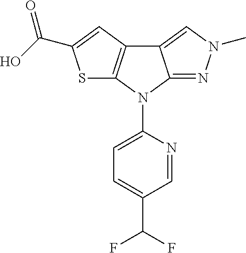 [SMILES: Cn1cc2c3cc(C(=O)O)sc3n(-c3ccc(C(F)F)cn3)c2n1] | ¹H NMR (700 MHz, DMSO-d₆) δ 12.85 (s, 1H), 8.80 (d, J = 2.2 Hz, 1H), 8.36 (d, J = 8.5 Hz, 1H), 8.32 (dd, J = 8.7, 2.3 Hz, 1H), 8.01 (s, 1H), 7.96 (s, 1H), 7.20 (t, J = 55.4 Hz, 1H), 4.08 (s, 3H), 1.08 (s, 0H). | Method H, Rₜ = 0.76 min, [M + H]⁺ = 349.2 |
| | 7-[5-(difluoromethyl)pyridin-2-yl]-4- | | |
| | methyl-9-thia-4,5,7- | | |
| | triazatricyclo[6.3.0.0^{2,6}]undeca- | | |
| | 1(8),2,5,10-tetraene-10-carboxylic acid | | |
| 118 | 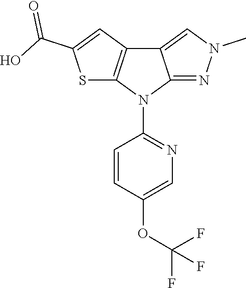 [SMILES: Cn1cc2c3cc(C(=O)O)sc3n(-c3ccc(OC(F)(F)F)cn3)c2n1] | ¹H NMR (700 MHz, DMSO-d₆) δ 12.85 (s, 1H), 8.73 (d, J = 2.7 Hz, 1H), 8.34 (d, J = 9.0 Hz, 1H), 8.26- 8.22 (m, 1H), 8.01 (s, 1H), 7.95 (s, 1H), 4.08 (s, 3H). | Method H, Rₜ = 0.83 min, [M + H]⁺ = 383.2 |
| | 4-methyl-7-[5-(trifluoromethoxy)pyridin- | | |
| | 2-yl]-9-thia-4,5,7-triazatri- | | |
| | cyclo[6.3.0.0^{2,6}]undeca-1(8),2,5,10- | | |
| | tetraene-10-carboxylic acid | | |
| 119 | 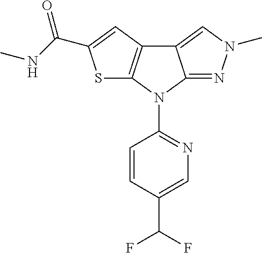 | ¹H NMR (700 MHz, DMSO-d₆) δ 8.78 (d, J = 2.2 Hz, 1H), 8.42 (q, J = 4.5 Hz, 1H), 8.35 (d, J = 8.6 Hz, 1H), 8.31 (dd, J = 8.6, 2.2 Hz, 1H), 8.04 (s, 1H), 7.92 (s, 1H), 7.19 (t, J = 55.5 Hz, 1H), 4.07 (s, 3H), 2.81 (d, J = 4.5 Hz, 3H). | Method H, Rₜ = 0.73 min, [M + H]⁺ = 362.2 |
| | 7-[5-(difluoromethyl)pyridin-2-yl]-N,4- | | |
| | dimethyl-9-thia-4,5,7-triazatri- | | |
| | cyclo[6.3.0.0^{2,6}]undeca-1(8),2,5,10- | | |
| | tetraene-10-carboxamide | | |
| 120 | 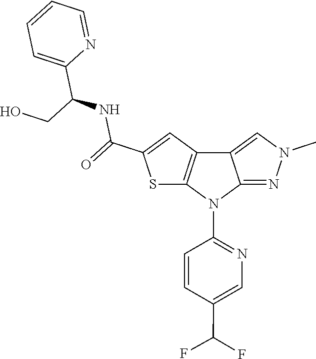 [SMILES: Cn1cc2c3cc(C(=O)N[C@@H](CO)c4ccccn4)sc3n(-c3ccc(C(F)F)cn3)c2n1] | ¹H NMR (700 MHz, DMSO-d₆) δ 8.79 (d, J = 2.2 Hz, 1H), 8.68 (d, J = 8.1 Hz, 1H), 8.55 (dt, J = 4.7, 1.5 Hz, 1H), 8.36 (d, J = 8.6 Hz, 1H), 8.31 (dd, J = 8.6, 2.2 Hz, 1H), 8.23 (s, 1H), 8.07 (s, 1H), 7.78 (td, J = 7.7, 1.8 Hz, 1H), 7.45 (d, J = 7.9 Hz, 1H), 7.28 (ddd, J = 7.5, 4.7, 1.1 Hz, 1H), 7.19 (t, J = 55.4 Hz, 1H), 5.16 (td, J = 7.7, 5.2 Hz, 1H), 4.96 (t, J = 5.9 Hz, 1H), 4.09 (s, 3H), 3.87 (dt, J = 10.9, 5.4 Hz, 1H), 3.80 (ddd, J = 11.0, 7.4, | Method H, Rₜ = 0.65 min, [M + H]⁺ = 469.3 |
| | 7-[5-(difluoromethyl)pyridin-2-yl]-N- | 6.1 Hz, 1H). | |
| | [(1R)-2-hydroxy-1-(pyridin-2-yl)ethyl]-4- | | |
| | methyl-9-thia-4,5,7-triazatri- | | |
| | cyclo[6.3.0.0^{2,6}]undeca-1(8),2,5,10- | | |
| | tetraene-10-carboxamide | | |
| 121 | 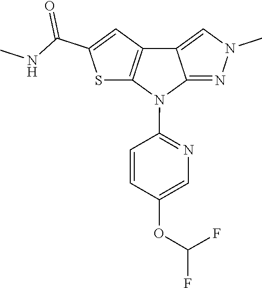 | ¹H NMR (700 MHz, DMSO-d₆) δ 8.52 (d, J = 2.8 Hz, 1H), 8.39 (q, J = 4.6 Hz, 1H), 8.28 (d, J = 9.0 Hz, 1H), 8.03 (s, 1H), 8.01 (dd, J = 9.0, 2.8 Hz, 1H), 7.91 (s, 1H), 7.31 (t, J = 73.5 Hz, 1H), 4.07 (s, 3H), 2.80 (d, J = 4.5 Hz, 3H). | Method H, Rₜ = 0.73 min, [M + H]⁺ = 378.2 |
| | 7-[5-(difluoromethoxy)pyridin-2-yl]-N,4- | | |
| | dimethyl-9-thia-4,5,7-triazatri- | | |
| | cyclo[6.3.0.0^{2,6}]undeca-1(8),2,5,10- | | |
| | tetraene-10-carboxamide | | |
| 122 | 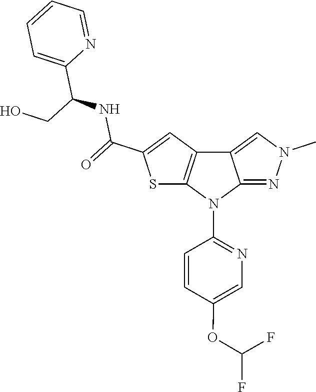 [SMILES: Cn1cc2c3cc(C(=O)N[C@@H](CO)c4ccccn4)sc3n(-c3ccc(OC(F)F)cn3)c2n1] | ¹H NMR (700 MHz, DMSO-d₆) δ 8.65 (d, J = 8.0 Hz, 1H), 8.55 (ddd, J = 4.8, 1.8, 0.9 Hz, 1H), 8.52 (d, J = 2.8 Hz, 1H), 8.29 (d, J = 9.0 Hz, 1H), 8.22 (s, 1H), 8.06 (s, 1H), 8.02 (dd, J = 8.9, 2.9 Hz, 1H), 7.78 (td, J = 7.7, 1.8 Hz, 1H), 7.45 (dt, J = 7.9, 1.1 Hz, 1H), 7.30 (t, 1H), 7.29-7.26 (m, 1H), 5.16 (td, J = 7.7, 5.1 Hz, 1H), 4.95 (t, J = 5.9 Hz, 1H), 4.08 (s, 3H), 3.86 (dt, J = 10.9, 5.5 Hz, 1H), 3.80 (ddd, J = 11.0, 7.5, 6.2 Hz, 1H). | Method H, Rₜ = 0.66 min, [M + H]⁺ = 485.3 |
| | 7-[5-(difluoromethoxy)pyridin-2-yl]-N- | | |
| | [(1R)-2-hydroxy-1-(pyridin-2-yl)ethyl]-4- | | |
| | methyl-9-thia-4,5,7- | | |
| | triazatricyclo[6.3.0.0^{2,6}]undeca- | | |
| | 1(8),2,5,10-tetraene-10-carboxamide | | |
| 123 | 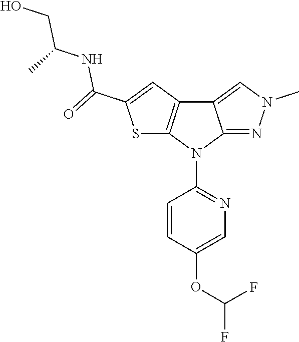 [SMILES: C[C@H](CO)NC(=O)c1cc2c3cn(C)nc3n(-c3ccc(OC(F)F)cn3)c2s1] | ¹H NMR (700 MHz, DMSO-d₆) δ 8.52 (d, J = 2.8 Hz, 1H), 8.28 (d, J = 9.0 Hz, 1H), 8.06 (d, J = 8.0 Hz, 1H), 8.04 (s, 1H), 8.03 (s, 1H), 8.01 (dd, J = 8.9, 2.9 Hz, 1H), 7.31 (t, J = 73.5 Hz, 1H), 4.74 (t, J = 5.7 Hz, 1H), 4.07 (s, 3H), 4.00 (dq, J = 8.0, 6.4 Hz, 1H), 3.48 (dt, J = 11.0, 5.7 Hz, 1H), 3.35 (dt, J = 10.6, 6.2 Hz, 1H), 1.16 (d, J = 6.7 Hz, 3H). | Method H, Rₜ = 0.71 min, [M + H]⁺ = 422.3 |
| | 7-[5-(difluoromethoxy)pyridin-2-yl]-N- | | |
| | [(2R)-1-hydroxypropan-2-yl]-4-methyl-9- | | |
| | thia-4,5,7-triazatricyclo[6.3.0.0^{2,6}]un- | | |
| | deca-1(8),2,5,10-tetraene-10- | | |
| | carboxamide | | |
| 124 | 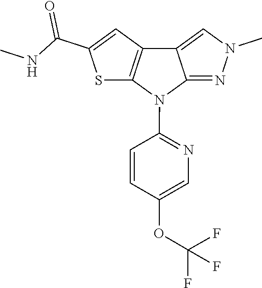 | ¹H NMR (700 MHz, DMSO-d₆) δ 8.71 (d, J = 2.8 Hz, 1H), 8.41 (q, J = 4.6 Hz, 1H), 8.33 (d, J = 9.0 Hz, 1H), 8.22 (dd, J = 9.0, 2.8 Hz, 1H), 8.04 (s, 1H), 7.92 (s, 1H), 4.07 (s, 3H), 2.81 (d, J = 4.5 Hz, 3H). | Method H, Rₜ = 0.80 min, [M + H]⁺ = 396.2 |
| | N,4-dimethyl-7-[5- | | |
| | (trifluoromethoxy)pyridin-2-yl]-9-thia- | | |
| | 4,5,7-triazatricyclo[6.3.0.0^{2,6}]undeca- | | |
| | 1(8),2,5,10-tetraene-10-carboxamide | | |
| 125 | 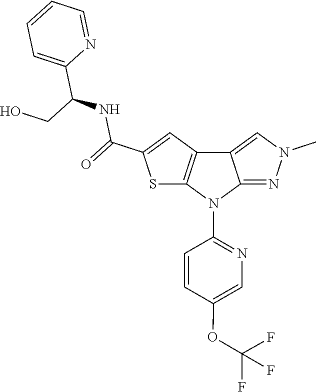 [SMILES: Cn1cc2c3cc(C(=O)N[C@@H](CO)c4ccccn4)sc3n(-c3ccc(OC(F)(F)F)cn3)c2n1] | ¹H NMR (700 MHz, DMSO-d₆) δ 8.72 (d, J = 2.7 Hz, 1H), 8.68 (d, J = 8.1 Hz, 1H), 8.55 (dt, J = 4.7, 1.6 Hz, 1H), 8.34 (d, J = 9.0 Hz, 1H), 8.24-8.21 (m, 2H), 8.07 (s, 1H), 7.78 (td, J = 7.7, 1.8 Hz, 1H), 7.46 (d, J = 7.9 Hz, 1H), 7.28 (ddd, J = 7.4, 4.8, 1.2 Hz, 1H), 5.16 (td, J = 7.7, 5.1 Hz, 1H), 4.96 (t, J = 5.9 Hz, 1H), 4.08 (s, 3H), 3.87 (dt, J = 10.8, 5.4 Hz, 1H), 3.80 (ddd, J = 11.0, 7.4, 6.1 Hz, 1H). | Method H, Rₜ = 0.72 min, [M + H]⁺ = 503.3 |
| | N-[(1R)-2-hydroxy-1-(pyridin-2-yl)ethyl]- | | |
| | 4-methyl-7-[5-(trifluoromethoxy)pyridin- | | |
| | 2-yl]-9-thia-4,5,7-triazatri- | | |
| | cyclo[6.3.0.0^{2,6}]undeca-1(8),2,5,10- | | |
| | tetraene-10-carboxamide | | |
| 126 | 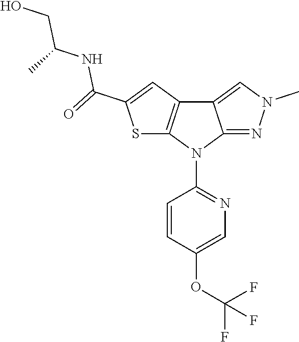 [SMILES: C[C@H](CO)NC(=O)c1cc2c3cn(C)nc3n(-c3ccc(OC(F)(F)F)cn3)c2s1] | ¹H NMR (700 MHz, DMSO-d₆) δ 8.72 (d, J = 2.8 Hz, 1H), 8.34 (d, J = 9.0 Hz, 1H), 8.22 (dd, J = 9.3, 2.9 Hz, 1H), 8.08 (d, J = 8.0 Hz, 1H), 8.05 (d, J = 3.2 Hz, 2H), 4.75 (t, J = 5.7 Hz, 1H), 4.07 (s, 3H), 4.00 (dq, J = 8.0, 6.5 Hz, 1H), 3.48 (dt, J = 11.0, 5.6 Hz, 1H), 3.35 (dt, J = 10.7, 6.1 Hz, 1H), 1.16 (d, J = 6.7 Hz, 3H). | Method H, Rₜ = 0.77 min, [M + H]⁺ = 440.3 |
| | N-[(2R)-1-hydroxypropan-2-yl]-4-methyl- | | |
| | 7-[5-(trifluoromethoxy)pyridin-2-yl]-9- | | |
| | thia-4,5,7-triazatricyclo[6.3.0.0^{2,6}]un- | | |
| | deca-1(8),2,5,10-tetraene-10- | | |
| | carboxamide | | |
| 127 | 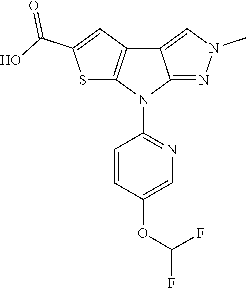 [SMILES: Cn1cc2c3cc(C(=O)O)sc3n(-c3ccc(OC(F)F)cn3)c2n1] | ¹H NMR (700 MHz, DMSO-d₆) δ 12.81 (s, 1H), 8.53 (d, J = 2.8 Hz, 1H), 8.29 (d, J = 8.9 Hz, 1H), 8.03 (dd, J = 9.0, 2.8 Hz, 1H), 8.00 (s, 1H), 7.95 (s, 1H), 7.32 (t, J = 73.5 Hz, 1H), 4.07 (s, 3H). | Method H, Rₜ = 0.76 min, [M + H]⁺ = 365.2 |
| | 7-[5-(difluoromethoxy)pyridin-2-yl]-4- | | |
| | methyl-9-thia-4,5,7-triazatri- | | |
| | cyclo[6.3.0.0^{2,6}]undeca-1(8),2,5,10- | | |
| | tetraene-10-carboxylic acid | | |
| 128 | 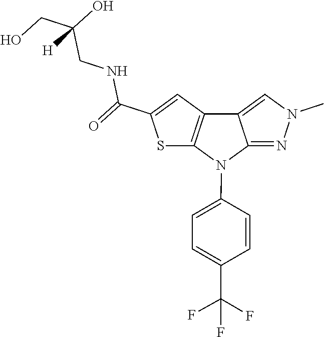 [SMILES: Cn1cc2c3cc(C(=O)NC[C@@H](O)CO)sc3n(-c3ccc(C(F)(F)F)cc3)c2n1] | ¹H NMR (700 MHz, DMSO-d₆) δ 8.52 (t, J = 5.8 Hz, 1H), 8.24 (d, J = 8.7 Hz, 2H), 8.11 (s, 1H), 8.06 (s, 1H), 8.02 (d, J = 8.5 Hz, 2H), 4.85 (d, J = 5.2 Hz, 1H), 4.59 (t, J = 5.7 Hz, 1H), 4.07 (s, 3H), 3.68-3.62 (m, 1H), 3.43 (s, 0H), 3.45-3.40 (m, 1H), 3.37 (t, J = 5.6 Hz, 2H), 3.20 (ddd, J = 13.1, 7.1, 5.7 Hz, 1H). | Method H, Rₜ = 0.71 min, [M + H]⁺ = 439.3 Method for chiral separation: SFC; column: ChiralPAK AY-H; eluent: CO2: Ethanol (85:15), wave length: 240 nm; flow: 5 mL/min Rₜ: 12.58 min |
| | N-[(2R)-2,3-dihydroxypropyl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| | Absolute configuration assigned | | |
| | arbitrarily | | |
| 129 | 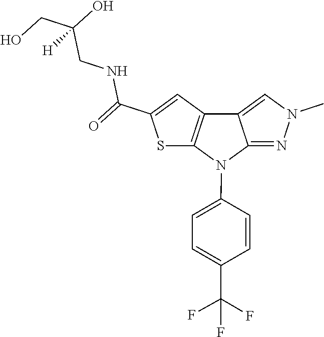 [SMILES: Cn1cc2c3cc(C(=O)NC[C@H](O)CO)sc3n(-c3ccc(C(F)(F)F)cc3)c2n1] | ¹H NMR (700 MHz, DMSO-d₆) δ 8.52 (t, J = 5.7 Hz, 1H), 8.26-8.22 (m, 2H), 8.11 (s, 1H), 8.06 (s, 1H), 8.02 (d, J = 8.7 Hz, 2H), 4.86 (d, J = 5.3 Hz, 1H), 4.59 (t, J = 5.8 Hz, 1H), 4.07 (s, 3H), 3.65 (dp, J = 7.1, 5.3 Hz, 1H), 3.42 (dt, J = 13.5, 5.6 Hz, 1H), 3.37 (t, J = 5.6 Hz, 2H), 3.20 (ddd, J = 13.1, 7.1, 5.6 Hz, 1H). | Method H, Rₜ = 0.71 min, [M + H]⁺ = 439.3 Method for chiral separation: SFC; column: ChiralPAK AY-H; eluent: CO2: Ethanol (85:15), wave length: 240 nm; flow: 5 mL/min Rₜ: 16.39 min |
| | N-[(2S)-2,3-dihydroxypropyl]-4-methyl- | | |
| | 7-[4-(trifluoromethyl)phenyl]-9-thia- | | |
| | 4,5,7-triazatricyclo[6.3.0.0^{2,6}]undeca- | | |
| | 1(8),2,5,10-tetraene-10-carboxamide | | |
| | Absolute configuration assigned | | |
| | arbitrarily | | |
| 130 | 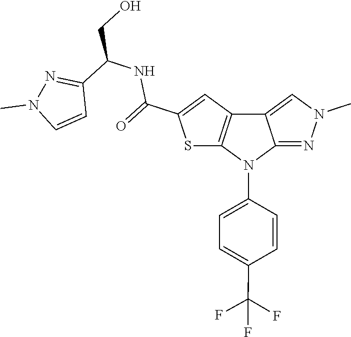 | ¹H NMR (700 MHz, DMSO-d₆) δ 8.61 (d, J = 8.6 Hz, 1H), 8.25 (d, J = 8.7 Hz, 2H), 8.23 (s, 1H), 8.06 (s, 1H), 8.02 (d, J = 8.7 Hz, 2H), 7.59 (d, J = 2.2 Hz, 1H), 6.21 (d, J = 2.2 Hz, 1H), 5.15 (td, J = 8.3, 5.2 Hz, 1H), 4.87 (t, J = 5.9 Hz, 1H), 4.07 (s, 3H), 3.80 (s, 2H), 3.78 (s, 0H), 3.72 (ddd, J = 11.2, 8.2, 6.1 Hz, 1H). | Method H, Rₜ = 0.76 min, [M + H]⁺ = 489.3 Method for chiral separation: SFC; column: ChiralPak IC; eluent: CO2: Ethanol (55:45), wave length: 240 nm; flow: 5 mL/min Rₜ: 3.55 min |
| | N-[(1S)-2-hydroxy-1-(1-methyl-1H- | | |
| | pyrazol-3-yl)ethyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| | Absolute configuration assigned | | |
| | arbitrarily | | |
| 131 | 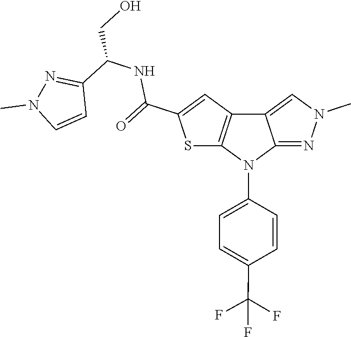 | ¹H NMR (700 MHz, DMSO-d₆) δ 8.60 (d, J = 8.7 Hz, 1H), 8.25 (d, J = 8.6 Hz, 2H), 8.23 (s, 0H), 8.06 (s, 1H), 8.03- 7.98 (m, 2H), 7.59 (d, J = 2.2 Hz, 1H), 6.21 (d, J = 2.2 Hz, 1H), 5.15 (td, J = 8.3, 5.3 Hz, 1H), 4.87 (t, J = 5.9 Hz, 1H), 4.07 (s, 3H), 3.80 (s, 3H), 3.76 (dt, J = 10.9, 5.4 Hz, 1H), 3.72 (ddd, J = 11.1, 8.1, 6.1 Hz, 1H). | Method C, Rₜ = 0.76 min, [M + H]⁺ = 489.4 Method for chiral separation: SFC; column: ChiralPak IC; eluent: CO2: Ethanol (55:45), wave length: 240 nm; flow: 5 mL/min Rₜ: 6.97 min |
| | N-[(1R)-2-hydroxy-1-(1-methyl-1H- | | |
| | pyrazol-3-yl)ethyl]-4-methyl-7-[4- | | |
| | (trifluoromethyl)phenyl]-9-thia-4,5,7- | | |
| | triazatricyclo[6.3.0.0^{2,6}]undeca- | | |
| | 1(8),2,5,10-tetraene-10-carboxamide | | |
| | Absolute configuration assigned | | |
| | arbitrarily | | |
| 132 | 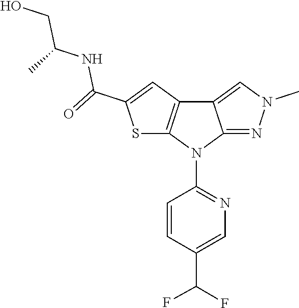 [SMILES: C[C@H](CO)NC(=O)c1cc2c3cn(C)nc3n(-c3ccc(C(F)F)cn3)c2s1] | ¹H NMR (500 MHz, DMSO-d₆) δ 8.81- 8.77 (m, 1H), 8.35 (d, J = 8.6 Hz, 1H), 8.31 (dd, J = 8.7, 2.2 Hz, 1H), 8.10 (d, J = 8.0 Hz, 1H), 8.06 (d, J = 1.2 Hz, 2H), 7.20 (t, J = 55.4 Hz, 1H), 4.76 (t, J = 5.8 Hz, 1H), 4.08 (s, 3H), 4.06-3.96 (m, 1H), 3.48 (dt, J = 11.0, 5.6 Hz, 1H), 3.39- 3.30 (m, 1H), 1.21- 1.13 (m, 3H). | Method H, Rₜ = 0.70 min, [M + H]⁺ = 406.3 |
| | 7-[5-(difluoromethyl)pyridin-2-yl]-N- | | |
| | [(2R)-1-hydroxypropan-2-yl]-4-methyl-9- | | |
| | thia-4,5,7-triazatricyclo[6.3.0.0^{2,6}]un- | | |
| | deca-1(8),2,5,10-tetraene-10- | | |
| | carboxamide | | |

### Method A:

Column: Kinetex EVO C18 2.6 μm, 3.0*50 mm, Column Oven: 40° C., Mobile Phase A: water/5 mM NH₄HCO₃, Mobile Phase B: MeCN, Flow rate: 1.2 mL/min, Gradient: 10% B to 95% B in 2.1 min, 254 nm

### Method B:

Column: HALO C18, 2 μM, 3.0*30 mm, Column Oven 40° C., Mobile Phase A: Water/0.05% TFA, Mobile Phase B: MeCN/0.05% TFA, Flow rate: 1.2 mL/min, Gradient: 5% B to 100% B in 1.2 min, 254 nm

### Method C:

Column: Kinetex EVO C18 5.0 μm, 50*4.6 mm, Mobile Phase A: H2O+0.1% TFA B: MeCN+0.1% TFA, 1%->99% B: 0->1.8 min, 99% B: 1.8->2.1 min, T: 40° C., Flow: 3.3 mL/min, 254 nM

### Method D:

Column: HALO C18, 2.0 μM, 3.0*30 mm, Column Oven: 40° C., Mobile Phase A: Water/0.1% TFA, Mobile Phase B: MeCN/0.1% TFA, Flow rate: 1.5 mL/min, Gradient: 5% to 95% in 1.2 min, 254 nm

### Method E:

Column: HALO C18, 2.0 μM, 3.0*30 mm, Column Oven: 40° C., Solvent A: Water+0.05% TFA, Solvent B: MeCN+0.05% TFA, Flow: 1.2 mL/min, Gradient: 20% B to 95% B in 2.5 min, 254 nM

### Method F:

Column: Kinetex EVO C18, 2.6 μm, 3.0*50 mm, Column Oven: 40° C., Mobile Phase A: 6.5 mM NH₄HCO₃+NH₄OH (pH=10), Mobile Phase B: MeCN; Flow rate: 1.2 mL/min, Gradient: 10% to 95% in 1.9 min, 254 nm

### Method G:

Column: HALO C18, 2.0 μM, 3.0*30 mm, Column Oven: 40C, Solvent A: Water+0.05% TFA, Solvent B: MeCN+0.05% TFA, Flow: 1.2 mL/min, Gradient: 30% B to 95% B in 1.2 min, 254 nm.

### Method H:

Column: Kinetex UPLC EVO C18, 1.7 μm, 2.1*50 mm, Column Oven: 40° C., Mobile Phase A: 6.5 mM H₂O+0.05% HCO₂H, Mobile Phase B: MeCN+0.04% HCO₂H; Flow rate: 0.9 mL/min, 1%->99% B: 0->1.0 min, 99% B: 1.0->1.3 min. 254 nm

### Biological Activity

### SK-HEP-1 Reporter Assay

To identify inhibitors of YAP-TEAD interaction, 8× TEAD responsive elements driving the NanoLuc® luciferase gene were stably integrated into SK-HEP-1 cells (ECACC #: 91091816).

For the assay, cells were treated in duplicates with the test compounds in a 10-point dose, with the top concentration starting at 30 μM (final concentration in assay). After a 24 hour incubation at 37° C., 95% rH, and 5% CO₂, a luciferase substrate/lysis reagent mix (NanoGlo™, Promega) was added to the cells, allowing the quantification of cellular luciferase activity.

Cell Media: The cells were cultured in the following media: MEM, +10% FBS, +1× GlutaMAX, +1 mM Sodium-Pyruvate, +100 μM Non-essential amino acids, +0.1 mg/ml Hygromycin. The media used for the assay was: MEM (w/o Phenol Red), +10% FBS, +1× GlutaMAX, +1 mM Sodium-Pyruvate, +100 μM Non-essential amino acids, +0.5% Pen/Strep

Reagents: The reagents used are listed below:

| Reagent | Manufacturer | Order No. |
|---|---|---|
| MEM | Sigma | 2279-500 ml |
| MEM (w/o Phenol Red) | Gibco | 51200-046 |
| FBS | PAN Biotech | P30-1502 |
| GlutaMAX | Gibco | 35050-038 |
| Sodium Pyruvate | Gibco | 11360 |
| NEAA | Gibco | 11140 |
| Hygromycin | Sigma | 10687-010 |
| NanoGlo ® Luciferase Assay System | Promega | N1150 |
| Penicillin/Streptomycin | Invitrogen | 15140 |
| DPBS (1x) | Gibco | 14190 |
| Accutase | PAN Biotech | P10-21500 |

Cell culture: The cells were examined using an inverted microscope to check for health and cell density. To dissociate adherent cells, the monolayer of cells was washed once with pre-warmed PBS. After removing the PBS, 3 ml pre-warmed Accutase® was added to a F75 flask, dispersed evenly and the flask was allowed to sit in incubator for ˜4-5 minutes.

When a single cell suspension was obtained, 7 ml of prewarmed growth media was added and resuspended with the cells. The cell suspension was transferred to a sterile 15 ml conical centrifuge tube, and spun for 5 min at 300×g, RT. The supernatant was discarded and the pellet was resuspended in 10 ml of pre-warmed growth media.

The total cell count was determined, and 20 μl of the desired cell number was added to each well of a 384 well plate using a Multidrop Combi. The plates were then incubated for 24 hours at 37° C., 95% rH, and 5% CO₂.

Compound treatment: 24 hours after seeding, the cells were treated with compounds.

A 1:333 dilution of compounds, diluted in DMSO, was made to get a final concentration of 0.3% DMSO per well. To transfer the compounds to the assay plate, 120 nl was shot from Labcyte low dead volume plates to the cell plates containing 20 μl media/well with the ECHO 555 liquid handling system. After treatment, the cells were fed with 20 μl fresh pre-warmed assay media using a Multidrop combi.

The assay plates were then incubated for another 24 h at 37° C., 95% rH, and 5% CO₂.

Luciferase readout: 24 h after treatment, the plates were taken out of the incubator and were allowed to equilibrate to RT. 30 μl of NanoGlo® reagent was added to the plates in the dark. Plates were shaken for 20 min on a Teleshake (˜1500 rpm) in the dark. The luminescence was then measured using an EnVision microplate reader. The IC50 values were generated using Genedata Screener®.

### Viability Assay in NCI-H226 (Yap-Dependent) and SW620 Yap KO (Yap Independent) Cells

The ability of YAP-TEAD inhibitors to inhibit tumor cell growth was evaluated using two different cell lines: NCI-H226, which is a YAP dependent cell line, and SW620 cells, where YAP and TAZ were knocked out using CRISPR to generate a YAP independent cell line.

For the assay, cells were treated in duplicates with the test compounds in a 10-point dose, 1:3 dilution steps, with the top concentration starting at 30 μM (final concentration in assay). After a 96 hour incubation at 37° C., 95% rH, and 5% CO₂, a cell-permeant DNA-binding dye that stains only healthy cells (CyQUANT®, Promega) was added to the cells, allowing the quantification of cell viability.

Cell Media: The NCI-H226 cells were cultured in the following media: RPMI 1640, +10% FBS, +1× GlutaMAX, +10 mM HEPES, +0.5% Pen/Strep. The SW620-KO cells were cultured in the following media: DMEM/F-12, +10% FBS, +1× GlutaMAX, +10 mM HEPES, +0.5% Pen/Strep.

Reagents: The reagents used are listed below:

| Reagent | Manufacturer | Order No. |
|---|---|---|
| DMEM/F12 | Gibco | 21331 |
| RPMI 1640 | Gibco | 31870 |
| FBS | PAN Biotech | P30-1502 |
| GlutaMAX | Gibco | 35050-038 |
| HEPES | Gibco | 15630 |
| CyQuant ® | Promega | C35012 |
| Penicillin/Streptomycin | Invitrogen | 15140 |
| DPBS (1x) | Gibco | 14190 |
| Accutase | PAN Biotech | P10-21500 |

Cell culture: The cells were examined using an inverted microscope to check for health, cell density, etc. To dissociate adherent cells, the monolayer of cells was washed once with pre-warmed PBS. After removing the PBS, 3 ml pre-warmed Accutase was added to a F75 flask, dispersed evenly and the flask was allowed to sit in incubator for ˜4-5 minutes.

When a single cell suspension was obtained, 7 ml of prewarmed growth media was added and resuspended with the cells. The cell suspension was transferred to a sterile 15 ml conical centrifuge tube, and spun for 5 min at 300×g, RT. The supernatant was discarded and the pellet was resuspended in 10 ml of pre-warmed growth media.

The total cell count was determined, and 20 μl of the desired cell number was added to each well of a 384 well plate using a Multidrop Combi. The plates were then incubated for 24 hours at 37° C., 95% rH, and 5% CO₂.

Compound treatment: 24 hours after seeding, the cells were treated with compounds.

A 1:333 dilution of compounds, diluted in DMSO, was made to get a final concentration of 0.3% DMSO per well. To transfer the compounds to the assay plate, 120 nl was shot from Labcyte low dead volume plates to the cell plates containing 20 μl media/well with the ECHO 555 liquid handling system. After treatment, the cells were fed with 20 μl fresh pre-warmed assay media using a Multidrop combi.

The assay plates were then incubated for 96 h at 37° C., 95% rH, and 5% CO₂.

### CyQuant® Measurement

96 h after treatment 30 μl of CyQuant® reagent was added to the assay plates using a Multidrop combi in the dark. The plates were then incubated for 1 hour at 37° C., 95% rH and 5% CO2. Thereafter, the assay plates were removed from the incubator and allowed to equilibrate to RT for 30 min in the dark without lid. Finally, they were measured using an EnVision microplate reader with a FITC bottom read program.

Experimental data in SK-HEP-1 reporter assay of the compounds shown in Table 1 and Table 1a are shown in Table 2 below and classified in the following groups:

| | |
|---|---|
| Group A | IC₅₀ is in the range of 1 nM to 10 nM |
| Group B | IC₅₀ is in the range of >10 nM to 100 nM |
| Group C | IC₅₀ is in the range of >100 nM to 10000 nM |
| Group D | IC₅₀ is in the range >10000 nM |
| n.d. | Not detectable below threshold given in parantheses |

Experimental data in the Viability assay of the compounds shown in Table 1 and Table 1a are shown in Table 2 below and classified in the following groups:

For the viability assay in NCI-H226 cells:

| | |
|---|---|
| Group A | IC₅₀ is in the range of 1 nM to 100 nM |
| Group B | IC₅₀ is in the range of >100 nM to 1000 nM |
| Group C | IC₅₀ is in the range of >1000 nM to 10000 nM |
| Group D | IC₅₀ is in the range >10000 nM |
| n.d. | Not detectable below threshold given in parantheses |

For the viability assay in SW620 Yap KO cells:

| | |
|---|---|
| Group A | IC₅₀ is in the range of 0.1 μM to 1 μM |
| Group B | IC₅₀ is in the range of >1 μM to 10 μM |
| Group C | IC₅₀ is in the range of >10 μM to 30 μM |
| Group D | IC₅₀ is in the range >30 μM |
| n.d. | Not detectable below threshold given in parantheses |

TABLE 2

| | SK-HEP-1 | NCI-H226 | SW620 |
|---|---|---|---|
| Compound No. | reporter | viability | viability |
| (Example No.) | IC₅₀ (nM) | IC₅₀ (nM) | IC₅₀ (μM) |
| 1 | C | D | |
| 2 | B | B | |
| 3 | B | B | |
| 4 | D | C | |
| 5 | D | C | |
| 6 | C | | |
| 7 | D | | |
| 8 | | | |
| 9 | C | | |
| 10 | B | B | n.d. (30) |
| 11 | A | A | n.d. (30) |
| 12 | D | | |
| 13 | D | | |
| 14 | D | | |
| 15 | | | |
| 16 | D | | |
| 17 | | | |
| 18 | | | |
| 19 | A | A | n.d. (30) |
| 20 | B | B | n.d. (30) |
| 21 | C | B | B |
| 22 | B | B | n.d. (30) |
| 23 | D | | |
| 24 | C | | |
| 25 | B | B | n.d. (30) |
| 26 | C | | |
| 27 | B | B | n.d. (30) |
| 28 | D | | |
| 29 | D | | |
| 30 | C | | |
| 31 | D | A | n.d. (3.2) |
| 32 | B | A | |
| 33 | B | A | n.d. (3.2) |
| 34 | C | B | |
| 35 | B | A | n.d. (3.2) |
| 36 | C | B | n.d. (30) |
| 37 | C | C | n.d. (30) |
| 38 | C | | |
| 39 | C | | |
| 40 | B | A | n.d. (30) |
| 41 | C | B | n.d. (3) |
| 42 | n.d. (9500) | C | B |
| 43 | n.d. (9500) | A | n.d. (3) |
| 44 | n.d. (9500) | A | n.d. (3) |
| 45 | n.d. (9500) | A | B |
| 46 | A | A | n.d. (3) |
| 47 | B | A | B |
| 48 | n.d. (30000) | A | C |
| 49 | B | A | n.d. (3) |
| 50 | C | A | n.d. (30) |
| 51 | C | ND | n.d. (30) |
| 52 | B | A | n.d. (30) |
| 53 | A | | |
| 54 | A | | |
| 55 | n.d. (9500) | | |
| 56 | n.d. (9500) | | |
| 57 | n.d. (9500) | | |
| 58 | D | | |
| 59 | n.d. (300) | B | |
| 60 | C | | |
| 61 | n.d. (9500) | B | |
| 62 | n.d. (300) | B | n.d. (3.0) |
| 63 | n.d. (9500) | A | n.d. (3.0) |
| 64 | n.d. (950) | A | n.d. (3.0) |
| 65 | n.d. (300) | B | n.d. (3.0) |
| 66 | n.d. (9500) | A | n.d. (3.0) |
| 67 | n.d. (9500) | B | n.d. (3.0) |
| 68 | n.d. (950) | A | n.d. (3.0) |
| 69 | n.d. (30000) | A | n.d. (3.2) |
| 73 | n.d. (3000) | | |
| 80 | n.d. (30000) | B | n.d. (30) |
| 81 | B | | |
| 82 | C | | |
| 83 | C | | |
| 84 | B | | |
| 85 | B | | |
| 86 | n.d. (30000) | | |
| 87 | n.d. (30000) | | |
| 88 | n.d. (30000) | 88 | n.d. |
| 89 | 980 | | |
| 90 | n.d | 1500 | n.d. |
| 91 | n.d. (30000) | A | n.d. (30) |
| 92 | n.d. (30000) | B | n.d. (3) |
| 93 | B | A | n.d. (3) |
| 94 | C | | |
| 95 | A | A | n.d. (30) |
| 96 | B | A | n.d. (30) |
| 97 | A | B | n.d. (30) |
| 98 | C | | |
| 99 | C | B | n.d. (30) |
| 100 | B | A | n.d. 30) |
| 101 | C | | |
| 102 | B | A | n.d. (30) |
| 103 | C | | |
| 104 | C | | |
| 105 | B | B | n.d. (30) |
| 106 | C | | |
| 107 | C | | |
| 108 | D | | |
| 109 | C | | |
| 110 | n.d. (30000) | | |
| 111 | C | | |
| 112 | n.d. (9500) | | |
| 113 | n.d. (9500) | | |
| 114 | n.d. (3000) | | |
| 115 | n.d. (300) | | |
| 116 | n.d. (30000) | | |
| 117 | C | C | n.d. (30) |
| 118 | B | B | n.d. (30) |
| 119 | n.d. (30000) | B | n.d. (30) |
| 120 | n.d. (300) | B | n.d. (30) |
| 121 | n.d. (30000) | A | n.d. (30) |
| 122 | C | A | n.d. (30) |
| 123 | n.d. (300) | A | n.d. (30) |
| 124 | C | A | n.d. (3) |
| 125 | C | A | n.d. (30) |
| 126 | B | A | n.d. (30) |
| 127 | B | | |
| 128 | n.d. (30000) | | |
| 129 | n.d. (950) | | |
| 130 | B | A | n.d. (30) |
| 131 | n.d. (95) | | |
| 132 | n.d. (300) | | |

The following examples relate to medicaments:

### Example A: Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### Example B: Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C: Solution

A solution is prepared from 1 g of an active ingredient of the formula I, 9.38 g of NaH₂PO₄·2 H₂O, 28.48 g of Na₂HPO₄·12H₂O and 0.1 g of benzalkonium chloride in 940 mL of bidistilled water. The pH is adjusted to 6.8, and the solution is made up to 1 l and sterilised by irradiation. This solution can be used in the form of eye drops.

### Example D: Ointment

500 mg of an active ingredient of the formula I are mixed with 99.5 g of Vaseline under aseptic conditions.

### Example E: Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### Example F: Dragees

Tablets are pressed analogously to Example E and subsequently coated in a conventional manner with a coating of sucrose, potato starch, talc, tragacanth and dye.

### Example G: Capsules

2 kg of active ingredient of the formula I are introduced into hard gelatine capsules in a conventional manner in such a way that each capsule contains 20 mg of the active ingredient.

### Example H: Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. A heteroaromatic compound of formula I, 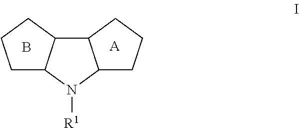
wherein
Ring A represents a five-membered heteroaromatic ring selected from the group consisting of the following ring moieties: 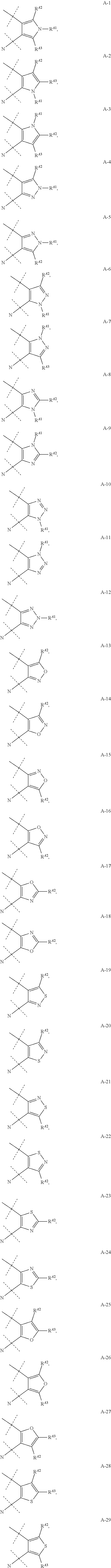 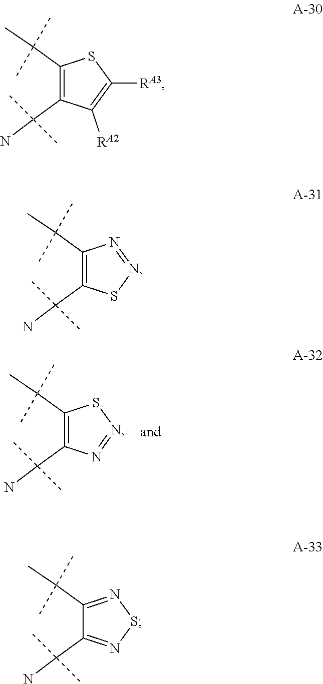
Ring B represents a five-membered heteroaromatic ring selected from the group consisting of the following ring moieties: 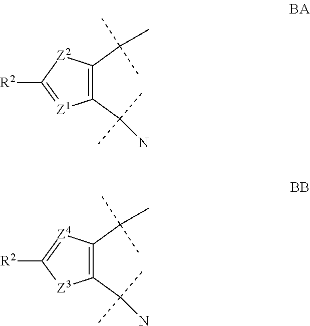
wherein
Z¹ is CR^{Z1} or N;
Z² is O, S, or NR^{Z2};
Z³ is O, S, or NR^{Z3};
Z⁴ is CR^{Z4} or N;
R¹ represents H, Ar¹, Hetar¹, Cyc¹, Hetcyc¹, L¹-Ar¹, L¹-Hetar¹, L²-Cyc¹, L²-Hetcyc¹, or unsubstituted or substituted C₁₋₈-aliphatic;
R² represents —C(═O)—OR^{2a}, —C(═O)—NR^{2b}R^{2c}, —(CH₂)_{w}—C(═O)—NR^{2b}R^{2c}, —(CH₂)ₓ—NR^{2d}—C(═O)—R^{2c}, —S—R^{2f}, —S(═O)—R^{2f}, —S(═O)₂—R^{2g}, —S(═O)₂—NR^{2h}R²ⁱ, —S(═O)₂—OH, —S(═O)(═NR^{2j})—OH, —S(═O)(═NR^{2j})—R^{2g}, —S(═O)(═NR^{2k})—NR^{2l}R^{2m}, F, Cl, Br, I, —CN, —(CH₂)ᵥ—CN, —P(═O)(OR^{2o})(OR^{2p}), —(CH₂)_{y}—NR^{2q}R^{2r}, —(CF₂)_{z}—NR^{2d}—S(═O)₂—R^{2g}, —N═S(═O)—R^{2s}R^{2t}, —C(O)—N═S(═O)—R^{2s}R^{2t}, —C(═O)—N═S(═N—R²ⁿ)—R^{2s}R^{2t}, —B(OH)₂ or Hetcyc^{X};
R^{A1} represents Ar³, Hetar³, Cyc³, Hetcyc³, L³-Ar³, L³-Hetar³, L⁴-Cyc³, L⁴-Hetcyc³, unsubstituted or substituted C₁₋₈-aliphatic;
R^{A2}, R^{A3} represent independently from each other H, halogen, Ar³, Hetar³, Cyc³, Hetcyc³, L³-Ar³, L³-Hetar³, L⁴-Cyc³, L⁴-Hetcyc³, unsubstituted or substituted C₁₋₈-aliphatic;
R^{Z1} represents H, C₁₋₆-aliphatic or halogen: or forms together with R² a divalent radical —S(═O)₂—N(H)—C(═O)—;
R^{Z2}, R^{Z3} represent independently from each other H or C₁₋₆-aliphatic;
R^{Z4} represents H, C₁₋₆-aliphatic or halogen;
Ar¹, Ar³ are independently from each other a mono-, bi- or tricyclic aryl with 5, 6, 7, 8, 9, 10, 11, 12, 13, or 14 ring carbon atoms, wherein that aryl may be unsubstituted or substituted with substituents R^{B1}, R^{B2}, R^{B3}, R^{B4}, R^{B5}, R^{B6} and/or R^{B7} which may be the same or different;
Ar^{2a}, Ar^{2b}, Ar⁴ are independently from each other a mono- or bicyclic aryl with 5, 6, 7, 8, 9, 10 ring carbon atoms, wherein that aryl may be unsubstituted or substituted with substituents R^{D1}, R^{D2}, R^{D3}, R^{D4} and/or R^{D5} which may be the same or different;
Ar^{X}, Ar^{Z} are independently from each other an un-substituted or substituted benzo ring;
Ar^{Y} is an un-substituted or mono- or di-substituted phenyl;
Hetar¹, Hetar³ are independently from each other a mono-, bi- or tricyclic heteroaryl with 5, 6, 7, 8, 9, 10, 11, 12, 13, or 14 ring atoms wherein 1, 2, 3, 4, or 5 of said ring atoms is/are a hetero atom(s) selected from the group consisting of N, O and S and the remaining are carbon atoms, wherein that heteroaryl may be unsubstituted or substituted with substituents R^{B1}, R^{B2}, R^{B3}, R^{B4}, R^{B5}, R^{B6} and/or R^{B7} which may be the same or different;
Hetar^{2a}, Hetar^{2b}, Hetar⁴, Hetar^{Y1} are independently from each other a mono- or bicyclic heteroaryl with 5, 6, 7, 8, 9, or 10 ring atoms wherein 1, 2, 3, 4, or 5 of said ring atoms is/are a hetero atom(s) selected from the group consisting of N, O and S and the remaining are carbon atoms, wherein that heteroaryl may be unsubstituted or substituted with substituents R^{D1}, R^{D2}, R^{D3}, R^{D4} and/or R^{D5} which may be the same or different;
Hetar^{Z} is pyrrole, N-methyl-pyrrole, pyrazole, imidazole, or triazole;
Cyc¹, Cyc³ are independently from each other a saturated or partially unsaturated, mono-, bi- or tricyclic carbocycle with 3, 4, 5, 6, 7, 8, 9, 10, 11, 12, 13, 14, or 15 ring carbon atoms, wherein that carbocycle may be unsubstituted or substituted with R^{B8}, R^{B9}, R^{B10}, R^{B11}, R^{B12} and/or R^{B13} which may be the same or different; and wherein that carbocycle may optionally be fused to Ar^{X} via 2 adjacent ring atoms of said Ar^{X} and wherein that fused carbocycle may be unsubstituted or substituted with R^{C1}, R^{C2}, R^{C3}, R^{C4}, R^{C5} and/or R^{C6} which may be the same or different;
Cyc^{2a}, Cyc⁴ are independently from each other a saturated or partially unsaturated monocyclic carbocycle with 3, 4, 5, 6 or 7 ring carbon atoms, wherein that carbocycle may be unsubstituted or substituted with R^{D6}, R^{D7}, R^{D8}, R^{D9} and/or R^{D10} which may be the same or different;
Cyc^{2b} is a saturated or partially unsaturated monocyclic carbocycle with 3, 4, 5, 6 or 7 ring carbon atoms, wherein that carbocycle may be unsubstituted or substituted independently from each other with R^{D6}, R^{D7}, R^{D8}, R^{D9} and/or R^{D10} wherein that carbocycle may optionally be fused to Ar^{Z} or Hetar^{Z} via 2 adjacent ring atoms and wherein that fused carbocycle may optionally further be substituted with independently from each other R^{C1}, R^{C2} and/or R^{C3};
Cyc^{Y1} is a saturated or partially unsaturated monocyclic carbocycle with 3, 4, 5, 6 or 7 ring carbon atoms, wherein that carbocycle may be unsubstituted or substituted with halogen, hydroxy, unsubstituted or substituted C₁₋₆-aliphatic;
Hetcyc¹, Hetcyc³ are independently from each other a saturated or partially unsaturated, mono-, bi- or tricyclic heterocycle with 3, 4, 5, 6, 7, 8, 9, 10, 11, 12, 13, 14, or 15 ring atoms wherein 1, 2, 3, 4, or 5 of said ring atoms is/are a hetero atom(s) selected from the group consisting of N, O and S and the remaining are carbon atoms, wherein that heterocycle may be unsubstituted or substituted with R^{B8}, R^{B9}, R^{B10}, R^{B11}, R^{B12} and/or R^{B13} which may be the same or different;
Hetcyc^{2a}, Hetcyc⁴ are independently from each other a saturated or partially unsaturated, monocyclic heterocycle with 3, 4, 5, 6, or 7 ring atoms wherein 1 or 2 of said ring atoms is/are a hetero atom(s) selected from the group consisting of N, O and S and the remaining are carbon atoms, wherein that heterocycle may be unsubstituted or substituted with R^{D6}, R^{D7}, R^{D8}, R^{D9} and/or R^{D10} which may be the same or different;
Hetcyc^{2b} is a saturated monocyclic heterocycle with 5 or 6 ring atoms wherein 1 or 2 of said ring atoms is/are a hetero atom(s) selected from the group consisting of N, O and S and the remaining are carbon atoms, wherein that heterocycle may be unsubstituted or substituted independently from each other with R^{D6}, R^{D7}, R^{D8}, R^{D9} and/or R^{D10} wherein that heterocycle may optionally be fused to Ar^{Z} or Hetar^{Z} and wherein that fused heterocycle may optionally further be substituted with independently from each other R^{C1}, R^{C2} and/or R^{C3};
Hetcyc^{X} is a saturated, partially unsaturated or aromatic, monocyclic heterocycle with 3, 4, 5, 6, or 7 ring atoms wherein 1, 2, 3, or 4 of said ring atoms is/are a hetero atom(s) selected from the group consisting of N, O and S and the remaining are carbon atoms, wherein said heterocycle may be unsubstituted or substituted with R^{X1}, R^{X2}, R^{X3}, R^{X4}, R^{X5}, R^{X6}, R^{X7} and/or R^{X8} which may be the same or different, and wherein that unsubstituted or substituted heterocycle is optionally a carboxylic acid bioisostere;
Hetcyc^{Y} is a saturated, partially unsaturated or aromatic, monocyclic heterocycle with 3, 4, 5, 6, or 7 ring atoms wherein 1, 2, 3, or 4 of said ring atoms is/are a hetero atom(s) selected from the group consisting of N, O and S and the remaining are carbon atoms;
Hetcyc^{Y1} is a saturated or partially unsaturated monocyclic heterocycle with 5 or 6 ring atoms wherein 1 or 2 of said ring atoms are heteroatoms selected from the group consisting of N, O, and S and the remaining are carbon atoms;
L¹, L³ are independently from each other a divalent radical selected from the group consisting of —S(═O)₂—, —C(═O)—, un-substituted or substituted, and straight-chain or branched C₁₋₆-alkylene or C₂₋₆-alkenylene, in both of which one of the carbon units of the alkylene or alkenylene chain may be replaced by —O—;
L², L⁴ are independently from each other a divalent radical selected from the group consisting of —C(═O)—, un-substituted or substituted, and straight-chain or branched C₁₋₆-alkylene or C₂₋₆-alkenylene, in both of which one of the carbon units of the alkylene or alkenylene chain may be replaced by —O—;
R^{2a} represents H, un-substituted or substituted C₁₋₈-aliphatic, Ar^{2a}, Hetar^{2a}, Cyc^{2a}, Hetcyc^{2a}, or Cat;
Cat represents a monovalent cation;
R^{2b}, R^{2c} both represent H;
or one of R^{2b} and R^{2c} represents H or unsubstituted or substituted C₁₋₈-aliphatic, while the other of R^{2b} and R^{2c} represents unsubstituted or substituted C₁₋₁₀-aliphatic, —OH, —O—C₁₋₆-alkyl, —CN, —S(═O)₂—R^{2g}, Ar^{2b}, Hetar^{2b}, Cyc^{2b} or Hetcyc^{2b};
or R^{2b} and R^{2c} form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms;
R^{2d}, R^{2j}, R^{2k}, R^{2o}, R^{2p} represent independently from each other H, un-substituted or substituted C₁₋₈-aliphatic;
R^{2e} represents H, halogen, un-substituted or substituted C₁₋₈-aliphatic, aryl, heteroaryl; saturated or partially unsaturated heterocyclyl;
R^{2f}, R^{2g} represent independently from each other un-substituted or substituted C₁₋₈-aliphatic;
R^{2h}, R²ⁱ represent independently from each other H, un-substituted or substituted C₁₋₈-aliphatic, Ar^{2b}, Hetar^{2b}, Cyc^{2b} or Hetcyc^{2b}; or form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms;
R^{2l}, R^{2m}, R^{2q}, R^{2r} represent independently from each other H, un-substituted or substituted C₁₋₈-aliphatic; or R^{2l} together with R^{2m} and/or R^{2q} together with R^{2r} form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms;
R^{2s}, R^{2t} represent independently from each other unsubstituted or substituted C₁₋₈-aliphatic; or form together an unsubstituted or substituted divalent C₃₋₆-alkylene radical;
R^{2a} represents hydrogen or unsubstituted or substituted C₁₋₆-aliphatic;
R^{B1}, R^{B2}, R^{B3}, R^{B4}, R^{B5}, R^{B6}, R^{B7} represent independently from each other un-substituted or substituted, straight-chain or branched C₁₋₆-aliphatic, C₁₋₆-aliphatoxy, —S—C₁₋₆-aliphatic; halogen, —CN, —SF₅, —S(═O)—R^{b1}, S(═O)₂—R^{b1}, —NR^{b2}R^{b3}, Ar⁴, —CH₂—Ar⁴, Hetar⁴, Cyc⁴ or Hetcyc⁴;
or two adjacent R^{B1}, R^{B2}, R^{B3}, R^{B4}, R^{B5}, R^{B6} and/or R^{B7} form together a divalent —C₂₋₄-alkylene radical in which one of the alkylene carbon units may be replaced by a carbonyl unit (—C(═O)—), or a divalent —O—C₁₋₃-alkylene radical or a divalent —O—C₁₋₃-alkylene-O—radical;
R^{B8}, R^{B9}, R^{B10}, R^{B11}, R^{B12}, R^{B13} represent independently from each other halogen, un-substituted or substituted C₁₋₆-aliphatic, C₁₋₆-aliphatoxy, Ar^{Y}; or
two of R^{B8}, R^{B9}, R^{B10}, R^{B11}, R^{B12}, R^{B13} which are attached to the same carbon atom of said carbocycle or said heterocycle form a divalent oxo (═O) group; or
two of R^{B8}, R^{B9}, R^{B10}, R^{B11}, R^{B12}, R^{B13} or four of R^{B8}, R^{B9}, R^{B10}, R^{B11}, R^{B12}, R^{B13} which are attached to the same sulfur atom of said heterocycle form a divalent oxo (═O) group thereby forming either an —S(═O)— or an —S(═O)₂—moiety:
R^{C1}, R^{C2}, R^{C3}, R^{C4}, R^{C5}, R^{C6} represent independently from each other un-substituted or substituted C₁₋₆-aliphatic;
R^{D1}, R^{D2}, R^{D3}, R^{D4}, R^{D5} represent independently from each other halogen, un-substituted or substituted C₁₋₆-aliphatic;
R^{D6}, R^{D7}, R^{D8}, R^{D9}, R^{D10} represent independently from each other halogen, hydroxy un-substituted or substituted C₁₋₆-aliphatic, unsubstituted or substituted —O—C₁₋₆-aliphatic, Hetar^{Y1}, CH₂-Hetar^{Y1}, Cyc^{Y1}, Hetcyc^{Y1}, or —CH₂-Hetcyc^{Y1};
and/or two of R^{D6}, R^{D7}, R^{D8}, R^{D9}, and R^{D10} which are attached to the same ring atom of that carbocycle or heterocycle form a divalent C₂₋₆-alkylene radical wherein optionally one or two non-adjacent carbon units of that alkylene radical may be replaced by independently from each other O, NH, or N—C₁₋₄-alkyl, and wherein that alkylene radical may optionally be substituted with OH, C₁₋₆-aliphatic or —O—C₁₋₆-aliphatic;
and/or two of R^{D6}, R^{D7}, R^{D8}, R^{D9}, and R^{D10} which are attached to two different ring atoms of that carbocycle or heterocycle form a divalent C₁₋₆-alkylene radical wherein optionally one or two non-adjacent carbon units of that alkylene radical may be replaced by independently from each other O, NH, or N—C₁₋₄-alkyl;
R^{X1}, R^{X2}, R^{X3}, R^{X4}, R^{X5}, R^{X6}, R^{X7}, R^{X8} represent independently from each other un-substituted or substituted C₁₋₆-aliphatic, C₁₋₆-aliphatoxy, halogen, —OH, —NR^{2d}—S(═O)₂—R^{2g}, Hetcyc^{Y}, or —O-Hetcyc^{Y}; and/or
two of R^{X1}, R^{X2}, R^{X3}, R^{X4}, R^{X5}, R^{X6}, R^{X7}, and R^{X8} which are attached to the same carbon atom of said heterocycle form a divalent oxo (═O) group; and/or two of R^{X1}, R^{X2}, R^{X3}, R^{X4}, R^{X5}, R^{X6}, R^{X7}, and R^{X8} or four of R^{X1}, R^{X2}, R^{X3}, R^{X4}, R^{X5}, R^{X6}, R^{X7}, and R^{X8} which are attached to the same sulfur atom of said heterocycle form a divalent oxo (═O) group thereby forming either an —S(═O)—or an —S(═O)₂-moiety;
R^{b1} represents un-substituted or substituted C₁₋₈-aliphatic;
R^{b2}, R^{b3} represent independently from each other H or un-substituted or substituted C₁₋₈-aliphatic; or
form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms:
halogen is F, Cl, Br, or I;
v is 1 or 2;
W is 1 or 2;
x is 0, 1, or 2;
y is 0, 1 or 2;
z is 0, 1 or
or any N-oxide, solvate, tautomer or stereoisomer thereof and/or any pharmaceutically acceptable salt of each of the foregoing, including mixtures thereof in all ratios.

2. The compound according to claim 1, or any N-oxide, solvate, tautomer or stereoisomer thereof and/or any pharmaceutically acceptable salt of each of the foregoing, including mixtures thereof in all ratios, wherein
Z¹ is CH or N; and
Z² is S;
or
Z³ is S; and
Z⁴ CH or N.

3. The compound according to claim 1, or any N-oxide, solvate, tautomer or stereoisomer thereof and/or any pharmaceutically acceptable salt of each of the foregoing, including mixtures thereof in all ratios, wherein
Ring B is

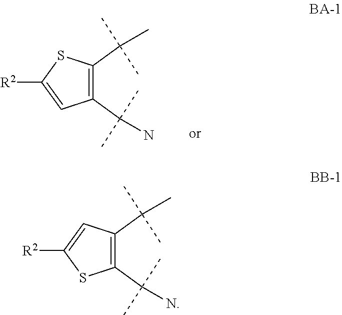

4. The compound according to claim 1, or any N-oxide, solvate, tautomer or stereoisomer thereof and/or any pharmaceutically acceptable salt of each of the foregoing, including mixtures thereof in all ratios, wherein
Ring A represents a five-membered heteroaromatic ring selected from the group consisting of the following ring moieties: 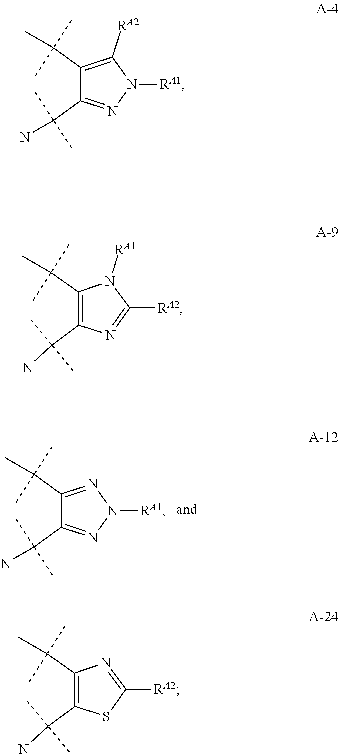
R^{A1} represents Ar³, L³-Ar³, straight-chain or branched C₁₋₄-alkyl which is optionally substituted with independently from each other 1, 2 or 3 halogen, straight-chain or branched C₂₋₄-alkenyl, or C₂₋₄-alkinyl;
R^{A2} represents H;
Ar³ represents phenyl which is optionally substituted with independently from each other R^{B1}, R^{B2} and/or R^{B3}
L³ represents —CH₂—;
R^{B1}, R^{B2}, R^{B3} are independently from each other halogen or —CN.

5. The compound according to claim 4, or any N-oxide, solvate, tautomer or stereoisomer thereof and/or any pharmaceutically acceptable salt of each of the foregoing, including mixtures thereof in all ratios, wherein
R^{A1} represents methyl.

6. The compound according to claim 1, or any N-oxide, solvate, tautomer or stereoisomer thereof and/or any pharmaceutically acceptable salt of each of the foregoing, including mixtures thereof in all ratios, wherein
R¹ represents Ar¹, Hetar¹, Cyc¹, Hetcyc¹, L¹-Ar¹, L¹-Hetar¹, L²-Cyc¹, L²-Hetcyc¹, C₁₋₆-alkyl, C₂₋₆-alkenyl or C₂₋₆-alkynyl, wherein said C₁₋₆-alkyl, C₂₋₆-alkenyl or C₂₋₆-alkynyl is straight-chain or branched and unsubstituted or substituted with 1, 2 or 3 halogen;
Ar¹ is a mono- or bicyclic aryl with 6 or 10 ring carbon atoms, wherein that aryl may be unsubstituted or substituted with substituents R^{B1}, R^{B2} and/or R^{B3} which may be the same or different;
Ar⁴ is phenyl;
Ar^{X} is an unsubstituted benzo ring;
Ar^{Y} is phenyl;
Hetar¹ is a monocyclic heteroaryl with 5 or 6 ring atoms or a bicyclic heteroaryl with 9 or 10 ring atoms wherein 1, 2 or 3 of said ring atoms is/are a hetero atom(s) selected from the group consisting of N, O and S and the remaining are carbon atoms, wherein that heteroaryl tray be unsubstituted or substituted with substituents R^{B1}, R^{B2} and/or R^{B3} which may be the same or different;
Hetar⁴ is a monocyclic heteroaryl with 5 or 6 ring atoms wherein 1, 2, 3, 4, 5 of said ring atoms is/are a hetero atom(s) selected from the group consisting of N, O and S and the remaining are carbon atoms;
Cyc¹ is a saturated or partially unsaturated, mono- or bicyclic carbocycle with 3, 4, 5, 6, 7 or 8 ring carbon atoms, wherein that carbocycle may be unsubstituted or substituted with R^{B8} and/or R^{B9} which may be the same or different; and wherein that carbocycle may optionally be fused to Ar^{X} via 2 adjacent ring atoms of said Ar^{X} and wherein that fused carbocycle may be unsubstituted or substituted with R^{C1} and/or R^{C2} which may be the same or different;
Cyc⁴ is cyclopropyl, cyclobutyl, or cyclopentyl, each of which may be unsubstituted or mono-substituted with R^{D6} or di-substituted with independently from each other R^{D6} and R^{D7};
Hetcyc¹ is a saturated or partially unsaturated, monocyclic heterocycle with 5 or 6 ring atoms wherein 1 or 2 of said ring atoms is/are a hetero atom(s) selected from the group consisting of N, O and S and the remaining are carbon atoms, wherein that heterocycle may be unsubstituted or substituted with R^{B8} and/or R^{B9} which may be the same or different, wherein, if one of the heteroatoms is S, then that heterocycle may also be substituted with R^{B8}, R^{B9}, R^{B10} and R^{B11}
Hetcyc⁴ is pyrrolidinyl, piperidinyl, each of which may unsubstituted or mono-substituted with R^{D6} or di-substituted with independently from each other R^{D6} and R^{D7};
L¹ is a divalent radical selected from the group consisting of —S(═O)₂—and un-substituted or substituted, straight-chain or branched C₁₋₆-alkylene or C₂₋₆-alkenylene, in both of which one of the carbon units of the alkylene or alkenylene chain may be replaced by —O—;
L² is a divalent radical selected from the group consisting of un-substituted or substituted, straight-chain or branched C₁₋₆-alkylene and C₂₋₆-alkenylene, in both of which one of the carbon units of the alkylene or alkenylene chain may be replaced by —O—;
R^{B1}, R^{B2}, R^{B3} represent independently from each other straight-chain or branched C₁₋₆-alkyl, Which C₁₋₆-alkyl may be unsubstituted or monosubstituted with —CN or substituted with 1, 2 or 3 halogen; straight-chain or branched C₁₋₄-alkoxy, which C₁₋₄-alkoxy may be unsubstituted or substituted with 1, 2 or 3 halogen, —O—CH₂—C≡CH; or straight-chain or branched —S—C₁₋₄-alkyl, which —S—C₁₋₄-alkyl may be unsubstituted or substituted with 1, 2 or 3 halogen, F, Cl, Br, —CN, —S(═O))—C₁₋₃-alkyl, S(═O)₂—C₁₋₃-alkyl, —N(C₁₋₃-alkyl)₂, Ar⁴, —CH₂—Ar⁴, Hetar⁴, Cyc⁴, or Hetcyc⁴;
or two adjacent R^{B1}, R^{B2} and/or R^{B3} form together a divalent —C₃₋₄-alkylene radical in which one of the alkylene carbon units may be replaced by a carbonyl unit (—C(═O)—), or a divalent —O—C₂₋₃-alkylene radical;
R^{B8}, R^{B9} represent independently from each other F or C₁₋₂-alkyl, which C₁₋₂-alkyl may be unsubstituted or substituted with 1, 2 or 3 F, C₁₋₂-alkoxy, or Ar^{Y}; or
R^{B8} and R^{B9} are attached to the same carbon atom of said carbocycle Cyc¹ or said heterocycle Hetcyc¹ and form a divalent oxo (═O) group; or
R^{B8} and R^{B9} and R^{B10} and R^{B11} are attached to the same sulfur atom of said heterocycle and form two divalent oxo (═O) groups thereby forming an —S(═O)₂—moiety;
R^{C1} and R^{C2} represent independently from each other C₁₋₆-alkyl which may be independently from each other be substituted with 1, 2, or 3 F atoms;
R^{D6}, R^{D7}, represent independently from each other C₁₋₆-alkyl which may be substituted with 1, 2, or 3 F atoms or 1 hydroxy group; or hydroxy;
halogen is F, Cl, or Br.

7. The compound according to claim 1, or any N-oxide, solvate, tautomer or stereoisomer thereof and/or any pharmaceutically acceptable salt of each of the foregoing, including mixtures thereof in all ratios, wherein
R¹ represents 4-trifluoromethylphenyl or 4-trifluoromethoxyphenyl.

8. The compound according to claim 1, or any N-oxide, solvate, tautomer or stereoisomer thereof and/or any pharmaceutically acceptable salt of each of the foregoing, including mixtures thereof in all ratios, wherein
R² represents —C(═O)—OR^{2a} or Hetcyc^{X}.

9. The compound according to claim 8, or any N-oxide, solvate, tautomer or stereoisomer thereof and/or any pharmaceutically acceptable salt of each of the foregoing, including mixtures thereof in all ratios, wherein
R^{2a} represents H, straight-chain or branched, unsubstituted or substituted C₁₋₄-alkyl or Cat;
Cat represents a monovalent cation selected from the group consisting of lithium (Li), sodium (Na) and potassium (K);
Hetcyc^{X} represents 1H-1,2,3,4-tetrazol-5-yl, 2H-1,2,3,4-tetrazol-5-yl, 2-methyl-2H-1,2,3,4-tetrazol-5-yl, 5-oxo-2,5-dihydro-1,2,4-oxadiazol-3-yl (2H-1,2,4-oxadiazol-5-on-3-yl), 5-oxo-4,5-dihydro-1,2,4-oxadiazol-3-yl (4H-1,2,4-oxadiazol-5-on-3-yl), 3-bromo-4,5-dihydro-1,2-oxazol-5-vi, 3-chloro-4,5-dihydro-1,2-oxazol-5-yl, 3-(1H-1,2,3-triazol-1-yl)-4,5-dihydro-1,2-oxazol-5-yl, 3-(2H-1,2,3-triazol-2-yl)-4,5-dihydro-1,2-oxazol-5-yl, 3-(pyrimidin-5-yloxy)-4,5-dihydro-1,2-oxazol-5-yl, 3-hydroxy-oxetan-3-yl, 5-hydroxy-4H-pyran-4-on-2-yl, 3,3-difluoropyrrolidin-2-on-4-yl, 3,3-difluoropyrrolidin-2-on-5-yl, 3,3-difluoro-2,3-dihydro-1H-pyrrol-2-on-4-yl, or 3,3-difluoro-2,3-dihydro-1H-pyrrol-2-on-5-yl.

10. The compound according to claim 8, or any N-oxide, solvate, tautomer or stereoisomer thereof and/or any pharmaceutically acceptable salt of each of the foregoing, including mixtures thereof in all ratios, wherein
R² represents —C(═O)—OR^{2a};
R^{2a} represents H or Na.

11. The compound according to claim 1, or any N-oxide, solvate, tautomer or stereoisomer thereof and/or any pharmaceutically acceptable salt of each of the foregoing, including mixtures thereof in all ratios, wherein
R² represents —C(═O)—NR^{2b}R^{2c};
and wherein further
(a)
R^{2b} and R^{2c} both represent H;
or
(b)
one of R^{2b} and R^{2c} represents H, while the other of R^{2b} and R^{2c} represents Cyc^{2b}, Hetcyc^{2b}, straight-chain or branched C₁₋₁₀-alkyl which may be unsubstituted or substituted with R^{E1}, R^{E2}, R^{E3}, R^{E4} and/or R^{E5} which may be the same or different, wherein in that C₁₋₁₀-alkyl 1 or 2 non-adjacent and non-terminal methylene moieties may be replaced by independently from each other —O— and/or —S— and/or —NH— and/or —N(C₁₋₄-alkyl)-;
Cyc^{2b} is a saturated monocyclic carbocycle with 3, 4, 5, 6 or 7 ring carbon atoms, wherein that carbocycle may be unsubstituted or substituted independently from each other with R^{D6}, R^{D7}, R^{D8}, R^{D9} and or R^{D10} wherein that carbocycle may optionally be fused to Ar^{Z} or Hetar^{Z} via 2 adjacent ring atoms and wherein that fused carbocycle may optionally further be substituted with independently from each other R^{C1}, R^{C2} and/or R^{C3};
Hetcyc^{2b} is a saturated monocyclic heterocycle with 5 or 6 ring atoms wherein 1 or 2 of said ring atoms is/are a hetero atom(s) selected from the group consisting of N, O and S and the remaining are carbon atoms, wherein that heterocycle may be unsubstituted or substituted independently from each other with R^{D6}, R^{D7}, R^{D8}, R^{D9} and/or R^{D10} wherein that heterocycle may optionally be fused to Ar^{Z} or Hetar^{Z} and wherein that fused heterocycle may optionally further be substituted with independently from each other R^{C1}, R^{C2} and/or R^{C3};
R^{E1}, R^{E2}, R^{E3}, R^{E4} and/or R^{E5} represent independently from each other halogen, —NR^{Ea}R^{Eb}, OR^{Ec}, Ar^{E}, Hetar^{E}, Cyc^{E}, or Hetcyc^{E};
Ar^{E} is a mono- or bicyclic aryl with 6 or 10 ring carbon atoms, wherein that aryl may be unsubstituted or substituted with substituents R^{F1}, R^{F2} and/or R^{F3} which may be the same or different;
Ar^{Z} is benzo;
Hetar^{E} is a monocyclic heteroaryl with 5 or 6 ring atoms or a bicyclic heteroaryl with 9 or 10 ring atoms wherein 1, 2, 3, or 4 of said ring atoms is/are a hetero atom(s) selected from the group consisting of N, O and S and the remaining are carbon atoms, wherein that heteroaryl may be unsubstituted or substituted with substituents R^{F1}, R^{F2} and/or R^{F3} which may be the same or different;
Hetar^{Y1} is a 5 or 6 membered monocyclic heteroaryl wherein 1, 2, 3, or 4 ring atoms are hetero atoms selected from the group consisting of N, O and S and the remaining are carbon atoms, wherein that heteroaryl may be unsubstituted or substituted with F or C₁₋₄-alkyl which may optionally be substituted with OH;
Hetar^{Y2} is a 5 or 6 membered monocyclic heteroaryl wherein 1, 2, 3, or 4 ring atoms are hetero atoms selected from the group consisting of N, O and S and the remaining are carbon atoms, wherein that heteroaryl may be unsubstituted or substituted with halogen and/or C₁₋₄-alkyl which may optionally be substituted with OH;
Hetar^{Z} is pyrrole, N-methyl-pyrrole, pyrazole, imidazole, or triazole;
Cyc^{E} is a saturated or partially unsaturated, mono- or bicyclic carbocycle with 3, 4, 5, 6, 7 or 8 ring carbon atoms, wherein that carbocycle may be unsubstituted or substituted with R^{G1} and/or R^{G2} which may be the same or different;
Cyc^{Y1} is a saturated or partially unsaturated monocyclic carbocycle with 3, 4, 5, 6 or 7 ring carbon atoms, wherein that carbocycle may be unsubstituted or substituted with halogen, OH, C₁₋₄-alkyl,
Hetcyc^{E} is a saturated or partially unsaturated, monocyclic heterocycle with 4, 5 or 6 ring atoms wherein 1 or 2 of said ring atoms is/are a hetero atom(s) selected from the group consisting of N, O and S and the remaining are carbon atoms, wherein that heterocycle may be unsubstituted or substituted with R^{G1} and/or R^{G2} which may be the same or different;
Hetcyc^{Y1} is a saturated or partially unsaturated monocyclic heterocycle with 5 or 6 ring atoms wherein 1 or 2 of said ring atoms are heteroatoms selected from the group consisting of N, O, and S and the remaining are carbon atoms;
Hetcyc^{Y2} is a saturated or partially unsaturated monocyclic heterocyle with 5 or 6 ring atoms wherein 1 or 2 of said ring atoms are heteroatoms selected from the group consisting of N, O, and S and the remaining are carbon atoms;
R^{C1}, R^{C2}, R^{C3} represent independently from each other C₁₋₄-alkyl;
R^{D6}, R^{D7}, R^{D8}, R^{D9}, R^{D10} represent independently from each other halogen, hydroxy, or C₁₋₄-alkyl optionally substituted with —OH and/or halogen,
and/or two of R^{D6}, R^{D7}, R^{D8}, R^{D9}, R^{D10} which are attached to the same ring atom of that carbocycle or heterocycle form a divalent C₂₋₆-alkylene radical wherein optionally one or two non-adjacent carbon units of that alkylene radical may be replaced by independently from each other O, NH, or N—C₁₋₄-alkyl, and wherein that alkylene radical may optionally be substituted with OH, C₁₋₄-alkyl or —O—C₁₋₄-alkyl, and/or two of R^{D6}, R^{D7}, R^{D8}, R^{D9}, R^{D10} which are attached to two different ring atoms of that carbocycle or heterocycle form a divalent C₁₋₆-alkylene radical wherein optionally one or two non-adjacent carbon units of that alkylene radical may be replaced by independently from each other O, NH, N—C₁₋₄-alkyl, in —(CH₂)₃—, —O—(CH₂)₂—, or —O—(CH)₃—;
R^{Ea}, R^{Eb} represent independently from each other H, C₁₋₄-alkyl, —C(═O)—C₁₋₄-alkyl, or —C(═O)—OC₁₋₄-alkyl;
R^{Ec} represents H or C₁₋₄-alkyl;
R^{F1}, R^{F2}, R^{F3} represent independently from each other straight-chain or branched C₁₋₆-alkyl, which C₁₋₆-alkyl may be unsubstituted or monosubstituted with —CN, OH, or —O—C₁₋₄-alkyl or substituted with 1, 2 or 3 halogen; straight-chain or branched C₁₋₄-alkoxy, which C₁₋₄-alkoxy may be unsubstituted or substituted with 1, 2 or 3 halogen; straight-chain or branched —S—C₁₋₄-alkyl, which —S—C₁₋₄-alkyl may be unsubstituted or substituted with 1, 2 or 3 halogen; Or C₃₋₇-cycloalkyl optionally substituted with halogen, OH and/or C₁₋₄-alkyl; F, Cl, Br, —CN, —S(═O)—C₁₋₃-alkyl, S(═O)₂—C₁₋₃-alkyl, —NH₂, —NH(C₁₋₃-alkyl), —N(C₁₋₃-alkyl)₂, —OH;
and/or two of R^{F1}, R^{F2}, R^{F3} which are attached to two different ring atoms of that aryl or heteroaryl form a divalent C₁₋₆-alkylene radical wherein optionally one or two non-adjacent carbon units of that alkylene radical may be replaced by independently from each other O, NH, or N—C₁₋₄-alkyl, CH₂—O—(CH₂)₂—;
R^{G1}, R^{G2} represent independently from each other halogen; hydroxy; unsubstituted or substituted C₁₋₆-aliphatic; C₁₋₆-aliphatoxy; —C(═O)—O—C₁₋₄-alkyl; Hetar^{Y2}; —CH₂-Hetar^{Y2}; Hetcyc^{Y2};
and/or R^{G1} and R^{G2} which are attached to the same ring atom of that carbocycle or heterocycle form a divalent C₂₋₆-alkylene radical wherein optionally one or two non-adjacent carbon units of that alkylene radical may be replaced by independently from each other O, NH, or N—C₁₋₄-alkyl, and wherein that alkylene radical may optionally be substituted with OH, C₁₋₄-alkyl or —O—C₁₋₄-alkyl;
and/or R^{G1} and R^{G2} which are attached to two different ring atoms of that carbocycle or heterocycle form a divalent C₁₋₆-alkylene radical wherein optionally one or two non-adjacent carbon units of that alkylene radical ray be replaced by independently from each other O, NH, or N—C₁₋₄-alkyl,
or
(c)
one of R^{2b} and R^{2c} represents straight-chain or branched alkyl C₁₋₁₀-alkyl optionally substituted with OH or halogen, while the other of R^{2b} and R^{2c} represents Cyc^{2b}, Hetcyc^{2b}, or straight-chain or branched C₁₋₁₀-alkyl which may be unsubstituted or substituted with R^{E1}, R^{E2}, R^{E3}, R^{E4} and/or R^{E5} which may be the same or different, wherein Cyc^{2b}, Hetcyc^{2b}, R^{E1}, R^{E2}, R^{E3}, R^{E4} and R^{E5} are as defined above under (b), and wherein in that C₁₋₁₀-alkyl 1 or 2 non-adjacent and non-terminal methylene moieties may be replaced by independently from each other —O—and/or —S— and/or —NH— and/or —N(C₁₋₄-alkyl)-;
or
(d)
R^{2b} and R^{2c} form together with the nitrogen atom to which they are attached to a saturated or partially unsaturated heterocycle optionally substituted with independently from each other R^{Y1}, R^{Y2}, R^{Y3}, R^{Y4} and/or R^{Y5}; wherein that heterocycle may optionally be fused with Hetar^{Z}; and wherein that heterocycle is selected from the group consisting of azetidine, pyrrolidine, piperidine, piperazine, and morpholine;
R^{Y1}, R^{Y2}, R^{Y3}, R^{Y4}, R^{Y5} represent independently from each other halogen, —NH₂, —N(H)—C₁₋₄-alkyl, —N(H)—C(═O)—O—C₁₋₄-alkyl, —N(C₁₋₄-alkyl)₂; —OH; C₁₋₄-alkyl optionally substituted with —OH, —O—C₁₋₄-alkyl, —O—C₃₋₇-cycloalkyl, —O—CH₂—C₃₋₇-cycloalkyl, —CH₂OH, —(CH₂)₂OH, —(CH₂)₃H, —CH₂OCH₃, —(CH₂)₂OCH₃, cyclopropylmethoxy, —O—C₁₋₄-alkyl, Hetar^{Y2}; —CH₂-Hetar^{Y2}; or Hetcyc^{Y2};
and/or two of R^{Y1}, R^{Y2}, R^{Y3}, R^{Y4}, R^{Y5} which are attached to the same ring atom of that heterocycle form a divalent C₂₋₆-alkylene radical wherein optionally one or two non-adjacent carbon units of that alkylene radical may be replaced by independently from each other O, NH, N—C₁₋₄-alkyl, —(CH₂)₂—O—(CH₂)₂—, or —(CH₂)₂—O—(CH₂)₃—;
and/or two of R^{Y1}, R^{Y2}, R^{Y3}, R^{Y4}, R^{Y5} which are attached to two different ring atoms of that heterocycle form a divalent C₁₋₆-alkylene radical wherein optionally one or two non-adjacent carbon units of that alkylene radical may be replaced by independently from each other O, NH, or N—C₁₋₄-alkyl;
Hetar^{Y2} is a 5 or 6 membered monocyclic heteroaryl wherein 1, 2, 3, pr 4 ring atoms are hetero atoms selected from the group consisting of N, O and S and the remaining are carbon atoms, wherein that heteroaryl may be unsubstituted or substituted with halogen or C₁₋₄-alkyl which may optionally be substituted with OH;
Hetar^{Z} is pyrrole, N-methyl-pyrrole, pyrazole, imidazole, or triazole;
Hetcyc^{Y2} is a saturated or partially unsaturated monocyclic heterocycle with 5 or 6 ring atoms wherein 1 or 2 of said ring atoms are heteroatoms selected from the group consisting of N, O, and S and the remaining are carbon atoms.

12. The compound according to claim 1, or any N-oxide, solvate, tautomer or stereoisomer thereof and/or any pharmaceutically acceptable salt of each of the foregoing including mixtures thereof in all ratios, wherein
R² represents —(CH₂)ₓNR^{2d}—C(O)—R^{2e}, —S—R^{2f}, —S(═O)—R^{2f}, —S(═O)₂—R^{2g}, —S(O)₂—NR^{2h}R²ⁱ, —S(═O)₂—OH, —S(═O)(═NR^{2j})—OH, —S(═O)(═NR^{2j})—R^{2g}, —S(═O)(═NR^{2k})—NR^{2l}R^{2m}, —(CH₂)_{z}—NR^{2d}—S(═O)₂—R^{2g}, —N═S(═O)—R^{2s}R^{2t}, —C(═O)—N═S(═O)—R^{2s}R^{2t}, or —C(═O)—N═S(═N—R^{2u})—R^{2s}R^{2t};
R^{2e} represents H, C₁₋₆-alkyl optionally substituted with —OH or a monocyclic 5- or 6-membered heteroaryl; C₃₋₇-cycloalkyl, or monocyclic 5- or 6-membered heteroaryl;
R^{2f}, R^{2g} represent independently from each other un-substituted or substituted C₁₋₈-aliphatic;
R^{2h}, R²ⁱ represent independently from each other H, un-substituted or substituted C₁₋₈-aliphatic, aryl, heterocyclyl, or heteroaryl; or form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms;
R^{2d}, R^{2j}, R^{2k} represent independently from each other H or un-substituted or substituted C₁₋₈-aliphatic;
R^{2l}, R^{2m} represent independently from each other H or un-substituted or substituted C₁₋₈-aliphatic; or form together with the nitrogen atom to which they are attached to an unsubstituted or substituted saturated, partially unsaturated or aromatic heterocycle with 3, 4, 5, 6, or 7 ring atoms wherein 1 of said ring atoms is said nitrogen atom and no or one further ring atom is a hetero atom selected from the group consisting of N, O and S and the remaining are carbon atoms;
R^{2s}, R^{2t} represent independently from each other C₁₋₆-alkyl which may optionally be substituted with —OH, O—C₁₋₄-alkyl, NH₂, NHC₁₋₄-alkyl, N(C₁₋₄-alkyl)₂, pyrrolidinyl, piperidinyl, morpholinyl, or piperazinyl; or form together a divalent C₃₋₄-alkylene radical which may optionally be substituted with —NH₂, —CN, or a divalent C₂₋₅-alkylene radical wherein optionally one of the carbon units of said C₂₋₅-alkylene radical may be replaced by O, NH or N—C₁₋₄-alkyl;
R^{2u} represents hydrogen or C₁₋₄-alkyl;
x represents 0 or 1;
z represents 0 or 1.

13. The compound according to claim 1, or any N-oxide, solvate, tautomer or stereoisomer thereof and/or any pharmaceutically acceptable salt of each of the foregoing, including mixtures thereof in all ratios, wherein
Ring A represents 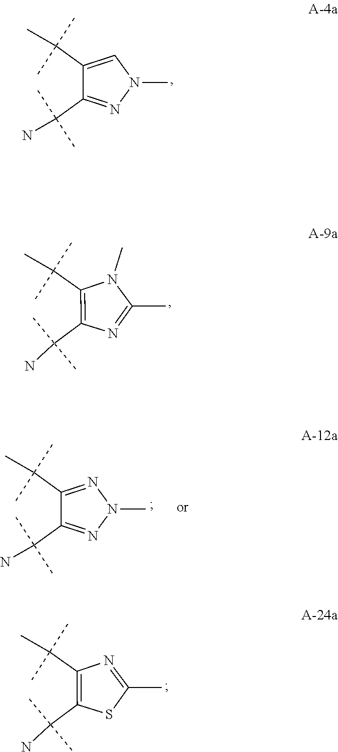
Ring B represents either 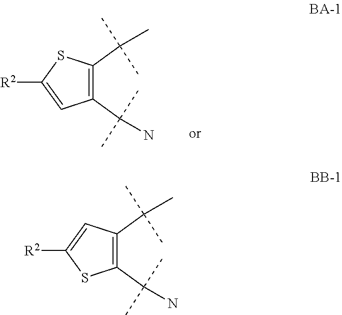
R¹ is 4-trifluoromethylphenyl or 4-trifluoromethoxyphenyl; and
R² is C(═O)—OH or C(═O)—ONa.

14. A compound, or any N-oxide, solvate, tautomer or stereoisomer thereof and/or any pharmaceutically acceptable salt of each of the foregoing, including mixtures thereof in all ratios, selected from the group consisting of the following compounds:

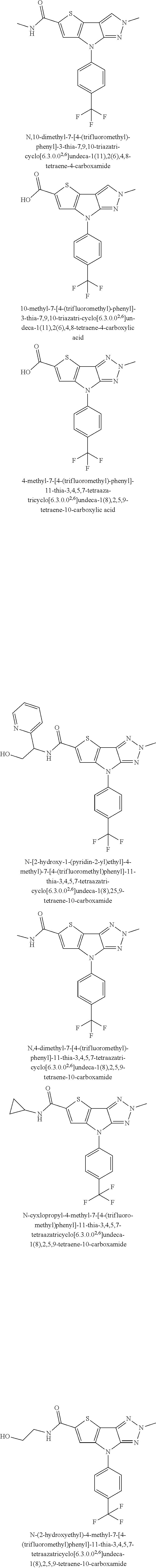

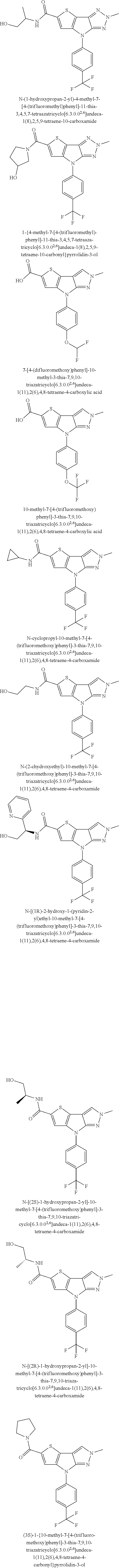

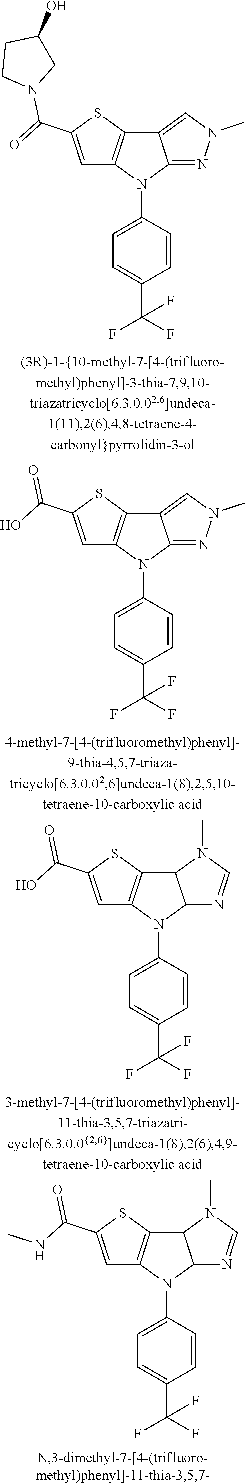

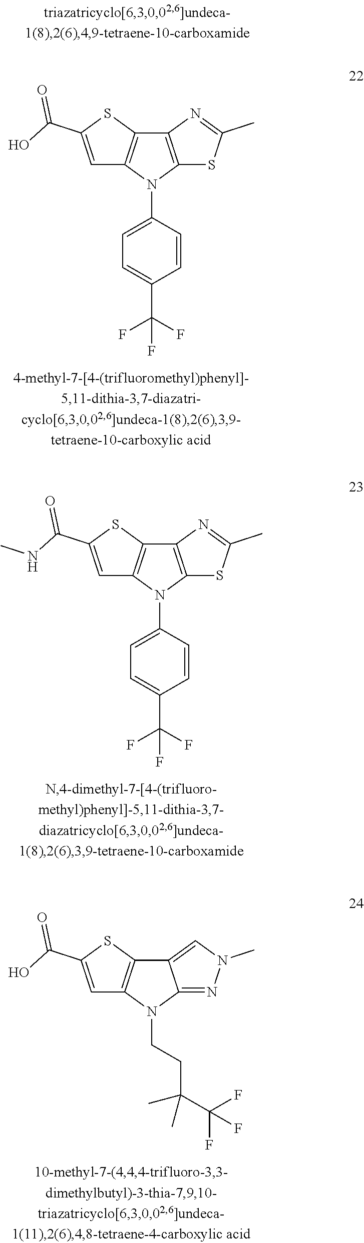

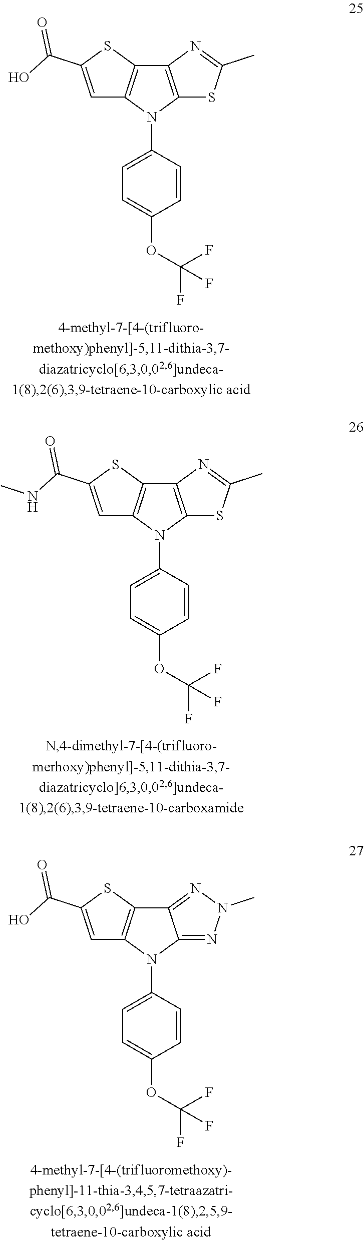

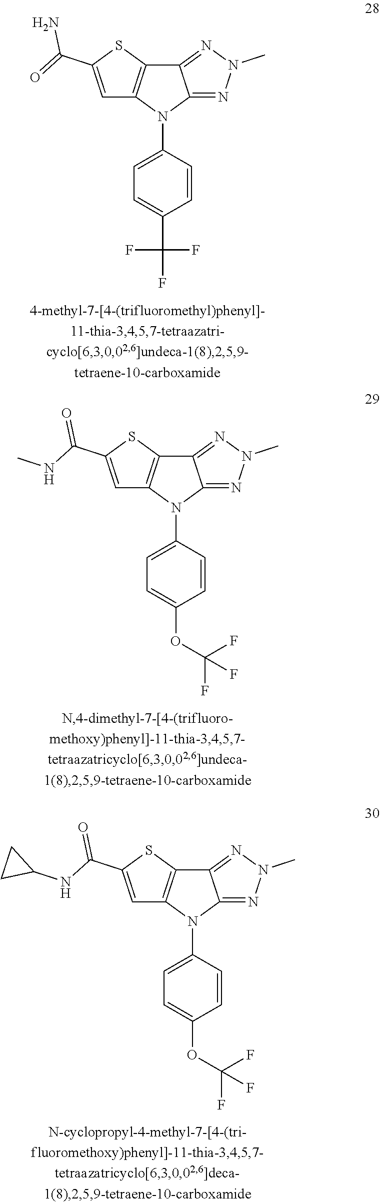

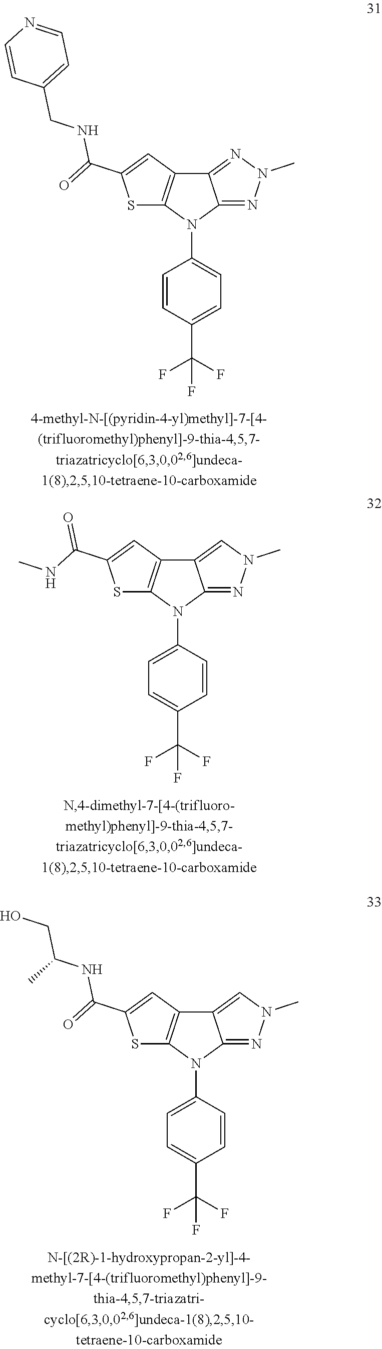

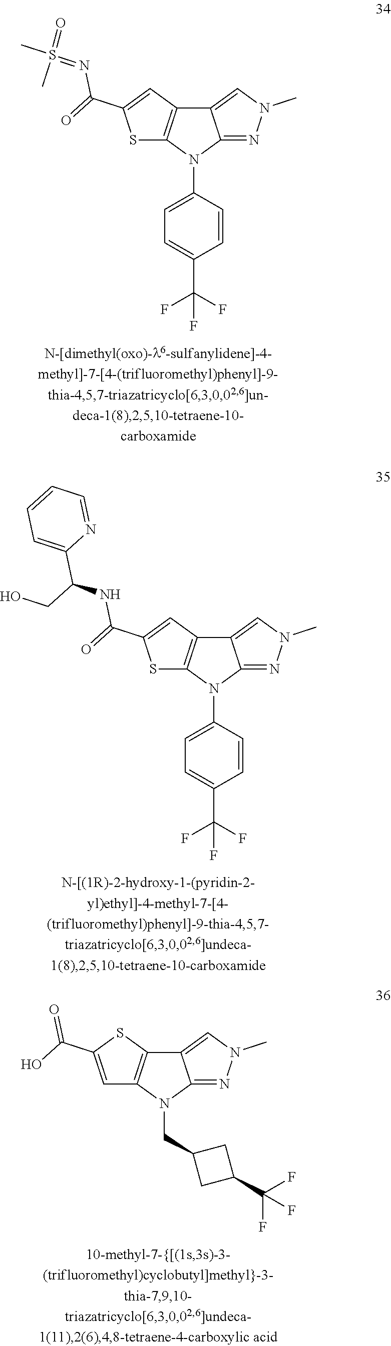

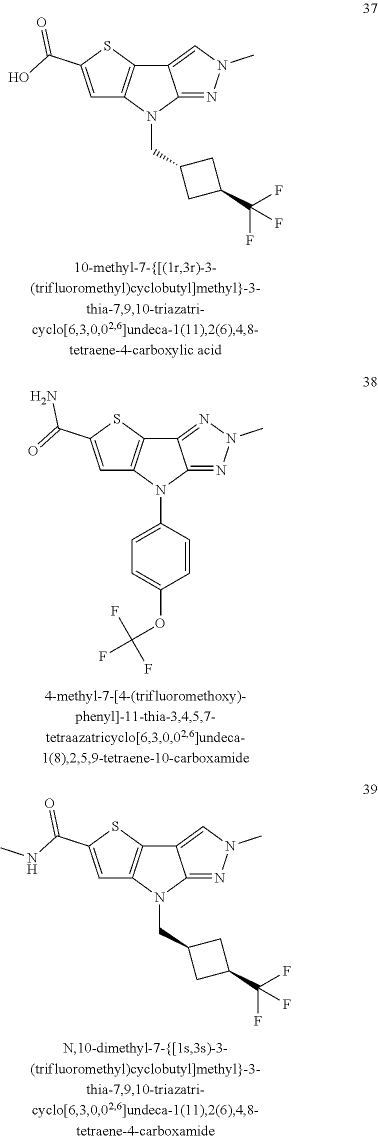

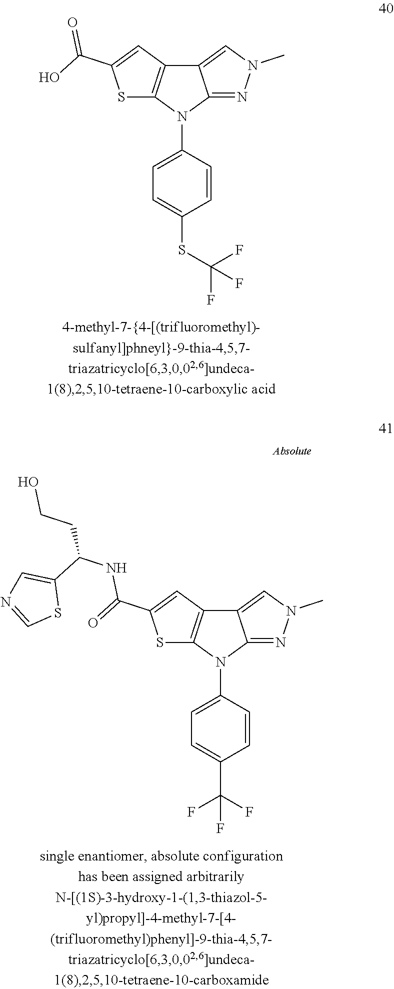 [SMILES: Cn1cc2c3cc(C(=O)O)sc3n(-c3ccc(SC(F)(F)F)cc3)c2n1 ; Cn1cc2c3cc(C(=O)N[C@@H](CCO)c4cncs4)sc3n(-c3ccc(C(F)(F)F)cc3)c2n1]

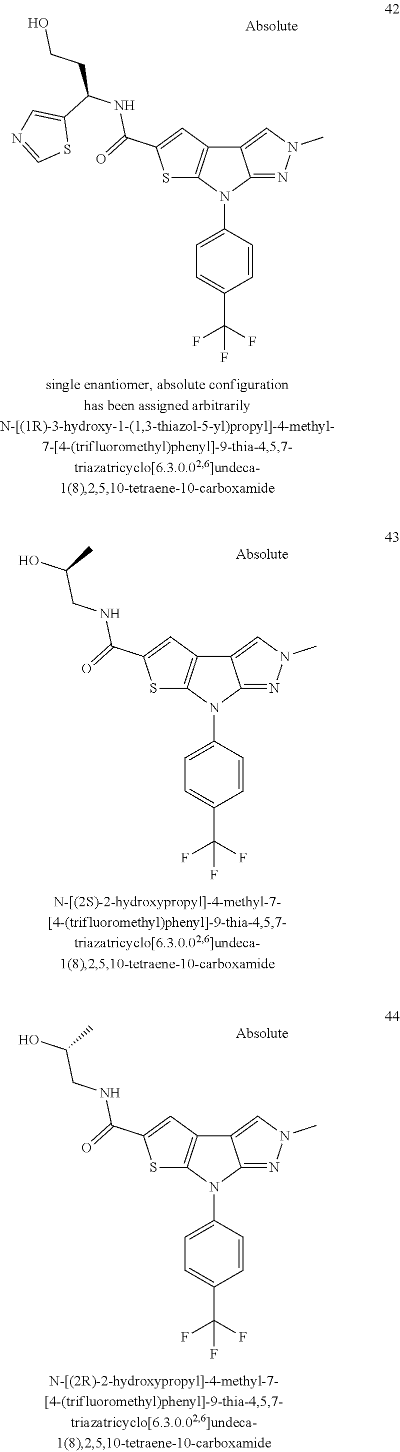 [SMILES: Cn1cc2c3cc(C(=O)N[C@H](CCO)c4cncs4)sc3n(-c3ccc(C(F)(F)F)cc3)c2n1 ; C[C@H](O)CNC(=O)c1cc2c3cn(C)nc3n(-c3ccc(C(F)(F)F)cc3)c2s1 ; C[C@@H](O)CNC(=O)c1cc2c3cn(C)nc3n(-c3ccc(C(F)(F)F)cc3)c2s1]

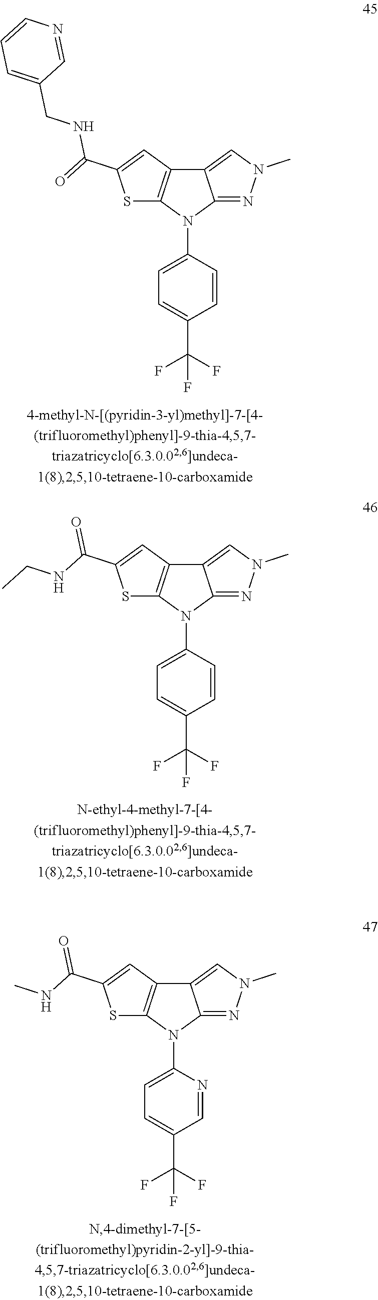

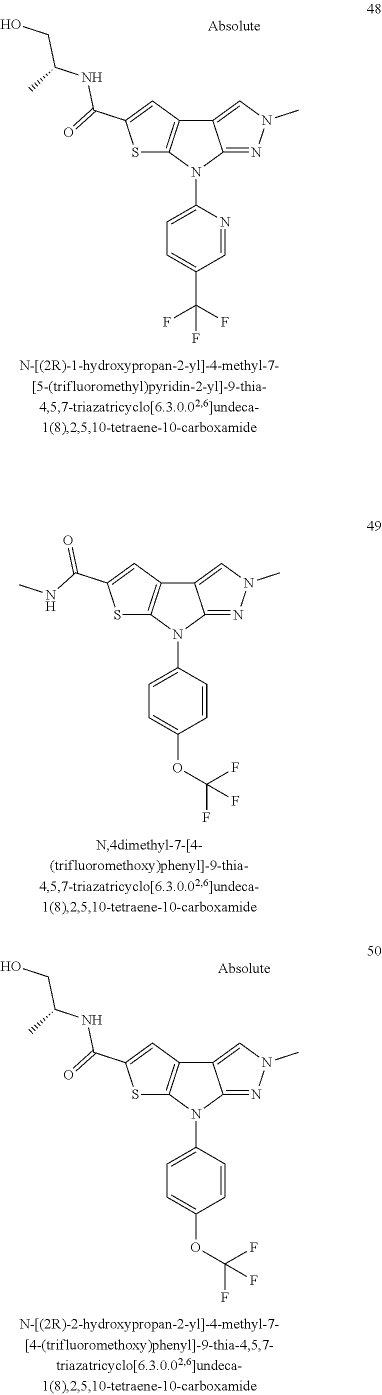

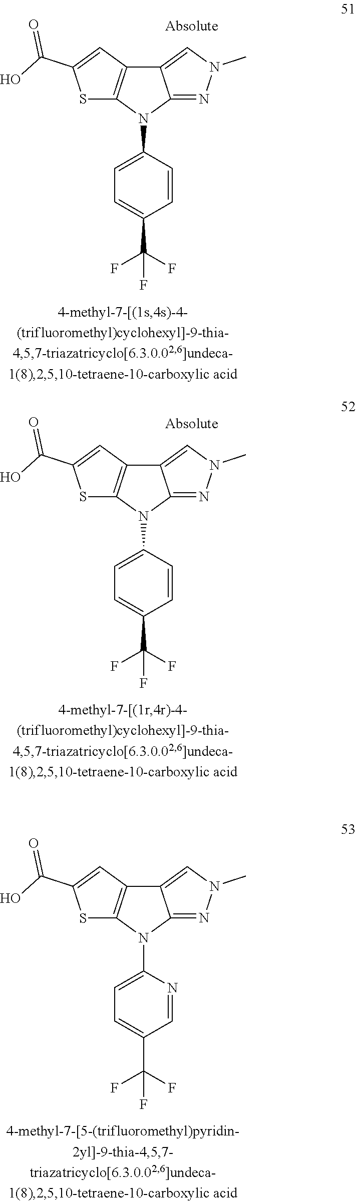

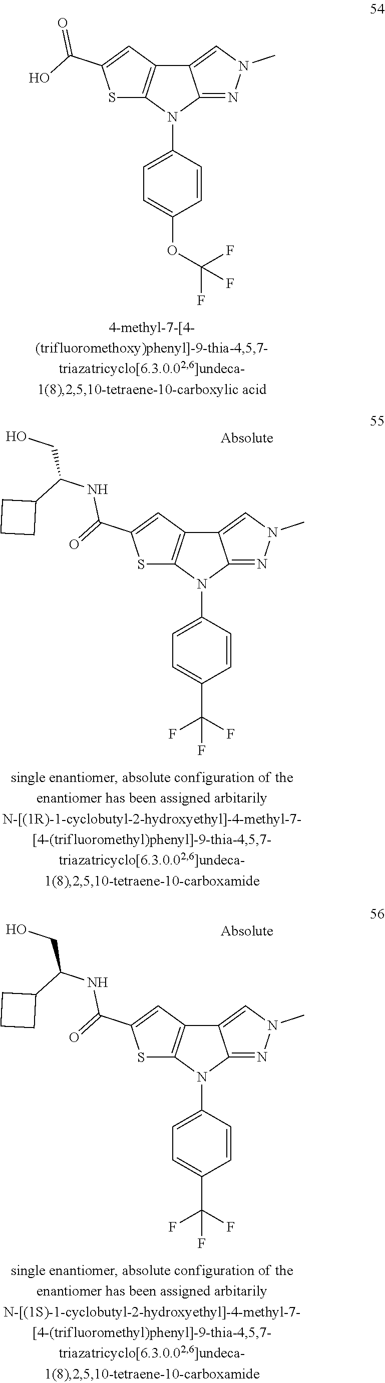 [SMILES: Cn1cc2c3cc(C(=O)O)sc3n(-c3ccc(OC(F)(F)F)cc3)c2n1 ; Cn1cc2c3cc(C(=O)N[C@@H](CO)C4CCC4)sc3n(-c3ccc(C(F)(F)F)cc3)c2n1 ; Cn1cc2c3cc(C(=O)N[C@H](CO)C4CCC4)sc3n(-c3ccc(C(F)(F)F)cc3)c2n1]

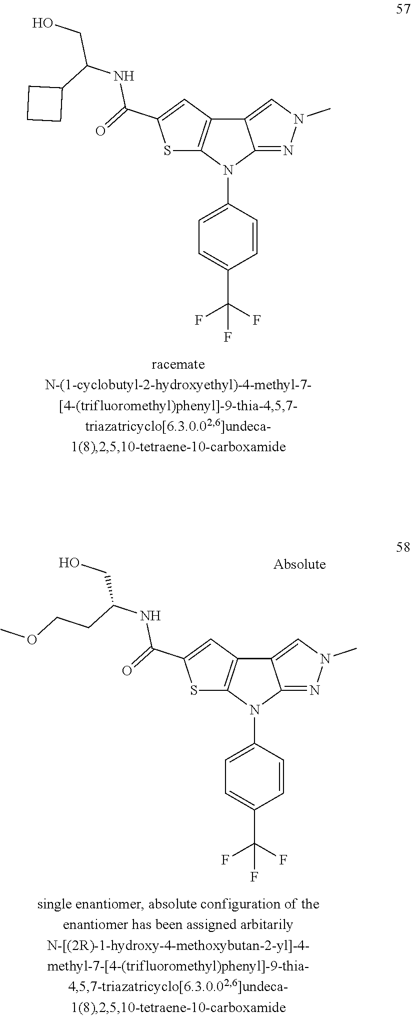

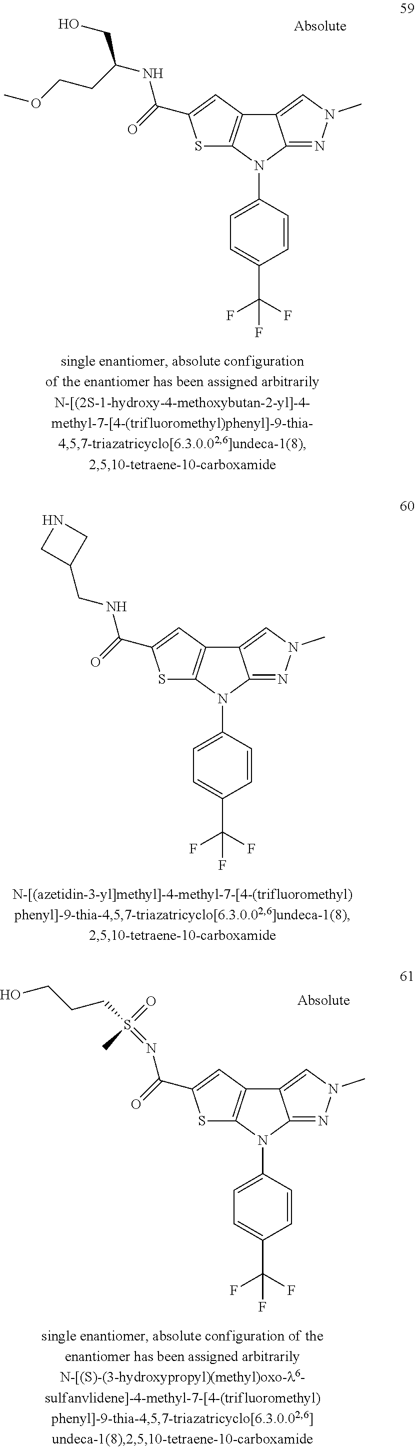

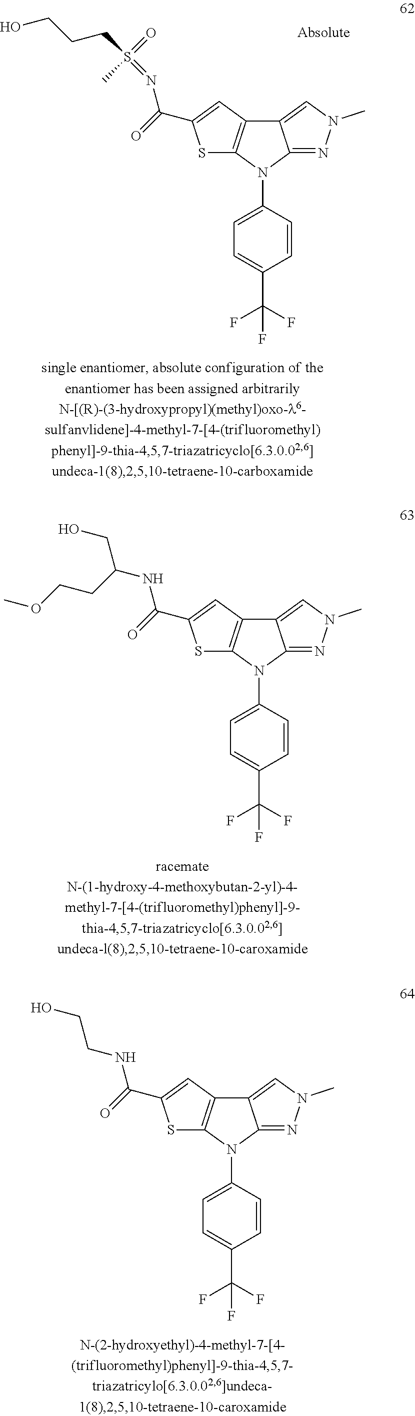

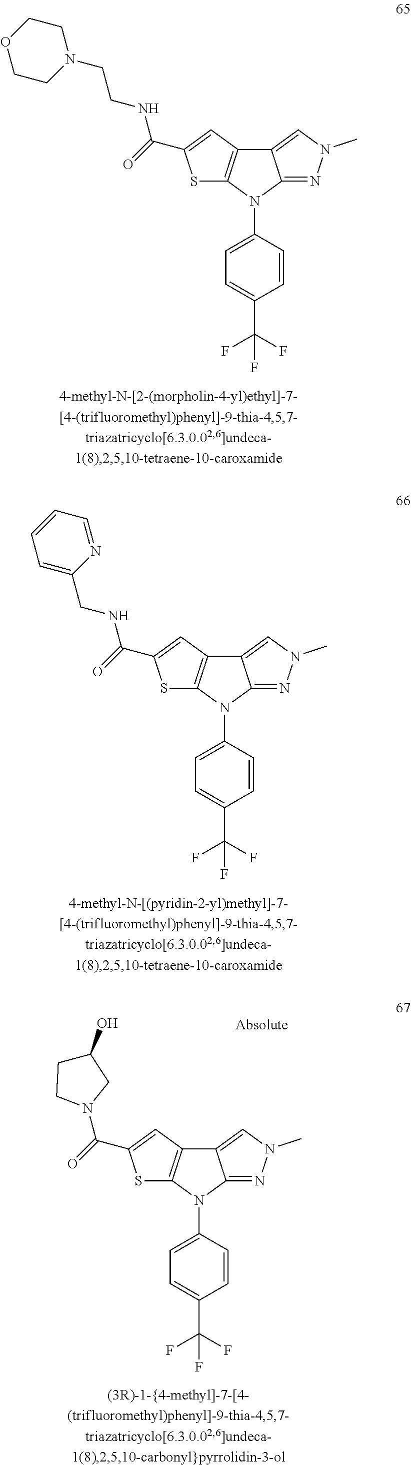 [SMILES: Cn1cc2c3cc(C(=O)NCCN4CCOCC4)sc3n(-c3ccc(C(F)(F)F)cc3)c2n1 ; Cn1cc2c3cc(C(=O)NCc4ccccn4)sc3n(-c3ccc(C(F)(F)F)cc3)c2n1 ; Cn1cc2c3cc(C(=O)N4CC[C@@H](O)C4)sc3n(-c3ccc(C(F)(F)F)cc3)c2n1]

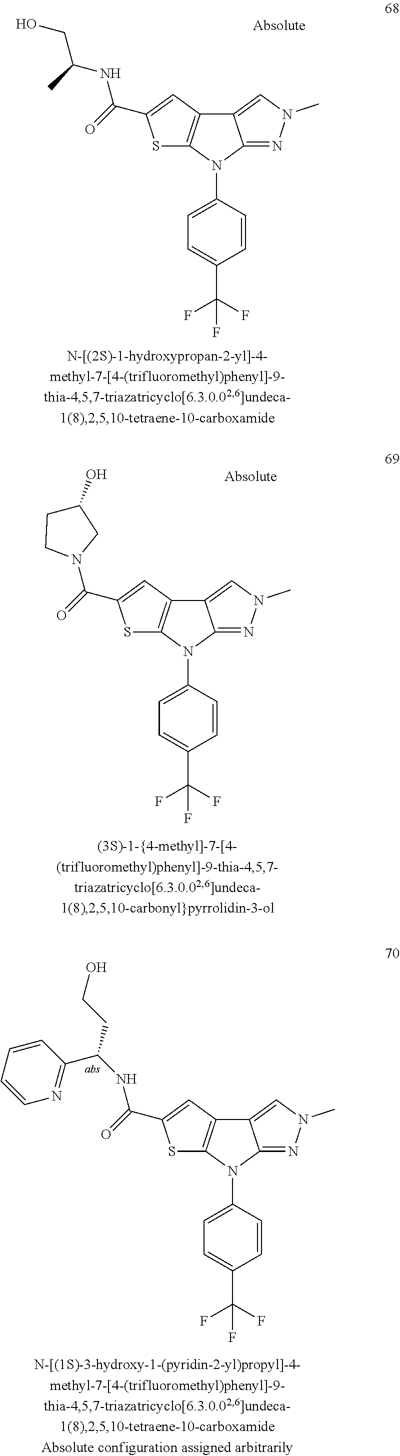 [SMILES: C[C@@H](CO)NC(=O)c1cc2c3cn(C)nc3n(-c3ccc(C(F)(F)F)cc3)c2s1 ; Cn1cc2c3cc(C(=O)N4CC[C@H](O)C4)sc3n(-c3ccc(C(F)(F)F)cc3)c2n1 ; Cn1cc2c3cc(C(=O)N[C@@H](CCO)c4ccccn4)sc3n(-c3ccc(C(F)(F)F)cc3)c2n1]

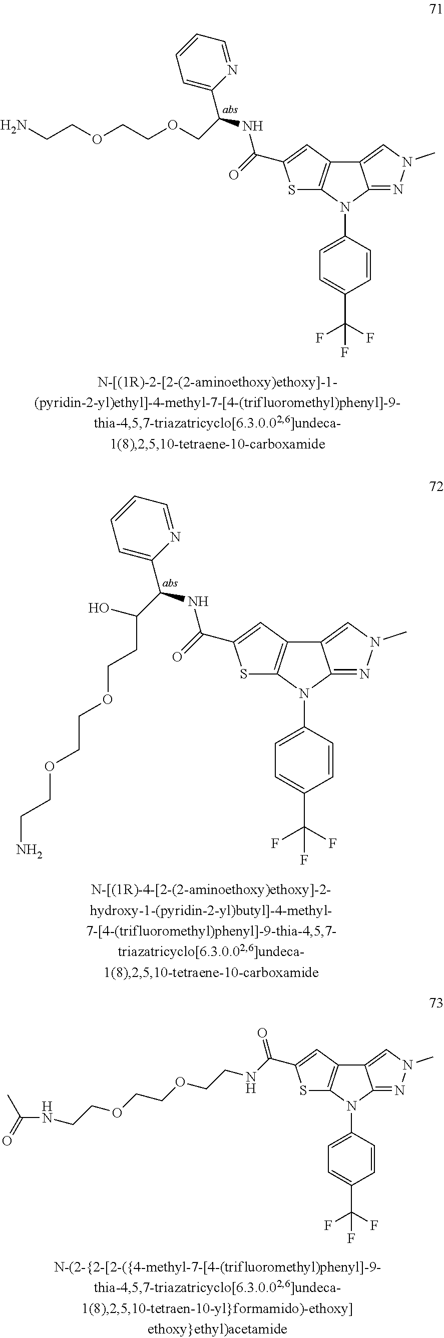

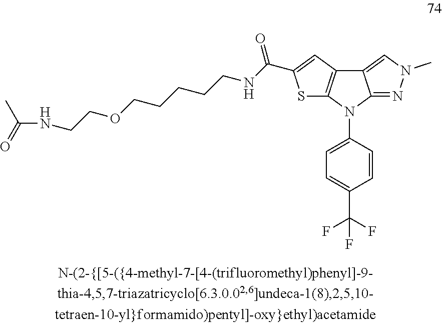 [SMILES: CC(=O)NCCOCCCCCNC(=O)c1cc2c3cn(C)nc3n(-c3ccc(C(F)(F)F)cc3)c2s1]

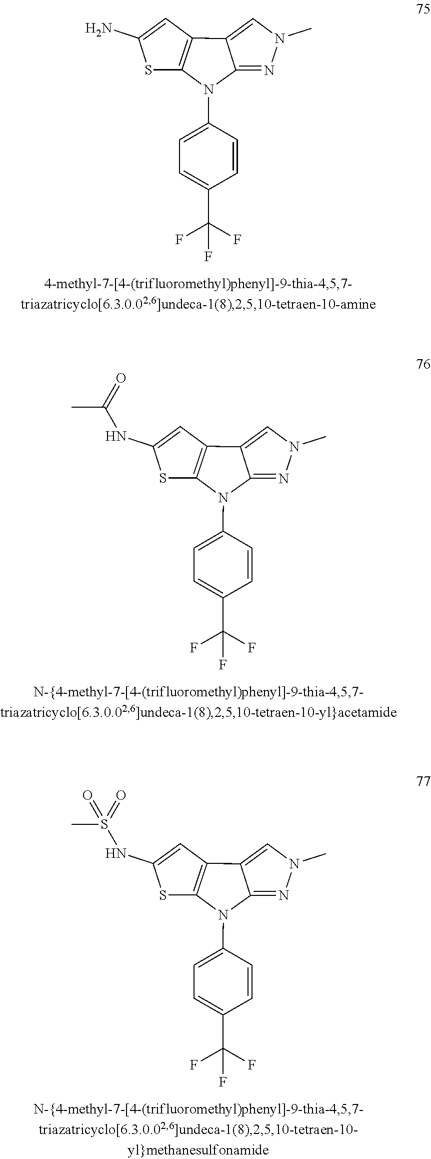

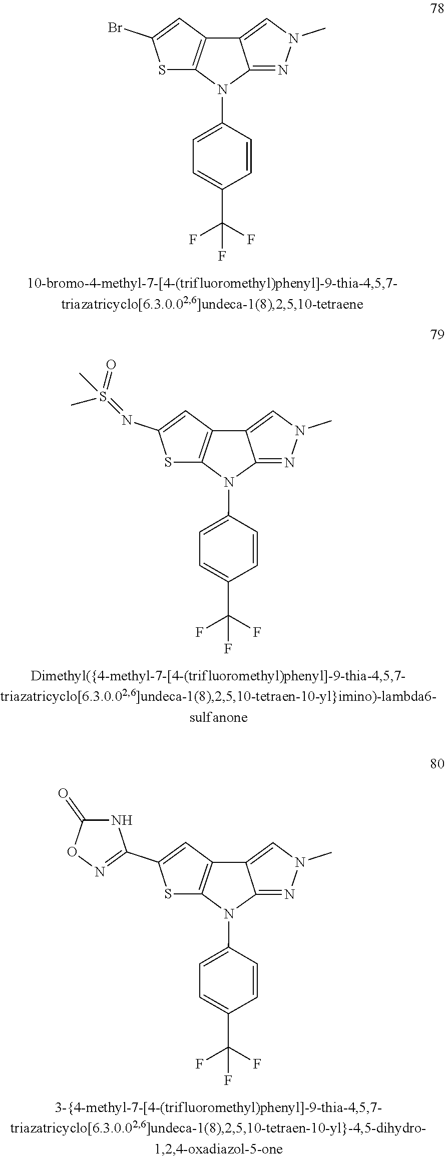

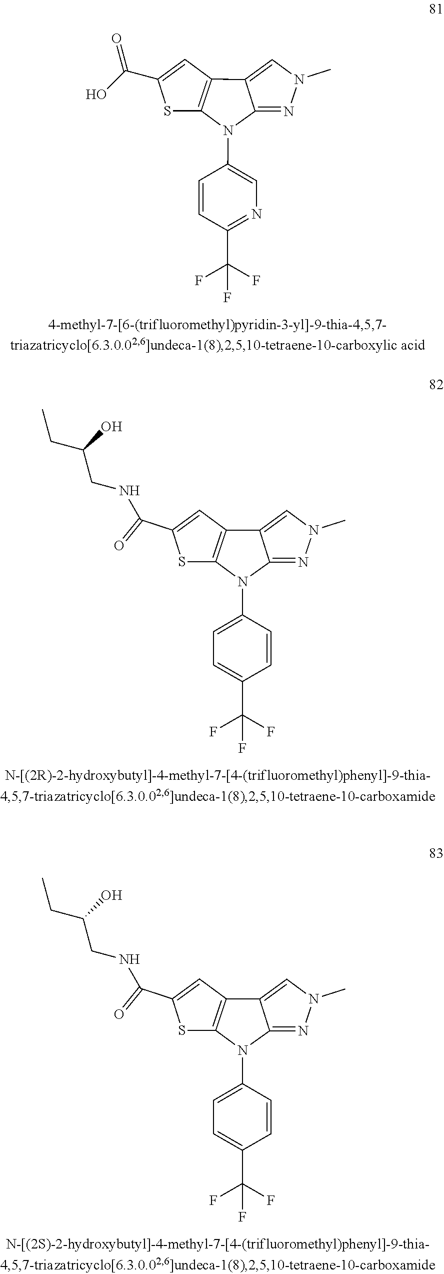

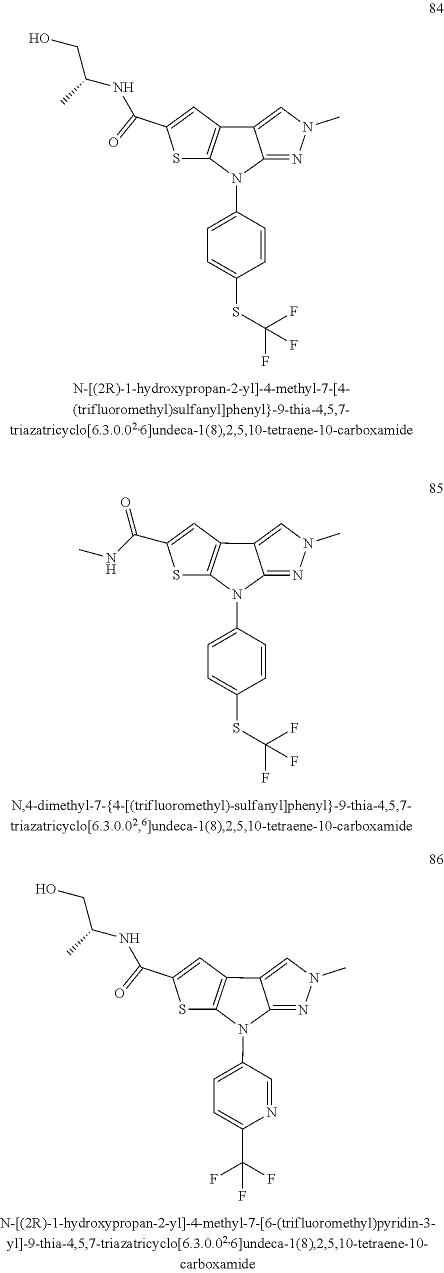

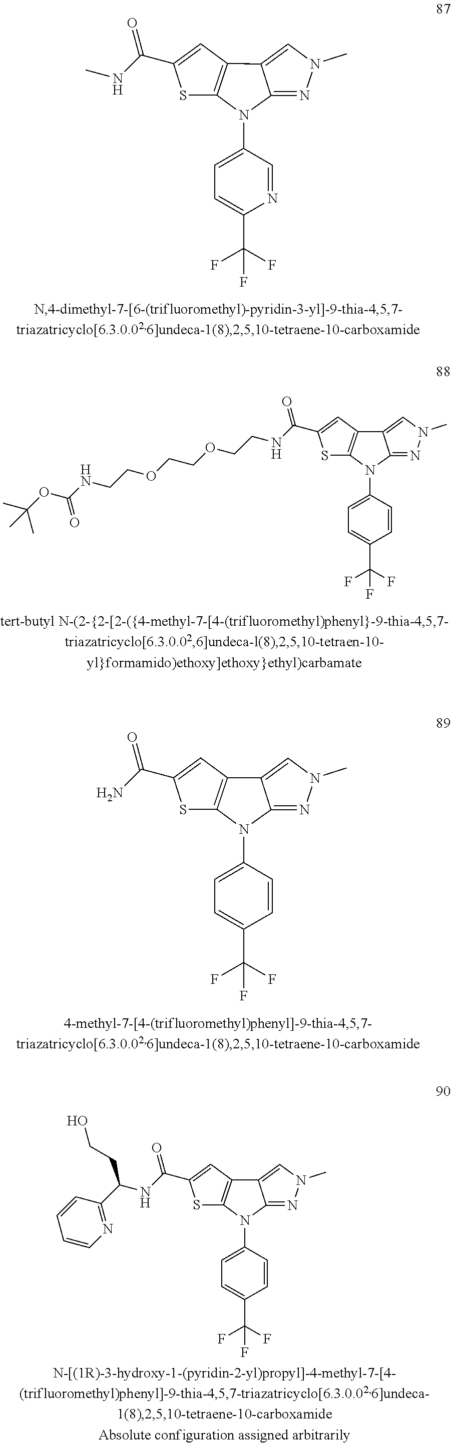

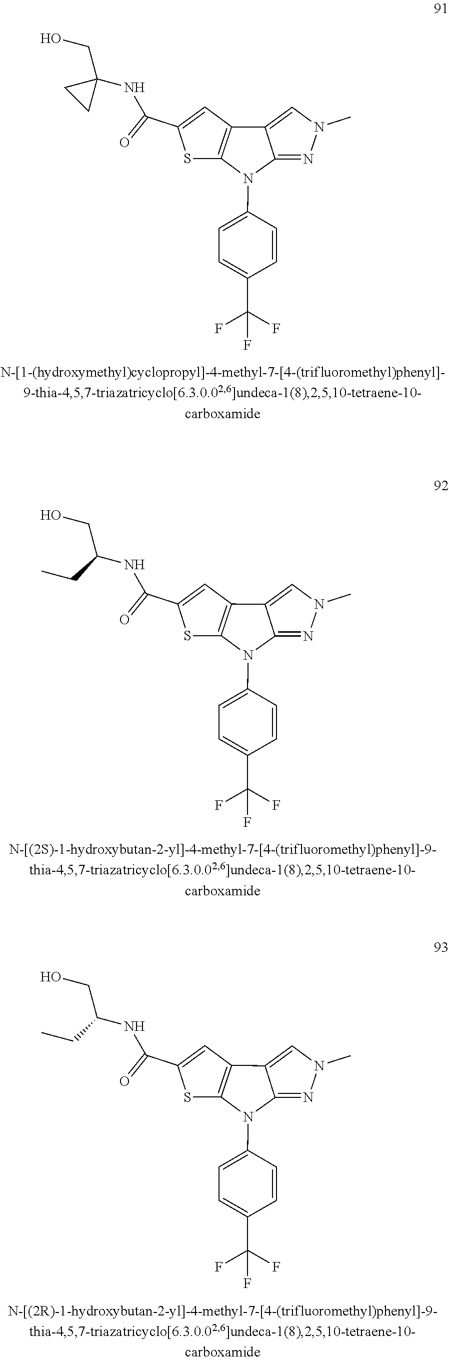

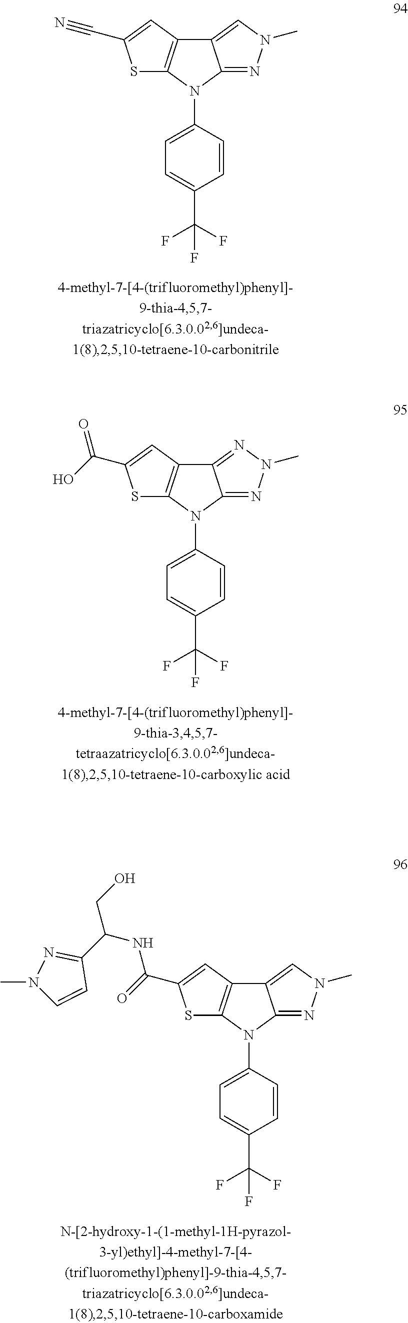

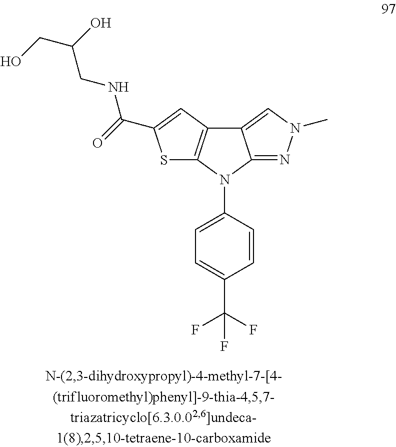 [SMILES: Cn1cc2c3cc(C(=O)NCC(O)CO)sc3n(-c3ccc(C(F)(F)F)cc3)c2n1]

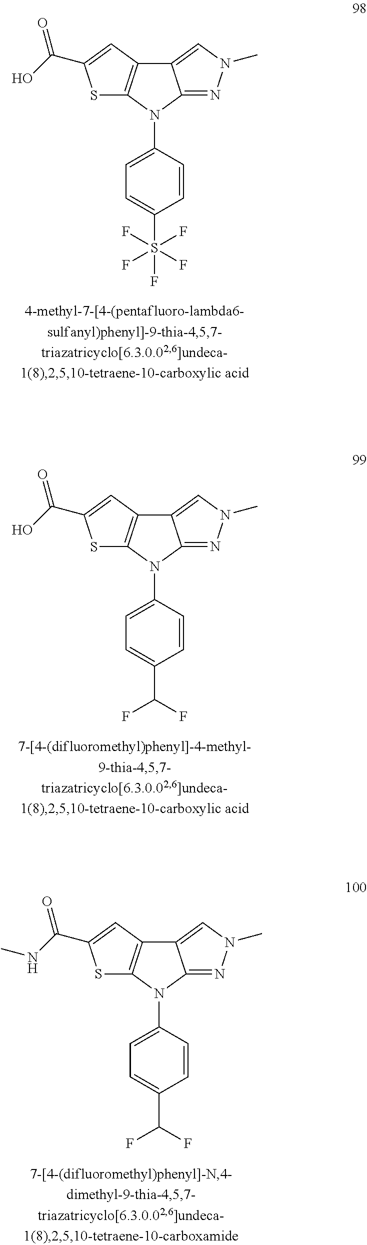

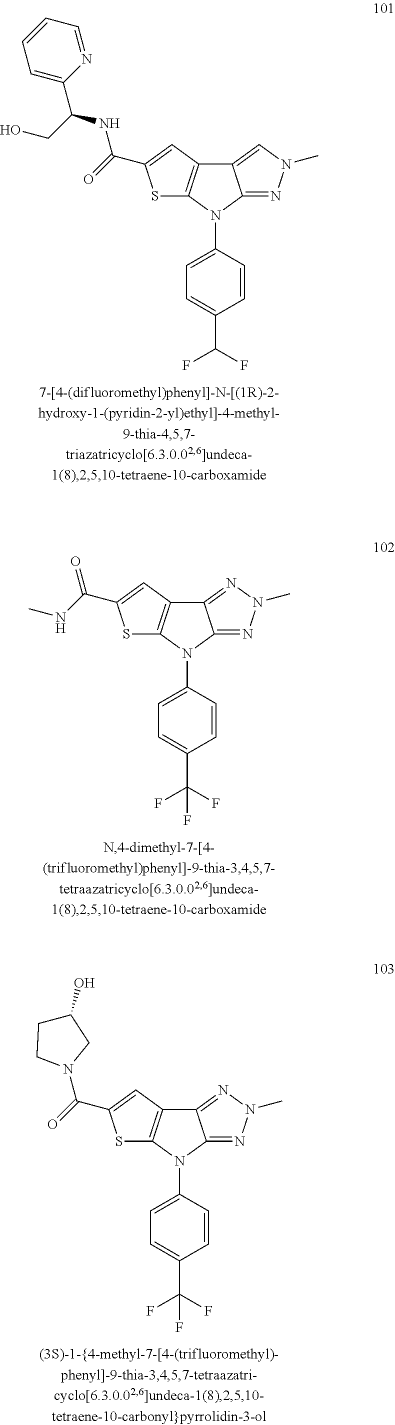

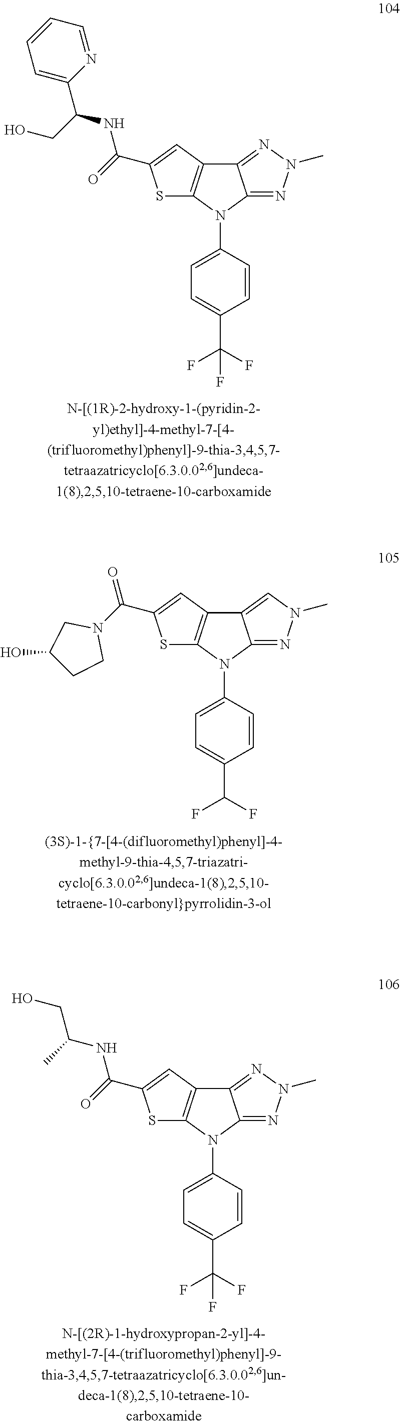 [SMILES: Cn1nc2c3cc(C(=O)N[C@@H](CO)c4ccccn4)sc3n(-c3ccc(C(F)(F)F)cc3)c2n1 ; Cn1cc2c3cc(C(=O)N4CC[C@H](O)C4)sc3n(-c3ccc(C(F)F)cc3)c2n1 ; C[C@H](CO)NC(=O)c1cc2c3nn(C)nc3n(-c3ccc(C(F)(F)F)cc3)c2s1]

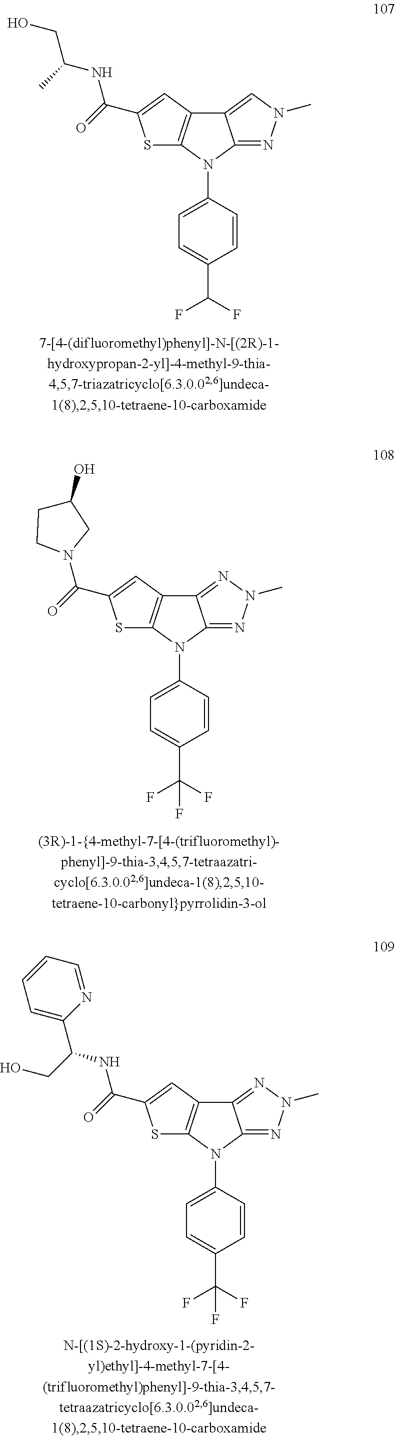 [SMILES: C[C@H](CO)NC(=O)c1cc2c3cn(C)nc3n(-c3ccc(C(F)F)cc3)c2s1 ; Cn1nc2c3cc(C(=O)N4CC[C@@H](O)C4)sc3n(-c3ccc(C(F)(F)F)cc3)c2n1 ; Cn1nc2c3cc(C(=O)N[C@H](CO)c4ccccn4)sc3n(-c3ccc(C(F)(F)F)cc3)c2n1]

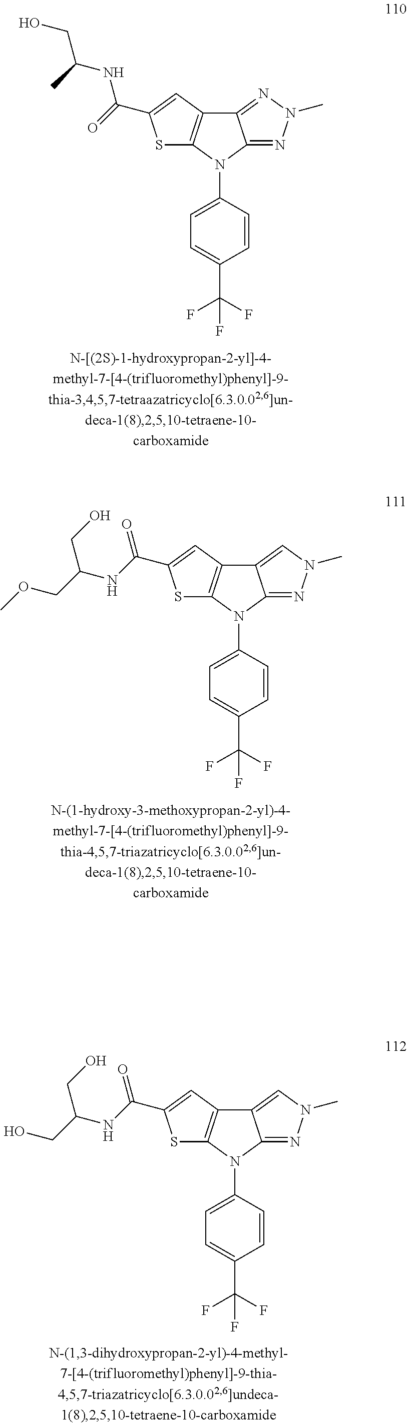

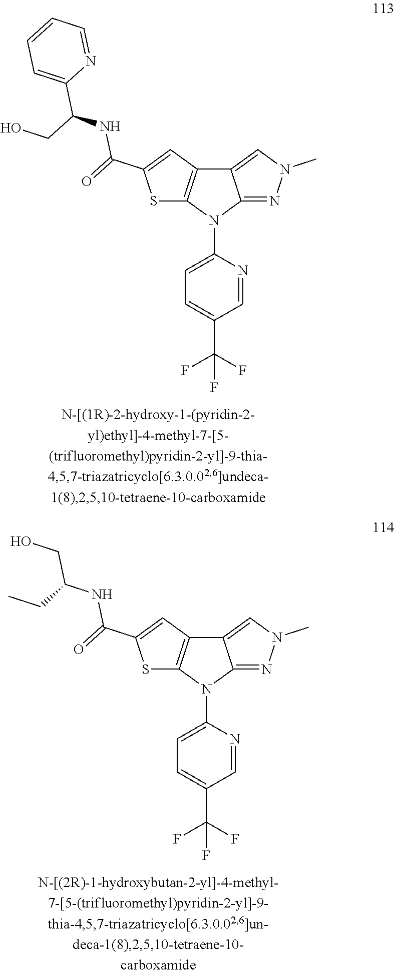

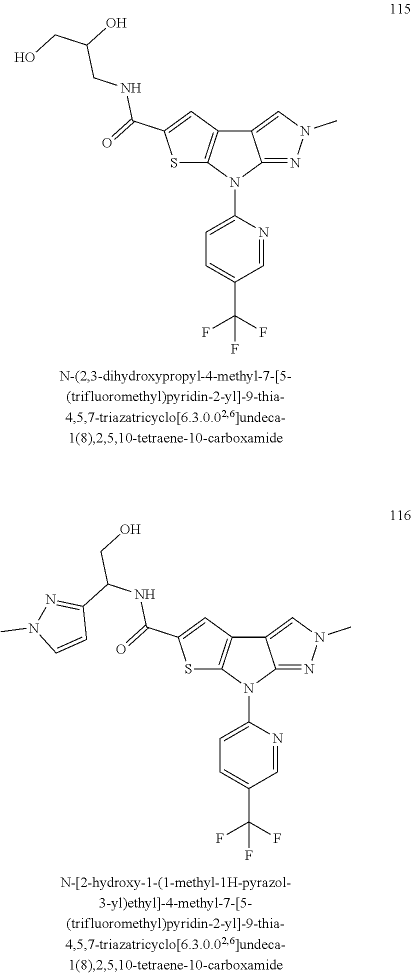

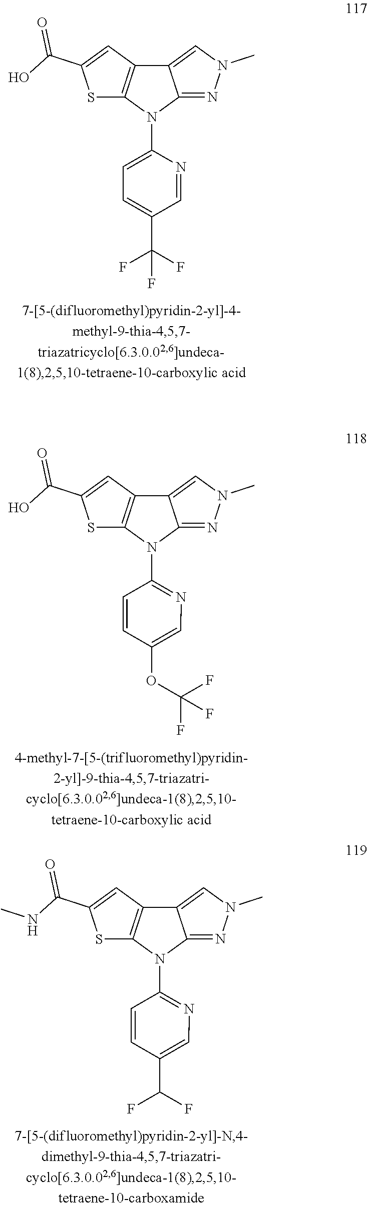

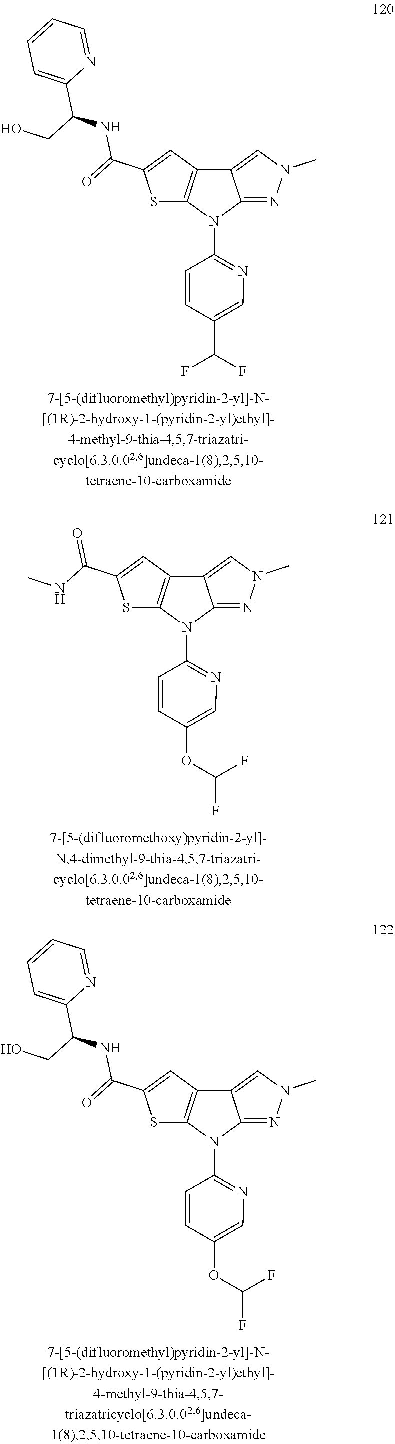

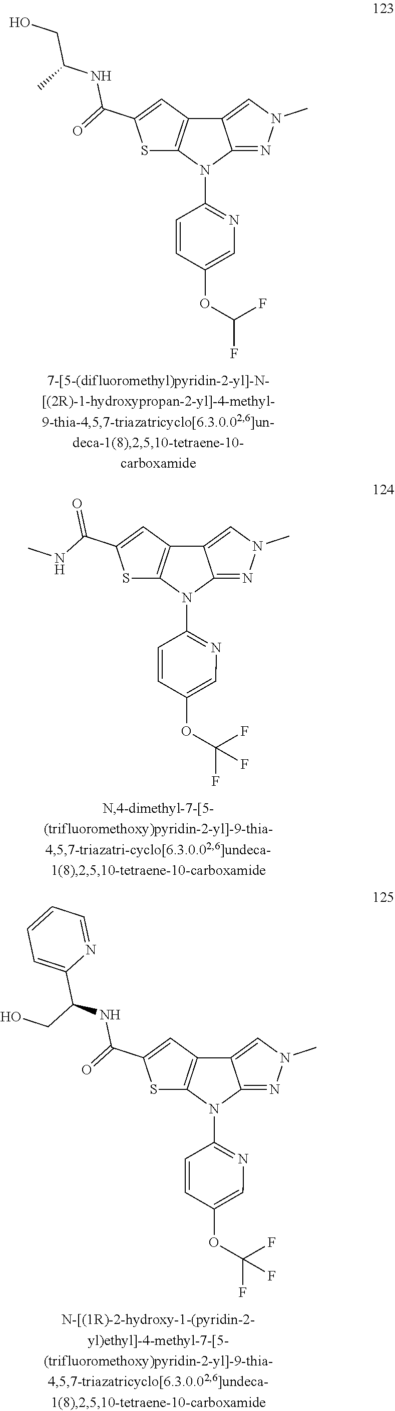

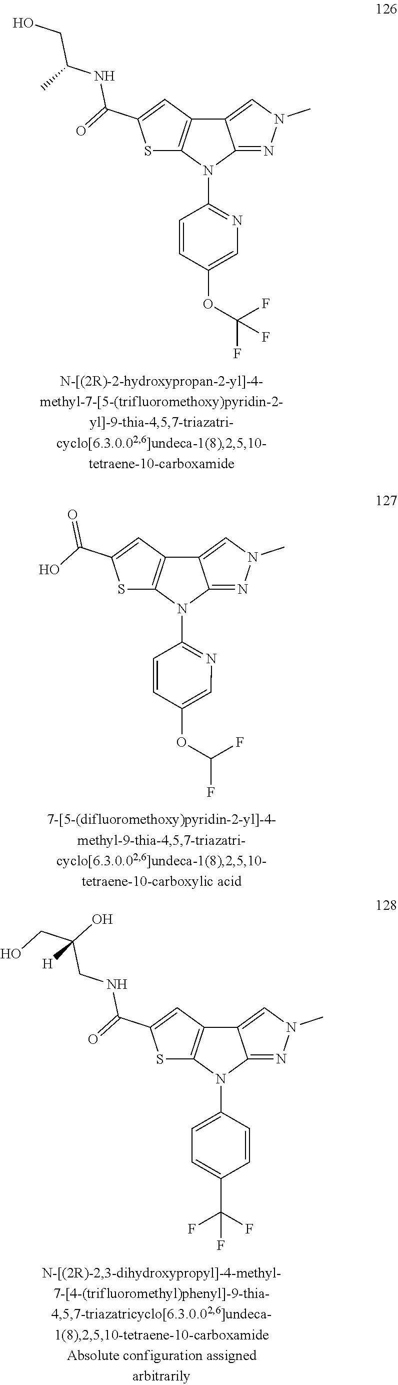 [SMILES: C[C@H](CO)NC(=O)c1cc2c3cn(C)nc3n(-c3ccc(OC(F)(F)F)cn3)c2s1 ; Cn1cc2c3cc(C(=O)O)sc3n(-c3ccc(OC(F)F)cn3)c2n1 ; Cn1cc2c3cc(C(=O)NC[C@@H](O)CO)sc3n(-c3ccc(C(F)(F)F)cc3)c2n1]

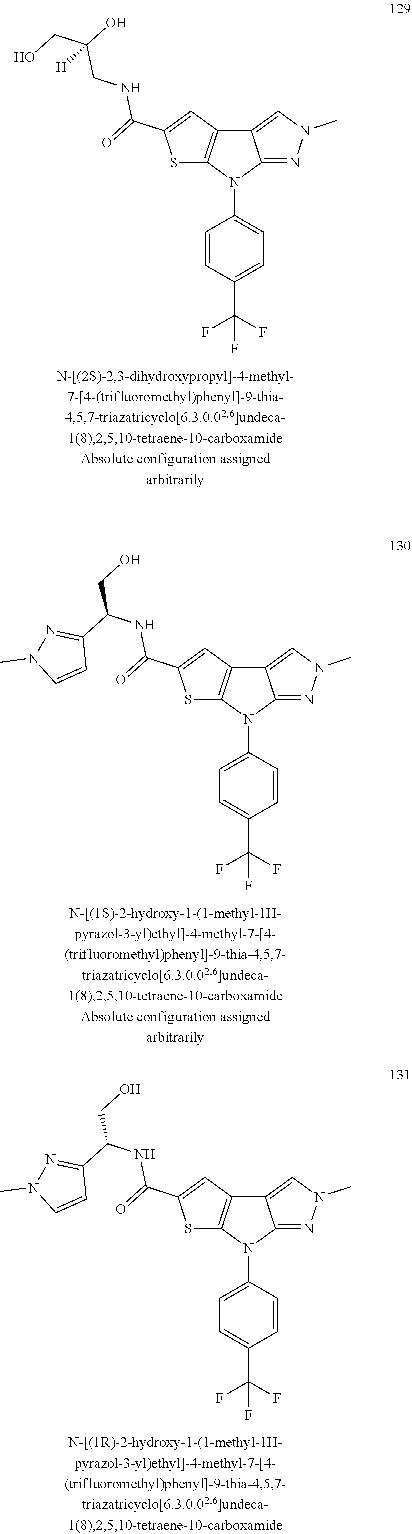

15. A medicament, comprising:
the compound according to claim 1, or any N-oxide, solvate, tautomer or stereoisomer thereof and/or the pharmaceutically acceptable salts of each of the foregoing, including mixtures thereof in all ratios.

16. A method of preventing or treating a medical condition or disease that is affected by inhibited YAP-TEAR and/or TAZ-TEAD interaction, comprising:
administering the compound according to claim 1, or any N-oxide, solvate, tautomer or stereoisomer thereof and/or the pharmaceutically acceptable salts of each of the foregoing, including mixtures thereof in all ratios to a subject in need thereof.

17. A method of treating cancer, comprising:
administering the compound according to claim 1, or any N-oxide, solvate, tautomer or stereoisomer thereof and/or the pharmaceutically acceptable salts of each of the foregoing, including mixtures thereof in all ratios, to a subject in need thereof.

18. A pharmaceutical composition, comprising:
at least one compound according to claim 1, or any N-oxide, solvate, tautomer or stereoisomer thereof and/or the pharmaceutically acceptable salts of each of the foregoing, including mixtures thereof in all ratios, as active ingredient, together with a pharmaceutically acceptable carrier.

19. The pharmaceutical composition according to claim 18, further comprising:
a second active ingredient or any N-oxide, solvate, tautomer or stereoisomer thereof and/or the pharmaceutically acceptable salts of each of the foregoing, including mixtures thereof in all ratios, wherein that second active ingredient is other than the compound of formula I.

20. A set (kit), comprising:
separate packs of
a) an effective amount of a compound of formula I according to claim 1, or any N-oxide, solvate, tautomer or stereoisomer thereof and/or the pharmaceutically acceptable salts of each of the foregoing, including mixtures thereof in all ratios; and
b) an effective amount of a further active ingredient that further active ingredient not being a compound of formula I as defined in claim 1.

21. A process for manufacturing the compound according to claim 1, or any N-oxide, solvate, tautomer or stereoisomer thereof and/or the pharmaceutically acceptable salts of each of the foregoing, including mixtures thereof in all ratios, wherein
a compound of formula II 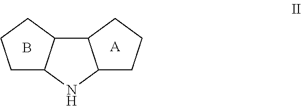
wherein Ring A and Ring B are as defined for the compound of formula I in claim 1
is
(a) reacted with a compound of formula III 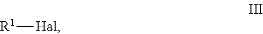
wherein R¹ is as defined for the compound of formula I in claim 1 and Hal represents Cl, Br or I,
in a C—N cross coupling reaction under suitable reaction conditions;
to provide
a compound of formula I as defined in claim 1; and
optionally
(b) if in the compound of formula I R² is —C(═O)—OR^{2a} with R^{2a} being unsubstituted or substituted C₁₋₈-aliphatic, then this compound of formula I is subjected to a saponification reaction under suitable conditions to provide the respective compound of formula I with R² being —C(═O)—OH or —C(═O)—OCat; and
optionally
(c) that compound of formula I with R² being —C(═O)—OH or —C(═O)—OCat is reacted with a compound of formula IV 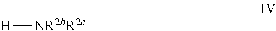
wherein R^{2b} and R^{2c} are as defined for the compound of formula I in claim 1 under suitable reaction conditions;
to provide a compound of formula I with R² being —C(═O)—NR^{2a}R^{2b} wherein R^{2b} and R^{2c} are as defined for the compound of formula I in claim 1.
